(19) United States
(12) Patent Application Publication
(10) Pub. No.: US 2023/0011644 A1
(43) Pub. Date: Jan. 12, 2023

(54) X-RAY IMAGING SYSTEM

(71) Applicant: Sail SV LLC, Newport Beach, CA (US)

(72) Inventor: Ying Zhao, Newport Beach, CA (US)

(21) Appl. No.: 17/664,855

(22) Filed: May 24, 2022

Related U.S. Application Data
(63) Continuation of application No. PCT/US2020/062426, filed on Nov. 25, 2020.
(60) Provisional application No. 62/940,682, filed on Nov. 26, 2019.
(60) Provisional application No. 62/941,728, filed on Nov. 28, 2019.
(60) Provisional application No. 62/948,290, filed on Dec. 15, 2019.
(60) Provisional application No. 62/951,458, filed on Dec. 20, 2019.
(60) Provisional application No. 62/954,508, filed on Dec. 29, 2019.
(60) Provisional application No. 62/962,959, filed on Jan. 18, 2020.
(60) Provisional application No. 62/981,545, filed on Feb. 26, 2020.
(60) Provisional application No. 62/990,449, filed on Mar. 17, 2020.
(60) Provisional application No. 62/993,726, filed on Mar. 24, 2020.
(60) Provisional application No. 62/994,869, filed on Mar. 26, 2020.
(60) Provisional application No. 63/019,214, filed on May 1, 2020.
(60) Provisional application No. 63/026,741, filed on May 19, 2020.
(60) Provisional application No. 63/031,573, filed on May 29, 2020.
(60) Provisional application No. 63/040,003, filed on Jun. 17, 2020.
(60) Provisional application No. 63/042,013, filed on Jun. 22, 2020.
(60) Provisional application No. 63/046,712, filed on Jul. 1, 2020.
(60) Provisional application No. 63/050,122, filed on Jul. 10, 2020.
(60) Provisional application No. 63/063,976, filed on Aug. 11, 2020.
(60) Provisional application No. 63/066,170, filed on Aug. 15, 2020.
(60) Provisional application No. 63/073,945, filed on Sep. 3, 2020.
(60) Provisional application No. 63/076,914, filed on Sep. 10, 2020.
(60) Provisional application No. 63/078,004, filed on Sep. 14, 2020.
(60) Provisional application No. 63/078,946, filed on Sep. 16, 2020.
(60) Provisional application No. 63/081,344, filed on Sep. 22, 2020.
(60) Provisional application No. 63/084,019, filed on Sep. 28, 2020.
(60) Provisional application No. 63/087,185, filed on Oct. 3, 2020.
(60) Provisional application No. 63/087,227, filed on Oct. 4, 2020.
(60) Provisional application No. 63/093,320, filed on Oct. 19, 2020.
(60) Provisional application No. 63/105,912, filed on Oct. 27, 2020.
(60) Provisional application No. 63/106,908, filed on Oct. 29, 2020.
(60) Provisional application No. 63/107,462, filed on Oct. 30, 2020.
(60) Provisional application No. 63/108,291, filed on Oct. 31, 2020.
(60) Provisional application No. 63/110,986, filed on Nov. 7, 2020.
(60) Provisional application No. 63/113,258, filed on Nov. 13, 2020.

(51) Int. Cl. A61B 6/00; A61B 6/03; A61B 6/02

(52) U.S. Cl. CPC A61B 6/4266; A61B 6/032; A61B 6/027; A61B 6/54

(57) ABSTRACT

An x-ray system and method can improve speed of imaging and/or reduce radiation dosage compared to conventional imaging technique, such as CT. The system can identify a volume of interest within a subject. The system can include scatter removal algorithms and/or a beam selection device. Material decomposition of the imaged subject can be based on the dual energy decomposition method which can be iterative to solve the energy response function equation system. X-rayx-rayx-rayx-rayx-rayX-rayX-rayX-ray

### INCORPORATION BY REFERENCE TO ANY PRIORITY APPLICATIONS

This application claims the benefit under 35 U.S.C. § 120 and 35 U.S.C. § 365(c) as a continuation of International Application No. PCT/US2020/062426, designating the United States, with an international filing date of Nov. 25, 2020, titled “X-RAY IMAGING SYSTEM,” which claims the benefit of U.S. Provisional Patent Application No. 62/940,682, filed on Nov. 26, 2019; U.S. Provisional Patent Application No. 62/941,728, filed on Nov. 28, 2019; U.S. Provisional Patent Application No. 62/948,290, filed on Dec. 15, 2019; U.S. Provisional Patent Application No. 62/951,458, filed on Dec. 20, 2019; U.S. Provisional Patent Application No. 62/954,508, filed on Dec. 29, 2019; U.S. Provisional Patent Application No. 62/962,959, filed on Jan. 18, 2020; U.S. Provisional Patent Application No. 62/981,545, filed on Feb. 26, 2020; U.S. Provisional Patent Application No. 62/990,449, filed on Mar. 17, 2020; U.S. Provisional Patent Application No. 62/993,726, filed on Mar. 24, 2020; U.S. Provisional Patent Application No 62/994,869, filed on March 26, 2020; U.S. Provisional Patent Application No. 63/019,214, filed on May 1, 2020; U.S. Provisional Patent Application No. 63/026,741, filed on May 19, 2020; U.S. Provisional Patent Application No. 63/031,573, filed on May 29, 2020; U.S. Provisional Patent Application No. 63/040,003, filed on Jun. 17, 2020; U.S. Provisional Patent Application No. 63/042,013, filed on Jun. 22, 2020; U.S. Provisional Patent Application No. 63/046,712, filed on Jul. 1, 2020; U.S. Provisional Patent Application No. 63/050,122, filed on Jul. 10, 2020; U.S. Provisional Patent Application No. 63/063,976, filed on Aug. 11, 2020; 2020; U.S. Provisional Patent Application No. 63/066,170, filed on Aug. 15, 2020; U.S. Provisional Patent Application No. 63/073,945, filed on Sep. 3, 2020; U.S. Provisional Patent Application No. 63/076,914, filed on Sep. 10, 2020; U.S. Provisional Patent Application No. 63/078,004, filed on Sep. 14, 2020; U.S. Provisional Patent Application No. 63/078,946, filed on Sep. 16, 2020; U.S. and U.S. Provisional Patent Application No. 63/081,344, filed on Sep. 22, 2020; U.S. and U.S. Provisional Patent Application No. 63/084,019, filed on Sep. 28, 2020; U.S. Provisional Patent Application No. 63/087,227, filed on Oct. 4, 2020; U.S. Provisional Patent Application No. 63/087,185, filed on Oct. 3, 2020; U.S. Provisional Patent Application No. 63/093,320, filed on Oct. 19, 2020; U.S. Provisional Patent Application No. 63/105,912, filed on Oct. 27, 2020; U.S. Provisional Patent Application No. 63/106,908, filed on Oct. 29, 2020; U.S. Provisional Patent Application No. 63/107,462, filed on Oct. 30, 2020; U.S. Provisional Patent Application No. 63/108,291, filed on Oct. 31, 2020; U.S. Provisional Patent Application No. 63/110,986, filed on Nov. 7, 2020; U.S. Provisional Patent Application No. 63/113,258, filed on Nov. 13, 2020; the entire disclosure of each of which is hereby incorporated by reference and made part of this specification.

### FIELD

This application is related to X-ray imaging systems and related technology for diagnosis, monitoring, surveillance and image guidance, identification and characterization in medicine, drug discovery and life science research, non-destructive testing (NDT), field inspection, characterization of minerals and security.

### BACKGROUND

CT imaging allows better dissection of information buried in overlapping materials, such as different tissues in a human body. Currently, in order to dissect information buried in overlapping tissues, CT imaging is often needed.

However, CT radiation poses a safety concern and is generally not routinely used for frequent surveillance and monitoring.

Conventional CT scanner are not only bulky and complex, difficult to transport, spatially restrictive for patient and phycisian accessibility. CT is inflexible in terms of the type of imaging method it provides, and it is a closed system in that extension of hardware capabilities are generally not available.

CT reconstruction methods generally are time consuming and requires extensive artifact correction and motion correction due to scatter, beam hardening and its complex robotics motion requirement during image acquisition.

Purchasing a CT and making a payment is a slow and difficult process as it is an expensive capital capital equipment. Time and effort required for a facility to acquire and maintain a CT system are not a small undertaking.

### SUMMARY

Tomography imaging combined with spectral imaging, with less than 1% or less than 5% of Scatter to Primary Ratio (SPR), which can be achieved through scatter removal techniques, enables improved x-ray imaging and tomography using flat panel detectors or 2D detector and related automated assembly and software methods fordiagnosis and monitoring and tracking applications previously only achievable through CT.

With the data provided by spectral imaging or tomographic measurement in for example, point, 1D, 2D, 3D, 6D or 6D and time (sometimes referred to as “7D”), the requirements for sensitivity, resolution, volume and spatial position of “volume of interest” and of Rregion of interest “ROI”, ype of data and data completeness may vary in order to deliver the results demanded by a particular application.

Due to a myriad variety of demands by each imaging scenario in diagnosis and in intervention procedures and other applications suitable for x ray imaging, it is desirable to have an x-ray measurement system and method that allows a user to fine-tune the system configuration in real time for each application and/or to customize an imaging method for each sample or patient to achieve fast, improved and sufficiently accurate and precise results while at the same time limiting x-ray exposure and time required for imaging procedures.

For example, in surgical tracking, low resolution spectral x-ray measurements in point, 1D, 2D or 3D may be sufficient. While in cancer diagnosis, a very high resolution tomographic image in a limited VOI may be desired In another o example in intervention procedures, tracking and monitoring by combining fast measurements of low resolution tomography imaging of selected VOI and component internal to VOI and or spectral imaging in 2D can precisely characterize components in time, space and frequency domain with a level of precision needed for implant placement and or minimum invasive surgeries..

For each specified application and imaging condition, in present disclosure, the choice of hardware and software, and/or the spatial location of the x-ray tubes, detectors, x-ray optics, optical measurement optics and/or robotics may have different configurations than a typical x-ray imaging system like, for example, a general x-ray imaging system with one x-ray tube and one detector pair, or a CT or a densitometer, or a tomosynthesis system or a C arm or a U arm or an O ring x-ray could allow.

In order to achieve variable resolution and/or quantitative spectral imaging in 2D or 3D for a specified VOI, it may be desired, for example, to have the x-ray source emitting from the same spatial location relative to the sample and/or detector. However, presently one source or one detector may not be capable of delivering energy levels or resolution or speed needed for accurate spectral imaging or tomography measurements.

Present disclosure enables flexible dynamic system configurations to accommodate a wide variety of imaging needs and demands for various applications while reduce form factor and weight compared to a conventional CT, thereby enabling portability and accessibility at point of care.

Point to 7D x-ray imaging systems, or or 3D real time fluoroscope systems may include a number of software image processing capabilities and/or hardware components, such as x-ray optics, modulation modules, optical optics, x-ray sources, detectors, collimators, beam particle stopper plate, beam selector, filters, grating systems, beam splitters, choppers, various movers, a beam steering apparatus, and/or the like to further extend imaging capabilities.

Each hardware element may be moved in and out of the x-ray illumination pathway by one or more moving mechanisms, independently or synchronically with one or more other elements of hardware apparatus, driven by software control on a microprocessor or by a user on per need basis to manipulate the x-ray beam in different ways depending on the nature of the different demands for each application. In some cases, such an spectral x-ray tomography device is combined with parts of or with a complete unit of a separate x-ray 2D or 3D imaging device and/or a sample holding device to expand imaging capabilities and analysis required for a specific application.

In some cases, one or more x-ray sources in the same x-ray emitting position at different times and/or at various x-ray emitting positions may be used.

Variable x-ray attenuation and interference properties in space or frequency domain may be measured when interrogating samples to reveal additional information needed for diagnosis or tracking.

Various x-ray sources or detectors or x-ray or optical optics may allow accurate x-ray spectral imaging in 2D or 3D format and enable the manipulation of x-ray beam or electron beam used to generate an x-ray beam in different systems and/or spatial configurations to allow flexibility and optimization of the number of imaging modes, or image presentations for procedures the x-ray systems can operate under.

An x-ray multiple dimensional imaging or spectral imaging or tomography system which can accommodate requirements for varied imaging demands for selected ROI has room for flexibility such as described below:

VOI may be imaged by a region on the detector corresponding to the projected image of the ROI which can be selected, for example by the x-ray coming from x-ray source collimated by a collimator or a MAD filter such that the x-ray beam size sufficiently illuminates the ROI on the subject. The ROI can be selected to be measured spatially by a region on the detector which may be called ROI of the detector. Such ROI of the detector can be determined prior or during the imaging process, by for example, calibration ahead of time of the x-ray source FOV through sized or restricted or selected beam size, or location of beam.

For example, a mover (manual or automatic, for example, mechanical) may move the x-ray source to a location where the x-ray cone beam center axis is properly aligned for optimized imaging angle of of the VOI.

A mover may also independently or synchronically move one or more detectors to collect the projected image of the VOL The same mover may move both source and detector pair. The source and/or detector may be moved by one or more movers to illuminate and image other VOIs in an object.

The x-ray cone beam size may be selected by a user or digital program controlling a collimator shutter, which may be located downstream from the x-ray source. The region of detector which collects only x-ray signal passing through ROI to reach the detector may be selected to be measured and/or processed since the region surrounding the ROI on the detector may have different measurements or signal levels. This correlation may also be determined mathematically based on the x-ray source spatial location relative to the detector. ROI of the detector may be adjusted for example, to to be normalized selectively, and to optimize the imaging speed as the x-ray emitting beam size and/or beam location are adjusted.

On the other hand, sometimes a large VOI is required, such as in case of whole body imaging. Two or more detectors and their corresponding x-ray source(s) may be used, and/or a source and detector pair may be moved to illuminate and image more ROIs in order to image a larger FOV. Multiple sources or x-ray emitting locations may be used together, and they may sometimes be moved synchronically or asynchronically to image one VOI, in order to increase the speed of image acquisition and data acquisition needed for tomography reconstruction.

Two or more VOI may be illuminated by an x-ray beam, and their projections may be collected by different regions of the detector which may be distributed across the detector or a different detector in the x-ray illumination path. The detectors may be collecting x-ray measurement sequentially and/or simultaneously. The image measurement and processing can be optimized by selectively measuring multiple ROI after the different ROI are determined, which in some cases can entail omitting regions outside of ROIs on the detector.

Interference Removal and Better Visualization

Image presentation of x-ray images which are separated by multiple energy mechanisms or single energy material decomposition method may be accomplished by image processing after image acquisition of a 3D CT system. The image reconstruction may also be done while x-ray measurements or images are acquired image reconstruction may be prioritized and customized based on analysis of the images already acquired. When some of the images are combined to reconstruct a final presented image that is processed for display, in some instances, different materials or components may be presented in different colors and or by adjusting intensity or dynamic range to enhance visual presentation and illustrate dynamic component movements and distribution relative to each other and to the background.

High throughput system

The present disclosure includes high throughput X-ray system including systems capable of 2D and/or 3D spectral measurements of ROI in the same object or ROIs in two or more objects at the same or different times..

In case of x-ray imaging of human or animal, in or ex vivo and in vitro samples, generally there is only one sample which is placed on a sample holder or table, with one or dual x-ray sources, and one or two detectors corresponding to the source(s). In case of small animal imaging, or non-destructive testing (NDT), there is a limitation to how many samples can be imaged at any one time. It is generally limited by the rotational design of conventional CT, which leads to limited FOV, sample size and number of samples to be imaged.

The present disclosure, which allows for high resolution spectral and spatial domain 2D imaging as well as nonrotational tomography, allows accessibility and FOV making it now possible for a larger number of samples to be imaged at the same time than conventional CT.

The x-ray systems and methods disclosed herein may be used, for instance, in case of imaging of two or more live objects that may sometimes be in motion or be static, in alarger achievable FOV than current conventional CT systems.

The x-ray systems and methods disclosed herein may also be used in case of imaging in more natural environments to diagnose, monitor and track two or more live static or moving objects than can be done with a conventional CT scanner. For example, these objects can be two mice moving in a cage or other area in an animal handling or housing facility.

Spatial location and/orientation of one or multiple VOIs or one or more components within an VOI inside each object may be monitored simultaneously.

X-ray tomography and/or spectral imaging may be combined with a camera and/or with AI capability to track motion, identify movement characteristics and simultaneously image one or more VOIs using x-ray imaging in one or more objects. The object may be moving and not be static.

In drug discovery, diagnostics and life science research, quality inspection and failure analysis in Information and Communication Technology (ICT) production and security applications, fast data acquisition of many different samples are essential to collect data for analysis and fact finding.

In some cases X-ray systems with one or multiple x-ray systems are running simultaneously on different samples of the same kind or on different samples. For example, in 3D tissue studies on a microfluidic chip, or in case of drug testing or lead screening of small animals or ex vivo animal tissues, or in digital pathology where, for example, simultaneous screening of multiple tissues or samples from different patient scan be performed.

In some instances, the detector may be capable of measuring x-ray, optical signals such as UV or near infrared (NIR) signals at the same time or substantially the same time. The same pixels may be used for all measurements. Different pixels of the same detector or different detectors may be used for measurements of different modalities.

ROI Identification

With conventional CT system, generally the entire ROI or subject needs to be imaged and the 3D image reconstructed in order to obtain the spatial position and or orientation of a component embedded internally in a VOL The present disclosure provides optimized and customized methods to adjust x-ray measurements of VOI and components in terms of resolution, speed, FOV, type of measurement, spectral imaging and to improve reconstruction methods and presentations to optimize outcome and at the same time reduce radiation.

The x-ray imaging system and apparatus disclosed herein improves and expands on disclosures in reference international patent applications (“PCT Applications”) including International Patent Application Nos. PCT/US2019/044226, PCT/US2019/014391 and PCT/US2019/022820, the entirety of each of which are incorporated by reference herein and should be considered part of the disclosure.

The x-ray systems and methods disclosed herein may incorporate scatter removal methods using a beam particle stopper array plate, dual or multiple detectors and/or spectral imaging and material decomposition methods where an interpolated plot is generated by utilizing measurements of multiple known substances at varied density and thickness and inverse energy response function system look-up table method to obtain accurate density and thickness information needed for material decomposition of at least one substance. Example of such an imaging apparatus and method are illustrated in FIGS. 43a-b. The imaging method may include the process of identification of ROI using any method of imaging disclosed herein, for example low resolution or single, dual or multiple energy imaging methods, and 2D or 3D imaging as described in the aforementioned PCT Applications and in this disclosure.

A customized ROI may be further established by one or more imaging methods. The apparatus may be selected by the user(s) or automated system(s) controlled by one or more software programs run by a computer or work station, based on the specific application requirements and radiation levels in order to further investigate the ROI. The process of identification and/or analysis and/or characterization of ROI may be achieved by an iterative method on a case-by-case basis

Selection of ROI and/or imaging methods and/or processing may be customized for each object and/or each application. Depending on the results from an earlier investigation using x-ray and/or other methods or other modalities, further adaptation and fine-tuning of the imaging method can be performed to better analyze the subject.

Apparatus and methods for customized tomography and/or spectral imaging may be determined by prioritizing data and application requirements. For example, a measurement and/or reconstruction of higher spatial, temporal or spectral resolution may be done with the same or a different set of hardware, software and chemistry components. Such a process may be iterative to ultimately achieve the target specifications of image measurements and analysis, which may be diagnosis, inspection, image guidance or tracking and monitoring. Each of the hardware, software or chemistry component described or referenced in the x-ray imaging system and apparatus disclosed here are selected and/or combined or mix-matched based on the application and requirements of the user or the digital or software program.

Some aspects of the present disclosure include quantitative Spectral X-ray 2D/3D/Tomography using multiple axis matrix image acquisition and reconstruction with less than 1% or less than 5% SPR, capable of real time 2D and/or 3D and/or 6D fluoroscopy and dimension measurement uses the following techniques:

Fast primary X-ray Image measurements enabled by scatter removal to less than 5% SPR or less than 1% SPR using one pulse image acquisition process.

Use of Beam Particle Stopper Array to remove scatter in a two detector or one detector configuration to one or two exposures. Spectral Imaging enabled by Energy Response Function Equation System establishment and solving nonlinear energy response equation system by inverse look up table.

Fast Tomography and highly quantitative 3D image and/or Spectral 3D reconstruction enabled by almost complete scatter removal at less than 1% SPR or less than 5% SPR, material decomposition from spectral Imaging and/or density measurement, simplified system matrix, which result in dramatical improvements in model based iterative reconstruction, fourier transform based reconstruction, reconstructions based on Analytical and or Deterministic, iterative algorithms, SART, SIRT techniques, ray tracing method, monte carlo simulation methods and enablement of image reconstruction of ROI and its individual components despite the complexity involved in expanded hardware configurations, intervention device design, and related chemistry and contrast agents. Determination of ROI before, during and after both image acquisition and reconstruction. Differential presentation of overlapping substances in 2D and/or 3D format, amplification of dynamic range, intensity, selective color presentation and enhanced contrast representation of selected substances separately from or against the background images of other substances. Contrast agents, suitable for the spectral 2D and 3D tomography, may be made less toxic by using dramatically lesser amount, for example, 2× to 10,000× less, by using quantitative imaging method in point, structural, 1D- 7D imaging. Design of intervention devices to be better controlled and visualized and enable better guidance and/or monitoring of intervention procesures and treatment levels. Portable Device based on the aforementioned x-ray system, enabled by autonomous driving mechanisms. High throughput device enabled by spatial configuration of the x-ray tomography system enables high throughput monitoring of activities and lives of live animals in their nature environment. AI enabled x-ray tomography image acquisition and tomography reconstruction and analysis speed up imaging process and improve precision and personalization. Standardization Methods and scatter removal methods above to enable dramatic improvement and adoption of quantitative data analysis and AI analysis and recontruction of tomographic images, or more specifically, quantitative personalized x-ray imaging/tomography system, the implementation of high resolution (sub micro range), and/or high sensitivity, great than 10⁻³ molar, and/or high spectral resolution (multiple energy), and/or less than one second per 3D image acquisition and/or less than is reconstruction, in human clinics.

The present disclosure provides an example improved computed tomography imaging system. The system can include at least one x-ray source configured to produce a plurality of divergent beams; and a plurality of detectors configured to receive x-ray beams emitted from a plurality of emitting positions and attenuated by at least a portion of a subject to be imaged, wherein the plurality of emitting positions comprise a first positions relative to a volume of interest (“VOI”) in the subject, the beams emitted from the first emitting position being projected onto at least one x-y plane two axis out of a 6D space or any possible projection geometry combined.

In a configuration, the plurality of emitting positions can comprise a second position, beams emitted from the second position being projected onto the at least one plane or another 2D or 3D dimension, at least one voxel in the VOI being in on a projection path traveled by the beams emitted from the first position, wherein a distance between the first and second emitting positions is approximately equal to a resolution desired in a z-axis.

In a configuration, approximately the voxels in the VOI can be located in the projection path

In a configuration, the plurality of emitting positions can comprise a third position.

In a configuration, beams emitted from the third positionare are configured to follow a trajectory outside of a 6D space required for tomography and to increase a size of r of a field of view of the subject so that a different VOI can be selected.

In a configuration, beams emitted from the third positionare are configured to follow a trajectory outside of a 6D space needed to reconstruct a complete image, and are configured to provide a different angle in a sparse projection situation or for projection from the x-ray source that has at least one different energy level, and/or different focal spot sizes or different field of view, different frame rate or modulated differently by energy means or electronics means or optical means.

In a configuration, a path traveled by beams emitted from one or more of the plurality of emitting positions can be traveled by beams emitted from a different x-ray source, wherein the different x-ray source can have a plurality of different energy levels and focal spot sizes, or a plurality of different frame rates, or comprises a different type of source.

In a configuration, the system can further comprise a controller that includes: an acquisition system configured to acquire from the a plurality of detector x-ray attenuation data; and an image reconstructor configured to receive a first data set derived from the x-ray attenuation data and perform algorithms to reconstruct a first reconstructed image.

In a configuration, the first data set can include primary x-ray data with Scatter to Primary of less than 1% or less than 5%.

In a configuration, the first data set can include primary x-ray data derived from scatter removed data using a scatter removal method that includes time of flight x-ray measurements where primary x-ray is separated from scatter in the time domain.

In a configuration, the first data set can include primary x-ray data with less 1% or less than 5% SPR derived from using movable a beam particle stopper array and/or a adjustable or movable beam selector and using interpolation of low resolution scatter to give rise to high resolution scatter images.

In a configuration, the first data set can include primary x-ray data with less 1% or less than 5% SPR derived from a front detector, a beam particle stopper array and a rear detector using interpolation of low resolution scatter to give rise to high resolution scatter images at the front detector or the rear detector.

In a configuration, the front detector can be a movable front detector.

In a configuration, the first data set can include data derived from projection imaging databy the plurality of detectors corresponding to the plurliaty of emitting positions and VOI.

In a configuration, the first data set can include data derived from projection imaging data from a dual energy material decomprosed substsance dataset, which can be derived from inverse energy function system look-up measured by the selected detector regions at one or both of the first or second positions.

In a configuration, the first data set can include a Housefied value derived from a dual energy material decomprosed substsance dataset, which is derived from inverse energy function system look-up measured by the selected detector regions at two or more of the plurality of emitting positions.

In a configuration, the controller can be further configured to execute a material decomposition to provide attentiation data for at least one substance.

In a configuration, wherein the controller can be further configured to generate a material decomposition based on 2D dual energy or multiple energy measurements of the VOI from x-ray emitted at one or both of the first or second emitting positions.

In a configuration, the material decomposition method can include using measurements from a time of flight sensor or a camera or a previous x ray exposure for measurement of a VOI thickness.

In a configuration, the time of flight sensor and or controller can be configured to determine an exposure level of x-ray measurements generating at least some of the first set data and/or a second data set.

In a configuration, the reconstruction method can comprise algorithms or derivatives of the algorithms for tomographic reconstruction for CT, tomosynthesis, MRI, electron tomography, optical tomography, thermo imaging, PET, or SPECT.

In a configuration, the first reconstructed image can be reconstructed using a reconstruction methodincluding an original or derivatives of fourier transform, ray tracing method, model or contour based iterative reconstruction, material decomposed method based, spectral CT, ART, Monte Carlo Simulation based, non space based reconstruction method, iterative algorithms and their derivatives, filtered methods, method at least one modifieddual variable, or a splitting-based subproblem method.

In a configuration, the controller can be configured to generate the first reconstructed image by: backprojecting the x-ray attenuation data for each beam to form an array of data points therealong, weighting each backprojected data point by a weighting factor w(r), where r is the distance between the backprojected data point and a source location of the divergent beams to form weighted backprojected data points, Fourier transforming and processing an array of data which includes the weighted backprojected data points to form an acquired k-space data set; aligning the acquired k-space data set with a reference k-space, and reconstructing an image from the referenced k-space data by performing an inverse Fourier transformation thereon.

In a configuration, said system can be integrated with an autonomous driving device.

In a configuration, said system can be configured to fit through a standard door, the plurality of detectors configure to be placed between a patient and a patient bed, surgical table, or imaging table.

In a configuration, said system can be a spectral tomographic mammography system.

In a configuration, said system can further comprise a hand switch, a display, handheld display, foot pedal, display membrane, joy stick, voice recognization, speaker, acoustic noise hardware and electronics and software, the controller configured to control some of the hardware and sync software for integrating hardware and software processes.

In a configuration, said system or its components can be a portion of a kit.

In a configuration, said system can comprise methods, softwareand hardware to decompose metal materials.

In a configuration, said system can include methods and hardware to material compose intervention devices or one or more portion of such device, implant or contrast agents, microcalcification, contrast labeled blood vessels, plaster cast mixed with contrast agents.

In a configuration, the contrast agents can comprise barium or bismuth.

In a configuration, the contrast agents can be administered at concentration levels and/or molarity levels at 2× to 1000,000× less than that of contrast agents used in conventional CT and general x-ray and MRI and PET and/or magnetic particle based imaging.

In a configuration, the contrat agents can comprise calcium chloride, calcium glutonate, iodinated reagents, barium, bismuth, strontium, gadnolium, the contrast agents used in PET and/or MM.

In a configuration, the intervention device can comprise an artificial heart valve, an RF ablation catheter, a cage, a stent, an implant, or surgical tool.

In a configuration, said system can comprise a C arm, U arm, CT system, or has a foot print similar to that of a general x-ray or tomosynthesis system.

In a configuration, the system can further comprise a first system matrix configured to integrate one or more of the x-ray sources and one or more of the plurality of detectors.

In a configuration, the first position can comprise in area of less than 2 cm ², or less than 5 cm² or less 1 degee , or less than 2 degrees, or less than 3 degrees, or less than 4 degrees, or less than 5 degrees, or less than 6 degrees, or less than 7 degrees, less than 8 degrees or less than 10 degrees, from a center axis connecting original positions of the plurality of detectors and the at least one x-ray source.

In a configuration, the distance can be less than 1 um, or less than 5 um, or less than 10 um, or less than 50 um or less than 100 um, or less than 160 um, or less than 250 um, or less than 500 um, or less than 1 mm, or less than 2 mm. or less than 5 mm, or less than 1 cm or less than 2 cm, or less than 5 cm.

In a configuration, the controller can be configured to generate the first reconstructed image inless than 10s, or less than 5s or less than 2.5s, or less than ls.

In a configuration, the system can be configured to reduce radiation exposure by 2×, or by 5× or 10×, or 100×, or 1000× or or 10,000× or or 100, 000, or 1000,000× compared to conventional CT.

In a configuration, the system can comprise a second system matrix configured to integrate additional imaging modalities including optical, thermo, PET, SPECT, ultrasoundand/or MM.

In a configuration, the reference detector can be placed in the x an ray x-ray beam path.

In a configuration, the first data set and the second dataset can be used to train AI algorithms for reconstruction and determining said VOI for data acquisition.

In a configuration, the the controller can be configured to use the second data set, either after or during the reconstruction of the first image.

In a configuration, if the second data set is used after the reconstruction of the first image, the first reconstruction can provide model or contour or data which is used in a second reconstruction incorporating the second data set.

In a configuration, if the second data set is used during the reconstruction of the first image, the controller can be configured to use the same or different system matrix and modified variable and split subproblem method.

In a configuration, the second data set can comprise data derived from a different detector of the plurality of detectors taking at the same time as time of acquisition for one or more x-ray images generating the first data set.

In a configuration, the different detector can comprise at least one detector placed upstream or downstream or at the same spatial location of the first detector from which the first data set was acquired.

In a configuration, the second data set can comprise data from x-ray measurements taken at a time different from the time of acquisition for one or more x-ray images generating the first data set.

In a configuration, the second data set can comprise data taken at a different time by the first detector from which the first data set was acquired.

In a configuration, the first and/or second data sets can be configured to be denoised during, before, or after image reconstruction on a case by case basis.

In a configuration, the denoising process can be selectively done on a substance or the VOI.

In a configuration, the first and/or second data sets can be normalized.

In a configuration, the acquisition system can be configured to selectively acquire data during image reconstruction.

In a configuration, the selective data acquisition can be based on a reconstruction result of first data set, or a selected VOI, wherein the reconstruction is prioritized for the selected VOI.

The present disclosure provides an example payment and transaction electronic system for an x-ray imaging, and related product and services. The system can include: a software platform for purchaser and users including: an electronic database containing metered information for x-ray images or related procedures taken at at least one location; data encryption mechanisms configured to to encrypt data, and currency transfer, and communication; digital currency or exchange media agreed by a buyer and aseller, the digital currency comprising cytocurrency; a server configured to collect the meter information from at least one facility; data collection mechanisms configured to the meter information onsite of the imaging location or via cloud, wherein an amount charged in digital currency periodically can be based on a subscription and/or pay per image model out of purchaser's account.

In a configuration, the system can further comprise a software platform for seller including: a front end presentation comprising a mobile app, the desk top app or the web portal which allows username and password input and sign in and registration and related information, and a developer portal; a back end comprising a product layer where sits a core banking system, client data and other back-offices related processes; a middle-ware comprising an intermediary layer orchestrating information between the front end and the back end and API layer.

In a configuration, the software platnform for sellers can be configured to enable connections to external and/or third party applications include accounting software, customer and/or user accounts, loans, payments, market place, digital onboarding, payment networks, cards and card management.

In a configuration, the seller can be a digital bank or has partnered with a digital bank to enable wiring, ACH transfer, and/or digital bank transfer via email, phone based on a user and/or customer's account number.

In a configuration, the x-ray images can include images produced by scatter removed x-ray imaging system, spectral x-ray imaging system, CT, spectral CT, spectral CT with one or more radiology services, AI related software, pac, image storage, and/or image processing.

The present disclosure provides an example method of reconstructing a 3D image of a VOI of an object using an x-ray system, the x-ray system comprising at least one x-ray source and at least one detector. The method can comprise translating and/or rotating the at least one x-ray source and/or one or more of the plurality of detectors; correlating projection measurements with various positions of the at least one x-ray source and at least one detector using a system matrix, wherein for at least a one 2D projection image, the at least one x-ray source can be configured to emit beams illuminating at least a majority of or approximately an entirety of the VOI so that for each voxel within the VOI, there can be new projection path reaching one of the plurality of detectors, and wherein there can be m×n projection paths approximately, with each movement between the emitting positions, the movement being approximately a resolution desired in along an axial axis connecting an x-ray tube of the at least one x-ray source and the at least one detector passing through the VOI, so that the new projection path can be different from a remainder of the m×n projection path by at least approximately one voxel, or each voxel within VOI can have a projection path differ than other path by at leat approximately one voxel.

In a configuration, a total number of projections can be approximated by a thickness of the VOI.

In a configuration, a total number of projections can be approximated by a geometry measurement of a sensor, a camera or an x-ray image exposure value, or a time of flight sensor, the approximation comprising: determining at least a distance from a top of the subject containing the VOI to the at least one source, and subtracting the distance from the top of the subject to the at least one x-ray source from a source-to-detector distance (“SID”); and deriving the thickness of VOI.

In a configuration, an x-ray exposure level can be approximated by an automatic exposure method and apparatus, the time of flight detector, and/or a reference detector.

In a configuration, the total rotational x-ray emitting position angle from the center axis by less than 5 degrees or, less than 4 degrees, or less than 3 degrees or less than 3 degrees or less than 2 degrees or less than 1 degree.

In a configuration, the method can be configured to be combined with another movement trajectory, tube rotating angle, or detector angle to either expand a field of view of an x-ray emitting beam volume or to combine projected images, and/or to expand flexibility of movement due to pre-existing application requirement.

In a configuration, the requirement can comprise angular and translational movement of the subject or movement of the VOI.

In a configuration, each movement can be configured to introduce a new projection path for each voxel of the VOI.

In a configuration, the x-ray can be emitted from the same location or a different emitting location.

In a configuration, the x-ray system can comprise more than one source, each source is capable of tomography.

In a configuration, the more than one source can be configured to be used and represented in the same system matrix, each source having a plurality of emiting positions or are configured to move to generate projecting images of the VOI, wherein the projected images are combined with other imges to reconstruct the 3D image of the VOI.

In a configuration, each source can be configured to project projected images of at least one portion of VOI, and a 3D reconstruction can be derived from two or more set of projected images, each set produced by at least each source.

In a configuration, the same system matrix can comprise different sources, the measured data being combined to establish a more accurate provisional 3D reconstruction.

In a configuration, the 3D reconstructed image can comprise the VOI, which is determined through earlier 3D reconstruction of different resolution, or energy level or spectral imaging or single energy image or 3D reconstruction at at least one or more different x ray emitting positions.

In a configuration, the projected image can be imaged processed with a scatter removal method involving interpolation in the spatial domain and/or using a movable beam particle stopper array and/or stacked detector method with a beam particle stopper plate or movable beam selector.

In a configuration, the attenuation value and or density information derived for at least one substance of interest or composite substance of interest can be in reconstruction of the 3D image.

In a configuration, a final 3D reconstruction can be used to determine the VOI.

In a configuration, the x-ray sytem can be mounted upright.

In a configuration, the x-ray sytem can be mounted in a C arm or U arm.

In a configuration, the projected images can be located at a different VOI on the subject combined 3D reconstructed image resulting in a 3D image with a larger volume.

The present disclosure provides an example x-ray imaging apparatus comprising a controller configured to: obtain projection data representing an intensity of radiation having illuminated a VOI and exited out of VOI of an object detected at a plurality of detectors, or a ratio of the intensity over a radiation intensity entering the VOI derived from radiation detected in a first detector and radiation detected at a reference detector, and generate the first data sets and at least a second dataset based on the obtained projection data, wherein thefirst data sets can comprise data generated by the first detector, and the at least a second data set can comprise data generated by the first detectors or a second detector, wherein the projection data can be from a different radiation emitting position, energy level, exposure level, and/or different system configurations.

In a configuration, the controller can be configured to generate more data set comprising data generated by the same first detectors, or the same second detectors or additional detectors.

In a configuration, the apparatus can comprise a single radiation source, which have different emitting position, different focal spot sizes, and/or different fields of view due to a field of view restricting device or collimators.

In a configuration, the apparatus can comprise first and second radiation source, the second radiation source being a different radiation source than the first radiation source but travelking in the same area of emitting positions of the first radiation source, wherein the radiation emitted by the second source can be of a different focal size, and/or different energy level and/or speed of pulse generation.

In a configuration, the apparatus can comprise first and second detectors, the first detectors having a different detector configuration than the second detectors.

In a configuration, the apparatus can comprise a 3rd or more detectors, wherein the respective detector configurations of the first detectors and second detectors, and the third or more detectors are determined by a detector type.

In a configuration, a projection geometry and/of pixel elements can be arranged within the respective first detectors and second detectors, and the controller is configured to reconstruct a combined image using the plurality of datasets.

In a configuration, each dataset of the plurality of datasets can correspond to a respective system-matrix equation representing respective projection geometries corresponding to the plurality of datasets.

In a configuration, each dataset of the plurality of datasets can correspond to approximately the same or similar system matrix equation or a different system matrix equation representing respective projection geometries corresponding to the plurality of datasets.

In a configuration, the image can be reconstructed using the same system matrix for a plurality of datasets comprising data with scatter to primary ration less than 1% or less than 5%, by one or more of: a low scatter VOI, using time of flight primary measurement by removal of scatter in the time domain, using scatter removal method comprising primary x-ray image derived from subtraction of high resolution scatter derived from interpolation of low resolution scatter image, using ART or its derivative algorithms, and/or iterative methods.

In a configuration, the image can be reconstructed using different system matrices for a plurality of datasets, at least one modified-dual variable and using a splitting based subproblem method.

In a configuration, the image can be reconstructed using the same system matrix for a plurality of datasets, at least one modified-dual variable and using a splitting based subproblem method.

In a configuration, subproblem can be performed on the datasets separated by time of data generation.

In a configuration, the apparatus can further comprise at least one more addition dataset.

In a configuration, the system matrix can incorporate the use of optical sensors and camera, guided by AI to use surface image and AI to select the ROI.

In a configuration, the apparatus can comprise AI software used to reduce noise.

In a configuration, the iamges can be scatter removed to less than 1% SPR or less than 5% SPR, avoiding a need to consider scatter in simulation.

In a configuration, a distance moved by an x-ray source from a first position to a second position can be less than 5 cm, or/or less than 2 cm squared or less than 5 cm squared or less than 1 cm squared and less than 4 cm squared or less than 3 cm squared and/or less than 3 cm squared from the first positions.

In a configuration, x-ray emitted at the second position can be configured to travel in the same volume or 6D spatial position as x-ray from the first position.

In a configuration, the x-ray source can be field emitting to emit x-ray at the same spatial position as the x-ray filament tube or other type of x-ray source, or the various type of source or its modulated version with same or different parameters including focal spot size, energy level, frame rate, and/or geometry, or manipulated by different x-ray optics or steered by different mechanisms may be used, wherein a same spatial matrix, a modified dual or multiple variable method, or a split subproblem method is used.

In a configuration, an optical method can be used in conjunction with the present x-ray systems, using the system matrix.

In a configuration, vectors can be used in the system matrix.

In a configuration, the controller can be configured to use dual energy or multiple energy x-ray to determine an approximate area and distribution in the projected image on a pixel by pixel basis.

In a configuration, the data sets can be used to reconstruct a 3D image.

In a configuration, the controller can be configured to aegment out the material volume and space distribution, and/or perform material decomposition.

In a configuration, the controller can be configured to determine the ROI before and/or after reconstruction for further spectral imaging.

In a configuration, the controller can be configured to combine movement of source and/or detector with that of a tomography system.

In a configuration, the controller can be configured to perform Contrast Agent decomposition.

In a configuration, the controller can be configured to perform dual energy or multiple energy decomposition to distinguish an X-ray absorbing material.

In a configuration, the x-ray absorbing material can comprise: a metal or plaster cast mixed with barium, a catheter and/or implant with one or more materials and/or having lumen and sheath made of different x rayx-ray absorbing properties or atomic z, or made with distributed x-ray absorbent material at certain spatial locations interlaced with x-ray transparent material, sufficient to determine its spatial distribution compared to the background and other segments in the same catheter or implant, or including well-characterized x-ray absorption properties on a pixel basis, sufficient to differentiate one segment to another segment, a plaster cast, a blood vessel, a contrast labeled blood vessel, microcalcification, and/or contrast-agent labeled molecules.

In a configuration, the controller can be configured to denoiseusing AI software trained to remove noise.

In a configuration, the controller can be configured to use data generated in training of an AI algorithms for reconctruction.

In a configuration, the apparatus can be part of a tomography device.

In a configuration, the subject can be loaded on a table or bed which is x-ray transmissive, the table or bed being placed on top of a detector gantry of the tomography device.

In a configuration, a patient can be configured to lay on a surface of a detector gantry, which is transparent to x-ray.

In a configuration, the device or a portion thereof can be portable by connecting to an autonomous driving device to be transported inside a clinic or to remote location outside the hospital.

In a configuration, the device can be less than dimensions of an opening of a standard door.

In a configuration, the device can be used as a point of care device, and/or used in a patient's room.

In a configuration, the device can comprise a detector module that is movable and can be placed in between the patient's bed and the patient.

In a configuration, the controller can be configured to perform material decomposition using a beam particle stopper reconstruction method.

In a configuration, the beam particle stopper reconstruction methods can comprise filling a data gap from an image taken at the same x-ray emitting position and with a different beam particle stopper array position where primary x-rays are blocked.

In a configuration, the beam particle stopper reconstruction methods can comprise filling a the data gap during the reconstruction process, each projection path which is missed from the beam particle stopper being described as having no data input, therefore requiring extra projection data to be generated from the same x-ray emitting position or using sparse data 3D reconstruction algorithms.

In a configuration, the material decomposition can be performed for metal and/or other absorbing material in a catheter or an implant comprising one or more substances overlapping each other, if the controller knows the approximate density and/or thickness of the catheter or the implant.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure. Corresponding numerals indicate corresponding parts.

FIG. 1 illustrates two sources of various focal spot in one emitting position, scatter removed with single detector assembly including beam particle stopper.

FIG. 2 illustrates a side view of X-ray source turret, with four different x-ray sources.

FIG. 3 Illustrates a bottom view of x-ray source turret with a number of x-ray source, for example, five different x-ray sources are illustrated here.

FIG. 4 illustrates two or more x-ray sources and moving such sources in a linear axis.

FIG. 5 illustrates an implementation where a rotating motion device such as rotational stage, moves x-ray sources along a rotating axis

FIG. 6 illustrates a top down view of an xy mover combined with a rotational stage to move one or multiple sources on a xy planeor by xy translation stage as well as the rotational stage.

FIG. 7 illustrates the flexibility and expandibility of the imaging system with ttwo or more detectors, in one example, for imaging of a ROI, positioned downstream from a first detector.

FIG. 8 illustrates an X-ray imaging system described in this disclosure enclosed in an enclosure.

FIG. 9 illustrates igh resolution front detector method or dual detector method where the front detector can be moved to a selected VOI after VOI is selected, and imaging of VOI can be simulatniously done with both front and back detectors.

FIG. 10 illustrates a flow diagram of data replacement method.

FIG. 11 illustrates a flow diagram listing examples of image processing methods pre imaging, during image acquisition, post image acquisition pre reconstruction and post reconstruction

FIG. 12 illustrates a high throughput X-ray system including systems capable of 2D and/or 3D spectral measurements.

FIG. 13 illustrates another high throughput X-ray system including systems capable of 2D and/or 3D spectral measurements.

FIG. 14 illustrates a high throughput X-ray system including systems capable of 2D and/or 3D spectral measurements.

FIGS. 15a-b illustrate external Reference Component ERC used to spatially position a Component 2C-1 Internal to a Subject 2R01-1 being illuminated and imaged by the detector assembly 22.

FIG. 16 illustrates an x-ray measurement deviceintegrated with an energy modulation for measurement of elasticity of ROI.

FIG. 17 illustrates an x-ray system with a near real time phase contrast and/or capable of Fourier transform device.

FIG. 18 illustrates a typical x-ray measurement device combined with a shear force generator for elasticity measurements.

FIG. 19 illustrates magnification of X-ray Beam passing through the region of interest.

FIG. 20 illustrates voxels in Single Beam Path.

FIG. 21 illustrates a method to expand large field of view of x-ray imaging apparatus and methods referenced and described in the x-ray imaging system and apparatus and methods disclosed presently.

FIG. 22 illustrates electron Beam Steering in Electronic Phase Stepping.

FIG. 23 illustrates a Portable x-ray system comprsing the entire x ray system or its submodule attached to or integrated with a motorized gear.

FIG. 24 illustrates another portable x-ray system.

FIG. 25 illustrates an Apparatus for imaging of various body parts or tissues or organs with the large field of view x-ray system by moving source and detector pair.

FIG. 26 illustrates a front view of a spectral tomographic mammography system.

FIG. 27 illustrates a side view of a mammography support device attached to or detached from the x-ray system

FIG. 28 illustrates a side view of a spectral tomographic mammography device attached to or detached from the x-ray system.

FIG. 29 illustrates a tomography system configuration and method, nMatrix or n²Matrix.

FIG. 30 illustrates an example of an x-ray imaging system, or a x-ray tomography system or an spectral imaging system or spectral tomography system, in some cases with a large field of view. It is a dual or multiple detector imaging system with the use of separate beam particle stopper array plate for each detector. Imaging of one detector help to select for ROI and/or for ROI to be measured by a second detector.

FIG. 31 illustrates schematically a Fourier-transform spectrograph generated by x ray measurement system.

FIG. 32 illustrates a holographic x ray imaging system.

FIG. 33 illustrates one example of a system configured to perform a matrix x-ray tomography reconstruction method.

FIG. 34a illustrates using sensor placed downstream from x-ray source, upstream from the imaged subject to measure x-ray input intensity as a reference value.

FIG. 34b illustrates Sensor in Collimator to monitor x-ray exposure.

FIGS. 35a, b, c and d, each illustrates an example of an intervention device, such as RF ablation probe or the catheter which has an opening for injecting therapeutic reagents and or liquid or contrast reagents onto VOI.

FIGS. 36-40 illustrate examples of beam particle stopper plate with beam particle stoppers in distributed region to block primary x-ray beam.

FIG. 41 illustrates an example of intervention device comprising an implant inside an catheter, in which one or more regions or components are designed for differential x ray measurements for better visualization and therefore control of each component.

FIG. 42 illustrates an x-ray source module.

FIG. 43a is a graph of X-ray energy photon number per energy interval.

FIG. 43b is a graph of X-ray energy number per 1 Kev.

FIG. 44 illustrates an example of material decomposition method based on spectral imaging.

FIGS. 45a and b are graphs illustrating energy response function system for two materials—establish interporlation plot to correlate density to from measurements at dual energy levels of a composite material comprising two materials or two substances.

FIGS. 45c and d are graphs illustrating solving energy response function by using inverse energy response function, look up the corresponding density value of each material based on dual energy measurements.

### DETAILED DESCRIPTION

Aspects of the disclosure are provided with respect to the figures and various embodiments. One of skill in the art will appreciate, however, that other embodiments and configurations of the apparatus and methods disclosed herein will still fall within the scope of this disclosure even if not described in the same detail as some other embodiments. Aspects of various embodiments discussed do not limit scope of the disclosure herein, which is instead defined by the claims following this description.

Qualitative and/or quantitative measurements of 2D, 3D X-ray applications and CT computation can require large amounts of memory and computation. A detector module or assembly or a submodule attached onto an existing detector via wireless or tethered communication, may contain memory storage and/or database storage or database capability, with one or more microprocessors for localized storage and computation, processing, image reconstruction and/or storage at the detector side. The display may be done locally or directly from the microprocessor or done remotely via wireless or Ethernet or tethered communication methods to a second microprocessor for display and in some cases, additional computation and storage.

Image reconstruction may involve algorithms and methods of conventional rotational CT and/or tomosynthesis electron tomography, Mill, PET, SPECT and Transmission Optical Tomographyand it may include material decomposition, attenuation value measurements and density measurements for each material in the imaged object spectral 2D or 3D images to improve accuracy, precision and speed for reconstruction. It may involve optimization of image acquisition and reconstruction via derivation of density data for individual substances on a normalized pixel basisand via identification of the region of interest (ROI) or selected regions of ROI for reconstruction before and during acquisition and after acquisition.

An x-ray measurement or computer tomography device, in addition to acquiring and presenting images, measurements and features based on new functional capabilities, features and resolutions demanded by the application, may extract from a tomograghic image and related data to provide a display mode that presents measurement values and images through user interface and procedures that are familiar to users such as a CT slice image and densitometer measurement values.And it may present images and values previously not available for example for a virtual reality display device which allows a number of presentation mode not available using CT images.

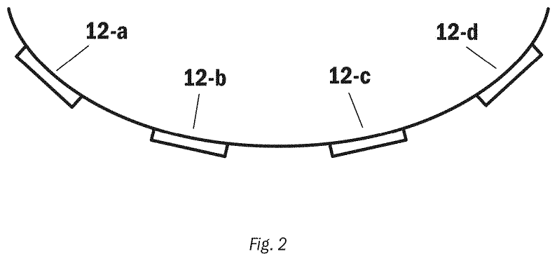

An x-ray imaging system may have one, two or more x-ray sources of various types, such as x ray source with multiple source configurations, for example, varied x ray emitting areas, number of emitting positions, energy levels, field of view or different sources of different technology platforms, varied focal sizes and/or other varied values in parameters such as preparation time, exposure, speed, power, energy level, number of energy levels, spectral wave form characteristics, pulse duration, pulse characteristics and/or form factor in approximately one or more x-ray emitting locations at the same time or at varied time frames. FIG. 2-6. illustrates system configurations where one or more x ray source 12-a to 12-d, 12-e, 13-1 to 13-4 may be movable to be placed above an VOI, 2., so that the same VOI may be interrogated by different type of x ray sources. For example, 12-a may be a hot filament conventional tube with wide field of view. 12-b may be a field emitter source; 12-c may be a source with limited field of view and or emitt only low energy level x rays; 12-d may be a high flux, ultra bright, source may be able to generate x rays to be filtered to have selected energy bandwidth and maintain flux needed for imaging a VOI.

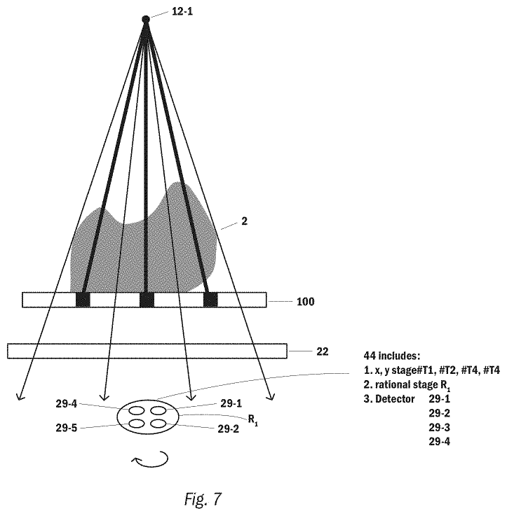

FIG. 7 illustrates that one or more detectors can be moved to image the same ROI depending on the demand of the application requirements using the same or different x-ray source.

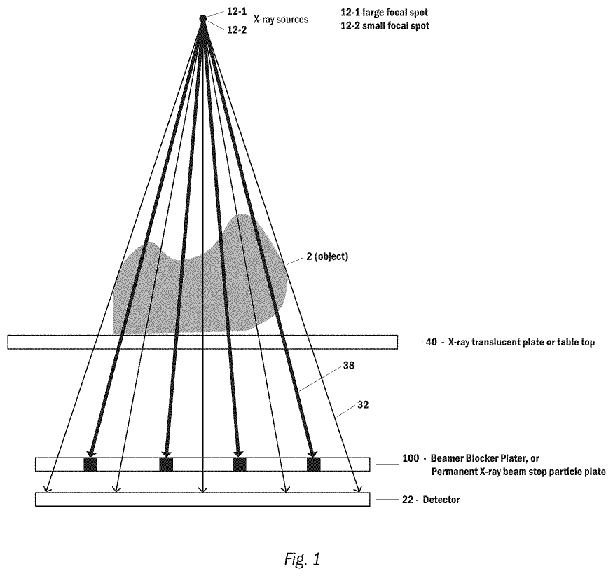

For example, FIG. 1 illustrates two overlapping emitting positions 12-1, 12-2 of a single source, each emitting position may have varied focal spot size.

The x ray system illustrated is capable of scatter removalwith a single detector assembly 22 x-ray radiation emitted out of the source as a cone beam or multiple beams with divergent 3D shape, some of the projected beam passing through VOI are blocked by beam stopper particles on the beam particle stopper plate 100. There can be an x-ray sample holder 40, which is x-ray transmissive. Image processing may remove scatter, and the attenuating effect of the sample holder 40 if there is any.

In some cases, two or more sources are moved together for faster 3D reconstruction. For example, each source may illuminate a volume of interest (VOI) and two or more such VOI are combined to form the final VOI. Sources may be moved independently relative to each other to optimize imaging procedure and speed of operation which depends on the application requirement, for example resolution requirement in the Z axis. X-ray sources may be steered in a synchronized or asynchronized manner to image the same 3D VOL An X-ray imaging system with one or more detectors pairing with one or more of the x-ray sources may be movable upstream or downstream of the X-ray detector assenbky 22 or in a position to to image VOI in place of detector 22

Some or essentially all the projected images from each project geometry configuration can be represented and incorporated into the system matrix with adjusted number of coordinates and or vectors for recontruction image processing and data analysis.

Tomography, or 3D imaging, or multiple dimensional imaging, spectral 3D, spectral tomography, nonrotational CT, personalized CT, nMatrix method Spectral X-ray 2D/3D/Tomography using multiple axis matrix image acquisition and reconstruction with less than 1% or less than 5% SPR, capable of real time 2D and/or 3D and/or 6D fluoroscopy and dimension measurement can include the following apparatus and methods.

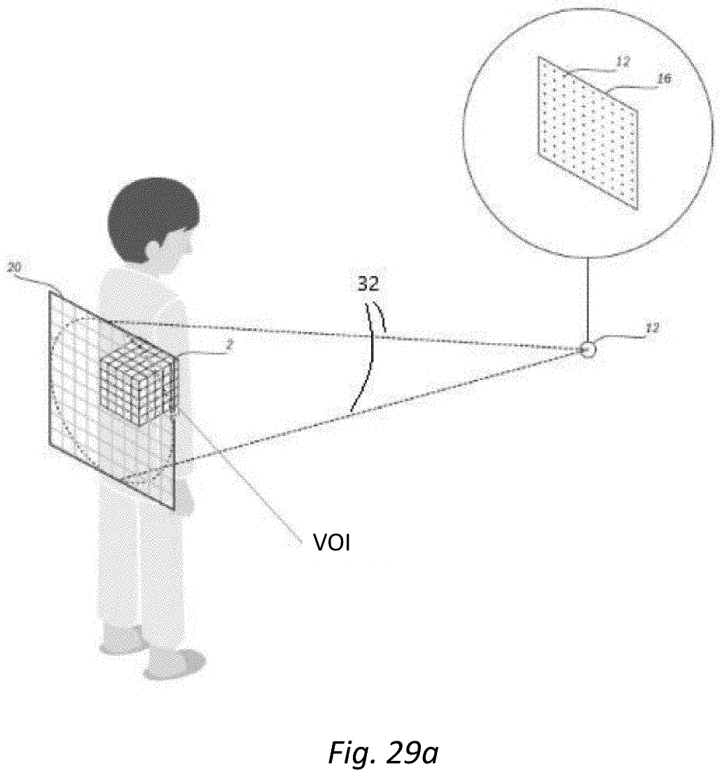

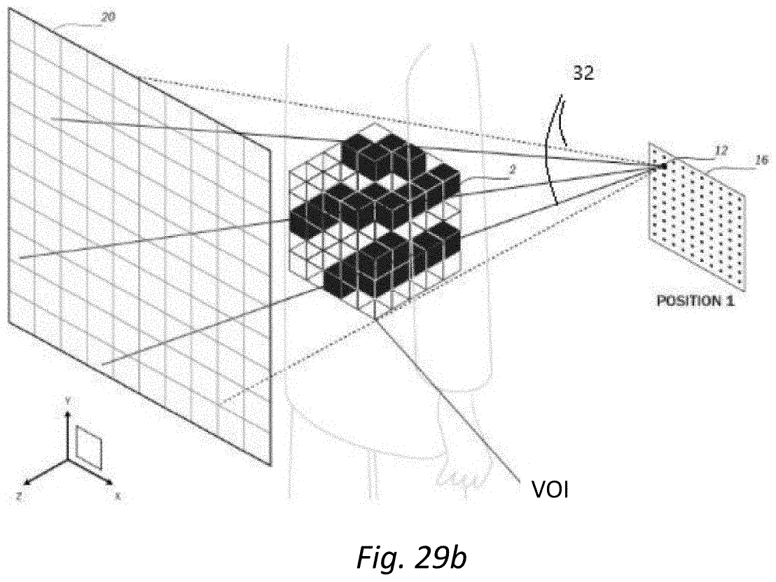

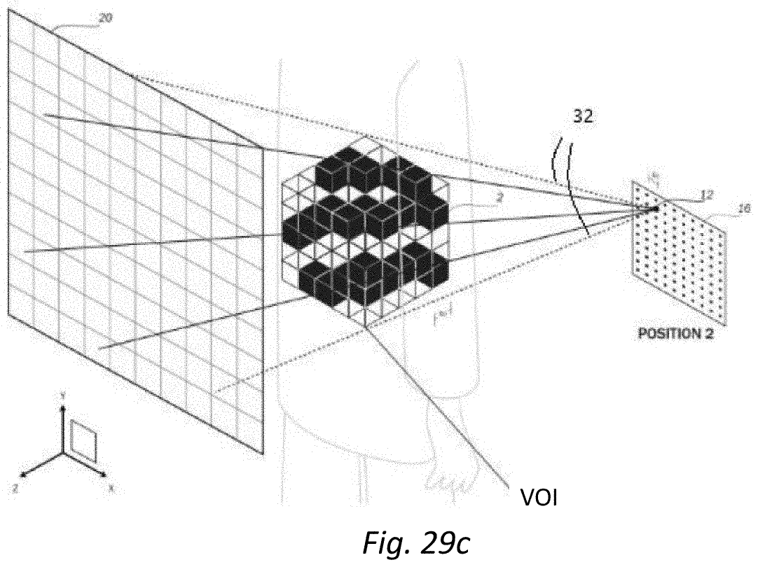

FIG. 29 illustrates one example of apparatus, can be referred to as a system of Tomography, or 3D imaging, or multiple dimensional imaging, or spectral 3D, or spectral tomography, or nonrotational CT, or personalized CT using nMatrix, n²Matrix method and spectral imaging method.

The can include Source 12; 2D area of X-ray emitting positions, 16. the X-ray emitting positions 16 can also be in 3D-6D spatial locations or volumes. The apparatus can also include imaged Subject, 2, and/or Volume of Interest, VOI 2, Detector or detector assembly or detector module, 20.

Resolution along the Z axis in VOI is approximately Xc. Xc is the approximate distance between Position 1 and Position 2 of x-ray emitting positions in the spatial location or volume or 2D area of emitting positions, 16.

Position 1 and 2 may be referred to as first positions for X-ray Tomography. The smaller distances between emitting positions may be possible to further image the VOI and/or imaged subject 2 to provide additional images for tomography or image analysis and processing purposes. Smaller distances between emitting positions, or emitting position other than first positions, sometimes, referred to as second positions, may be used to resolve the newly introduced unknowns outside of VOI if needed. Alternatively, second positions may be used to resolve voxels with higher resolution, along the depth of the object, perpendicular to the detector or detector module 20.

The imaging system of the present disclosure can optionally be an non-rotational, non-contact imaging system configuration capable of true or complete 3D volumetric reconstruction. Additional details of this technology are described in International Patent No. WO/2019/183002. The basis for this method is that only very small area movements or small angle movements and/or total movements of the X-ray source are needed to reconstruct a complete 3D image of a VOI if 2D projected images are captured at integer multiples of Xc in a 6D volume, or at least in a 2D area, for example, on a 2D plane parallel to the detector or detector module 20. As noted above, Xc is the resolution desired for imaging the VOI 2 in the z axis or depth, which in some cases, isparallel to the center axis of the x-ray illumination. An imaging geometry may be configured to reduce or minimize area of movement of the X-ray source/X-ray emitting position in a 2D plane parallel to the detector relative to the object or Vol 2. To resolve the unknown voxels in the VOI 2, the newly introduced unknown voxels outside of VOI, 2, denoted as “Voxelni”, may be much smaller than the total number of unknown voxels in the VOI 2. In turn, the number of newly introduced unknown voxels in a particle projection path may be significantly smaller than that of the unknown voxels in the ROI. It is therefore acceptable to neglect the contribution made by the unknown voxels in the regions outside of ROI, Voxelni. Using this method and system configuration, a minimized total number of 2D images, denoted as “NTT” can to be acquired for a complete 3D image or a complete tomography image to be reconstructed.

In some cases, when highly accurate or precise tomography measurement is needed, resolving of the newly introduced unknown voxels outside of ROI can be achieved by moving of first positions in the same area or same volume to second positions, but emit x-ray at a number of second positions at spatial positions different than first positions. And the distance between second positions may be varied, which can be smaller than Xc or larger than Xc or the same as Xc. In addition, the x-ray illumination volume may be reduced, for example, by a collimator, so that only the volume involving the newly introduced unknown voxels and corresponding voxels within the ROI in the projection path of the x-ray beam are illuminated repeatedly, and the rest of the x-ray beam may be cropped, to avoid additional exposure unneccearily to the portion of ROI which is not in the illumination path of the newly introduced unknown voxels outside of ROI—Voxelni.

FIG. 29 illustrates the method. The theoretical basis of this geometry, “nMatrix” is that with each X-ray emitting position, such as Position 1 or Position 2, a unique set of X-ray illuminated paths differentiated based on the spatial positions of the voxels (highlighted in black) in each path in the VOI are measured by the corresponding pixels on the detector 20. As the distance between the X-ray emitting positions is as small as Xc, if the total area traveled 16 on a 2D plane is at least equal to the depth of the VOI, the total movement angle in 2D area 16 relative to the original position can be less than 1 degree, and this way the number of unknown voxels introduced outside of VOI due to the movement of X-ray emitting position is minimized. Resolution achievable theoretically can be as high as single digit micron meter in XYZ dimensions, achievable using commercially available detectors. 3D image acquisition can take less than one second to achieve resolution similar to, or greater than that of a CT slice.

In resolving voxel attenuation values, each voxel may be given a value of 0 or 1, 0 if transmitted, 1 if not transmitted, or attenuated at a certain value.

each voxel may be given a value of transmission from 0 to a certain attenuation value, or 1, could be set for transmission at another range of attenuation value.

In some instances, therefore, approximately a range of values may be set as 0, and another range of values may be set at 1. For example, in human diagnostics, if air attenuation is defined as 0, the rest of the body tissue volumetric regions may approximately to be set at 1.

In another instance, voxel filled with bone material may be 1 and the rest of the body tissue volumetric regions may approximately to be set at 0.

Multiple set of linear equations can be used to resolve unknown voxels with 1, or 0 set with different approximate ranges and threshold.

The method can again sett 1 or 0 to correspond to a range of different attenuation values, and resolving the unknown voxel values to be either 1 or 0. The method can repeat or iterate the process with different ranges of attenuation values.

In some instances, such methods may be combined with dual energy or multiple energy decomposition methods, such as disclosed in the aforementioned PCT Applications.

In some instances, multiple energy measurements may be used to characterize and identify each voxel. One or more voxels may be segmented from other voxels in the VOI based on its attenuation value range. Thickness or spatial volume and position of each segment may be derived. If there is a reference database which corresponds to single or multiple energy measurements of the voxel, and/or each substance and/or composite substances, the exact density may be determined.

A tomography method may be include the following, with the numbering not necessarily indicating the sequence of the step, but rather a list of possible steps.

1) The 2D images measured can be separated into primary image and scatter interference and therefore, can achieve the highest resolution and quantitative 2D primary images attainable by the detector.

2) If the X-ray emitted by the source is a cone beam illuminating the imaged subject, and if at a first position (position 1) the X-ray illuminates the VOI, for measurement of each pixel on the detector or each detector region, there are a number of voxels within the VOI in the projected path that can be traced back to the X-ray emitting position. As illustrated in FIG. 29, when the X-ray emitting position is next moved to another location, position 2, for example on an xy plane that is as small as one pixel pitch away from position 1, then a new set of illumination paths differentiated from the first set of projection paths are measured as they include voxels with distinct combinations of spatial positions in each of new projected paths, as illustrated in FIG. 29. The distance between Position 1 and Position 2 can be Xc, the desired resolution of VOI in the Z axis, perpendicular to the detector. To resolve all the unknown voxels in a VOI in a volumetric dimension of (m×n×p), where m, n, p indicate number of voxels along each axis, and m and n correspond to the number of voxels in the x and y direction respectively, a p set number of illumination paths, each different from one another based on the spatially distinct voxels involved in each projected path, may be minimally required to be measured by the detector to resolve a complete 3D volumetric structure.

3) Minimization of unknown voxels outside of the VOL To minimize the number of unknowns introduced outside of the VOI during the imaging process, it is preferred that the X-ray emitting position movement from the original starting position is minimized. One configuration is to move in the emitting position in the xy plane, and limit adjacent emitting positions to only pixel pitch or unit measurement=Xc apart. Xc, as seen in FIG. 29, may be equal or greater than Xa and Xb, which are the pixel pitch dimensions along the x and y direction of the detector respectively. Alternatively, X-ray emitting position may move in 3D or more dimensions to minimize the total movement angle for tomography.

4) The total number P of the needed X-ray emitting locations is the total number of Xc along the Z axis. The needed X-ray emitting locations may be arranged in a 2D plane to minimize the movement and number of newly introduced unknowns during the imaging process. For example, to move linearly, 20 cm is required to resolve unknown voxels in the VOI, but at the same time, more unknowns will be introduced in the imaging process due to the illumination of regions outside of VOI when the X-ray cone beam moves. However, if the X-ray emitting position is moved in the xy dimension, the total illuminated volume may be minimized, the total movement angle can be less than one degree if the source to detector distance (SID)=1 meter. As a consequence, the number of unknown voxels outside the VOI are dramatically reduced. For example, only an area of 20×20 mmA2 is required to resolve unknowns in the complete 3D volumetric region if the desired resolution is 0.5mm. These features greatly reduce the design complexity of the moving mechanism for the X-ray source for a complete 3D image reconstruction. The hardware to implement the X-ray emitting position movement can include a) a motorized mechanical translation stage, b) an electromagnetic steering device for electron beams in X-ray generation, and/or c) a pixelated X-ray source where multiple X-ray pixel size sources are located next to each other on a 2D plane. Electron Beam Steering device is favored more than motorized implementation due to the following: a) the size of step and resolution can be small and low cost while in micron range, and b) the electron beam steering device moves the X-ray emitting location without a mechanical moving part which greatly reduces complexity, minimizes aberrations induced by dynamic flexibility and vibration and improves compactness and noise performance. The Electron Beam steering device can be mounted onto the X-ray tube, such as described in the aforementioned PCT Applications.

To move the beam selector to align with the X-ray emitting location as electron beam steering device steers, a mechanical mover may be used. Moving the selector may not be required for the alignment with X-ray source with every movement of X-ray emitting position as the size of primary beam on the rear detector is in the mm range.

When a single detector is used, such movement of detector may not be needed for tomography.

Tomography or 3D Image Acquisition—Rotation

In some cases, the source or x-ray emitting position, or the source and detector pair may be rotated relative to the object, in pitch or yaw or roll around the subject, preferably in at least two directions of rotation.

The rotational movement of x-ray emitting position and detector pair may be combined with x-ray emitting position movement in the direction of one axis on a 2D plane parallel to the detector. Detector in this case may or may not move with the x-ray emitting position.

To reduce the total number of projected images generated to achieve the resolution required for the thickness of ROI, each position is different from the other positions to allow a new set of illumination path across the ROI, and the step (that is, movement from one position to another) size may be approximately one unit of resolution along the z axis, the axis vertical to the detector. The variation in illumination paths is at least approximately one voxel from the corresponding path of collected by the same pixel or detector region.

The x-ray source may generate parallel beam or x-ray fan beam or cone beam, which may be converted to a presentation in a format of parallel beam or fan beam. The movement and step size can be minimized, to reduce system complexity and increase speed of image acquisition. Each time the new set of projection paths may be unique compared to the projection image set taken before and after.

The 3D x-ray imaging system disclosed herein can include the x-ray emitting positions being moved relative to the subject in at least two axis, or two dimensions, for example in each of the six degrees of freedom, x y z rotate, yaw, pitch, in reduced and/or minimized number of steps. Each step approximately is one pixel pitch of the detector resolution needed in the z direction, The movement can be effected by, for example, magnetic or electron lens or mechanical or motorized, or electromagnetic methods, which may be included in or attached to each of the x-ray sources.

An x-ray system may include the x-ray source(s) and its corresponding detector(s) which may both move in one or multiple dimensions, optionally synchronized. Alternatively, the detector or source may stay stationary, and the VOI or imaged subject may move. Alternatively, the source may move independently of the corresponding detector.

An X-ray system including the x-ray source and its corresponding detector(s) may move in one or multiple dimensions, because the sensor assembly (which may include one or more detectors and a beam selector) may be relatively small and the movement can align the focal point of the source with the sensor, especially when the sensor is the triple layer version involving a beam selector sandwiched between two detector as disclosed herein, or stacked detectors, with low energy detector on the front and high energy detector in the rear.

The X-ray illumination volume, for example, a cone beam or parallel beam, and therefore the x-ray emitting position or the x-ray tube emitting x-ray may rotate in pitch, yaw or roll or in at least one axis relative to the object.

Correspondingly, detector or detector assembly may rotate, to align with the center axis of the x-ray illumination, facing the object, or VOI, 2 in FIG. 29. In some cases, such rotation is not needed if the movement or rotational movement of the source 12 or the object 2 is such that the x-ray illumination passing through the VOI can be captured by the detector or detector module 20.

Detector and detector module 20 may move, along with the movement of x-ray emitting position or x-ray tube, in the x y z volume relative to the object for tomography or spectral tomography image acquisition.

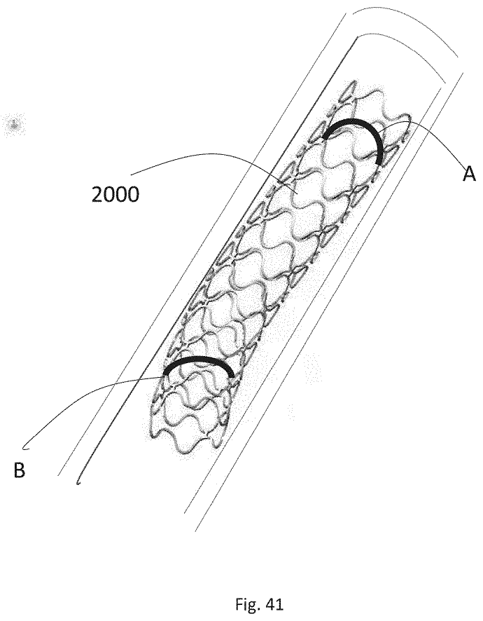

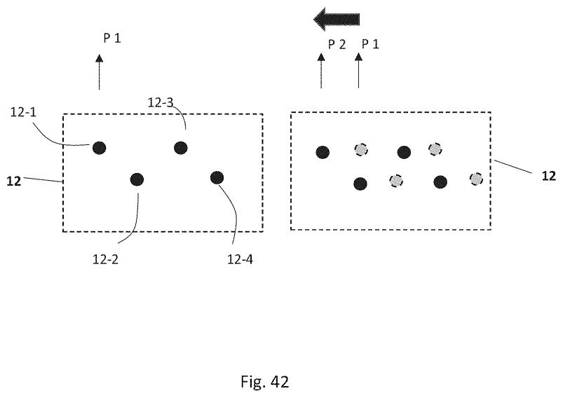

FIG. 42 illustrates multiple sources of an x ray tomography system. In some cases, the sources are moved together for faster 3D reconstruction. Each source will illuminate a volume of interest (VOI), and two or more such VOI are combined to form the final VOI. sources may be moved independently relative to each other to optimize imaging procedure and speed depending on the application requirement, for example, the resolution requirement in the Z axis. X-ray sources may be steered in a synchronized or asynchronized manner to image the same 3D VOL An X-ray imaging system with one or more detectors pairing with one or more of the x-ray sources may be movable upstream or downstream of an x-ray detector.

The apparatus and method minimize time required for a complete tomography image as minimum number of exposures are taken for the reconstruction of a complete tomographic image without or with little missing data gaps.

Spectral CT

The present tomography method may be combined with material decomposition method utilizing for example, a dual energy or multiple x-ray system including broad band x-ray source paired with flat panel detector and/or 2D detector, using inverse energy response function equation method to material decompose and solving for density and thickness of each substance

Alternatively, the material decomposition method may utilize spectral CT method using conventional CT system with energy sensitive detectors, such as a multi unit pixel detector, similar to medipix, each with an energy sensitivity with energy threshold, or photon counting detectors or a stacked dual energy detector, a filtered quasimonochromatic or monochromatic source with a detector

Variation of spectral CT may involve using full field imaging using flat panel or 2D detector and a set of source and detector or detector module pair for tomography or spectral tomography or spectral imaging. The detector may optionally be a spectrometer or spectroabsorptiometer for example energy dispersive grating and spatial sensitive detectors.

In some cases, tomography images may not need to be reconstructed, such as with a low resolution tomography image, and a volume of interest may be identified. And appropriate spectral imaging may be used to further interrogate the substances within the VOI, with anything external to the selected VOI layers where the substances of interest is situated, denoted “ secondary VOI” or “ VOI2nd”. In this case, less number of images may be needed to identify, characterize and determine the substances within secondary VOL In cases where tomography is needed, the layers below secondary VOI and above secondary VOI may be well characterized as an integrated unit, and images can be acquired to zoom in and resolve VOI of small dimensions with desired resolution in the secondary VOI.

Derivatives of the Tomography Systems and Dual Energy and Spectral Imaging System

Due to modular capabilities of the x-ray systems and methods of the present disclosure, modifications and/or extension of the system and/or the measurement system disclosed herein, and/or modifications and/or extension of a portion or complete aforementioned x-ray imaging system hardware and/or software to form hybrid measurement systems with analysis, measurements, diagnosis and inspection capabilities, is also part of the present disclosure.

Existing x-ray measurements and imaging system including CT, O-ring, C arm, Densitometer, microCT, digital tomosynthesis, general x-ray Imaging of various configurations, fluoroscope, surgical guidance systems for robotics, and minimum invasive intervention procedures, spectral imaging, radiotherapy, helical CT, k-edge imaging, portable imaging, mobile imaging systems and various spatial system configuration may be improved by integrating a portion or a complete x-ray imaging system of the present disclosure to increase resolution and/or frame rate and/or accuracy and/or lower radiation and/or reduce time required for acquisition and reconstruction in tomography and offer spectral imaging capabilities to include material decompositions, and/or offer scatter removal capabilities to reduce scatter to less than 1% or less than 5% SPR for quantitative imaging.

In some instances, application requirement is such that the information of ROI needed in order to achieve inspection, diagnosis or monitoring or tracking is much less than high resolution 3D. In those cases, the total number of images to be acquired may be less than NTT, which is the total number of images needed to be taken (m×n×p) in order for a complete tomography to be reconstructed.

Material decomposed measurements and/or measurements of point to 3D and any number of measurements in between for ROI, or distributed portion of ROI may be measured to sufficiently identify and/or characterize ROI or one or more portion of ROI, based on predetermined information and/or tomography image by using methods and configurations of the present disclosure and/or methods of the present disclosure in combination with conventional CT and/or its derivatives, or its variations or tomosynthesis or other modalities.

The method to ensure sufficiency to identify and characterize ROI may be determined by a user or a digital program or established data, in a stored preexisting database. For example, for each application or each type of measurement or a predetermined ROI, a user may decide or a digital program may decide on a number measurements needed in order to reconstruct an image of ROI based on the characteristic of ROI and requirement of the application.

AI or deep learning may be used for identify and characterize ROI based on a limited number of measurements. AI or deep learning algorithms, in some instances, combined with a digital program without deep learning or combined with a user input, may be used to determine an optimized procedure and/or methods for selection of measurements required for identification and/or reconstruction of ROI based on the characteristic of ROI and requirements of the application.

Deep learning or AI program may be trained by a set of data which includes the predetermined number of measurements and measurement steps and/or procedures to identify or reconstruct or characterize a ROI or multiple ROIs with reduced exposure and/or minimized exposures. Such an algorithms can thereby be used at the time of imaging an ROI of unknown voxels to identify, and/or characterize and/or reconstruct ROI of the unknown voxels.

Such a set of measurements may be different from current measurement methods where a compressed and sparsed imaging method is used in place of high resolution CT image, which was reconstructed once prior to the imaging process. The differences include:

The number of measurements and/or measurement steps are much less than compressed and sparsed image set as a CT image or a sliced CT image does not need to reconstructed in order to identify or characterize the ROI.

Measurement of material decomposition at a pixel level may be used to identify a ROI in the present disclosure.

Characterization and identification of ROI may be done prior to reconstruction in the present disclosure whereas reconstruction needs to be done before a ROI is sufficiently characterized.

Quantitative measurements such as density values are used in identification, characterization and determination of ROI and/or in the deep learning process which is not used in the current CT method relating to sparse and compressed imaging method.

In the sparsed tomography method, the entire VOI is illuminated in each projection image, and the total number of projection may be reduced. In the present disclosure, each project measurement may be just one point or 1D or distributed 2D images in the selected region of VOI, and the total number of projections may be reduced depending on the application requirements. In some cases, the number of projection images is reduced to such a level that the images themselves may not directly result in tomograpy reconstruction, and additional data independent of the measurement data may be used in tomography reconstruction if needed, thereby providing a method to reduce exposure and/or time for image acquisition or reconstruction significantly.

The radiation level is significantly less in the present disclosure due to much less complex geometry configuration, optimized measurement steps, as reduced projection numbers are needed in tomography and optimization of procedures, which may include measurements of different types, and/or dimensions and/or varied spatial locations within a VOI.

Prior methods developed for image reconnections of CT and tomosynthesis for compressed and/or sparsed imaging conditions may still be used. Compressed and/or sparsed imaging method may be used, where the total number of images acquired is less than NTT, or where each 2D image is lower in resolution than that of Xc, the desired resolution in Z, or

the step size is significantly larger than Xc.

In the compressed and sparsed imaging method, x-ray source only moves in one axis, relative to the object. Newly introduced unknown in the region out of the ROI, Voi, is proportionally larger compared to the number of voxels in the ROI, especially in each x-ray beam path.

Preparation of Quantitative Images and Standardization

Material Decomposition and/or image reconstruction or analysis of quantitative data and images may require the data measured to precisely and accurately reflect the composition and construction of the imaged object regardless of the x-ray system configuration.

To accomplish this goal, before, during and post quantitative data acquisition and reconstruction, there may be standardization between x-ray systems as well as development of preparation procedures, processing models, algorithms and procedures of measurements.

Normalization and Data/Image Processing

Image processing may be needed for data and images acquired before and/or after data & image acquisition and/or after reconstruction for denoise, dark noise, white image, flat field, gain, detector consistency, artifacts, dead pixels, beam hardening, field of view correction, phase retrieval, and/or to be normalized and/or using other preprocessing and post processing method used in CT and tomosynthesis. Artifacts induced by geometric movement and computation may be removed as well in post image reconstructions.

Measurements in terms of photon counting and/or in absorption and/or in transmission may be used in the processing.

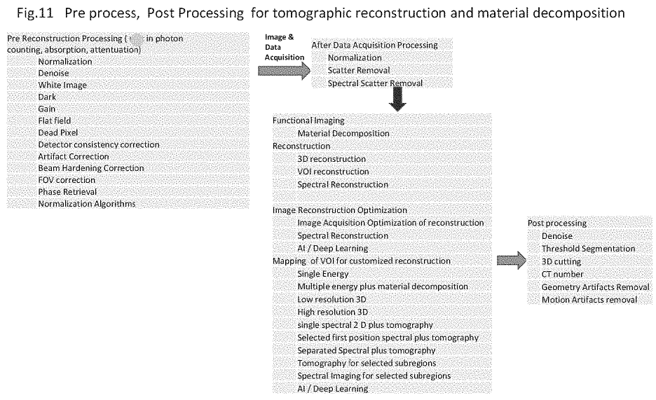

The preprocessing and post processing methods are to prepare data and images for consistent measurements throughout the use of the same x-ray system and/or for consistency between the use of the x-ray system with others like it, especially those from the same manufacturers of system, and/or components used in the system. It may also be used for quantitative analysis, functional imaging processing such as spectral imaging material decomposition, fluidics dynamic analysis and modeling, and 3D reconstruction. FIG. 11 illustrates a flow diagram listing examples of various procedures of data and image processing pre-imaging processing, processing after image acquisition and processing after tomography reconstruction. In an example, the scatter removal reduces scatter to less than 1% SPR.

The data preprocessing (PCP)and post processing model (PRP) may be created to utilize standards and algorithms based on databases, energy response function equation systems and/or plot systems, or reference data library built on x-ray imaging systems including comparable hardware or incomparable hardware such as source, detector, filter, scintillator, detector types, collimator, scatter removal hardware such as beam particle stopper array plate, sample holder between the subject and the detector, and/or x-ray optics, active x-ray modulators, passive modulators, optics used to steer, manipulate and pertube the x-ray, or optical signal derived from the x-ray, each of which affects measurements on the detector, and other modalities.

For example, one model allows the system to be able to correlate to another standard x-ray system via measurement of one or more known materials or one or more phantoms with known spatial or material composition. The deviation from the physical characteristics and property values derived from the measured data correlates the x-ray system to the standard x-ray system, compared to the derivation from that of the standard x-ray system.

The standard x-ray system may make a number of measurements and build database and quantitative analysis models such as material decomposition for single, dual or multiple energy systems.

The correlation allows the x-ray imaging system to make use of the database, or quantitative analysis algorithms and models based on the standard x-ray system to be useful.

Pre and post processing algorithms and models are developed to correlate x-ray imaging systems between different manufacturers, between the same type of system made by different manufacturers and different manufacturers, and between different type of systems made by the same or different manufacturers.

Traceability

A reference x-ray measurement system including source and detector and a set of hardware may be used to set the standards for what the measurements should be. Such a reference x-ray system or standard x-ray system may be compared to another x-ray standard which is made available on a most frequent basis, allowing wide adoption and usage of such standards to make measurements of different type of samples and/or establish quantitative analysis models and algorithms more functional, and/or accurate, and/or precise and/or traceable.

Such a traceability may be established on a standard x-ray system or a reference x-ray system, which is calibrated, denoised and data corrected with pre and post processing procedures. The process is similar to temperature measurement or time measurement at NIST.

Such a traceability, and/or standardization, combined with data and/or image processing (normalization, denoise, and other aforementioned PCP and PRP processes) and/or scatter removal before or after and/or during data and image processing and/or scatter removal, and/or image construction and/or material decomposition and/or quantitative analysis, and/or fluidic dynamics, may enable AI and deep learning algorithms to train on well-prepared datasets for diagnosis, image guide, monitoring, inspection and testing and tracking across a wide variety of x-ray imaging systems and hybrid systems independent of manufacturers and builders.

Pre processing and/or normalization methods for quantitative imaging may include one or more of the following:

Deviation of white image measurements on a number of pixels at the same time and/or at one time or multiple time intervals, or at a set sequence of imaging and measurements, similar to the actual measurements, for example, pulsed measurement at Hign, Low, Medium level of x-ray energy level, similar to that in actual measurements, with the average deviation from the average or mean value for each pixel at the same or similar energy level.

When the measured x-ray data downstream from ROI and the input x-ray data are known, the interpolation of the plot for any specific x-ray system may be based on the adjustment of deviation of measurement based on the quantitative relationship between the x-ray system and a standard x-ray system where such a standard system has been used to establish for example, an energy response function equation system, where solving of nonlinear multiple energy equation for material decomposition is established through inverse energy response function system look up table. In some cases, the correlation may be achieved through measurements of x-ray source and detector pair for each pixel of the detector or detectors using approximately the same sample standards.

The intensity ranges in ROIs may be smaller than the intensity range of the whole image when ROI is smaller than the field of view of the detector. Therefore in some cases, it is preferred to only normalize the measured value in the ROI.

In case of scatter removal in the spatial domain using interpolation methods, scatter removal may be performed on regions slightly larger than ROI. For example, when ROI is very small, 15 mm2, scatter removal may be performed on an area of approximately 2 cm2 or more.

Normalization or reduction of intensity levels can often be performed simultaneously.

In one example, the number of intensity levels is reduced to the range (0, . . . , 2k−1). The procedure can be described by the below equation:

$$Inorm ( x , y ) = \{ 2 k - 1 for N ( x , y ) > 2 k - 1$$

$$\{ N ( x , y ) for 0 \leq N ( x , y ) \leq 2 k - 1$$

$$\{ 0 for 0 < N ( x , y )$$

where:

N(x,y)=round_to_int (((I(x,y)-minnorm))/(maxnorm-minnorm))(2k-1)))

minnorm is minimum normalized value,

maxnorm is maximum normalized value,

k is no. of image bits per pixel after normalization.

The image normalization technique or method examples are for example one of the following:

min-max—in this type of normalization, the minnor and maxnorm from Eq. (1) are the minimum and maximum intensities taken directly from the histogram.

1%-99%—for such normalization C=arg (cumulative histogram=1%) and maxnorm=arg(cumulative histogram=99%).

This type of normalization should be useful, for example, for range-uniform intensity distributions with limited numbers of outliers. Such outliers are very often caused by dead pixels in imaging sensors or spurious noise.

±3σ−the range of intensities for this normalization can be defined as

minnorm=μ−3σ

and

maxnorm=μ+3σ

where μ is the mean intensity and σ is the standard deviation of the image intensities in the ROI. Such normalization is useful when the intensity histogram of the texture is close to a Gaussian distribution.

For cases where the imaged sample does not occupy the entire image, the digital program or the user includes an option to automatically detect the area of the tissue, thus avoiding image processing or normalization of irrelevant areas of the image. This feature is especially important when the relative size of ROI changes dramatically. The automated detection option is based on principal component analysis (PCA) followed by either the application of a Gaussian mixture Expectation Maximization (E.M.) algorithm or K-mechanisms clustering to detect pixels that belong to ROI. This step minimizes possible normalization artifacts due to borders across the image and accounts for changes in tissue size across the image.

One of the barriers to large-scale adoption of AI in diagnosis and assistance in therapeutic procedures is lack of standard measurements due to variation in x-ray systems and imaging methods by the same manufacturer or different manufacturers.

The x-ray imaging method and apparatus, based on the methods in this disclosure for normalization, calibration, correlation between x-ray systems, scatter removed to less than 1% Scatter to primary ratio (SPR), in some cases, and as well as 3D tomography and/or spectral imaging in point 1D, 2D to 3D and to 6 D in time domain, may generate a standardized imaging systems across multiple x-ray imaging systems. The measurement of selected sample standards correlate the x-ray system used to measure ROI in a subject to an x-ray imaging system standards. Images generated by the method and apparatus in this disclosure may be used to train AI algorithms, especially an AI method including the use of density, time and other critical quantitative measurements in addition to visual parameters such as shape and pattern, to identify, characterize, monitor and track and select a region of interest or a subject for diagnosis, inspection, image guided a surgery or a medical procedures, and/or delivery of therapeutic treatments. Artificial intelligence based on the x-ray imaging may be used more widely, adopting the disclosed set of standardization methods.

For cases where the imaged sample does not occupy the entire image, the digital program or the user includes an option to automatically detect the area of the tissue, thus avoid image processing or normalization of irrelevant areas of the image. This feature is especially important when the relative size of ROI changes dramatically.

For example, the automated detection option is based on principal component analysis (PCA) followed by either the application of a Gaussian mixture Expectation Maximization (E.M.) algorithm or K-mechanisms clustering to detect pixels that belong to ROI. This step minimizes possible normalization artifacts due to borders across the image and accounts for changes in tissue size across the image.

Pre and Post Image Acquisition Processing—Noise Removal

To remove noise of the image coming from the detector, for example, measurement of dark noise, or white image or gain calibration, the flat field, detector consistency may be done before each measurement or throughout procedures similar to actual measurements in terms of sequencing and timing and VOI selected.

Alternatively, based on the imaging methods, for example in dual or multiple energy imaging, image tracking or tomography imaging, different sets of images or noise measurements may be taken and recorded prior to actual measurements of the imaged subject. Noise removal processing can select more sets of noise measurements and/or correction algorithms without the subject for corresponding set of measurements of imaged subject. The software can compute and remove noise for each measurement.

For example, in spectral 3D images, multiple energy x-ray images are taken of the imaged subject at multiple locations. The total number of measurements at each energy level may be hundreds or thousands, with noise measured at each energy level with multiple images. In some cases, hundreds or thousands of measurements may be taken and algorithms are applied to eliminate or reduce noise under the same imaging condition prior to the measurement of the imaged subject. During the image processing, measurements of the imaged subject are processed at corresponding noise correction value pertaining to each energy level to remove the noise for the measurements done of the region of interest of the imaged subject.

A database may need to be established for quantitatively correlating different x-ray systems, or x-ray system of the same type, and/or x-ray systems of the same configurations, but with varied components from different manufacturers, relating to each other based on the measurements of the same or similar phantom or phantoms or known samples.

A database of x-ray systems may include detailed listing of different hardware elements in an x-ray system, correlating actual measurements of one or more designated phantoms or samples with each other. Data processing algorithms, methods, and procedures for Preprocessing and/or Post processing and during processing data algorithms and methods, may be performed at appropriate times so that quantitative measurement and derivations may be achieved for training and comparison of results of different x-ray systems due to the specified imaging procedures.

Scatter Removal, the Use of Beam Particle Stopper Array

Currently, two exposures are required to acquire high resolution primary images using either beam particle stopper array or collimator selected primary image method to remove scatter.

The “beam particle stopper” plate may also be referred to “beam particle stopper” plate or beam stop particle plate. The “beam particle stopper” or also called “beam particle stopper”, attenuates primary x-ray at distributed locations so that the signals measured on the detector, will have, correspondingly, distributed regions capturing only scatter x-ray.

The selected shape and size of beam particle stopper may allow the attenuation of x-ray to be approximately consistent from all directions, from example, as the x-ray source moves into different locations in a x, y plane for tomography imaging, or while there are two or more x-rays illuminating the subject from multiple spatial locations relative to the subject and/or the detector. For example, a spherical shape or the ball shape may be used so that there is always a center axis or volume going through the ball which attenuates the x-ray the most and attenuates approximately the same level x-rays.

Such a beam particle stopper may also be designed of materials which enables attenuation of at least two or more energy levels, for example, at 99.99%.

And the collected the shadow of the beam stopper particle relative to the x-ray emitting position may be used to derive the location of the center axis of the x-ray tube or the cone beam emitted by the x-ray source.

beam particle stopperAs shown in FIG. 29, a detection module may include multiple elements, for example, detectors 29 downstream from the subject, 2, and upstream of the beam particle stopper plate 100, and another detector 22 downstream to the beam particle stopper plate 100. Detector 29 may be collecting low energy x-rays or may be of higher resolution compared to detector 22. And multiple locations or distributed location or regions of 29 and 22 may be quantitatively correlated by x-ray measurements on the first detector 29 and the second detector 22. The quantitative relationship may be established by spatial x-ray projection path on detector 29 and detector 22, and measurements of substances of known density and composition of single or multiple material types and each material with certain density and thickness at single energy, or dual energy or multiple energy levels of x-ray beam.

This design allows for not only scatter removal but also high resolution of primary x-ray measurements on both front detector 29 and rear detector 22, as illustrated in FIG. 9.

For example, x-ray measurements on the rear detector with the beam particle stopper allows for low resolution scatter measurements on the rear detector at distributed location (l,m), interpolation of low resolution scatter measurements gives rise to high resolution scatter signal on the rear detector, which gives rise to high resolution primary measurement on the rear detector after the substraction of the high resolution rear detector scatter image from the composition image on the rear detector (excluding beam particle stopper shadow area)

FIG. 9 illustrates an example of scatter removal apparatus and methods of dual or multiple detector configuration and beam particle stopper array with one time exposure to remove scatter to less than 1% SPR or less than 5% SPR.

FIG. 9 illustrates an example of a high resolution front detector method. In between the subject 2 and the beam particle stopper plate, or beam particle stopper plate 100, or beam absorber plate, there is an x-ray translucent table for the subject. In between the translucent table and the plate 100, a detector, 29 is placed. The detector 29 has for example, higher resolution pixel elements or higher frame rate measurement, or may be a photon counting sensor or may be energy sensitive detector. Or in case of a dual detector assembly, with collimator in the middle layer, the detector 29 may be directly stacked on top of the rear detector 22. Rear detector 22 may have energy sensitive measurement or may be a photon counting detector, or may be a low resolution detector. If the quantitative relationship between the primary signal on selected region of such detector 29, and the primary signal on the corresponding location on detector 22, are derived, low resolution scatter image on the detector 29 can be obtained by subtracting a composite signal of the primary signal plus scatter signal at the selected regions on 29 with the calculated primary signal on detector 29 at these regions. The high-resolution scatter can then be obtained by interpolation of the low-resolution scatter on detector 29. And the final high-resolution primary signal on detector 29 can then be obtained by subtracting high-resolution scatter signal of detector 29 from the high-resolution composite signal measured on detector 29 which contains both primary and scattered signal. Detector 29 may be small in form factor compared to detector 22. Detector 29 may be moved into a location by a mover such as a linear actuator or two-axis actuator 210 on a plane parallel to detector 22 to capture x-ray passing through a region of interest on the subject after a first image is taken by detector 22.

Detector 22 may be placed on a translation stage, or a mechanical fixture, movable by a one axis or a two axis translation stage or multiple axis stage, or actuator, which may move longitudinally to move detector 22 and sometimes, plate 100, or in some instances, dual detector and collimator assembly with detector 22 as the front detector moves away from the subject 2. The distance between the detector 22 to plate 100 may be sub inch. And there may or may not be a spacer between the plate 100 and detector 29 or between detector 29 and detector 22 for the dual detector assembly designed for scatter removal.

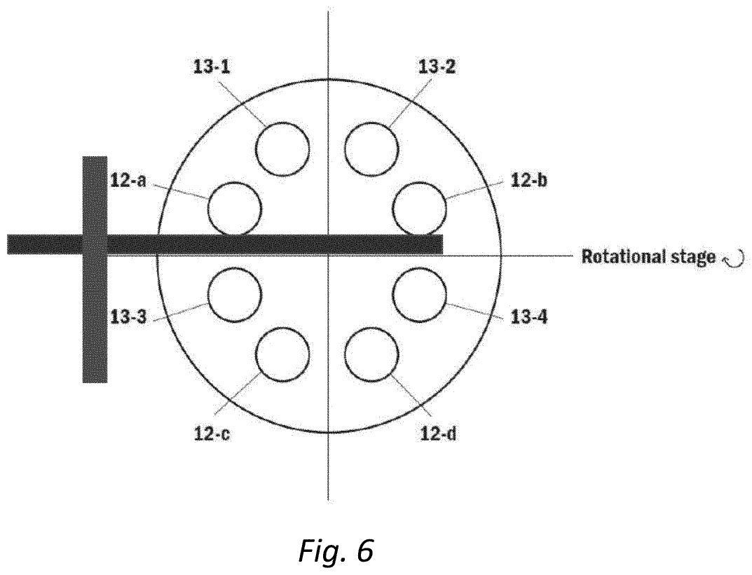

As seen in FIG. 6, in some instances, additional one or more x-ray source and detector pairs, source 13 and its corresponding detector 27 may be placed in the same plane as that of source 12 and detector 22 pair, and x-ray source 13 may be placed up to 90 degrees from the center axis of the original x-ray source 12 and detector 22 pair to view the VOI from an oblique angle, for example, to derive thickness of the VOI or to have a different perspective view of what is internal to VOI, for example, to view soft tissue only region without bone or a metal object in the projection path.. X-ray source 13 illuminates the subject and may pass through a beam particle stopper plate 100-2 and reaches detector 27.

Such a setup can increase the speed of image acquisition or give another geometric information of the subject or increase accessibility of certain region of interest in the subject. As such additional x-ray source and detector may have different or same parameter values as the source 12 and detector 22 pair, such as in resolution, image acquisition speed, focal spot size, mobility, form factor, or spectral wavelength or energy level. The setup can include additional hardware pieces or additional x-ray optics or two or more combination of all of the above.

Alternatively, there may be two or more detectors placed on various angle relative to each other, sharing one x-ray source.

Data acquired by one detector guide the data acquisition process and method of the second or other detectors and vice versa. Alternatively, summation of x-ray measurements and image sets from all detectors are required for data analysis.

This is different from x-ray tomography configuration of prior art where different detectors are used for multiple dimensional image reconstruction by providing projection images at varied angled and combined images from multiple locations to reconstruct one single tomography image. In the present disclosure, each x-ray source is capable of x-ray tomography measurements, with multiple sources at different spatial locations to combine measurements, especially spectral measurements to ensure fast acquisition, or for measurements of multiple dimensions. Multiple x-ray sources may be moved at the same time to increase the speed of tomography, for example, by combining the number of measurements needed. The difference from tradition tomography or tomosynthesis methods is that the x-ray source and detector pair center axis of one x-ray source or emitting position are preferably close to those of another, for example, by less than 10 or 5 or 4 or 3 or 2 or 1 degree, relative to the ROI and center axis of x-ray source and detector.

Another difference is in the use of beam particle stopper plate 100, when image data gaps exist due to the region of the subject illuminated by the x-ray beams, which are attenuated by the x-ray beam particle stopper. Having another detector or another set of x-ray source and detector at a different angle capture the data information missing by looking at it at a different angle, at least some if not all of the missing data can be retrieved, especially for example, in scanning for presence at a location of a specific component, for example, diseased tissue or contrast labeled tumor or stem cell. If some small amount of area is missing, the second set of data may be acquired of the region contributing to the missing information at the same time thereby reducing system motion requirement and increasing data type flexibility and the likelihood of additional type of information to be obtained.

FIGS. 41-45 illustrate examples of beam particle stopper plate with beam particle stoppers in distributed region. Beam particle stoppers are materials which may attenuates x-rays which are embedded or placed on a plate or in a plate of x-ray transmissive material, which may be of rigid structure to place the beam particle stopper in one position when x-ray measurement is done.

Such as plate can be moved mechanically, electrically or electromagnetically or magnetically, or driven by an energy source to move in 1D or 2D and 3D dimensions so that beam particle stoppers may be in different positions in space.

Each beam particle stopper may move within the plate, such as being driven mechanically or electrically or magnetically or electromagnetically by motors or electromagnetic steerers so that x-ray measurements may be taken with each beam particle stopper at varied locations in space to block the x-ray beam.

Beam particle stopper may be used to block the beam for scatter removal, or it may be used to filter x-ray beam energy or it may be used to reduce radiation as it may be placed between the subject and the source, or between the subject and the detector. Other optics and x-ray optics may be placed any where in the beam path to further manipulate, or steer or filter x-ray or optical light converted from the x-ray.

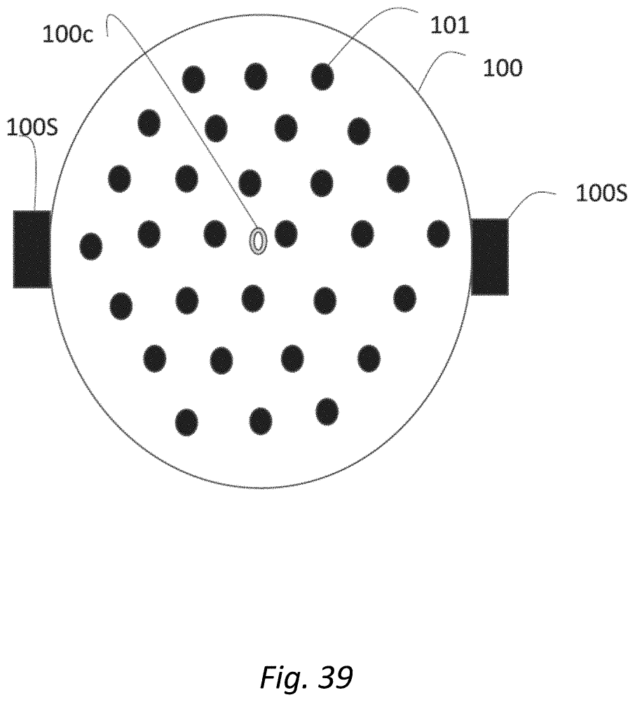

FIG. 39 illustrates a beam particle stopper design including a Rotating Disk with one or two rotating motors or moving gears. 100s is the rotation gear to move the beam particle stopper plate, and 100c is the center of the rotation.

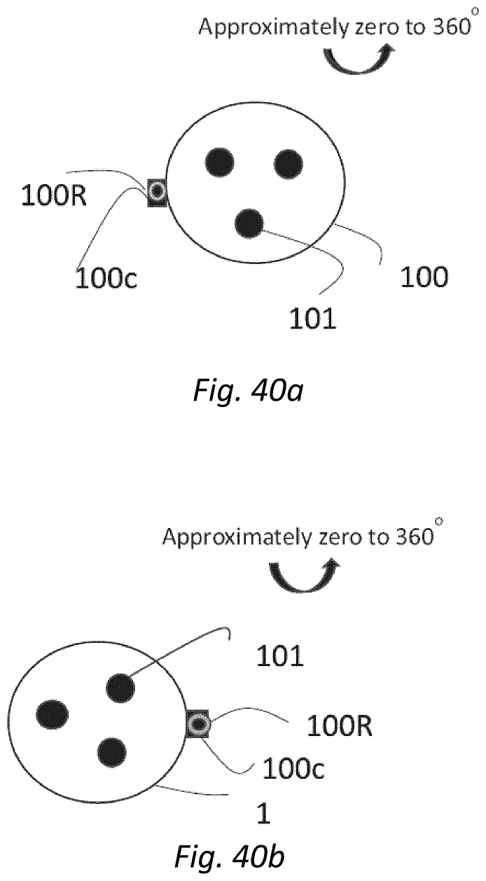

FIG. 40 illustrates an example of moving method of a beam particle stopper motor design including a Rotating Disk with one spinning motors or moving gears. 100 R is the moving rotating gear to spin the beam particle stopper plate. 100 c is the center of rotation. The beam particle stopper plate 100 may be spinned around the center at 100R at approximately 0-360 degrees.

With continued reference to FIG. 9, (i′,j′) on the front detector 29, corresponds to (i,j) on the rear detector given the illumination path of an x-ray emitting position. (i,j) are distributed locations, which may be between 100 to 10,000 locations distributed on both rear detector 22 and front detector 29. The beam particle stopper may be 0.5mm to 10 mm in size. The x-ray measurements on the rear detector 22 may remove the measurements due to the front detector 29 and the beam particle stopper plate 100 to give rise to the primary x-rays measurements on all regions except at the shadow regions (l,m) corresponding to each of the beam particle stoppers on the rear detector. (i,j) are selected from regions outside of the beam particle stopper shadow on the rear detector 22. As soon as the quantitative relationship is established between each (i,j) on the rear detector 22 and its corresponding (I′,j′) on the front detector 29, the primary x-ray measurements at (i′,j′) can be derived from primary x-ray signal (i,j) from the rear detector. The C composite measurement at (i′ j′) can be derived from the direct measurement on the front detector 29.

Cfl(I′, j′)−Pfl(I′j′)=Sfl(I′,j′) (1)

which is a low resolution scatter signal on the front detector 29. Interpolation of low scatter signal on the front detector 29 gives rise to high resolution scatter image. C composite measurement minus the high resolution scatter image on detector 29 gives rise to high resolution primary x-ray image on the front detector 29. Therefore in one x-ray illumination, two high resolution primary measurements at the front detector 29 and rear detector 22 are taken and derived at the same time.

A low resolution densitometer may be created by using x-ray thin beam generated by rotating the anode target or using optical collimator which is downstream from the source 12 but upstream of the imaged object 2, which has one or more leaves, each including complete or partial transmissive regions. Each leaf may have various distributed transmissive regions, of a certain density. Thereby x-ray thin beams are generated and modulated instead of using the beam particle stopper plate. Certain leaf may restrict resolution, and radiation level and selected region of interest.

Such features may be enabled by specific anode designs.

X-ray thin beam may also be used to calibrate the system and remove scatter as described in the aforementioned PCT Applications. Such system may work with one or more layers of detecting mechanisms, such as stacked detectors for relevant measurements including, for example, at different energy levels or different resolution or different imaging speeds.

Various scatter removal apparatus and software may be used. For example, implementations with front and back detector, with a beam selector sandwiched in between. Examples of such configurations are described in U.S. Pat. Nos. 5,648,997, 5,771,269, and 6,052,433 and PCTs international patent application No. including PCT/US2019/044226, PCT/US2019/014391, PCT/US2019/022820, the entirety of each of which is hereby incorporated by reference herein and should be considered a part of the disclosure. Such designs may combine with various x-ray sources configurations. In additional, additional x-ray assemblies with separate x-ray source and detector may be used where x-ray detector may be moved to image selected region of interest after a first image or first set of images are taken by the dual detector assembly.

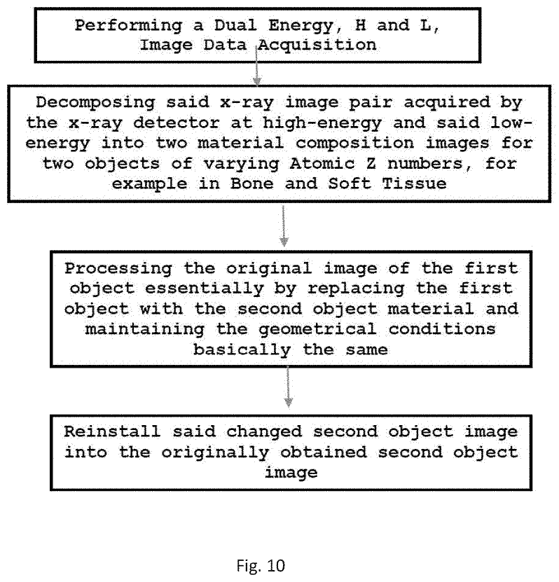

In one example of the present system, obtaining the missing imaging data in the beam path illuminating the subject, which is attenuated the beam particle stoppers in the beam particle plate 100, the plate containing the beam particle stopper plate is moved after each image is taken. For example, if after each image taken at one energy level, the plate is moved in the xy plane, another image is taken at a different energy level. As a result, data gap may be obtained by x-ray measurements at energy level different than the first one. Alternatively two images can be taken at the same energy level, each at a different beam particle stopper plate position on the xy plane, each position may be different from or located distant from the other by approximately dimensions of one beam particle stop, or some times larger. FIG. 10 illustrates a flow diagram of an example data replacement method.

Data Gaps in Beam particle stopper Plate or Beam Particle Stopper Plate or Beam Particle absorber plate based tomography method

In some tracking and density measurement applications, data gap may be ignored.

In high-resolution diagnostic applications, the measurement can be repeated at a different beam particle stopper position after the x-ray source is moved relative to the beam particle stopper plate, or vice versa.

Real time Scatter removal can use Beam particle stopper Plate for imaging with one detector downstream the beam particle stopper, measured with one image at one x-ray emitting position and one beam particle stopper plate location, or real time scatter removal with two detectors can measure at the same time once for each x-ray emitting position while beam particle stopper sandwiched in the middle, quantitative analysis, tomography and spectral imaging, may allow low radiation and fast imaging acquisition at resolution higher or equivalent to CT. In some cases, resolution required may be less than CT scanner offers. The present disclosure may adjust the resolution target so that radiation can be minimized in the process of imaging due to either selecting limited number of measured locations or reducing number of images needed to reconstruct and/or track an ROI or a component and/or a substance in the ROI.

The following method and apparatus may be used:

After the first image of the ROI is acquired using one xx-ray source and its corresponding detector, the second x-ray source may illuminate a collimator which blocks x-ray from reaching most regions of the subject, especially x-ray from regions of the subject being blocked by the beam particles in the beam particle stopper plate . For instance, each of such a beam particle stopper or may be 0.1 mm to 10 mm in dimension. The x-ray coming from the first source may not generate projected signal from a volumetric region or multiple volumetric regions within the subject illuminated by the x-ray due to the beam particle stopper plate. The second x-ray source illuminates such volumes V, and due to the emitting position designed to be different from that of the first x-ray, x-ray from the second source illuminates VOI where the missing data gap caused by beam particle stoppers and may now reach the detector and be collected.

An alternative configuration would be moving the beam particle block plate to a different position or a second position than the first position, which does not overlap with the first position, after the first image generated by the first source is taken, so that the missing data gap can be provided by a second image of the first source at the second position.

In some instances, beam particle stopper plate is moved to a reference position, or a home position, then it is moved to a first position. After an image is taken, it is moved to a second position, and a second image is taken.

In some cases, the first image of a certain exposure time plus second image of certain exposure time may provide a complete image with enough exposure needed to visualize or quantitative measure for further image processing.

Beam particle stopper plate 100 may be designed to have approximately equal dimension transparent region and opaque region for x-ray coming from the source. As the plate moves by approximate the dimension of each transparent region or each opaque region, the complete data set is acquired. The plate 100 may be placed in between the source or in between the subject and detector.

In order to derive the complete multiple dimension image, the subject is relative to the first source by a distance or in least 2D or more dimensions, so that when the first image generated by the first x-ray source, passing through the subject, is taken, some image data gaps may exist due to the beam particle stoppers in the plate 100. As the subject moves, additional image, or at least additionally one more image or x-ray measurement is taken to fill in the data gap with measurements in order to complete the data set required for a 2D or for construction of multiple dimension or 3D images of the subject 2.

At least two x-ray sources may be used, as illustrated in FIGS. 1-4. Sometimes, each source may move in and out of an emitting position which is able to illuminate region of interest and the projected signal is captured by a first detector. Such sources may be of multiple energy sources or single energy source, or quasi monochromatic source. Such sources may be of different energy levels. For example, the first one source may be at 40-150 KeV, and the second source may be at 20-40 Key.

The mover to move the x-ray sources in and out of the emitting position or emitting positions may be a rotating turret or a linear stage, or two dimensional stage or three or more dimensional stage, a rotating moving stage. Alternatively, such x-ray sources are modulated to move in and out an emitting position by steering the electron beam, for example, via electron beam deflection, by, for instance, a set of electro-optic lens, in some cases, by electromagnetic or magnetic methods such as using magnetic plates or solenoid coil.

In FIGS. 5-8, in the examples illustrated, the x-ray imaging system and apparatus disclosed here can include more than one detector, sometimes referred to as second detector or detectors, downstream or upstream of a first detector relative to the source or the volume of interest or the imaged subject.

Additionally, with continued reference to FIG. 9, a second or third or fourth detector may be moved in and out of the emitting position where the source may illuminate the region of interest. Measurements may be taken based on the application need. Such detectors may be mounted on a stage, manual or motorized, may be rotated to reach each quadrant downstream or upstream the first detector 22. In each quadrant, the detector or detectors may be moved with a linear, or 2D or multiple dimensional translation stage within the quadrant. Such detectors may be without scatter removal devices, or may be used with a beam particle stopper plate 100 downstream from VOI or upstream of the VOL Such detectors may be moved into the position of the first detector after the first detector is moved out of illumination path of the VOL In some cases, when the second or third detectors are used, each detector may include a beam selector sandwiched between two detectors as described in the aforementioned PCT Applications.

Scatter Removal for Spectral X-ray Measurement

Scatter removal for quantitative spectral imaging or tomography may be done when there is less than 5% SPR or less than 1% SPR.

Each x-ray measurement may go through image processing methods using a scatter removal process such as “time of flight” using a time domain method. For instance, ultrafast source or picosecond source is used to capture primary x-rays within different time windows, or primary modulator-based scatter removal in frequency domain is used, or the scatter removal may be based on selective spatial measurements of scatter only or primary x-rays only measurements that involve a beam particle stopper plate, or beam particle stopper array plate or beam selector, respectively.

Beam particle stopper or Beam particle stopper Array or Beam Particle Stopper Plate (BPSP) or Beam Stopper Particle Plate, all refer to a hardware plate with distributed x-ray attenuating elements embedded in an x-ray transmissive plate which is generally a light and rigid structure plate including, for example, a polymer material. In some cases, such element or a portion of such an element may attenuate x-ray almost completely, typically at one or multiple x-ray spectrum energies, by mixing multiple different and/or same attenuating materials capable of attenuating x-ray at multiple energies, and/or by having sufficient thickness. The attenuating properties may be adjusted by using a modulator to orient each element such as in a MEM device, or may be tunable by using a modulator such as ultrasound or crystal or a tunable grating system. Each element and/or the BPSP may be moved by a mechanical or motorized device between data acquisition so that the missing data gap caused by the attenuation of the primary x-ray may be recovered and/or extracted from another acquisition event at the same x-ray emitting position and/or at a different x-ray emitting position at the same or different position of the BPSP. Recovery of the data gap may also be accomplished through acquisition of an x-ray image at a different energy level at a different BPSP location.

For some applications, for tomography or a near complete tomography using BPSP for x-ray scatter removal to less than 1% or less than 5% SPR x-ray attenuation may only be measured once at one x-ray emitting location and at one position of the BPSP. The total number of projection 2D images that need to be acquired to reconstruct a complete 3D image may be denoted by Tj. The missing data may be complemented by interpolation or extraction of measured data at other BPSP positions. The total number of projection 2D images that need to be acquired to reconstruct a complete 3D image with no or little missing data may be approximately >2Tj. The missing data due to use of the BPSP may be complemented by moving BPSP to a different position where x-ray is attenuated at a different location of the projection image on the detector at the same x-ray emitting location, or by moving BPSP to a different position as well as moving the x-ray emitting location. In the latter cases, the total number of x-ray measurements may be increased for tomography but typically no more than 2×Tj, which is equal to the total measurements at each BPSP position for an approximately completely reconstructed tomography image. For example, if there are 4 possible different positions of BPSP, at each position, the attenuated primary x-ray in each position does not overlap with any of the other positions. If three x-ray images are taken at 3 of the 4 positions, at each position, there are Tj/3 images taken. In that case, the total number of images that need to be acquired to reconstruct a complete tomography of a VOI, with no or virtually no data gap, is approximately ((4*Tj)/3). The 4th set of projections may be taken with x-ray emitting position travel in the same 2d area that is traveled by the first 3 sets of projection. The 4th set of projections may be taken with x-ray emitting position at a different emitting position than those of the first three sets. This 4th set of projections may be used to resolve the new unknown voxels introduced outside of the ROI as the x-ray emitting position moves in the first three set of projections.

For example, as shown in FIG. 1, subject (also referred to as “object” throughout the disclosure) 2 is downstream from the source or sources, and there may be a sample holder 40 which is supported by a hardware fixture to support the subject 2.

As shown in FIG. 1, there are at least two sources, one source which may be called the first source, generates x-rays, illuminates a subject, and x-rays pass through a plate with distributed regions containing beam particle stoppers or beam particle stoppers or beam particle stopper arrays 100. Each particle may have a shape suitable for a specific application, for example a spherical or a ball shape (such as shown in FIGS. 41-44), suitable for the attenuation of x-rays coming from multiple directions and the non-attenuated x-ray beam reaches the detector 22 and then gets collected and registered by the detector 22.

Currently beam stopper plate may have attenuation regions of a different geometry, such as a disk, than what is presented in this disclosure. Current beam stopper array was specifically designed for relatively fixed geometry between x-ray source, beam stopper array and/or detector. However, such design is not suitable or preferred for the tomography method, because as the x-ray emitting position moves, the attenuation values for each portion of the disk may change for the primary x-ray passing through the disk with resulting non-uniformity of primary x-ray attenuation. In contrast, in the present disclosure, the preferred shape for each of the beam particle stoppers or beam particle stopper is spherical, so that when the x-ray emitting position moves, there is always a region where the primary x-ray beam is completely or virtually completely blocked. At the same time, the size and shape of the ball allows minimization of regions of blockage of primary x-ray at each attenuation position. This ensures that there are sufficient transmitted x-ray passing through without attenuation by the Beam particle stopper elements.

The present disclosure also describes a method where the beam particle stopper may be moved in order to recover or extract a missing data gap from image data acquired at other BPSP positions, as shown in FIG. 40.

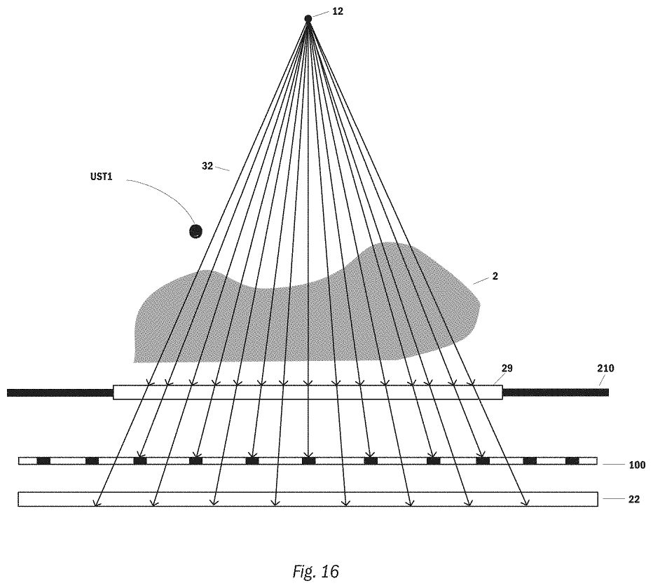

There may be one detector stacked on top of BPSP plate, as described in FIGS. 9 and 16, with or without a mover. Such a configuration allows derivation of a high resolution 2D image without a missing data gap at the highest resolution that the detector is capable of, with <1% SPR or <5% SPR for all spectral measurements, using only images acquired at any one x-ray emitting position with one single exposure.

Such a method minimizes time required for a complete tomography image as a minimal number of exposures are acquired for the reconstruction of a complete tomographic image without or with relatively minor missing data gaps.

In contrast, traditionally, two exposures are required to acquire high resolution primary images using either beam particle stopper array or a collimator-selected primary image method to remove scatter.

In the present disclosure, the beam particle stopper attenuates primary x-ray at distributed locations so that the signals measured on the detector will have corresponding distributed regions capturing only scatter x-ray.

The selected shape and size of beam particle stopper may allow the attenuation of x-rays to be approximately consistent from all directions, for example as the x-ray source moves into different locations in an x, y plane for tomography imaging, or when there are two or more x-rays illuminating the subject from multiple spatial locations relative to the subject and/or the detector. In another example, a spherical or ball shape of beam particle stopper may be used to ensure that there always is a center axis or volume going through the ball that attenuates the x-ray the most and attenuates approximately the same level of x-rays.

Such a beam particle stopper may also be designed of materials that enable attenuation of at least two or more energy levels, for example, at 99.99%.

The collected shadow of the beam stopper particle relative to the x-ray emitting position may be used to derive the location of the center axis of the x-ray tube or the cone beam emitted by the x-ray source.

Spectral Imaging—Establishment of Database for Materials or Units of Measurements such as Voxels or Subunit of Voxels

Measurements of one material or combination of two or more materials using x-rays in one or multiple energies such as a broad spectrum x-ray with one or more energy peaks may be stored in a database.

There is a unique number of thickness and attenuation coefficients of one substance ,or one material, or two or more substances or materials, that correspond to one measurement or a unique combination of values at single, dual or more energies.

This unique number may be further defined as a value of voxel attenuation, which is of single substance, or two or more substances, where the weighted contribution of each substance may be determined by reducing the size of the voxel to even smaller units, for example to 1 um, 100 nm or smaller than 100 nM, and measurements of single or multiple energies of each material of one subunit volume, and combination of two or more subunits, each of one substance.

To gain reasonable accuracy and a unique look-up value of thickness or attenuation, or identification of one or more materials, many combinations of measurements may be needed to establish a functional database.

A unique relationship may be established by plotting one measurement or combinations of measurements at different energy levels of one or more substances. Depending on the number of materials to be measured, or the required resolution, or the energy levels of x-rays to be measured, a plotted spatial relationship which relates unique values of measurements of one material to a unique value of one or a combination of measurements at different energy levels may be established by a number of measurements that is smaller than the number of possible measurements within the range of interest of different types of materials, thicknesses, voxel dimensions and/or subunit dimensions. This is achieved by plotting a unique correlation of attenuation value, subunit measurement value or voxel measurement value of each material and/or combination of two or more materials corresponding to measurements at different energy levels. Such values may be derived or measured directly.

A plot may be interpolated based on a number of measurements, for example, 6 or 8 or 10 or 12 , such as 6×6×6 for a triple energy system or 8×8×8×8 or a multiple energy system, which may be derived so that the predicted value of thickness or voxel attenuation value or subunit attenuation value may be determined as long as a unique one-to-one relationship is established with relative high accuracy. For example, even with a larger number of measurements or measurements set at more possible settings and combination of energy levels, the algorithm used to link the two values will not provide a variant which is different from the predicted value from the plot within an error rate or deviation, or standard deviation of for example, <0.05% or <0.5%.

Even if the algorithms used to link the two values are not derived, such as in a black box, as long as a unique set of values can be derived, as long as the relationship of any single value of measurements of one material corresponding to one or more energy levels is such that one value of measurement corresponds to a unique value of energy levels.

Examples of Spectral Imaging System

The source may be a broad spectrum source, in some instances, with energy peaks at one or more energy levels, or a source with single energy or monochromatic source.

The detector may be a flat panel detector or an energy sensitive detector or dual energy stacked detector or a detector with subunits of pixels which are energy sensitive at different energy levels compared to each adjacent pixel.

Collimator may be used or controlled to restrict or expand field of view of x-ray beam to illuminate region of interest (ROI) or volume of interest (VOI) by the user or computer.

A second or third detector with larger FOV may be placed in front of or downstream of the detector.

Spectral imaging includes those of k-edge, or spectral imaging may be based on dual or multiple energy response function equation system-based material decomposition, in which inverse derivation is based on interpolation and a functional response equation system.

An inverse linear equation system may be established by deriving corresponding substance and material quantitative information based on an energy functional response equation system that was established from prior measurements, at dual or multiple energies corresponding to unique values of density and/or thickness of each material and its composite value.

Scatter Removal at Different X-ray Energies

Scatter is removed using a software program that interpolates the scattered image at selected, distributed locations on the front detector to derive a high-resolution scatter image, which is then subtracted from a composite image on the front detector to generate a high-resolution primary X-ray image.

Single detector-based scatter removal methods may also be possible using a beam particle stopper plate.

Frequency or primary modulator-based scatter removal may also possible.

Time of flight scatter removal using ultrafast x-ray source and detector are also possible.

For quantitative imaging, it is preferred to reduce scatter to 1% of the primary X-ray, or less than 5% of the primary X-ray.

In certain situations, the imaged subject or VOI is not highly scattering, in which case the scatter removal step may be omitted.

Calibration

In traditional CT scanner or general x-ray imaging, calibration may often be used as a term to describe data cleaning up and noise removal, etc. However, in quantitative imaging, the demand for consistent data and precise data is much higher, and often times, the number of processes and algorithms applied pre, during and post imaging acquisition and during image reconstruction, material decomposition, densitometer, fluidic dynamics, precise motion measurements, and quantitative imaging analysis and AI related procedures and measurements are interlaced, and therefore the term calibration may not sufficiently or accurately describe all of the processes involved.

Thereby, the term PCP, preprocessing, and PRP, post processing, are used to describe various steps. These steps may include calibration.

In some cases such as quantitative imaging, quantitative measurements, qualitative measurements, density measurements, spectral imaging or multiple dimensional imaging, or 3D imaging or tomography applications, or 2D imaging, the noise of the detector may be eliminated using known methods, such as known calibration methods based on the disclosure herein. Noise in measurements may include dark current, gain, dark noise, white image, interference of ambient light or x-ray from the environment, optical light, spurious noise, or any interference or noise that may affect quantitative noise and/or flat field. Noise measurements or calculations may be done using software and/or algorithms, and in some cases, it may involve known calibration based on the disclosure herein. In some cases, a filter or x-ray optics or optics or other type of filter involving hardware and/or software used to attenuate or manipulate electromagnetic waves may be used. In some cases, a filter or x-ray optics or optics or other type of filter involving hardware and/or software used to attenuate, manipulate, electromagnetic waves may be used.

In traditional CT or general x-ray imaging, generally noise calibration is done infrequently, or calibration may be done right before a measurement is done. Therefore generally calibration data is saved only once and sometimes override prior calibration.

It is, therefore, part of the present disclosure for calibration to include methods to save calibration data of one or more measurements or one or more set of measurements with the same or different settings in, for example, energy, speed, exposure time, gain values or off-set and the number of frames on a local or remote microprocessor to the detector. During the image processing for image analysis, one or more, or one set or more sets of calibration data are selected to remove noise based on the type of measurement done on a subject. In some cases, calibration methods used in the present disclosure and scatter removal methods, material decomposition method, multiple dimensional imaging methods and apparatus, one or more x-ray optics or optics or hardware, or software may be added or removed, for example, for filtering, for steering, or manipulate the light in space, spectral or frequency or time domain, and may be being used with other methods of imaging or positioning, or measurements, including that of AI for diagnosis, tracking, characterization, monitoring and surveillance, inspection, quantification, or visualization.

Dual Energy Material Decomposition, Triple Energy or more energy spectral imaging and measurements and material decomposition can measure density and characterize fluid dynamic with or without contrast labels for the fluid.

The basic principle is that for a laboratory X-ray or broad band source, the detector measurement of a substance can be unique for a certain density and thickness. Solving mathematically this differential equation is difficult and current mathematical methods have not yielded accurate or satisfactory results. However, there is a unique relationship between substance density or composite substance, and detector measurements at various energy levels. This relationship is based on scatter free measurements of substances with a broadband X-ray source. Thus, instead of trying to solve the differential equation mathematically, a database based on a plot that is called an inverse energy response function equation system can be created to correlate detector measurements at different energy levels with different densities of a substance or substances. Moreover, the number of measurements for each substance or material with two or more substances may be measured with variation in density and thickness. The number of measurements may be <10 or <20 to establish a database for —5000 density variations through use of interpolation. A plot is derived by interpolation such as spline method based on measurements at different energy levels and varied densities of the substance(s) at a fixed or varied thickness. When the number of measurements increases beyond a certain point, the accuracy of the predicted value from the derived plot does not improve significantly and neither does it reduce the 0.5% error rate compared to measured values. Therefore, a limited number of measurements may be needed to establish an accurate database or an energy response function system to correlate substance density with detector measurements at different energy levels. For example, in a triple energy inverse energy function equation establishment, each material or each component may have 5 different measurement samples, each with different thickness and/or density. For example, when there are two different substances, there may be 6×6 different variations and combinations of variation to be measured to establish a plot at two different energy levels, H and L. When there are three different substances, there can be 6×6×6 , or a total of 216 different combinations of variations of thickness and/or density. Each combination may be measured at High energy, H, Medium energy, M, Low energy, L or a total of 216×3=648 different measurements. The database can be described as a plot that is called an inverse energy response function equation system and may be established with the detectors because different detectors have different response functions.

In some cases, the goal may not be to have the most accurate material composition possible at the 2D imaging level. Since layers of two or more tissues are interlaced, complexity of decomposition is far greater than simply determining the thickness or density of the substance. Instead, the goal may be to achieve the best density evaluation and material decomposition possible to separate at least one substance from the rest and determine the smallest VOI required for diagnosis, 3D imaging and/or other quantitative analyses. Due to the fact that tissues are generally slow varying, the VOI can also be selected based on sharp changes that are indicative of abnormality and deviation from the norm or caused by adjacent regions that are made of different tissues.

In some cases, such a plot of energy response function equation system is established to characterize the thickness or density of one or more distinct materials or sub stances.

The quantitative measurement and material decomposition and measurement can be done more accurately by combining measurements with 3D image reconstruction, because each voxel at a particular spatial position may be further characterized by its interaction with various energies. The end result is a more accurate density measurement of the substance in a voxel unit or multiple voxel units either for construction of a database, or for inverse look-up of material density based on detector measurements at various densities or a database or inverse energy response function equation system established based on a few measured results at different energy levels of different materials or combination of materials for various known density and thickness components that need to be interpolated to generate a plot. Such a plot may be extended to correlate detector measurements at dual or more energies with composite materials or individual materials or substances, with unique or distinct density or thickness values of certain material or materials or combinations of dual or more materials.

preprocessing, post processing, and/or data cleanup for 3D reconstruction and spectral imaging and quantitative imaging

2D detector measurements may be taken to correct for fixed-pattern variations in individual pixel responses.

The raw data recorded in DICOM format or as raw image is transferred from the imaging system server to the built-in picture archiving and communication system (PACS), where an automated image processing is performed.

Image processing such as noise removal, scatter removal, normalization of data, material decomposition or derivation of characteristics of the measured material or ROI, or multiple dimension image reconstruction or tracking over time, transfer and establishment of database, linking with other records, AI, correlation with other camera measurements and/or other modality measurements, colocation, quantification, diagnosis, image guidance, may be carried out by one or more microprocessors and/or done at one more locations.

Image processing may also be done at the microprocessor as a part of the detector, or be linked to the detector locally or remotely.

The raw data in DICOM format or raw image may be transferred from local storage or database in a workstation to an image system server and/or to the PACS, where additional automated image processing may be performed.

Image processing may also be done at the image system server.

Controlling, synchronization, external communication and triggering of events and activities internal or external of the hardware and software included in the imaging system may also be done at one or more microprocessors, or one or more controllers locally, in an imaging system server or PACS storage device or an electronic record system or a location dedicated for such a task based on the user's preference.

For example, one type of image processing process may include multiple stages or multiple steps: such as pre- processing or pre-image processing noise clean-up, or normalizing the data to remove exceptional data such as derived data which is out of ROI, or calibration of source input, for example, using an additional detector or a reference point to detect input x-ray intensity before reaching the VOI.

The reconstruction into volumes including transmissive voxels as well attenuating voxels can be performed at one or dual or multiple energy levels.

Decomposition into material volumes can be performed, each including either transmissive voxels or attenuating voxels.

Pre- processing may include identification and/or removal of bad pixels and/or flat field correction, or processing of gain, dark noise, white noise, spurious noise.

When multiple detector cells include various energy counters, attenuation volumes can be generated using overlapping, low-threshold counters. Within each pixel region, there are a set of energy counters. The entire detector can include repeating units of energy counters.

In one example of reconstruction, a low-resolution version of the polychromatic form of the Beer—Lambert Law is used in which the overlapping, low-threshold counters that measure the data are processed simultaneously to produce attenuation volumes representing non-overlapping energy bins across the measured spectrum.

For example, four individual counters representing the energy ranges 30-120 keV, 45-120 keV, 60-120 keV and 78-120 keV respectively would simultaneously produce four attenuation volumes representing the energy ranges 30-45 keV, 45-60 keV, 60-78 keV and 78-120 keV.

For example, counters representing the energy range 30-45 keV, 45-60 keV, 60-78 keV and 78-120 keV would simultaneously produce four attenuation volumes representing the energy ranges 30-45 keV, 45-60 keV, 60-78 keV and 78-120 keV.

In the case where multiple energy peaks exist in one x-ray pulse or one exposure, a detector including repeating units of energy sensitive detectors may be used. In the measurement of one pulse or exposure, multiple energy measurements may be performed for the VOI.

Alternatively, a series of single energy x -ray pulses or exposures at dual or multiple energy levels may be measured by one or more detectors.

For each energy measurement, the unknown voxel value may be set at 1 or 0, where 1 can be the voxel of which the attenuation value is most sensitive to the energy level, and 0 can be assigned to the other voxels.

After attenuation values of the voxel are determined for each unknown voxel in the VOI at various energy levels, the density and/or composition of the voxels may be derived. The derivation process can be a process of elimination, which may identify and eliminate voxels having specific attenuation value range at various energy levels. This process may also identify voxels filled with air or water. Voxels with mixed materials may be separated based on measurements at various energies, which may be at the interface between two tissue types, or at the interface of normal and diseased areas. Using the material decomposition described in this disclosure, a database or inverse linear equation system may be established to derive corresponding substance and material quantitative information relating to derived measurements and the value density and/or thickness of each material.

Segmentation may occur based on the attenuation value, linear coefficient of the voxel using a threshold value or threshold segmentation. Presentation of image data may be processed through CT cutting.

When monochromatic filters are used as repeating units downstream from the x-ray emitting position and upstream of the imaged subject, detecting regions of the detector corresponding to each filter will be able to collect x-ray measurements of the specific energy ranges used. Each repeating unit may include a number of filters, placed next to each other. For example, a filter may be a K edge filter. In some cases, filter regions may be of Coded aperture, which may be K-edge filters but may also be a monochromatic filter that is different from K-edge filter.

In another instance, the detector is a 2D detector, and inverse function response method described above is used. As described, the inverse function response method uses interpolation to establish a unique correspondence of quantitative values of detector measurements with one or more materials at one or more energy levels, material density and thickness and based on a small number of measurements at various density or thickness levels.

The number of measurements needed to establish the plot of detector measurements at material density from interpolated values can be determined by the deviation of the plotted value from actual measurements.

For example, if the deviation is less than 0.5% from a plot produced from a large number of measurements, this plot may be sufficient.

If the number of measurements can produce a reasonably smooth plot, the plot may be sufficient as well.

The reconstruction algorithm itself may be based on a statistical iterative technique. The statistical element is introduced by weighing the corrections to the volume by a normalized Poisson distribution to slow down convergence when approaching the solution, thereby reducing noise.

The reconstruction algorithm may also adopt a multi-stage approach where it initially reconstructs voxels that are eight times larger than requested. In the following steps this is repeatedly subdivided over a total of four stages until the requested voxel size is reached. This approach allows for the reconstruction to proceed relatively quickly. It is also a weak form of sparsity constraint because a large voxel is the same as a set of small voxels with the same value. Lastly, the larger the voxel, the more pixels from the projection images will contribute to the image, which reduces the effect of dead regions. This is particularly useful during the initial reconstruction stages where the impact of dead regions are the most significant.

Preprocessing

Gain, dark field, white noise are adjusted or removed. Dead pixels are noted or interpolated from adjacent pixels. If too many dead pixels are noted, imaging can be stopped. If less than a defined number are noted, the preprocessor can interpolate from adjacent pixel, or note that the pixel is dead. The dead pixel is removed from the data used for calculation.

K-edge coded aperture, or monochromatic filters each used for a different energy level, or repeated units of monochromatic filters, can be K-edge or optimized beforehand for optimized sensitivity for one or more components in the ROI.

Alternatively, multiple pixels can be processed where each is energy-selective, for example, at two or more energies, from 2q, where there are q number of pixels in one pixel region, and where each of the q selectively pixels measures an energy range.

Using scatter removal method with beam particle stopper plate or beam particle stopper, at least one pixel region is blocked on the detector which directly corresponds to one of the beam attenuating blocks. For example, if one pixel q of the pixel region reads only scatter for one energy level or one energy range, then the scatter image or data collected on that pixel q can be interpolated to other pixel regions but only at corresponding position q of the other pixel regions.

If 1-q energy selective filters are used in the filter region to selectively let one energy range to be transmitted to illuminate the subject at each filter position of the filter region, then the primary or scatter image on the detector corresponding to each of the filters in one filter region can be used to derive scattered image in other filter regions, although only in the corresponding filter position for the same energy level.

Spectral imaging for 3 energies, can use a 3D cubic spline as the computation method to establish the energy functional response inverse plot, or alternatively, a 4D cubic spline interpolation can be used. For 4 energies, 4D or 5D cubic spline interpolation can be be used, and so on, to establish the energy functional response inverse plot.

For more accurate imaging and measurements, the following procedures may be performed:

Verification of attenuating regions of beam particle stopper array position prior to taking a measurement.

Verification of scatter images during and after scatter removal process.

Verification of primary image.

If a dead pixel is detected, the dead pixel can either be removed, or be noted for calculations later, or adjacent pixel data can be used to interpolate to the dead pixel. This can be decided on a case-by-case basis.

For various parts of the system, for example, the electronic controller or detector where temperature maintenance and validation is necessary, temperature sensors may be used as the primary indicator or secondary verification system.

One or more reference points for measurements on the detector by itself and/or with a target may be used for verification of each measurement.

Examples of Spectral Imaging - Dual energy material decomposition and extension to multiple energy levels

Apparatus and methods for performing dual-energy and multiple energy x-ray imaging can include using large format, two-dimensional detectors. There are at least two goals for performing dual-energy x-ray imaging. The first is to use the dual-energy imaging method to remove the scatter. In cases where only one detector and beam particle stopper plate or beam particle stopper is used, as illustrated in FIG. 1, this method is not needed.

In another example, x-ray source is that of time of flight , and the scatter and primary x-ray images may be separated in the time domain.

In another example, a primary X-ray modulator downstream of an x-ray source that is upstream of a subject is used to separate scatter x-ray from the primary x-ray in the frequency domain.

The second goal is to determine two or more material composition images of the image subject when the subject includes at least two materials.

There can be several hardware components of the apparatus in physical sequence from front to back. (1) An x-ray source emits x-rays. (2) A front two-dimensional detector array receives primary x-rays and scatter x-rays. (3) A beam selection device blocks the passage of primary x-rays along a number of travel directions, while allowing passage of primary x-rays along other directions. Scatter x-rays are passed generally unaffected. (4) A rear two-dimensional detector assembly receives the scatter x-rays and primary x-rays passed through the beam selection device. Because of the operation of the beam selection device, the rear detector assembly receives only scatter x-rays at a number of detection locations, while at other detection locations, the rear detector assembly receives both primary and scatter x-rays.

A data decomposition method directly solving a dual-energy or multiple energy x-ray imaging equation system without relying on linearization approximations can be used. This method establishes a direct two-way relationship between the dual-energy primary x-ray image pair or multiple energy image set and the material composition image pair or the material composition image set. Based on the dual-energy or multiple energy data decomposition method, when a pair of dual-energy primary images or a set of multiple energy images are given, the material composition images can be automatically computed without user intervention. The dual energy decomposition image processing may be iterative on a subject including three or more material or component. After each dual energy decomposition computation processing step, at least one substance or one material is extracted from the subject image.

A summary of the dual-energy x-ray data decomposition method is provided below, with the multiple energy data decomposition method being an extension of that: The method directly solves the nonlinear dual-energy x-ray imaging fundamental equation system in its original form without relying on any linear or second order approximations. The method includes: (1) Constructing an explicit quantitative equation system D.sub.H=F.sub.DH (b,s) and D.sub.L=F.sub.DL (b,s) for each detector according to the nonlinear dual-energy x-ray imaging fundamental equation system in its original form and saving the constructed equation system for later use, where D.sub.H represents the low-energy primary x-ray signal and D.sub.L represents the high-energy primary x-rays signal. The two equations and all quantities therein are for a typical single detector cell; the entire detector array can be represented by a single detector cell after normalization. (2) Reconstructing a three-dimensional surface equation system b=b(D.sub.H, D.sub.L) and s.times.s(D.sub.H, D.sub.L) by numerically inverting the equation system of step 1 and saving the reconstructed equation system for later use. (3) Determining the desired values for b and s at each individual detector cell location by inserting the available data pair (D.sub.H, D.sub.L) into the numerical equations of step 2 or, conversely, when a pair of b and s values are given, determining the desired values for D.sub.H and D.sub.L at each individual detector cell location by inserting the available data pair (b,s) into the numerical equations of step 1. (4) Maintaining the accuracy at each step to be as high as real number analytical solutions can provide.

In the multiple energy method, the dual energy x-ray data composition method may be iterative, the dual energy x-ray data decomposition method is performed to separate each substance or each material at a time from the rest of the subject. Depending on the application requirement, the iterative process can go on until a number of substances are separated from the rest and individually, and when a sufficient number of substances are obtained, the process may stop, or such decomposition processing may continue until each one of the substances are separated as an individual image or decomposed data describing only one substance.

An example of procedures for performing dual-energy x-ray imaging include the following steps: (1) Acquire a pair of image data for the rear detector assembly (see, for example, FIGS. 9 and 16) at a higher energy level H and a lower energy level L of x-rays. Because of the function of the beam selection device, in the acquired image data, a number of detector cells contain only scatter x-ray signals, while the other detector cells contain a combination of primary x-ray signals and scatter x-ray signals. (2) Derive a pair of dual-energy primary image data for the rear detector assembly from the directly received data of step 1. Only primary x-ray image data can be used for dual-energy x-ray imaging. How the derivation is performed is explained below. (3) Use dual-energy data decomposition method to calculate a low-resolution primary image for the front detector from the dual-energy primary image pair of the rear detector. This is one of the most important aspects of the present disclosure. (4) Acquire a high-resolution image for the front detector (see, for example, FIGS. 9 and 16) either at the higher energy level H or at the lower energy level L according to the need. Because the low-resolution primary x-ray image on the front detector has been calculated using the acquired high-resolution image data together with the calculated low-resolution primary image data, the scatter image as well as the primary image with high spatial resolution can be calculated for the front detector. Upon completion of step 4, the image quality of the front detector would have been improved (the first goal described above) by removing the undesired scatter from the front detector signals. (5) To proceed, a pair of primary x-ray images of the front detector can be acquired at two energy levels L and H instead of only one image as in step 4. By further using the dual-energy data decomposition method, the two material composition images for the image subject at a high spatial resolution can be obtained. Thus, step 5 fulfills the second goal of dual-energy x-ray imaging as described above.

U.S. Pat. No. 5,648,997 and 5,771,269, incorporated by reference herein in their entirety, describe a different structure of the beam selection device. In these patents, the beam selection device blocks scatter x-rays to selected locations of the rear detector.

As described earlier, the beam selection device of the present disclosure blocks primary x-rays to particular locations of the rear detector. Because different signals are allowed to reach the rear detector, a different method for deriving the low-resolution primary x-ray image is used. Included in this disclosure, as described earlier the low-resolution primary x-ray image is acquired directly from the rear detector. The low-resolution primary x-ray image may be calculated from a low-resolution scatter x-ray image and a low-resolution scatter/primary composite x-ray image acquired from the rear detector.

The apparatus and a method for dual-energy or multiple energy x-ray imaging using large format two-dimensional detectors can provide two material composition images of a subject with a spatial resolution as high as the two-dimensional detector array can provide. In addition to enabling spectral imaging, the apparatus and method described herein provide 2D images used to reconstruct tomography images and multiple dimension imaging.

The dual-energy or multiple-energy image data decomposition can be automatically carried out by computer without user intervention.

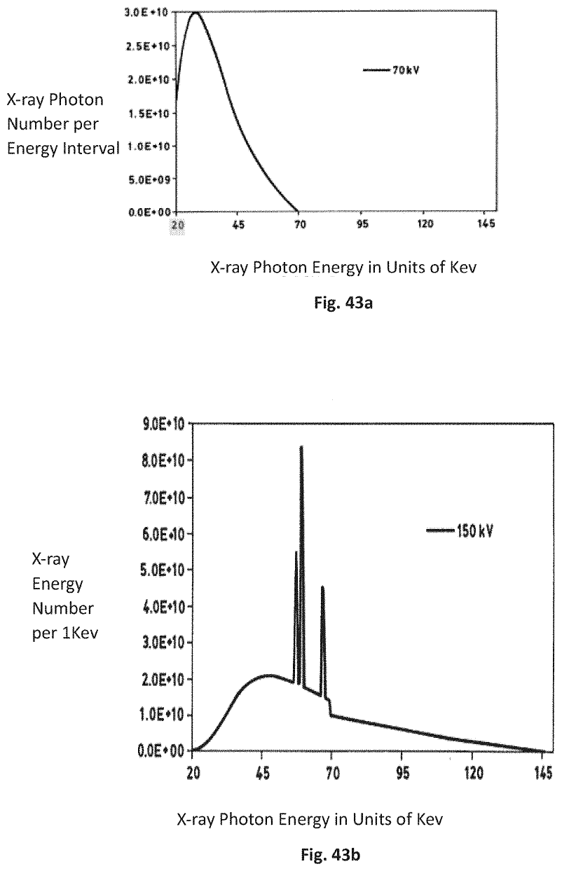

FIGS. 43a and 43b are curves describing typical x-ray source energy spectra at the higher energy level H and lower energy level L used in the present disclosure, where FIG. 43a presents an energy spectrum at a high voltage HV=70 kV and FIG. 43b presents an energy spectrum at a high voltage HV=150 kV.

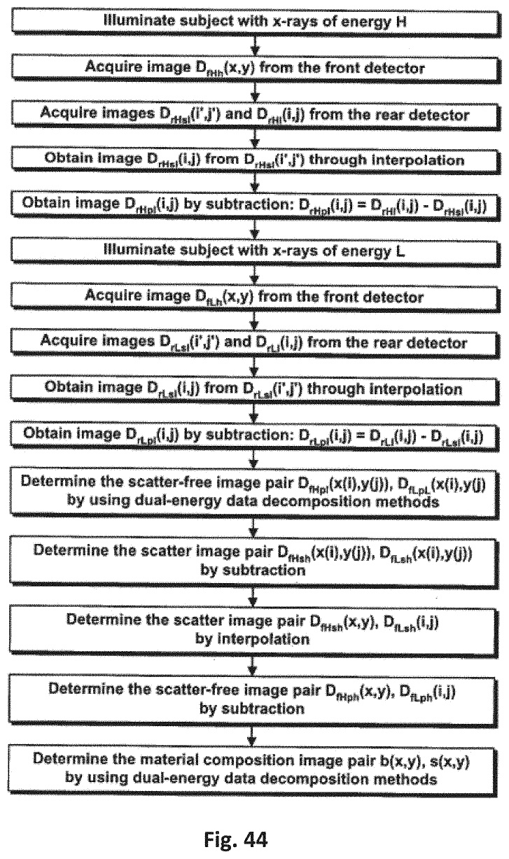

FIG. 44 is a flow diagram of the basic procedures using the dual-energy data decomposition method and the hardware described.

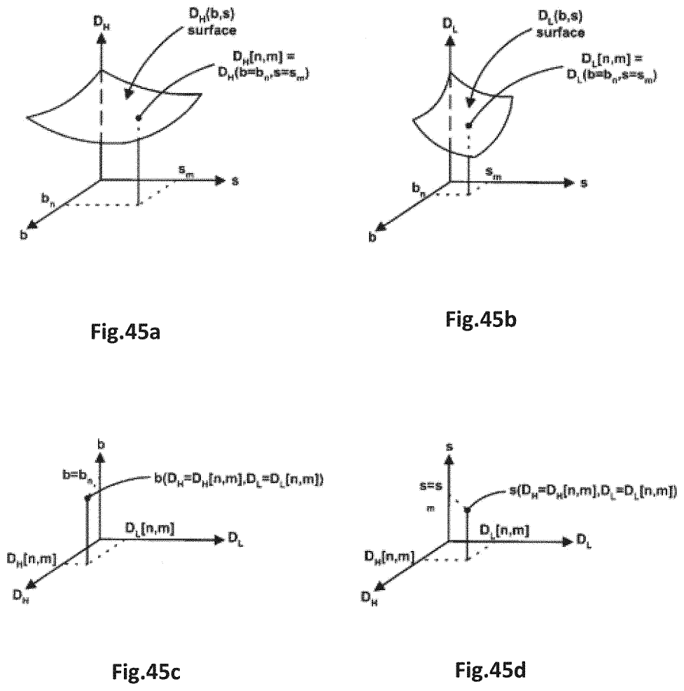

FIGS. 45a to 45d are graphical representations of a method for inverting the nonlinear dual-energy equation systems.

An example of Dual Energy Imaging

The subject under examination is located between the x-ray source and the front detector. The x-ray source emits two consecutive pulses, a high-energy pulse at an average energy level H followed by a low-energy pulse at an average energy level L. In an alternate configuration, the low-energy pulse is emitted first. Preferably, in both configurations, the high-energy pulse has an average x-ray energy from approximately 25 keV to approximately 250 keV and the low-energy pulse has an average x-ray energy from approximately 15 keV to approximately 60 keV, with the high-energy pulse always higher in energy than the low-energy pulse.

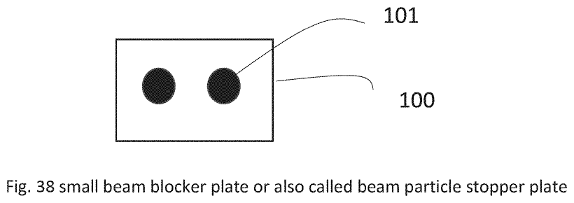

The x-ray source has an energy spectrum covering a broad energy range. In addition to the continuous bremstrahlung spectrum, the energy spectrum may also contain discrete line structure when the high voltage value is high enough, as shown in FIG. 38. Currently, there is no known effective method for providing mono-energetic x-rays for medical imaging. Therefore, all quantitative calculations must be carried out with an x-ray energy that covers a broad energy range. The x-ray source can include a point source, meaning that the x-rays appear to be emanating from a single point rather than from a larger area. A portion of the x-rays passes through the subject directly to the front detector assembly without a change in their direction of propagation. These x-rays are called the primary x-rays and convey true information about the subject . The remainder of the x-rays are randomly scattered as a result of interaction with the material of the subject . These x-rays are called scatter and cause a distortion of the true information.

The front detector contains a large number of individual detector cells in a two-dimensional array. Although the present disclosure is not limited to a particular type of x-ray detector array, there are two basic types. The first uses thin film amorphous silicon as photodetection medium. The amorphous silicon film has a typical thickness of 1 micrometer (.mu.m) and is sensitive to visible light. The electric charge induced by visible photons is collected by an array of electrodes. A scintillation screen, which is the x-ray sensitive medium, is placed in close contact with the entire photosensitive area of the photodetector array. X-rays cause the generation of visible photons in the scintillation screen, which are then detected by the amorphous silicon photodetector array, inducing an electric charge proportional to the x-ray energy absorbed in the screen. This type of x-ray detector array is called an external conversion type x-ray detector. Preferably, the detector array has dimensions of 20 centimeters (cm) by 20 cm or 40 cm by 40 cm for a single detector module. A number of such detector modules can be abutted to provide a larger detector. The cell size for this detector array can be in the range of from approximately 50 .mu.m by 50 .mu.m to approximately 1 mm by 1 mm.

A second type of detector array uses a semiconductor material with a medium high atomic number Z, such as an amorphous selenium film, selenium alloy film, CdZnTe film, or other amorphous or polycrystalline semiconductor films as the x-ray sensitive medium. The charge induced by x-rays directly in the detection medium is collected by an array of electrodes and is proportional to the energy of the x-rays striking the film. The typical thickness of the selenium film is in the range of from approximately 100 .mu.m to approximately 800.mu.m. This type of x-ray detector array is called an internal conversion type x-ray detector. A typical amorphous selenium or selenium alloy detector array module has dimensions of 20 cm by 20 cm or 40 cm by 40 cm with a cell size of from approximately 50 .mu.m by 50 .mu.m to approximately 1 mm by 1 mm. A number of such detector modules can be abutted to create a larger detector array.

Other typical two-dimensional detector arrays include charge-couple device (CCD) detectors, CMOS detectors, thin-film thallium-bromide-based detector arrays, avalanche silicon detector arrays, and phosphor-stimulatable computed radiography screens.

The cells of the front detector assembly have variations in their response characteristics. However, these variations are slight and can be normalized, so it is assumed that after normalization, all detector cells in the detector have the same response characteristics.

The combination of signals from all of the cells conveys an image of the x-ray intensity over the area of the front detector. Because the detector cells cannot distinguish between primary x-rays and scatter, the front detector conveys an image that is a combination of the primary x-rays and the scatter , and is denoted by

D.sub.fh (x,y)=D.sub.fph (x,y)+D.sub.fsh (x,y) (1)

where D.sub.f denotes an image in the front detector and (x,y) denotes the two-dimensional Cartesian coordinates of a cell of the front detector 16. For example, when the front detector 16 has a 1024-cell square matrix, x and y will each have integer values in the range of from 1 to 1024, inclusive. D.sub.fph (x,y) denotes the contribution from the primary x-rays and D.sub.fsh (x,y) denotes the contribution from the scatter.

The cylinders are fabricated such that their axes are aligned with the direction of the travel of the primary x-rays, such that the cylinders are not exactly parallel to each other, but are radial to the x-ray source. As the x-ray source is located farther away from the beam selector, the cylinders approach being parallel to each other. Preferably, the x-ray source is located between 20 cm and 150 cm from the rear surface of the beam selector. The disclosure holds equally true when the x-ray source has a finite size.

There may also be scatter x-rays from the sources other than the image subject, such as, for example, from the wall or floor of the building material. These scatter x-rays are excluded by using conventional methods.

Preferably, the rear detector cells are arranged in a rectangular matrix with from 8 to 1,024 cells on a side, where each cell is identified by the general two-dimensional coordinate (I,J). The image received by the rear detector assembly 22 contains two subsets of data, the first being scatter x-ray signals at the shadowed locations. These locations are identified by (i′,j′). The second subset of data includes a combination of primary and scatter x-rays at the non-shadowed locations. These locations are identified by (i,j).

In the present disclosure, these two data subsets are used to derive a low-resolution primary x-ray image data at the rear detector at selected locations. The procedures for the derivation are described below. The term “selected location” is defined as an array of locations on the rear detector, where, due to the function of the beam selector and to the use of the procedures of the present disclosure, the signals contain only derived primary x-rays.

The rear detector cells at the selected locations have a fixed geometric relation with some of the front detector cells. This relation is established by drawing a selected projection line from the x-ray source through the beam selector 18 to the selected location. This selected projection line intersects the rear detector surface at a rear detector cell at a coordinate (i,j), and intersects the front detector surface at a front detector cell at a coordinate (x(i),y(j)). Here (x(i),y(j)) denotes the coordinate (x,y) of the front detector cell closest to the selected projection line. An image file D.sub.rl (i,j) at the selected locations is a low-resolution image file. The data at the image pixel (i,j) is the data obtained either from a single detector cell or from a combination of a small number of detector cells around the selected projection line. Similarly, D.sub.fl (x(i), y(j)) denotes an image file from the front detector 26 having a low spatial resolution. In this disclosure, the word “resolution” is used only to represent spatial resolution, as opposed to amplitude resolution. The data at the image location (x(i),y(j)) is the data either of a single detector cell or of a combination of a small number of detector cells around the selected projection line. The relationship between (i,j) and (x(i),y(j)) is experimentally established and stored. The image data on the selected projection lines are low-resolution images and are represented by the subscript lower-case 1. The image data from all the front detector cells are high-resolution images and are represented by the subscript lower-case h.

In connection with the material composition of the image subject, four quantities are defined. b(i,j) and s(i,j) are defined as low-resolution images for the selected projection mass densities along the selected projection line (i,j). b(x,y) and s(x,y) are defined as the projection mass densities along the projection line (x,y). The “projection mass density” is defined as the integrated total mass of the image subject along the projection line per unit area. Because the projection mass density is not dependent on the size of detector cells, b(x(i),y(j))=b(ij) and s(x(i),y(j))=s(ij).

The mathematical and physical foundations of the dual-energy imaging procedures of the present disclosure are detailed as follows:

Preferably, following a high-energy x-ray pulse at an average energy level H and a low-energy pulse at an average energy level L, two images of the rear detector are acquired. The coordinates in each of these two images have a general notation (I,J), with 1=1,2,3, . . . N and J=1,2,3, . . . M, where M and N are integers. (I,J) has two subsets, (i,j) and (i′,j′). The data subset at (i′,j′) is the scatter-only x-ray signals identified as D.sub.rHsl (i′,j′) and D.sub.rLsl (i′,j′). The data subset at (i,j) has a combination of primary x-ray signals and scatter x-ray signals identified as D.sub.rHl (i,j) and D.sub.rLl (i,j). The locations (i,j) are selected to uniformly cover the entire image plane of the rear detector and close to the locations (i′,j′). Because images D.sub.rHsl (i′,j′) and D.sub.rLsl (i′,j′) are both scatter-only x-ray signals, they can be extended to the entire image plane of the rear detector by interpolation. The interpolation does not cause nonnegligible error because of the physical nature of the scatter x-rays. The scatter is mostly caused by Compton scattering, which has a substantially uniform angular distribution in the preferred x-ray energy range. Both empirical data and theoretical calculations show that scatter always has a substantially smooth distribution on a two-dimensional image plane. That is, the change in scatter intensity between adjacent cells is small and smooth. Thus, as long as there are a sufficiently large number of data points, the error incurred due to interpolation is negligible in comparison with other error sources, such as statistical fluctuations of x-ray photon numbers. Therefore, scatter-only signals at the selected location (i,j) are obtained by interpolation, and identified as D.sub.rHsl (i,j), and D.sub.rLsl (i,j). Accordingly, a pair of primary image signals D.sub.rHpl (i,j) and D.sub.rLpl (i,j) can be calculated as

D.sub.rHpl(ij)=D.sub.rHl(i,j)-D.sub.rHsl(i,j) (2a)

D.sub.rLpl(ij)=D.sub.rLl(i,j)-D.sub.rLsl(i,j) (2b)

where D.sub.rHl (ij) and D.sub.rLpl (i,j) are the directly acquired data and D.sub.rHsl (ij) and D.sub.rLsl (ij) are the interpolated data.

The next step is to calculate the primary images at the front detector from the primary image pair D.sub.rHpl (i,j), and D.sub.rLpl (i,j). The high-resolution image D.sub.fHh (x,y) is acquired from the front detector 29 following the high-energy x-ray pulse at an average energy level H. The high-resolution image D.sub.fLh (x,y) is acquired from the front detector following the low-energy x-ray pulse at an average energy level L. The high-resolution image pair of the front detector can be written as ##EQU1##. On the other hand, the low-resolution primary images of the rear detector derived from equation pair 2a, 2b can be written as ##EQU2## where the .PHI..sub.0H (E) and .PHI..sub.0L (E) are the energy spectra of the x-ray source at the higher energy level H and at the lower energy level L. The projection mass density b(i,j) and s(i,j) of the subject 12 are in units of grams/centimeter.sup.2 (g/cm.sup.2). .mu..sub.b (E) is the mass attenuation coefficient of bone tissue and .mu..sub.s (E) is the mass attenuation coefficient of soft tissue, with both .mu..sub.b (E) and .mu..sub.s (E) expressed in units of centimeter.sup.2 /gram (cm.sup.2 /g). Both of these values are known, having been determined experimentally and tabulated previously. The term [.PHI..sub.0 (E).times.exp(-(.mu..sub.b (E).times.b(x,y)+.mu..sub.s (E).times.s(x,y))] is the energy spectrum of the primary x-rays incident on the front detector after passing through the subject, where expo 0 denotes the value e raised to the power specified in the parenthesis. S.sub.f (E) is the x-ray spectral sensitivity (the electrical signal amplitude from the detector as a function of the number of x-rays with energy E after the x-rays passing through the image subject) of the front detector. S.sub.f (E) includes not only the x-ray spectral sensitivity of the detector itself, but also the x-ray transmission factor that accounts for the absorption of x-rays between the subject and the front detector. Such absorption is due, for example, to the front detector protective case material. The term .intg..PHI..sub.s (E).times.S.sub.f (E)dE represents the signal caused by scatter. The exact expression for the scatter is not known because the scattering process is too complicated to model accurately. The coordinate (x,y) corresponds to a front detector cell.

In the equation pair 2a, 2b, the low-resolution dual-energy image pair includes primary signals and is free of scatter distortion. By using the dual-energy data decomposition methods outlined above and described below, the simultaneous equation pair 2a, 2b is solved to find the solutions for the image pair of material composition b(i,j) and s(i,j). Because of the data decomposition method, solving the highly evolved equation system 2a and 2b can be performed by a computer software operation to produce a pair of b(i,j) and s(i,j) values as output for a given data pair D.sub.rHpl (i,j), D.sub.rLpl (i,j) as input.

As described above, because the rear detector cell (i,j) and front detector cell (x(i),y(j)) lie on the same selected projection line, the low-resolution front detector primary image pair D.sub.fHpl (x(i),y(j)), D.sub.fLpl (x(i),y(j)) can be further determined from the rear detector primary image pair D.sub.rHpl (i,j), D.sub.rLpl (i,j) by applying the data decomposition method again. Moreover, the front detector scatter image pair D.subl.fHsl(x(i),y(j)), D.sub.fLsl(x(i),y(j)) is found by the equations

D.sub.fHsl(x(i),y(j))=D.sub.fHl(x(i),y(j))D.sub.fHpl(x(i),y(j)) (3a) and

D.sub.fLsl(x(i),y(j))=D.sub.fLl(x(i),y(j))D.sub.fLpl(x(i),y(j)) (3b)

The next step includes interpolating the values for the low-resolution scatter image D.sub.fHsl (x(i),y(j)) and D.subfLs1 (x(i),y(j)) to include those detector cells that are not on selected projection lines, yielding two high-resolution scatter images D.sub.fHsh (x,y) and D.sub.fLsh (x,y). The interpolation does not cause loss of accuracy because of the nature of the physical scattering process, as described above. While the scatter image can be interpolated because of the nature of scatter, the primary image cannot be interpolated because the primary image changes with the subject 12 from detector cell to detector cell.

Continuing, the high-resolution primary images on the front detector are denoted as D.sub.fHph (x,y) and D.sub.fLph (x,y) and are

D.sub.fHph(x,y)=D.sub.fHh(x,y)-D.sub.fHph(x,y) (4a)

and

D.sub.fLph(x,y)=D.sub.fLh(x,y)-D.sub.fLsh(x,y) (4b)

The image pair D.sub.fHph (x,y), D.sub.fLph (x,y) is a pair of dual-energy x-ray images without scatter. This image pair in turn relates to the material composition of the subject by the equations ##EQU3##.

Unlike the simultaneous equation system 3a, 3b, the simultaneous equation system 4a, 4b has only primary x-ray signals, free of scatter distortion. This equation pair is the fundamental dual-energy x-ray imaging equation system with the unprecedented feature that scatter radiation has been essentially removed from the two-dimensional detector. In the equation pair 4a, 4b, the values of D.sub.fHph (x,y) and D.sub.fLph (x,y) are known from the above-described calculations conducted on the image pair D.sub.fHh (x,y), D.sub.fLh (x,y) directly measured from the front detector, and on the images D.sub.rHsl (i′,j′), D.sub.rLsl (i′,j′), D.sub.rHl (i,j), and D.sub.rLl (i,j) directly measured from the rear detector. The unknown values are the two material composition images b(x,y) and s(x,y).

The dual-energy x-ray data decomposition method can be further applied to the equation pair 4a, 4b. As a result, by using the quantitative relationships b=b (D.sub.H, D.sub.L) and s=s(D.sub.H, D.sub.L) provided by the data decomposition method, a pair of high-resolution images b(x,y) and s(x,y) can be obtained point by point for all front detector cells (x,y). The solution of the two-component material composition images b(x,y) and s(x,y) has a spatial resolution as high as the front detector 29 can provide.

Alternatively, an x-ray source having a switching high-voltage power supply can be used. The switching high-voltage x-ray source generates x-rays continuously, alternating between low-energy x-rays and high-energy x-rays. The switching high-voltage x-ray source can be treated as a repetitive double-pulse x-ray source.

Lo et al. and other journal articles were published regarding use of a beam stop method to reduce scatter effects. Lo et al. used a beam stop array sandwiched between two stimulated phosphor screens to acquire a scatter-only image for the rear screen. There is certain similarity in the detector geometry to the present disclosure, but the similarity is only superficial. One difference between the present disclosure and Lo et al. are as follows.

(1) Lo et al. uses a single energy method. The scatter-only image acquired at a single x-ray energy spectrum at the rear detector is multiplied by a constant, and then the product image is used as the scatter image of the front detector. Thus, the method of Lo et al. is different from the present disclosure. According to the mathematical and physical theory of the present disclosure, as described above, because the x-ray energy spectra have a broad energy distribution, no functional relationship exists between a single image of the front detector and a single image of the rear detector without knowledge of the unknown image subject. When the unknown image subject is included in the calculations, the usefulness of the relationships is very limited. So far, in conventional x-ray imaging, including the articles by Lo et al., there is no method that can establish such a functional relationship without a dependence on the unknown image subjects. The present disclosure establishes a well-defined functional relationship between the images of the front detector and the images of the rear detector through a pair of dual-energy primary x-ray signals. This situation can be expressed in the following formulas 8a-8f First,

D.sub.fp(x(i),y(j)).noteq.constant.times.D.sub.rp(i,j) (8a)

Any attempt to obtain the primary image of the front detector by multiplying the rear detector image by a calibration constant will not result in a true primary x-ray image of the front detector. The same is true for the scatter x-ray image, that is

D.sub.fs(x(i),y(j)).noteq.constant.times.D.sub.rs(i,j) (8b)

Second,

D.sub.fp(x(i),y(j)).noteq.F(D.sub.rp(i,j)) (8c)

where F represents any defined functional relationship.

Any attempt to obtain the primary image of the front detector by applying any mathematical operation on the image of the rear detector will not result in a true primary x-ray image of the front detector. The same is true for the scatter image, that is

D.sub.fs(x(i),y(j)).noteq.F(D.sub.rs(i,j)) (8d)

The only relationship that the fundamental physical law allows to establish in the first embodiment hardware system is in the following forms:

D.sub.fHp(x(i),y(j))=D.sub.fHp(D.sub.rH(i),D.sub.rL (i,j)) (8e)

D.sub.fLp(x(i),y(j))=D.sub.fLp(D.sub.rH(i),D.sub.rL (i,j)) (8f)

In other words, if a dual-energy x-ray imaging is performed, the low-energy primary x-ray image of the front detector has an accurate, rigorous, and unique relationship with the primary image pair of the rear detector (8e). The same is true for the high-energy primary image of the front detector (8f). These relationships are independent of the image subject, and hence can be established through calibrations when there is no image subject present. These relationships are universally true to the entire image on a pixel-by-pixel basis. The data decomposition method of the present disclosure is the method for quantitatively establishing these relationships.

(2) Because of the differences in the theory and in the method between Lo et al. and the present disclosure, the hardware is also different. One difference in the hardware is that, in the preferred example described above, the x-ray source is a dual-energy x-ray source, whereas in Lo et al., only a single energy x-ray source is used.

Another Example of Dual Energy Decomposition

Alternatively, while keeping the front detector and beam selection device the same, the x-ray source can emit a single-energy spectrum when illuminating the subject. In addition, the rear detector assembly is constructed as a dual-energy x-ray imaging detector assembly. The rear detector assembly can have a low-energy two-dimensional detector, an x-ray energy spectral filter, and a high-energy two-dimensional detector. The filter can operate in the conventional manner based on the disclosure herein. the filter can have a transmission function of exp(-.mu.(E).times.d), where E is the energy of the x-rays, .mu.(E) is the mass attenuation coefficient of the filter material, and d is the thickness of the filter. Because the absorption of x-rays is dependent upon the energy of the x-rays (the mass attenuation coefficient is a function of E), the filter absorbs more of the low-energy x-rays than high-energy x-rays. Thus, the proportion of high-energy x-rays to low-energy x-rays after the filter is larger than before the filter and the average normalized x-ray energy after the filter is larger than before the filter. Preferably, the low-energy x-rays have an average energy of from 10 keV to 100 keV and high-energy x-rays have an average energy of from 30 keV to 500 keV, with the high-energy x-rays having a higher energy than the low-energy x-rays.

Following x-ray illumination, two images of the rear detector can be acquired. The coordinates in each of these two images have a general notation (I,J), with I=1,2,3, . . . N and J=1,2,3, . . . M, where M and N are integers. (I,J) has two subsets of locations, (i,j) and (i′,j′). The data set at locations (i′,j′) is scatter-only x-ray signals identified as D.sub.rHsl (i′,j′) and D.sub.rLsl (i′,j′). The data set at locations (i,j) has a combination of primary x-ray signals and scatter x-ray signals identified as D.sub.rHl (ij) and D.sub.rLl (ij). The locations (i,j) are selected to uniformly cover the entire image plane of the rear detector and to be physically close to the locations (i′,j′). Because images D.sub.rHsl (i′,j′), D.sub.rLsl (i′,j′) include only scatter x-ray signals, they can be extended to the entire image plane of the rear detector by interpolation. The interpolation does not cause nonnegligible error, as explained above. Therefore, scatter-only signals at the selected location (i,j) are obtained by interpolation and identified as D.sub.rHsl (ij), D.sub.rLsl (i,j). Accordingly, a pair of primary image signals D.sub.rHpl (i,j), D.sub.rLpl (i,j) can be calculated:

D.sub.rHpl(i,j)=D.sub.rHl(i,j)-D.sub.rHsl (i,j) (9a)

D.sub.rLpl(i,j)=D.sub.rLl(i,j)-D.sub.rLsl (i,j) (9b)

where D.sub.rHl (i,j) and D.sub.rLl (i,j) are the directly acquired data at (i,j) and D.sub.rHsl (ij) and D.sub.rLsl (i,j) are the scatter data interpolated from subset (i′,j′).

The next step can be to calculate the primary images at the front detector from the primary image pair D.sub.rHpl (i,i), D.sub.rLpl (i,j). The high-resolution image of the front detector can be written as: ##EQU4##. .PHI..sub.s (E).times.S.sub.f (E)dE represents the signal caused by scatter.

The rear detector assembly can have two detectors, so there are two low-resolution primary images D.sub.rHpl (i,j) and D.sub.rLpl (i,j) as derived in (9a) and (9b), which are ##EQU5##. As described above, S.sub.rH (E) and S.sub.rL (E) include the x-ray transmission factors that account for the absorption of x-rays between the subject 12 and the respective rear detectors 22. Such absorption for S.sub.rH (E) is due, for example, to the front detector assembly, the spectral filter, the rear detector protective case, and the rear low-energy detector.

Equations 9a and 9b constitute a simultaneous equation system, where the values for the signal pair D.sub.rHpl (i,j), D.sub.rLpl (i,j) are known quantities. The energy dependent functions .PHI..sub.0 (E).times.S.sub.rH (E) and .PHI..sub.0 (E).times.S.sub.rL (E) are not directly known but can be determined in a calibration process. The data decomposition method described below provides a way to determine these quantities in advance of image operations. b(i,j) and s(i,j) are the unknown quantities for which equation pair 9a, 9b can be solved, as described below.

Accurate b(i,j) and s(i,j) are calculated by the data decomposition method of the present disclosure, as will be described below. Now that the values for b(i,j) and s(i,j) are known, the front low-resolution scatter-free image D.sub.fpl (x,y) can be obtained for those front detector cells (x(i),y(j)) that are on the selected projection lines by ##EQU6##, where the energy dependent function .PHI..sub.0 (E).times.S.sub.f (E) is given in calibration, as will be described in the data decomposition section below.

Next, the low-resolution front scatter image D.sub.fsl (x(i),y(j)) is determined by applying equation 1,

D.sub.fsl(x(i),(j))=D.sub.fl(x(i),y(j))-D.sub.fpl(x(i),y(j))

Because of the physical nature of scatter, as described above, the low-resolution scatter image D.sub.fsl (x(i),y(j)) can be extended to the entire (x,y) plane through interpolation without losing accuracy, yielding the high-resolution scatter image D.sub.fsh (x,y), which is then subtracted from the experimentally measured image D.sub.fh (x,y), yielding the high-resolution primary image D.sub.fph (x,y). The dual-energy imaging can be performed for the purpose of improving the image quality of the front detector and removing the scatter from the front detector image.

Data Decomposition Method

The following is a step-by-step description of the data decomposition method summarized above.

The first step is to construct the two simultaneous numerical surface equations D.sub.H=F.sub.DH (b,s) and D.sub.L=F.sub.DL (b,s) in three-dimensional space. A preferred method to do this is to determine the detection system energy-dependent functions and use these functions to calculate the numerical arrays for D.sub.H and D.sub.L.

There is a difference between equation pair 9a, 9b and equation pair 2a, 2b. If a unified notation is used, the two pairs have the same form. The system energy-dependent function of a detector, denoted sps(E), is defined as

sps(E)=.PHI..sub.0 (E).times.S(E) (13)

where .PHI..sub.0 (E) is the x-ray energy spectrum emitted from the x-ray source 12 and S(E) is the energy response function of the detector. The equation pair 4a, 4b becomes

sps.sub.H(E)=.PHI..sub.0H(E).times.S.sub.f(E) (14a)

and

sps.sub.L(E)=.PHI..sub.0H(E).times.S.sub.f(E) (14b)

and in the second embodiment, the equation pair 11a, 11b becomes

sps.sub.H(E)=.PHI..sub.0(E).times.S.sub.fH(E) (15a)

and

sps.sub.L(E)=.PHI..sub.0 (E).times.S.sub.fL(E) (15b)

The function sps(E) contains the complete energy-dependent features of the dual-energy imaging system. One advantage of determining sps(E) is that all subsequent data processing methods are made independent of the subject 2.

A preferred method for determining the energy-dependent function sps(E) of the imaging system is to use the well-established absorption method. An absorption curve is measured by using a collimated narrow primary x-ray beam. An absorption plate composed of a known material, such as aluminum, Lucite.®., or copper, is placed between the x-ray source and the detector. The electrical signal from a single detector cell D(t) as a function of the absorption plate thickness t is experimentally determined and is related to sps(E) through the equation

D(t)=.intg.sps(E).times.exp(−.mu.(E).times.t)dE (16)

Since the mass attenuation coefficient .mu.(E) of the absorption plate material is known, the function sps(E) can be determined to the accuracy required by the dual-energy x-ray imaging. This method is especially convenient for the internal conversion type of two-dimensional x-ray detectors. In these detectors, the detection efficiency and detector energy response function can be expressed in a simple analytical expression with few unknown parameters to be solved. The energy response function for internal conversion type of detectors is written as ##EQU7## where S.sub.0 (E)=[1-exp(−.mu..sub.0 (E).times.d)].times..alpha.E is the electrical signal amplitude induced by x-ray photons with energy E, .mu..sub.0 (E) is the mass attenuation coefficient of the detector's conversion layer, d is the thickness of the conversion layer of the detector cell, and where S.sub.1 (E)=exp(−.mu..sub.1 (E).times.d.sub.1−.mu..sub.2 (E).times.d.sub.2)is the x-ray transmission after leaving the image subject to the detector surface, .mu..sub.1 (E) and .mu..sub.2 (E) are the attenuation coefficients of two given materials, and d.sub.1 and d.sub.2 are the thickness values of these materials.

When the x-ray energy spectrum .PHI.. sub.0 (E) is separately measured, these unknown parameters .alpha., d, d.sub.1, and d.sub.2 are determined by using standard least square parameter-fitting techniques through equation 16. Then, the energy-dependent function sps(E) is obtained to a high degree of accuracy for a single cell. After normalization, the energy dependent function sps(E) of one cell represents that of all the cells of the same detector.

Once the value for sps(E) is determined to the desired accuracy, the dual-energy signals as a function of the material composition of the subject are calculated through the equations

D.sub.H=.intg.sps.sub.H(E).times.exp(−(.mu..sub.b(E).times.b+.mu..sub.s(E).times.s))dE (18a)

and

D.sub.L=.intg.sps.sub.L(E).times.exp(−(.mu..sub.b(E).times.b+.mu..sub.s(E).times.s))dE (18b)

where .mu..sub.b (E) and .mu..sub.s (E) are the well-documented mass attenuation coefficients for bone tissue and soft tissue, respectively. The mass surface densities b and s are assigned values that sufficiently cover the real range of the subject.

Another preferred method for constructing the quantitative explicit functions D.sub.H=F.sub.DH (b,s) and D.sub.L=F.sub.DL (b,s) is to conduct direct measurements of signals D.sub.H and D.sub.L at a number of selected b and s values. The number of data points for b and s is in the range of approximately 5 to approximately 30. The more data points that are used, the higher the accuracy of the results. However, the number of data points is limited by the acceptable amount of work. The entire functions D.sub.H=F.sub.DH (b,s) and D.sub.L=F.sub.DL (b,s) are obtained from the directly measured data points by using standard two-dimensional interpolation algorithms. After interpolation, there are from approximately 50 to approximately 50,000 data points for b and s. The interpolation in this case is valid because the functions D.sub.H=F.sub.DH (b,s) and D.sub.L=F.sub.DL (b,s) are continuous, smooth, and monotonous.

The second step is to determine the material composition images b and s as functions of the image pair D.sub.H, D.sub.L. The procedures for obtaining a simultaneous equation system for b (D.sub.H, D.sub.L) and s(D.sub.H, D.sub.L) are shown graphically in FIGS. 45a to 45d. To do so, the simultaneous equation pair D.sub.H=F.sub.DH (b,s) and D.sub.L=F.sub.DL (b,s) must be inverted. A preferred method of inversion is as follows: (1) as in FIGS. 45a and 45b, assign a pair of values in the desired range to b and s corresponding to one of the coordinate points in the (b,s) plane so that b=bn, and s=s.sub.m, where n=0,1,2, . . . , N, and m=0,1,2, . . . , M. Typical N and M values are in the range of between approximately 50 and approximately 5,000. The larger N and M, the higher the accuracy of the results. However, the largest values for N and M are limited by the available capacity of computer memory and computing speed. From the two numerical equations representing the three-dimensional surfaces F.sub.DL (b,s) and F.sub.DH (b,s), determine a pair of D.sub.H and D.sub.L values so that D.sub.H [n,m]=D.sub.H (b=b.sub.n, s=s.sub.m) and D.sub.L [n,m]=D.sub.L (b=b.sub.n, s=s.sub.m), where D.sub.H [n,m] and D.sub.L [n,m] are two specific real numbers, and (2) as in FIGS. 45c and 45d, replot the four numbers D.sub.H [n,m], D.sub.L [n,m], b.sub.n, and s.sub.m to provide a pair of data points on the three-dimensional surfaces b(D.sub.H, D.sub.L) and s(D.sub.H, D.sub.L). The data point on the three-dimensional surface b(D.sub.H, D.sub.L) is D.sub.H=D.sub.H [n,m], D.sub.L=D.sub.L [n,m], b=b.sub.n, and the data point on the three-dimensional surface s(D.sub.H, D.sub.L) is D.sub.H=D.sub.H [n,m], D.sub.L=D.sub.L [n,m], s=s.sub.m. After going through all the b=b.sub.n values (b.sub.0,b.sub.1,b.sub.2, . . . ,b.sub.N) and all the s=s.sub.m values (s.sub.0, s.sub.1,s.sub.2, . . . ,s.sub.M), one part of the inversion task is complete. However, for the purpose of storing the inverted arrays b=b(D.sub.H, D.sub.L) and s=s(D.sub.H, D.sub.L), the step sizes of D.sub.H=D.sub.H [n,m] and D.sub.L=D.sub.L [n,m] may need to be adjusted. In the inverted space, D.sub.H and D.sub.L are basis coordinates. From the N.times.M data points, only J data points are selected for D.sub.H and only K data points are selected for D.sub.L, where J and K are approximately in the same range as N and M. In the final form after the second step, two two-dimensional arrays are obtained and stored: b=b(D.sub.H, D.sub.L) and s=s(D.sub.H, D.sub.L), where D.sub.H=D.sub.H [j], D.sub.L=D.sub.L [k]; j=0,1,2, . . . , J, D.sub.H [j]>D.sub.H [j+1] and k=0,1,2, . . . , K, D.sub.L [k]>D.sub.L [k+1]. Two additional one-dimensional arrays D.sub.H [j] and D.sub.L [k] are also stored. Arrays D.sub.H [j] and D.sub.L [k] can be saved so that accuracy as high as real number calculations can provide is maintained.

The theoretical foundation for the numerical inversion process will now be described. It can be generally proven, using mathematics and physics arguments, that under reasonable dual-energy imaging conditions, a unique solution that corresponds to true physical reality always exists. A feature used for the mathematical proof includes the fact that each equation in the dual-energy fundamental equation system in its original form is continuous, continuous for their derivatives up to any high order, and uniformly monotonous with respect to both variables b and s. Because of the uniqueness of the solution, the above inversion process is meaningful and gives a correct solution.

The third step is to find the desired results from the input data according to the established equations. The desired values for b and s at each cell location is determined by inserting the available data pair (D.sub.H, D.sub.L) into the numerical equations of step 2. Conversely, the desired values for D.sub.H, D.sub.L, or only one of them if only one is needed, at each discrete cell location is determined by inserting the available data pair (b,s) into the numerical equations of step 1.

The fourth step is to maintain the accuracy of the values for b and s in order to maintain a continuous domain function. The accuracy of the calculations is maintained at a level as high as the result that would be given by real number analytical calculations. Because of the digital nature of computers, the data arrays stored in computers have finite steps, which are assumed here to have integer values as indices of the real number arrays. The following procedures ensure elimination of the errors in connection with these finite steps in data processing.

In step 1, in the process of constructing the equation pair for D.sub.H [n,m]=D.sub.H (b=b.sub.n, s=s.sub.m) and D.sub.L [n,m]=D.sub.L (b=b.sub.n, s=s.sub.m), for each pair of values of b.sub.n and s.sub.m, the D.sub.H [n,m] and D.sub.L [n,m] are measured or calculated to an accuracy of real numbers. D.sub.H [n,m] and D.sub.L [n,m] are stored in computer as real number arrays.

In step 2, the inversion process, including replotting in D.sub.H space and D.sub.L space, introduces no errors due to the data processing. The step sizes can be changed without losing any accuracy as long as values for D.sub.H=D.sub.H [j] are selected that are exactly equal to one of the D.sub.H [n,m] values that satisfies the condition D.sub.H [j−1]>D.sub.H [j]>D.sub.H [j+1], and values for D.sub.L=D.sub.L [k] are selected that are exactly equal to one of the D.sub.L [n,m] values that satisfies the condition D.sub.L [k−1]>D.sub.L [k]>D.sub.L [k+1].

In step 3, for each measured dual-energy signal data pair (D.sub.HEX, D.sub.LEX), the closest j and k values are found out according to the criteria: D.sub.H [j].gtoreq.D.sub.HEX .gtoreq.D.sub.H [j+l] and D.sub.L [k].gtoreq.D.sub.LEX.gtoreq.D.sub.L [k+1]. From the index values j and k, the closest b and s are first determined as b.sub.0=b.sub.0 (D.sub.H [j], D.sub.L [k]) and s.sub.0=s.sub.0 (D.sub.H [j], D.sub.L [k]). The following equations give b and s values an accuracy as high as real number calculations can provide: ##EQU8##, where the values for the higher order terms can be found in standard calculus textbooks.

Also in step 3, if the image pair D.sub.L and D.sub.H from a given material composition data pair (b.sub.ex,s.sub.ex) is to be found, D.sub.H and D.sub.L are obtained to an accuracy of real numbers by using similar standard Taylor expressions.

Thus, the procedures described above provide methods for directly solving the nonlinear dual-energy x-ray imaging fundamental equation systems in its original form with reasonably selected x-ray energy spectra at an accuracy as high as using real number analytical calculations can provide.

The following is a list of contemplated variations from the examples described above. The x-ray system and method disclosed herein can include any one or more of the following variations and any other variations based on the disclosure herein.

(1) According to current theory, in terms of interaction with x-rays, a broad range of image subjects with a material composition at low to medium atomic numbers can be decomposed into a broad range of two materials with different mass attenuation coefficients. For example, the soft tissue of human body can be decomposed into lean tissue and fat tissue by using dual-energy x-ray imaging methods.

(2) The entire process of constructing the (D.sub.H, D.sub.L) pair as functions of (b,s) may be carried out using a functional scale or grid steps other than a linear scale, such as a logarithmic scale.

(3) Some well-established computation tools, such as sorting algorithms or database procedures, can be used to carry out the inversion process described above.

(4) In the procedures described above, in some cases, conventional or current dual-energy x-ray data decomposition methods can also be used for obtaining the low-resolution front detector image D.sub.fpl or image pair D.sub.fHpl and D.sub.fLpl. These methods can be characterized as solving the nonlinear fundamental dual-energy x-ray equation systems through a linearization approximation method with corrections for beam hardening effects. The correction includes second-order approximations. However, in doing so, the results will be limited by the accuracy and capability inherent to these approximation methods used in the process.

(5) One or more, including all, of the steps described above, including the data decomposition method and the scatter elimination method, can be combined together to various degrees, from combining any two steps to combining all the steps into one procedure. For example, a four-equation system can be established for calculating (D.sub.fHp, D.sub.fLp) from (D.sub.rH, D.sub.rL) without explicitly determining (b,s). One way of doing so is to construct a pair of quantitative relationships D.sub.fHp=(D.sub.rH, D.sub.rL) and D.sub.fLp=(D.sub.rH, D.sub.rL) in a data base and storing the relationships. From the measured data pair (D.sub.rH, D.sub.rL) of the rear detector assembly, a new data pair (D.sub.fHp, D.sub.fLp) of the front detector assembly can be directly found.

The foregoing descriptions of the preferred examples of the disclosure have been presented for the purpose of illustration and description. They are not intended to be exhaustive or to limit the disclosure to the precise form disclosed. Many modifications and variations are possible in light of the above teaching.

A dual-energy or multiple x-ray imaging system for taking two-dimensional images of a subjectcan include:

(a) in physical sequence from front to back, an x-ray source, a front two-dimensional x-ray detector, a beam selection device, and a rear two-dimensional x-ray detector, said subject being located between said x-ray source and said front detector;

(b) said x-ray source being adapted to emit x-rays with two or more different energy spectra for passage through said subject, said x-rays including primary x-rays having their direction of travel unaltered by interaction with said subject and said x-rays including scatter x-rays having their direction of travel altered by interaction with said subj ect;

(c) said front detector receiving said primary x-rays and said scatter x-rays;

(d) said rear detector receiving those of said x-rays passed by said beam selection mechanisms and having a plurality of selected locations and a plurality of shadowed locations; and

(e) said beam selection device preventing passage of said primary x-rays to said shadowed locations, allowing passage of said scatter x-rays to said shadowed locations, and allowing passage of said primary x-rays and said scatter x-rays to said selected locations.

The x-ray source may alternately emit x-ray pulses of said two different energy spectra.

In the x-ray imaging system, the said beam selection device can include an array of cylinders having axes, said cylinders being composed of an x-ray-absorbent material and being supported by a material having negligible x-ray absorption characteristics, said axes being parallel to the direction of travel of said primary x-rays.

In the x-ray imaging system, the thickness of said beam selection device can be between approximately 0.5 mm and 5 cm.

in the x-ray imaging system the said cylinders can have a diameter of between approximately 1.0 mm and approximately 10 mm, and a pitch of between approximately 2 mm and approximately 50 mm.

In the x-ray imaging system, the said rear detector assembly can include a rear detector array having a plurality of x-ray-sensitive detector cells arranged in a substantially square or rectangular matrix with from approximately a number of detector cells on a side.

A dual-energy x-ray imaging system for taking two-dimensional images of a subject can include:

(a) in physical sequence from front to back, an x-ray source, a two-dimensional x-ray detector, a beam particle stopper device,

(b) said x-ray source being adapted to emit x-rays of a single energy spectrum for passage through said subject, said x-rays including primary x-rays having their direction of travel unaltered by interaction with said subject and said x-rays including scatter x-rays having their direction of travel altered by interaction with said subject;

(e) said beam particle stopper device preventing passage of said primary x-rays to said shadowed locations, allowing passage of said scatter x-rays to said shadowed locations, and allowing passage of said primary x-rays and said scatter x-rays to said selected locations; and

The energy spectrum can have an average energy in the range of from approximately 15 keV to approximately 250 keV.

The front detector array can include a plurality of x-ray-sensitive detector cells arranged in a substantially square or rectangular matrix with from approximately 2 to approximately 16,384 detector cells on a side.

The beam particle stopper device can include an array of elements composed of an x-ray-absorbent material and being supported by a material having negligible x-ray absorption characteristics.

The x beam particle stopper device can include an array of elements composed of an x-ray-absorbent material and being supported by a material having negligible x-ray absorption characteristics, and in some instances, configured to seal and position the beam absorbing material in position.

The x beam particle stopper device can be movable by a mover.

The x beam particle stopper device can bemovable by a mover, and the beam particle stopper device may have a homing position or reference position, one or more positions, such as position A or B or C, etc., where it can be moved to.

In some instances, at position A where the beam particle stopper absorbs the primary x-ray may be different from that of position B or position C.

The scattered image collected on in the shadow of the x-ray absorbing material or element when the beam particle stopper plate is at Position A or Position B may be interpolated to derive a high resolution scatter image. This image may be subtracted from the image acquired from the detector including both Primary and Scatter image to derive a primary only x-ray image. The image acquired in position A and B may be combined while the x-ray emitting position is in the same position, to derive a high resolution primary x-ray image without data gap produced by the beam particle stopper. Additional images such when the beam particle stopper at position C, D may be added.

In some instances, the exposure may be reduced significantly at each position of beam particle stopper so that the combined image of that of Position A or B or C etc. has the total exposure needed for sufficient signal on the detector.

The thickness of said beam selection or beam particle stopper device can be between approximately 0.5 mm and 5 cm.

The x beam particle stopper elements of absorbing material can have a diameter of between approximately 0.1 mm and approximately 10 mm and a pitch of between approximately 2 mm and approximately 50 mm.

the present disclosure can include a method for performing dual-energy x-ray imaging of a subject using an imaging system having two-dimensional x-ray detectors, said subject being composed substantially of at least two materials, M.sub.A and M.sub.B, that interact differently with x-rays, said material M.sub.A having a two-dimensional projection mass density A and said material M.sub.B having a two-dimensional projection mass density B. Said imaging system can include in physical sequence from front to back, a dual-energy or multiple energy x-ray source, a beam particle stopper plate with a plurality of beam absorbing material in distributed regions of the plate parallel to a two-dimensional x-ray detector having a plurality of detection locations identified by the notation (x,y), a beam selection device, and a plurality of shadowed rear detection locations identified by the notation (i′,j′). Said selected rear detection locations and said shadowed rear detection locations can be mutually exclusive. Said subject can be between said x-ray source and said front detector, said x-ray source being adapted to emit x-rays at at least two different average energy levels, H and L, for passage through said subject, said x-rays including primary x-rays having their direction of travel unaltered by interaction with said subject and scatter x-rays having their direction of travel altered by interaction with said subject. Said detector can have selected detection locations, identified by the notation (x(i),y(j)), that are intersected by x-ray projection lines extending from said x-ray source to said detection locations (i,j), said beam particle stopper device permitting passage of said primary x-rays and said scatter x-rays to said selected detection locations, preventing passage of said primary x-rays to said shadowed detector locations, and allowing passage of said scatter x-rays to said shadowed detection locations. The method can include the following steps:

(a) illuminating said subject with x-rays of said average energy level H; or said energy spectrum with a peak energy level H,

(b) acquiring a high-resolution image I.sub.fHh from said detection locations (x,y) and processing said image I.sub.fHh to normalize the image I.sub.fHh and to subtract noise, such as dark signals, and/or adjust for or subtract white noise, yielding an image D.sub.fHh (x,y) which includes said primary x-rays and said scatter x-rays;

(c) producing, from said image D.sub.fHh (x,y), an image D.sub.fHl (x(i),y(j)) representing said selected detection locations (x(i),y(j));

(d) acquiring a low-resolution scatter image I.sub.rHsl from said shadowed detection locations (i,j) and processing said image I.sub.rHsl to normalize said image I.sub.rHsl and to subtract dark signals, yielding an image D.sub.rHsl (i,j);

(e) Repeat (a)-(d) for x-ray of at least one different energy L,

(f) illuminating said subject with x-rays of said average energy level L or energy spectrum with an energy peak at L

(o) calculating a high-resolution scatter image D.sub.fHsh (x,y) by extending said low-resolution scatter image D.sub.fHsl (x(i),y(j)) to the entire image area of said detector through interpolation and calculating a high-resolution scatter image D.sub.fLsh (x,y) by extending said low-resolution scatter image D.sub.fLsl (x(i),y(j)) to said entire image area of said detector through interpolation;

p) calculating a high-resolution primary x-ray image D.sub.fHph (x,y) at said detector by subtracting said image D.sub.fHsh (x,y) from said image D.sub.fHh (x,y) and calculating a high-resolution primary x-ray image D.sub.fHph (x,y) at said detector by subtracting said image D.sub.fLsh (x,y) from said image D.sub.fLh (x,y);

(q) said images D.sub.fHph (x,y) and D.sub.fLph (x,y) can form a high-resolution, two-dimensional, dual-energy primary x-ray image pair of said subject at said front detector after said scatter x-rays have been substantially eliminated, said image pair having a spatial resolution substantially equal to the highest spatial resolution available from said front detector.

The two-dimensional projection mass densities A and B along said projection lines can be calculated from said image pair D.sub.fHph (x,y) and D.sub.fLph (x,_{Y}).

Along said projection, mass densities A and B can be calculated by solving a nonlinear dual-energy equation system for said projection mass densities A and B using the dual-energy data decomposition method.

The image pair D.sub.fHpl (x(i),y(j)) and D.sub.fLpl (x(i),y(j)) can be calculated by the steps of:

(a) solving a nonlinear dual-energy equation system for said projection mass densities A and B through a numerical inversion method using an equation system;

(b) inserting said A and B solutions into equations for said image.

The image pair D.sub.fHpl (x(i),y(j)) and D.sub.fLpl (x(i),y(j)) can be calculated from said image pair D.sub.rHpl (i,j) and D.sub.rLpl (i,j) by using direct quantitative relationships when front and rear detectors are used.

The image pair D.sub.fHpl (x(i), (j)) and D.sub.fLpl (x(i),y(j)) can be calculated from said image pair D.sub.rHpl (i,j) and D.sub.rLpl (i,j) by solving a dual-energy primary x-ray imaging equation system through a linearization approximation method with corrections for beam hardening and higher-order effects.

A method for performing dual-energy x-ray imaging of a subject using an imaging system having two-dimensional x-ray detectors is such that said subject can include two materials, M.sub.A and M.sub.B, that interact differently with x-rays, said material M.sub.A having a two-dimensional projection mass density A and said material M.sub.B having a two-dimensional projection mass density B. The detector assembly can include:

a front two-dimensional x-ray detector having a plurality of front detection locations identified by the notation (x,y), a beam selection device, and a rear two-dimensional x-ray detector assembly having a plurality of selected rear detection locations identified by the notation (i,j) and a plurality of shadowed rear detection locations identified by the notation (i′,j′), said selected rear detection locations and said shadowed rear detection locations being mutually exclusive. The detector assembly can alternatively include

a two-dimensional x-ray detector having a plurality of detection locations identified by the notation (x, y), a beam particle stopper device, its shadow regions on the detector being identified by the notation (I, j), in distributed regions of the detector.

Said subject can be between said x-ray source and said front detector, said x-ray source being adapted to emit x-rays for passage through said subject, said x-rays including primary x-rays having their direction of travel unaltered by interaction with said subject and said x-rays including scatter x-rays having their direction of travel altered by interaction with said subject.

Said front detector can have selected detection locations, identified by the notation (x(i),y)j), that are intersected by x-ray projection lines extending from said x-ray source to said selected rear detection locations (i,j). Said beam selection device can permit passage of said primary x-rays and said scatter x-rays to said selected rear detection locations, preventing passage of said primary x-rays to said shadowed rear detector locations, and allowing passage of said scatter x-rays to said shadowed rear detection locations. Said rear detector assembly can include, in physical sequence from front to back, a low-energy detector, an x-ray energy spectral filter, and a high-energy detector. A method can include the following steps.

(a) illuminating said subject with x-rays;

(b) acquiring a high-resolution image I.sub.fh from said front detection locations (x,y) and processing said image I.sub.fh to normalize it and to subtract dark signals, yielding an image D.sub.fh (x,y) which is composed of primary x-rays and scatter x-rays;

(c) producing, from said image D.sub.fh (x,y), a low-resolution image D.sub.fl (x(i),y(j)) representing said selected front detection locations (x(i),y(j));

(d) acquiring a low-resolution image Isub.rHl, from said selected rear detection locations (i,j) of said high-energy detector and processing said image Isub.rHl to normalize it and to subtract dark signals, yielding an image D.sub.rHl (ij);

(e) acquiring a low-resolution image Isub.rLl from said selected rear detection locations (i,j) of said low-energy detector and processing said image Isub.rLl to normalize it and to subtract dark signals, yielding an image D.sub.rLl (ij);

(f) acquiring a low-resolution scatter image I.sub.rHsl from said shadowed rear detection locations (i′,j′) of said high-energy detector and processing said image I.sub.rHsl to normalize it and to subtract dark signals, yielding an image D.sub.rHsl (i′,j′);

(g) acquiring a low-resolution scatter image I.sub.rLsl from said shadowed rear detection locations (i′,j′) of said low-energy detector and processing said image I.sub.rLsl to normalize it and to subtract dark signals, yielding an image D.sub.rLsl (i′,j′);

(h) calculating a low-resolution scatter image D.sub.rHsl (i,j) by extending said low-resolution scatter image D.sub.rHsl (i′,j′) to said selected rear detection locations (i,j) through interpolation and calculating a low-resolution scatter image D.sub.rLsl (i,j) by extending said low-resolution scatter image D.sub.rLsl (i′,j′) to said selected rear detection locations (i,j) through interpolation;

(i) calculating a low-resolution primary x-ray image pair D.sub.rLpl (i,j) and D.sub.rHpl (i,j) by subtracting said image D.sub.rHsl (i,j) from said image D.sub.rHl (i,j) to yield D.sub.rHpl (i,j) and subtracting said image D.sub.rLsl (i,j) from said image D.sub.rLl (i,j) to yield D.sub.rLpl (i,j);

(j) calculating a low-resolution primary x-ray image D.sub.fpl (x(i),y(j)) from said low-resolution dual-energy primary x-ray image pair D.sub.rHpl (i,j) and D.sub.rLpl (i,j);

(k) calculating a low-resolution scatter x-ray image D.sub.fsl (x(i),y(j)) by subtracting said image D.sub.fpl (x(i),y(j)) from said image D.sub.fl (x(i),y(j));

(l) calculating a high-resolution scatter image D.sub.fsh (x,y) by extending said low-resolution scatter image D.sub.fsl (x(i),y(j)) to the entire image area of said front detector through interpolation;

(j) calculating a high-resolution primary x-ray image D.sub.fph (x,y) at said front detector by subtracting said image D.sub.fsh (x,y) from said image D.sub.fh (x,y);

(k) said image D.sub.fph (x,y) can be a high-resolution, two-dimensional, primary x-ray image of said subject at said front detector after said scatter x-rays have been substantially eliminated, said image having a spatial resolution substantially equal to the highest spatial resolution available from said front detector.

The method for performing dual-energy x-ray imaging may be such that said image D.sub.fpl (x(i),y(j)) is calculated by the steps of:

(a) solving a nonlinear dual-energy equation system for said projection mass densities A and B through a dual-energy data decomposition method using an equation system

(b) inserting said A and B solutions into equations for said image D.sub.fpl (x(i),y(j))=.intg.[.PHI..sub.0 (E).times.S.sub.f (E)].times.exp(−(.mu..sub.A (E).times.A(i,j)+.mu..sub.B (E).times.B(i,j)))dE.

The method for performing dual-energy x-ray imaging may be such that said image pair D.sub.fHpl (x(i),y(j)) and D.sub.fLpl (x(i),y(j)) is calculated from said image pair (D.sub.rLpl (i,j), D.sub.rHpl (i,j)) by using direct quantitative relationships D.sub.fPl ((x(i),y(j))=D.sub.fLPl [D.sub.rLpl (i,j)), D.sub.rHpl (i,j)].

The method for performing dual-energy x-ray imaging may be such that said image D.sub.fpl (x(i),y(j)) is calculated from said image pair D.sub.rHpl (i,j) and D.sub.rLpl (i,j) by solving a dual-energy primary x-ray imaging equation system through using a linearization approximation method with corrections for beam hardening and higher order effects.

The present disclosure can include a method for performing data decomposition in dual-energy x-ray imaging of a subject using a two-dimensional imaging system, said imaging system including an x-ray source, a two-dimensional x-ray detector having a matrix of discrete detector cells identified by the notation (x,y), and detection mechanisms to determine a normalized, two-dimensional, dual-energy primary x-ray image pair of said subject at said detector cells, said subject being represented by two materials, M.sub.A and M.sub.B, that interact differently with x-rays, said material M.sub.A having a two-dimensional projection mass density A(x,y) at said typical cell and said material M.sub.B having a two-dimensional projection mass density B(x,y), said A(x,y) and B(x,y) being defined along a projection line connecting said x-ray source and said detector cell (x,y). Each of said detector cells (x,y) can becapable of being represented by a typical cell (x.sub.0,y.sub.0) in terms of x-ray signals as a function of said projection mass densities. Said data decomposition method can include the following steps.

(a) applying said detection to determine a two-dimensional primary x-ray image signal D.sub.H (x,y) at said detector cells at an average energy level H and a two-dimensional primary x-ray image signal D.sub.L (x,y) at said detector cells at an average energy level L different from said energy level H;

(b) constructing a first explicit quantitative function pair;

(c) numerically inverting said first function pair to obtain a second explicit quantitative function pair;

(d) calculating material compositions A(x,y) and B(x,y) for said subject by substituting said primary x-ray image signal pair D.sub.H (x,y), D.sub.L (x,y) for said values D.sub.H (x.sub.0,y.sub.0), D.sub.L (x.sub.0,Y.sub.0) in said second function pair for all of said detector cells (x,y);

(e) said material compositions A(x,y) and B(x,y) of said subject can represent a pair of two-dimensional projection mass density images along said projections lines at detector cell (x,y).

The method can include any one or more of the following features:

(a) said first function pair D.sub.H=F.sub.DH (A,B), D.sub.L=F.sub.DL (A,B) can be constructed through providing energy-dependent functions of said imaging system sps.sub.H (E) and sps.sub.L (E) in explicit quantitative forms to the fundamental dual-energy x-ray equations.

(b) said function sps.sub.H (E) can be separately determined by absorption method through using a reference material M of thickness t between said x-ray source and said x-ray detector, measuring a narrow-beam primary x-ray signal value P.sub.H (t) at said energy level H, and using a least-square parameter fitting method to obtain sps.sub.H (E) from the equation.

(c) said function sps.sub.L (E) can be separately determined by absorption method through using said reference material M of thickness t between said x-ray source and said x-ray detector, measuring a narrow-beam primary x-ray signal value P.sub.L (t) at said energy level L, and using a least-square parameter fitting method to obtain sps.sub.L (E) from the equation.

The method may be such that said first function pair D.sub.H=F.sub.DH (A,B), D.sub.L=F.sub.DL ((A,B) is obtained by directly measuring D.sub.H and D.sub.L values for said typical cell (x.sub.0,y.sub.0) at a number of points with known values of A and B in a desired range of (A,B) and analytically extending said D.sub.H and D.sub.L values to continuous domain.

The dual energy or multiple energy imaging method may be such that said numerical inversion from said first function pair D.sub.H=F.sub.DH (A,B), D.sub.L=F.sub.DL (A,B) to said second function pair A=F.sub.A (D.sub.H, D.sub.L), B=F.sub.B (D.sub.H, D.sub.L) can be conducted by the following steps.

(a) calculating a first pair of arrays of values from the simultaneous equations D.sub.H=F.sub.DH (A.sub.n,B.sub.m) and D.sub.L=F.sub.DL (A.sub.n,B.sub.m) on an integer grid (A.sub.n,B.sub.m), where A.sub.n=A.sub.0,A.sub.1,A.sub.2, . . . , A.sub.N and B.sub.m=B.sub.0,B.sub.1,B.sub.2, . . . , B.sub.m are integer indices of said first pair of arrays;

(b) numerically inverting said simultaneous equations D.sub.H=F.sub.DH (A.sub.n,B.sub.m) and D.sub.L=F.sub.DL (A.sub.n,B.sub.m) to obtain simultaneous equations A.sup.0=F.sub.A.sup.0 (D.sub.H [j],D.sub.L [k]) and B.sup.0=B.sub.B.sup.0 (D.sub.H [j],D.sub.L [k]);

(c) calculating a second pair of arrays of values from said simultaneous equations A.sup.0=F.sub.A.sup.0 (D.sub.H [j],D.sub.L [k]) and B.sup.0=F.sub.B.sup.0 (D.sub.H [j],D.sub.L [k]), where D.sub.H [j]=D.sub.H [0],D.sub.H [1], D.sub.H [2], . . . , D.sub.H [J] and D.sub.L [k]=D.sub.L [0],D.sub.L [1],D.sub.L [2], . . . , D.sub.L [K] are integers or real numbers and where D.sub.H [j]<D.sub.H [j+1] and D.sub.L [k]<D.sub.L [k+1], j, k, J, and K being integer indices of the coordinate arrays for said second pair of arrays;

(d) for each of said measured dual-energy signal data pair D.sub.H (x,y), D.sub.L (x,y), determining the closest indices j and k values according to the criteria D.sub.H [j].ltoreq.D.sub.H (x,y).ltoreq.D.sub.H [j+1] and D.sub.L [k].ltoreq.D.sub.L (x,y).ltoreq.D.sub.L [k+1], and then, from said closest indices j and k, determining said A(x,y) and B(x,y) from said simultaneous equations A.sup.0=F.sub.A.sup.0 (D.sub.H [j],D.sub.L [k]) and B.sup.0=F.sub.B.sup.0 (D.sub.H [j],D.sub.L [k]); and

(e) refining said A(x,y) and B(x,y) to an accuracy as high as real numbers as provided from the equations ##EQU9##.

ROI or VOI

Identification and selective measurement of ROI or VOI and/or iterative process involving this method can enable personalized or customized x-ray imaging or spectral imaging or tomography or CT to reduce radiation exposure and/or speed up image acquisition and reconstruction, and improve achievable resolution and sensitivity significantly.

Determination or Identification of ROI or VOI to be Further Investigated

Previously the region of interest may be identified or determined by single, or dual energy measurements, or multiple energy measurements, sometimes coupled with material decomposition or 3D or CT or multiple dimension imaging or CT slice or spectral tomography. In the x-ray imaging apparatus and methods disclosed here, determination of region of interest may be achieved by methods include one or more of the following:

User Input.

Digital Program.

Predetermined, for example, based on one criterion or a number of criteria.

Decisions derived from AI analysis of and/or measurements, or real time measurements of one or more x-ray image using one or more detectors, here referred to as the first detector or first detectors.

Decisions derived from analysis, or AI analysis of and/or analysis of, and/or measurements, or real time measurements of External Sensing Element, such as a positioning, and/or distance, and/or single, two or multiple dimension measurements using optical, ultrasound, MRI, PET, SPECT, physical measurements or chemical or electrochemical measurements.

Decisions derived from analysis, or AI analysis of and/or measurements, or real time measurements of one or more x-ray images which are scatter removed in the spatial, or frequency, or time domain to less than 1% or less than 5% of SPR, and/or reconstructed tomography images with less than 1% or less than 5% of SPR.

Decisions derived from analysis, or AI analysis of and/or measurements, or real time measurements of point, and/or one dimension, and/or two dimension or multiple dimensions using methods and apparatus disclosed herein.

Decisions derived from material decomposed data, for example, using the material decomposition methods and apparatus described in this disclosure (or other methods based on the disclosure herein), using energy sensitive detectors, synthesized, or deep learning images of separate image of single substance or combined substances.

Using multiple dimension, 3D, or up to 7D, and/or material decomposed images of x-ray to provide image guidance for intervention, treatment, monitoring, and/or tracking and/or diagnosis, and/or measurements using modalities other than x-ray.

Measurement of Region of interest can include one or more of the following features:

ROI may be a static ROI in a static subject, for example, approximately the same spatial location, unchanged chemical state or physical state;

ROI may be in a dynamic state, or approximately dynamic state, for example, for monitoring or tracking of a component or a target in an object or a subject.

The measurement of ROI may be achieved by apparatus and methods include one or more of the following items:

Using the first detector,

Using the same or different x-ray source,

Using the same x-ray source with a different setting, for example, a different focal spot size, or different x-ray emitting position,

Using the first detector, with a different setting for frame rate or resolution, or pitch size, for example, with or without binning, ,using a selected region of the first detector or detector position,

using the same detector combined with a different detector placed in front or in the back of the first detector,

using a different detector placed within approximately the spatial location of the first detector,

Using a different detector to be placed in the spatial location of the first detector after the first detector is moved to collect x-ray measurement of ROI, or

Using a different modality, such as optical, electrooptical, photoacoustic, nonlinear microscopy, OCT, PET, SPECT, MRI or pressure, kinetic measurements, temperature, electrophysiological, electrical measurements.

And the measurements of ROI and/or determination of ROI and identification of ROI in an object may be iterative and/or repeated, each time based on the same criteria or different criteria.

And each time, criteria for determination of ROI, and/or measurement of the ROI in the object may be different and/or measurement method or combination of measurement methods may be different or the same.

For example, a dual or triple energy or multiple energy imaging method and/or material decomposition methods are used to determine one or more ROIs.

A low resolution of 3D or 2D images may be taken of each ROI to reconstruct a 3D or multiple dimensional or synthesized 2D image of ROI. The result and/or information derived may be used to determine again the ROI for the next set of measurements and/or for determining which measurement methods may be used. For example, a different resolution, such as higher resolution image may be reconstructed by taking 2D images a greater number of times than the last time to achieve higher resolution by resolving higher number of unknown voxels of smaller dimensions than before along the depth of ROI. X-ray emitting positions can travel with smaller steps than with the first set of measurements used for the prior 3D image reconstruction.

And to resolve even further materials or substances within one or more voxels, spectral images or multiple energy images or measurements may be taken and/or 3D image reconstructed from multiple energy measurements may be derived to further reveal attributes of the voxel in the ROI and increase sensitivity of measurements for each voxel in the ROI.

Using of x-ray images as the first image and/or first measurements, in some instances, may co-locate or co-register with modalities of other imaging methods, by colocation of anatomic markers or targets recognized by contrast agents, or spatial proximity.

Using of methods of optical methods or other modalities may be combined with x-ray image method to track in a surgery.

VOI may be a subregion of a region or a tumor. VOI may be in the eye or parts of the eye.

Density measurements, interpolation to derive density information correlating to detector measurement of various energies may be done on various detectors, to correlate density measurements from two or more detectors. As long as there is a quantitative relationship established from a first detector to another detector, such as a second detector, using one or more common reference material or substances at various densities, a database and/or energy response function equation system may be established with first detector or first detectors and may be used for the density determination of substances which are measured with other form of detection or other type of x-ray detectors.

Large Region of Interest and/or imaging of Field of View can be larger than what one x-ray source allow.

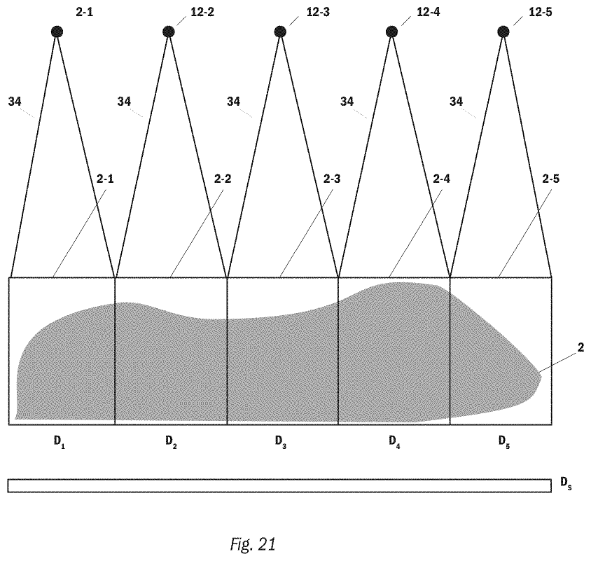

As illustrated in FIG. 21, where is a large region of interest, 2 for example, for human whole body imaging, two or more area detectors (for example D1, D2, D3, D4, D5), and/or two or more x-ray sources (for example, 12-1, 12-2, . . 12-5) may be needed for fast image acquisition of the entire region of interest, in point, or two dimensions, or multiple or 3D.

For example, each source or each source plus at least one detector may be used for 3D or multiple dimensional imaging of the corresponding portion of region of interest, 2-1, 2-2.. 2-5 which are illuminated by the x-ray sources.

The placement and/or movement of x-ray sources or x-ray emitting positions or steering of x-ray beam may be along a single axis or in 2D or in 3D or in each axis of or one or more combination of each axis in a 6D space.

The placement of x-ray sources may form a pattern, for example, spiral pattern facing the region of the interest.

Both x-ray source and the detector may move synchronously or asynchronously in one axis or in two dimensions or in at least two axis of 6D relative to the subject or the region of interest to complete the 3D or 2D measurements for a large region of interest.

FIG. 21 illustrates using more than one x-ray source, and correspondingly, more than one detector, or a very large detector including two or more detectors, or a large detector for measuring x-ray signals from all of the sources.

Each x-ray source and corresponding detector pair can be capable of point, linear, 2D and multiple dimension measurements or 3D measurements or other techniques described herein. Some of the measurements have <1% scatter over primary ratio, and some of the measurements have less than 5% scatter over primary ratio.

Large field of view 3D imaging can also be accomplished by moving a set of x-ray measurement module including at least one x-ray source and at least one area detector, relative to the subject, which can be placed on a table and be radiolucent, or be held by a sample holder.

X-ray measurements may be further extended by other imaging techniques, some of which have been described in the x-ray imaging apparatus and methods disclosed.

For additional measurements in different spatial resolution or spectral resolution, phase contrast, Fourier transform, different speed or different energy level measurements, additional x-ray source, x-ray optics or detectors of various kind and form factors may be used. In some cases, x-ray and light measuring device including intensifier or scintillator, optics and cameras may be added for detection.

In some cases, x-ray optics, such as collimating, steering or focusing devices may be used in between the x-ray source and the region of interest. A condenser or zonal plate may be used. A beam aperture of selected size may be placed immediately upstream of the region of interest. The x-ray passing through the beam aperture may be measured by an x-ray detector directly, or an objective may be placed right downstream from the subject, and a scintillator is placed either upstream of the objective or downstream of the objective and a detector is used to measure the x-ray output coming out of the objective or the scintillator.

An x-ray source, in some instances, may be placed immediately upstream of the subject, the output x-ray from the subject may be collected by an area detector or a intensifier, optics and objective and other elements which de-magnifies the x-ray output onto a camera.

Phase contrast or interferometer optics, either in the x-ray wavelength range or in the optical range, may be used.

Multiple Segments or Multiple Portions of ROI

X-ray source 2 and/or the corresponding detector 22 may move very small distances, in mm or sub mm to acquire multiple dimension or 3D or more dimension measurements. For example, x-ray source or the x-ray source and the detector pair more in an area approximately the same as the distance between the top region of interest layer and the bottom region of interest layer or the same as the depth, or the depth of the region of interest perpendicular to the detector.

X-ray source 2 and corresponding detector 22 may move in distances or area or volume larger than 1 mm in dimension, or in the dimension such that the same x-ray source may illuminate the entirety of the region of interest. For example, if the field of view of the x-ray source is not big enough to allow x-ray to project on the entire region of interest, then x-ray source and/or the corresponding detector may move relative to the region of interest after a complete 3D or one or more 2D measurements are taken of a first segment or a first portion of the region of interest. The first segment or first portion is what the x-ray illuminates at its first position relative to the region of interest. After the images of the first segment, or first portion is taken, at least one of such images has scatter removed to less than 1% SPR or less than 5% SPR. In some cases, multiple dimension or 3D images may be constructed for the first segment, or the first portion of the region of interest using the 3D reconstruction methods as described herein. In some cases, such measurements are of two or more energies, and/or are phase contrast imaging measurements and/or are measurements of the region of interest after or during energy perturbation as described in the x-ray imaging apparatus and methods disclosed here.

The x-ray source and/or corresponding detector may then move relative to the subject or to the region of interest to measure a different or a second segment of the region of interest. One or more similar measurements as performed for the first segment or first portion of the region of interest may be performed for the second segment. Similarly, a third segment or a third portion may be performed and so on.

The entire region of interest may be measured and image reconstructed by stitching together two or more segments or portions of ROI.

Stitching together the two or more segments may be accomplished by matching anatomic markers or based on matching overlapping measurements of two different segments or portions for a selected subregion of ROI on either segment or portion, if the two segments or portions are defined to have selected subregions overlapping with each other.

The movement of x-ray source and/or the detector may be precisely aligned so that the images taken are stitched together end to end. And if there is missing gap, values from adjacent pixel or pixels regions on the border of the segments can be interpolated to the missing pixels in the interface region between two segments or portions.

Segments or Portions are defined as a part of ROI. Combination of such portions may include the entire ROI. ROI may be divided into two or more units of portions and/or segments. Segments used here may not mean the same thing as in segmentation of ROI into different tissue components which are overlapping. Segments used here results in spatially the ROI are divided into portions or segments, each can be interrogated by x-ray beam independently from other portions or segments and by the same projection and collected on the detector. Such a segment or portion in this section of disclosure may contain a number of tissue types and components overlapping each other. In a more precise definition, the term portion may be used to better describe this scenario as segments may be used in other settings, such as in post image acquisition, material decomposition and segmentation into segments of ROI, each having distinct characteristics which can be separated from the rest, even of it is illuminated and measured by the same x-ray projection path, for example, a projection line reaching a corresponding pixel on the detector.

At least one additional x-ray sources and/or at least one or more additional detectors may be used and/or moved to measure one or more selected targets on the first portion of interest for further investigation. X-ray source or detector may vary in size, resolution, speed and energy level or wavelengths from the x-ray source or detector used in the first or first set of measurements performed on the region of interest.

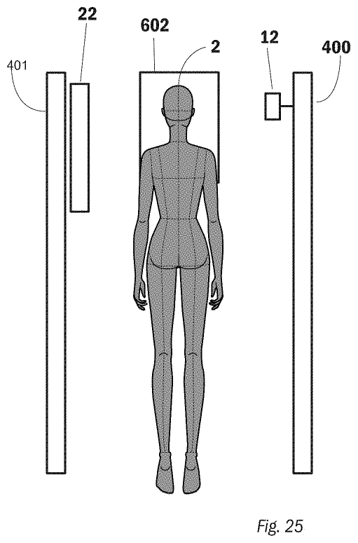

In some examples, each x-ray source may generate x-ray beams illuminating the region of interest, measured by multiple detectors. Or as illustrated in FIG. 25, one or more x-ray sources at one location can illuminate the whole body. A detector can be used by stitching together two or more detectors or a large detector can cover the entire subject, which is the whole body. Alternatively, a detector and/or a set of source and detector can be moved to cover different portions of ROI, which is the entire subject. For example, when a subject is a whole human body, the source and detector can be to image one portion of ROI at a time and the imaging process can be repeated to image other portions, until the entire subject is imaged. A portable large field of view x-ray imaging system is capable of expanded field of view. A radiolucent table or support mechanism such as radiolucent hospital bed or sample holder 40, where the imaged subject 2 is placed. The x-ray can be be emitted from the source 12, illuminate a subject 2 at the region of interest and pass through and reach the beam particle stopper plate 100 and the detector 22.

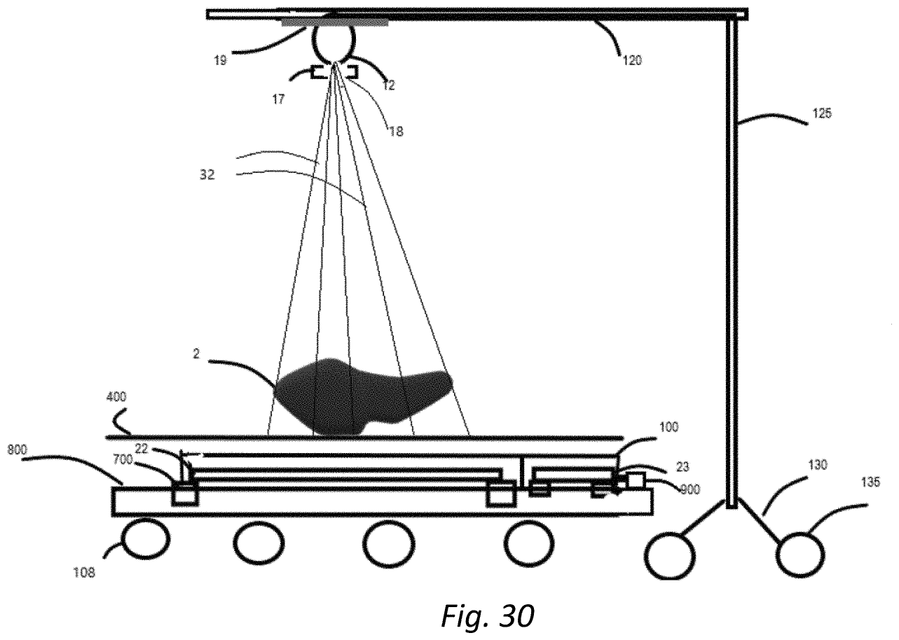

As illustrated in FIG. 30, the detector 22 may is motorized and/or thex-ray source 22 may is motorized spatially to image different region of the large subject, for example, of a whole human body or a cargo and a wafer production machine. A subject 2 is placed between source 12 and detector 22. There may be a mechanical structure (for example, a beam, an arm, or otherwise) 104 to connect the source, detector and the movers.

The motor that drives source 12 may move the source or x-ray emitting positions in small dimensions to construct 3D or multiple dimensional images as described in this disclosure.

A different motor may be mounted on the same spatial location of the supporting structure 104 as the first motor, to move the x-ray emitting positions in a finer step in some cases.

An electromagnetic steering device may be mounted to the same structure 104 and used to move the x-ray emitting position by steering electron beam prior to reaching the target.

For example, one large detector D5, may measure x-ray coming from multiple x-ray sources 2-1 . . . 2-5, each source illuminating a portion of the region of interest 12.

Data for 3D Print and Display of ROI

Printing, 3D printing using tangible substances and electronic display, transparent display such as computer monitor or screen display, projection and projection display or 3D projection display and 3D display may be based or derived or synthesized from the measurements using apparatus and methods described in this disclosure.

X-ray images measured or synthesized or reconstructed based on the method and apparatus disclosed presently may be used for 3D printing of the region of interest or the target or the subject using one or more material similar or variant of the original subject and/or region of interest. 3D printing may be done by a machine including a printing tool or construction tool including one or more materials to represent region of interest or the subject.

3D and Tomographic Reconstruction

The images used in reconstruction may include mainly primary x-ray images and/or measurements so that SPR is less than 1% or less than 5%.

Example of tomography reconstruction and preparation of the data for reconstruction will be described below.

While ROI is determined, and a command to take a tomographic image is sent to the microprocessor by the user or made by a digital program or command or an algorithm, and a single energy and/or spectral imaging command is made, the VOI is defined for tomography by determining boundary of VOI in a coordinate of x, y z. The thickness of the VOI may be determined by the user by measurement, or the use of a sensor, such as time of flight sensor, to measure the distance of the top surface of VOI to the x-ray emitting position. Since the distance from the top of the sample holder to the surface of detector is known, the thickness of VOI is therefore determined.

After projected images are taken, the predetermined contribution to the detector measurement, from sample holder, beam particle stopper plate, and any other attenuating matter in between the detector and the VOI, may be determined and characterized separately. In some cases, this may be on a pixel by pixel basis.

VOI may be an internal VOI embedded within the thickness of the object. In this case, Vai, the volume above the VOI, closest to the x-ray emitting position, or Vbi the material volume below the VOI closest to the bottom of the VOI may be determined separately or differently and/or with a technique at least partially different than that for VOI using a reconstructed image previously, or other reconstruction techniques.

Multiple aperture devices (MADs) are sequential binary filters that can provide a wide range of fluence patterns, and may be placed between the source and the object, and adjusted dynamically with relatively small motions to select VOI, some of which may be off axis.

Information related to reconstruction may be stored in the microprocessor, includinga spatial projection geometry.

At the time of taking a projected image, the spatial projection geometry is used to related the spatial position of x-ray emitting position to the center of VOI and to the detector. Such spatial projection geometry may be defined, for example, as explained below.

If the X-ray emitting position moves on a x y plane parallel to the detector, one coordinate with three degree of freedom may be used to describe all spatial location and relative movement of x-ray source, object and/or the detector. The distance of x-ray source to the center of VOI may be defined, and the distance of detector center axis to the center of VOI may be defined.

For more complex movements of x-ray source and/or detector, additional coordinates, such as if relative rotational motion is involved, rotational coordinates may be used, each for x-ray source or also for the detector,.

The x-ray source information such as focal spot size, energy level, current, exposure characteristics , center axis, detector pixel pitch size, number of elements, spatial location of detector, may be stored in a memory device of the x-ray system disclosed herein.

A meta file may be generated for storing information regarding the parameters involved in reconstruction for the projection image taken in the microprocessor,

A system matrix can be designed to model spatial positions or relative spatial position of x-ray emitting position, center axis of x-ray beam, for instance, center axis of x-ray cone beam, SID (source 12 to detector module 22 distance), movement and/or alignment of source and/or detector pair to illuminate the region of interest, setting of voxel location relative to the source and detector, for example, to determine the u, v, the projected location on the detector relative to the center axis, u0 and v0, the offset of the x-ray source center axis with the center axis of the detector. The expression for the movement of x-ray source may be a vector with its first and second element being coordinate transformation of the x-ray source.

Based on spatial location and/or movement of the x-ray source and/or x-ray detector, and the VOI movement, the microprocessor can determine the number of coordinates which can describe the degree of freedom for different hardware movement and relative spatial location.

Filters such as aluminum or copper, may be used downstream of x-ray emitting position and in between the source and the object, in some cases, to reduce beam hardening effect.

To reconstruct a tomographic image from the projection images can include the following steps. In the first step, each voxel in the VOI may be set at a value of zero or one to approximately define the attenuation coefficient range of the voxel. A threshold of the attenuation coefficient value may be used to set such values at one or zero. Depending on the application and required accuracy, various factors which may contribute to the actual attenuation coefficient value of a voxel at a certain spatial position relative to the x-ray source may be taken into account. For example, the factors may include the x-ray tube, anode type, design of the x-ray tube, the detector and contribution in attenuation of the voxels in the ROI upstream of a particular voxel and the magnification factor of cone beam through the sample of a certain thickness, photo influx variations in a given volume at certain spatial positions, and number of photon variations at certain distance from the source, interaction with substances or matters of certain composition or attenuation value.

Simulation methods such as Monte Carlo methods or other simulation tools used in radiotherapy, x-ray imaging, nuclear imaging, SPECT, electron microscopy and ray tracing methods may be used. For example, deterministic approaches based on ART may be utilized. Both Monte Carlo methods and deterministic methods may be combined for reconstruction methods. X-ray emitting position movements and x-ray measurements can be performed, and unknown voxels can be resolved based on a number of linear equations, where the unknown voxel values are either 1 or 0 depending on the definition based on the attenuation range of voxel. For example, bone may be assigned a value of 1 and soft tissue may receive a value of 0, equal to being transmissive.

The detector measures a 2D projection image of the ROI. Same ROI are measured at various x-ray emitting positions relative to the center axis of its corresponding detector. The total movement of the x-ray emitting position relative to the center axis of the detector may be less than 10 degrees, or less than 5 degrees or less than 2 degrees or less than 1 degree away from their first position of center axis defined by the x-ray cone beam, the x-ray emitting position, ROI and the detector. The attenuation coefficient value of the voxel may be resolved using ART or derivatives of ART and/or alternatives simulation methods to have a value of 1 or 0, each representing an attenuation value above or below a threshold.. A threshold may be set at a certain attenuation coefficient value that separates two materials, such as bone and soft tissue, or it may separate contrast agents from that of the background tissues. This process may be iterative to continue the adjustment of the threshold until there is convergence of the simulated projection measurement with the actual projection measurement.

Reconstruction of conventional CT images and tomosynthesis, c-arm or 0 ring, and derivatives of reconstruction methods may be used with the aforementioned projection geometry to reconstruct from the acquired images. Difference between the system and method disclosed herein and the conventional CT, and other x-ray imaging multiple dimension imaging can include:

the transformation and/or calculation of the projection geometry in the spatial geometric matrix or configuration. Conventional CT-like technologies typically has a more complex geometry.

the iterative algorithms for reconstruction may have noisy geometry artifacts in conventional CT-like technologies.

the projection image is noisy due to scatter in conventional CT-like technologies, therefore prolonging reconstruction time due to the initial estimated attenuation coefficient value for each voxel.

certain commonly accepted algorithms may not be used in conventional CT-like technologies as some of the algorithms are for large angle rotational projection geometry.

The X-ray emitting position can move in the xy plane, having approximately similar or same source to detector distance and relative plane position of the source to ROI and to the detector. Consequently the geometric matrix is modified to describe this motion. A vector expression with three variables can be obtained. The first two describe the movement in the x and y direction. The third variable may be related to x-ray emitting position movement in the z axis.

In some cases, more x-ray source, denoted as “second” or third “source” may be used for measurements of the same VOI at the same spatial location as the first source. When the second source is moved to project images of VOI from the same positions of the first source, for example, when the second source emits x-ray beam of different energy level, tomography reconstruction can be done with second set of projection images providing multiple energy measurements and therefore more accurate derivation of attenuation coefficient for each voxel and/or less iteration is needed.

When spectral x-ray imaging systems using source and detector pair similar to that of conventional spectral CT-like technologies, the dosage and radiation exposure may be obtained by the simulation method.

Each voxel's attenuation coefficient constants and/or attenuation of each voxel may be modified due to its actual weighted value based on its value obtained from the simulation tool. Depending on which layer in the ROI the voxel resides, the weighted value of each voxel combined with its original value can give rise to a modified attenuation coefficient or attenuation value X. However, such a value X is a function of attenuation value or attenuation coefficient constant.

At each energy, such a voxel value may be determined. The information may be used to segment volumes in the ROI, determine density and thickness, characterize unknowns and identify and visualize each substance and composite substances.

One example of a reconstruction model is to use reconstruction method based on ART and its derivatives, or Monte Carlo simulation method, or combination of both. . The exampleincludes statistical modeling performing a local signal to-noise ratio analysis to decompose data into information and noise according to the model. In this case, the data may not contain scatter noise compared to a conventional CT as in the present disclosure, since scatter interference has been removed to less than 5% or less than 1% of the primary. Scatter therefore may be taken into account when the scatter removal method is estimated to be more than 1% of the primary x-ray. However, due to the reduction of measured signal to noise ratio for the primary x-ray due to the contribution of scatter affecting dynamic range, this step may be needed in some cases.

There may be other type of noise existing, different than scatter. A post image acquisition and reconstruction processing may be needed to remove the noise other than scatter.

In some cases, the measured scatter value may be used to substitute the simulated scatter value to extract the information or expected projection image. It is likely in some cases, due to lack of scatter in the derived projection image, and material decomposition, the reconstruction may not require an iterative process for correction. The adjustment may be done on a case-by-case basis.

Reconstruction method may include Monte Carlo simulation or simulation methods used to do x-ray projection simulation and modeling, which may, in some example, be combined with setting the voxel value to zero or one for a range of attenuation values, and correlate the value in each Voxel unit to what is measured on the detector respectively.

Beam profile in spatial and temporal may be monitored.

Each voxel Vxyz may include a set number of subunits.

Each subunit Sub may be of a certain dimensions. Each voxel in the volume of interest or region of interest has a spatial relationship or position which may be described quantitatively, for example, by distance and/or in spatial coordinate in at least one axis, to the detector and/or specific pixel or pixels or measurement regions including one or more pixels, and/or x-ray source and/or the central axis of x-ray source, perpendicular to the detector or any reference and/or any spatial reference and/or a portion of or the entirety of the imaged or measured subject. Such quantitative relationship of each voxel may be one of the parameters which defines the voxel. Voxel may have an x-ray measurement value based on its composition. The composition of each voxel and its spatial location relative to what is around it can include the detector, anatomic markers of the imaged subject or imaged subject or an external reference object, which sometimes may be detector, or the source or an abstract object such as the central axis defined by the x-ray emitting position or the detector and/or a portion or the entirety of the imaged object.

In one example, as the x-ray emitting position moves in x, or y or z axis or 6d spatially or 7 d position that includes a temporal marker , the specific voxel Vxyz may not move. However, the central axis of x-ray emitting position may move. The corresponding pixel or regions of detector to a specific Vx,y,z may be the same or different.

The voxel of specific spatial position or a portion of voxel and the corresponding pixels or pixel regions on the detector which measures the x-ray signal passing through such voxels or a portion of voxels as x-ray emitting position moves are recorded and tracked. The portion of voxel may be described as a percentage of the voxel. The portion may be described by a number of subunits and may be contained in the portion which may contributes to the measurements of a pixel or a region of detector. For example, if a Voxel, Vxyz has 100 Sub, or 100 subunits of lum, a portion of a voxel may contain 64 or 10 subunits, which may contribute to the measurement on a pixel or region of detector determined by the x-ray illumination path defined by the voxel spatial position, x-ray emitting position and the region of the detector which makes the measurement.

The electromagnetic measurements and/or other physical or chemical characteristics or measurements or simulated properties of the specific voxel V xyz are correlated to the measurements of and spatial relationship to the pixels of the detectors. The data is used as a part of data for analysis such as quantitative imaging, qualitative imaging, material decomposition and/or reconstruction method for multiple dimensional imaging or tomography or tomosynthesis.

The volume of interest or the voxel in the volume of interest VOI or region of interested including the VOI and its boundary may be described by the x-ray illuminating path and the detector measurement region, for example, in a cross section described by boundary with corners at position a, b, c, d. The boundary spatial shape may or may not be symmetric. For example, dimensions described by the distance of b to C of the top layer closest to the source may be smaller than ad, which describes parts of the dimensions of the bottom layer closest to the detector.

Examples of Reconstruction—3D Construction of Volumetric Data

As x-ray beam are in a cone shape, the region of interest closest to the x-ray source may be smaller than that of region of interest closest to the detector. Or x-ray illuminating the voxel or voxels from region of interest closet to the x-ray source may project to a larger area on the detector when measured.

For example, a region of interest ROI total (x, y) may include a volume extending from the surface of the subject closet to the source to the surface of the subject closest to the detector.

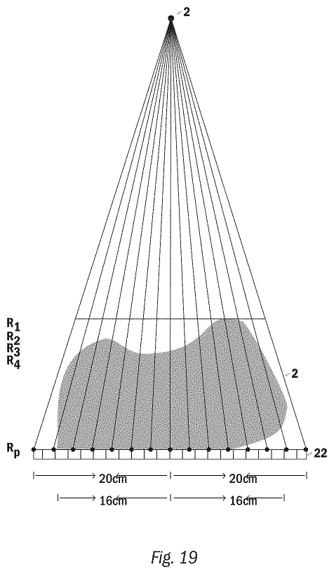

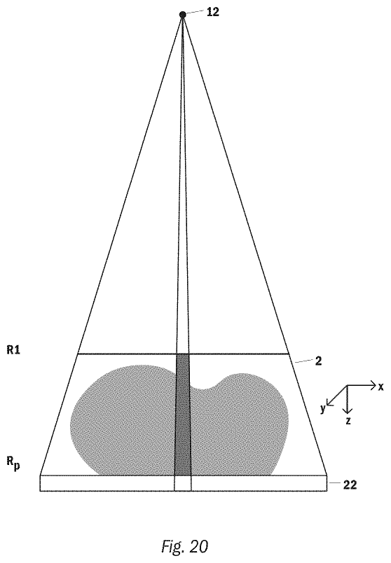

As shown in FIGS. 19 and 20, ROI total (x, y)=R1+R2+ . . . Rp, where each R is a distinct layer within region of interest along the x-ray projected path. For example, if the depth along the z is 20 cm, and resolution of voxels needs to be resolved along the Z, and Xz is 200 um, then 1000 data points or voxels unknown layers need to be resolved.

For example, if the source to detector distance, SID is 1 meter, the region of interest R1 closest to the x-ray source is 32 cm, and the region interest downstream of the ROI R1, Rn may be 40 cm, the detector region may be at least 40 cm in order to capture all the region of interest from R1. While R 2, the region of interest layer immediately downstream of R1 may be similar to R1, but may be slightly larger, for example, 1.00004 x of rl, which may be negligible in some cases, Rp is significantly bigger, where p is 1000 if there are 1000 layers of region of interest, each of equal thickness or resolution.

In one example as illustrated in FIG. 19, the X-ray cone beam angle α=ArcTan (20 cm/100 cm)=0.1974

Along each axis, if each pixel is 200 um , in the last layer in the region of interest,there are then 20 cm/200 um=1000 pixels on the detector or 1000 voxels in the layer in the region of interest closest to the detector. The area size xy of the detector pixel is approximately 200 um×200 um=40,000 um2.

Ratio of the dimension size in the x direction in the top layer of the region of interest, closest to the x-ray source to that in the bottom layer Rp of the region of interest is

Ratio 1=16 cm: 20 cm=0.8/1

The ratio of the area size in the xy dimension in the top layer R1 of the region of interest closest to the x-ray source to that in the bottom layer Rp is

Ratio a=0.82:1 2=0.64:1

In this example, the area size ratio of voxel on the top layer to the voxel on the last layer of interest is approximately 0.64 of the voxel size on the bottom layer in the region of interest, as illustrated in FIG. 19. For instance, when x-ray beams passes 64 adjacent voxels on the top layer, the same beam path becomes magnified at it travels through the region of interest due to the nature of cone beam, and projects on an area region of approximately 100 pixels on the detector. The bottom layer along the z direction in the region of interest, illuminated by the same beam path is approximately 100 voxels.

In this example, 0.64×M1000=1 Where M is the magnification factor from a layer in the region of interest to the layer adjacent to it and on top of it. As (M−1) may be very small, the layers right next to each other along the z may be similar approximately.

However, the magnification factor is much larger when it is between the top layer and bottom layer of the region of the interest.

In FIG. 20, the relative size of voxel in xy direction of the top layer, or of first layer along z closest to x-ray source versus the relative size of bottom layer, the pth layer Rp along the z direction in the region of interest for the same projected x-ray beam, which produces signal on approximately one pixel on the detector, is 0.64:1.

There are more than one method to correlate voxels from different voxels from R1 . . . R2.

For example, if it is assumed each voxel Rfl in R1 is made of 64 different secondary voxel units Rs that are adjacent to one another, each voxel in Rp, Rfp, may include approximately 100 different secondary voxel units Rs. The summation of attenuation value of the x-ray beam by all of Rs in one voxel Rfl, correspondingly summation of attenuation of x-ray beam passing through Rs in the voxel Rfp of Rp , illuminated by the same input x-ray beam, and any voxels in between the Rfl and Rpt, can contribute to the final signal level of x-ray output, which is then projected and measured by a detector region d (x, y) approximately one pixel pitch in size and immediately below Rfp. In some cases the center of x-ray projection passing through Rfl will land in the center of detector region, which may be the active center of a pixel.

In some instances each of secondary voxel units can be further divided into even smaller voxel units, Rt, or Rq.

Each secondary voxel may be numbered or may have an identifier associate with it to designate its relationship and location relative to the source, the voxels in R1 . . . Rp, and to other secondary voxels.

In cases of solving unknown voxels Rf of each layer, combinations of one or multiple secondary voxels, or segment of a Rf voxel may be solved, and in some cases, each or all of which, may be used to combine with secondary voxels in the adjacent areas or adjacent regions of interest to derive the complete individual RF in one or more region of interest layers.

A different example for multiple dimension image reconstruction may assume the largest voxel nearest to the detector or one detector pitch as the voxel unit. Voxels in region of interest closer to the source may be a portion of the voxel units. Such segmented voxel designation may be used, such as Rfl=0.64 Rfp.

Each detector pixel may measure x-ray signal as a result of x-ray input beam passing through voxels in each layer of the region of interest. The xy position of the secondary voxel or first voxel may be correlated to a detector pixel, which may have x y coordinate values, and x-ray measurement or x-ray measurements, corresponding to a particular x-ray projection path.

In some instances, there may be a third value designating which region of interest layer along the z is the voxel RF.

As illustrated in FIG. 20, each Rf to be solved may therefore be described with a three axis coordinate (X, Y, Z). However, the x, y values may not be the same as its true spatial location relative to the detector, but is adjusted value taken into consideration the magnification factor or demagnification factor M compared to the voxel size Rf on the top layer of region of Interest, R1 or the voxel size Rp on the bottom layer in the region of interest.

Alternatively or additionally, RF of each voxel in region of interest can also be represented with true spatial coordinates. A database C can contain the relationship between each voxel and its adjacent voxels in the region of interest layer where it is located and relative to voxels in regions of interest layer other than where it is resided. Each of voxels RF from each layer may be correlated to that of other layers.

As x-ray emitting locating or x-ray source moves, the database C will correlate a new set of relationships between voxels of the region of interest.

Each voxel Rf or Rs may also have a value which may be the attenuation coefficient or a value based on attenuation coefficient and the size of voxel, associated with each wavelength or energy level. And there may be another reference database which associates quantitatively and deterministically or statistically a value or a set of value or a range value with one or more material, substance, component, or a synthesized matter of one or more simulated matter or composition voxel.

In some cases, when the x-ray source may move in one direction, for example linearly in x, y, z, pixel by pixel, or the x-ray source may be moved in angular fashion space, each time, different x-ray projection paths across the subject are introduced. The system disclosed herein can at the same time minimize the amount of new unknowns introduced, or at least reduce number of unknown voxels in the projection paths as much as possible. Such projection based geometry calculation may be used for 3 D image reconstruction.

As long as in each x-ray source movement, or x-ray steering angle, there are new projection lines involving new sets of x-ray illumination path that are landing in the detector and measurable at the same time, the number of unknown introduced in each step of movement or the aggregated number unknown voxels introduced in the entire 3D imaging process can be reduced and/or minimized compared to those introduced in the 3D imaging using flat panel detectors by conventional methods.

When the unknown number voxels that are introduced while imaging for multiple dimension and 3D imaging, or time based 3d and/or to 6D imaging, and tracking , are reduce and/or minimized, the methods to measure x-ray signals therefore will introduce less radiation. The reduction of radiation dosage may be achieved by applying radiation dosage only onto a specific voxel of defined dimension in the region of interest, and/or reducing total dosage of radiation to the region of interest, and/or radiation dosage to the voxels or regions in proximity or adjacent to the region of interest.

The radiation dosage may be reduced by defining the region of interest or component of interest to be imaged or multiple dimension to be reconstructed and further investigated by single or multiple energy x-ray imaging or other modalities or by other electromagnetic measurement methods, which may be time based, such as using ultrafast pulse, or using steering, magnification, demagnification mechanisms, or by interferometer based techniques. The process of defining one or more region of interests and/or removal of regions in the subject to be imaged, which is not of interest in temporal domain as well as frequency domain as well as spatial domain, therefore determining regions of interest to be interrogated by x-rays or other modalities, may be iterative and/or repeated. After each measurement, the data acquired in the previous measurements and/or some or all of the past measurements, and all the relevant data, such as chemistry based, and/or personal genomics based, and/or immunity profile based, and/or environmental based, and/or statistically based, may be taken into account to be analyzed for determining where to image or measure next.

The selection of region of interest may be done by a user or could be done by a microprocessor with software either from a preprogrammed digital process or an AI trained algorithm or AI driven or deep neural network learned or trained process. The measurements of x-ray in nature can be a point measurements, linear measurements or 2D measurements or >2D dimension measurements.

A database can be built for each relevant voxel or one or more sub voxel units in each voxel in the region of interest or in the subject, based on measurements and/or interpolated values corresponding to approximately the density and/or characteristics of substance or composite substances in a voxel, to that of measurements at one or more energy levels.

The interpolated plot at various energy levels for substances or composite substances at voxel or sub voxel dimensions of various densities relevant to the range of interest is generated by taking a number of measurements of substance or composite substances with two or more density values within the range of interest at various relevant energy levels.

The result of x-ray measurements may be used in AI or deep machine leaning as one of the parameters to train or identify or diagnose or prognosis a selected region of VOL VOI may be a tumor or tissue or organ or surgical tool.

Material decomposition may be iterative based on the requirement of imaging process or based on requirement of diagnosis or prognosis or predication of the outcome or planning or monitoring of treatment or therapy.

Material decomposition may be based on a database which includes measurements in range for each parameters. For example, for soft tissue, there is a range of value which may apply when measured at certain energies.

Once region of interest is determined, the x-ray source or emitting position may be moved or stay stationary, and a collimator or the anode target or the x-ray beam may be manipulated to illuminate only the field of view containing ROI. When the x-ray source or emitting position is moved by a mechanical or motorized positioner or in some cases by a deflector which may include electromagnetic steering mechanisms, the x-ray emitting position can be right above the center axis of the x-ray emitting region and/or center axis or center region of ROI.

To reconstruct a 3D image from measurements from a detector of point, 1D or 2D dimensions, the following factors may be considered:

An energy-resolved imaging system probes the object at two or more photon energy levels. In a generic imaging system, the projected signal in a detector element are at energy levels of Ω∈ {E1 . . . En} If q is the number of incident photons, ϕ is the normalized incident energy spectrum, and r is the detector response function, Linear attenuation coefficients and integrated thicknesses for materials that make up the object are denoted μ and t (attenuation according to Lambert—Beers law). Two conceivable ways of acquiring spectral information are to either vary with q, x ϕ, or to have Ω-specific r, here denoted as incidence-based and detection-based methods, respectively.

In consideration of each pixel or detection region of the detector corresponding to a column or an x-ray illumination path coming from the x-ray emitting position, if an attenuation based linear equation is made up, for example

X1+X2+X3 . . . Xp=DΩ or nΩ

Each X1, . . . , Xn is a volume of VOI which attenuates x-ray in each corresponding Xc layer in VOL Each x-ray attenuating volume in each layer may have an magnifying factor compared to the layer upstream, closest to the x-ray emitting position, therefore the number of subunits representing the size of the volume may have a magnifying factor as well. And in each layer, there may be sub-voxel of one or more voxels with a specific spatial position which is in the same layer of the VOI that contributes to the attenuation of the x-ray beam which projects on to the region of detector and generates a measured signal used in the construction of one of linear equations to solve the unknown values of each voxel in the projection path.

Each X1, if represented by a number of subunits, can have a number of incident photon passing through. And it is expected that photon influx or density passing through each layer are going to be less as the x-ray beam travels downstream to reach the detector due to the nature of cone beam. It is therefore to be expected in some cases, the representation of contribution to attenuation by each voxel in each layer of ROI in the x-ray beam path to have a weighing factor takes into account the magnification factor, therefore reducing photon density or influx downstream of ROI layers compared to that of upstream ROI layers.

Therefore it may be represented as the following

X1+(1/M)X2+(1/M²) X3 . . . Xp=DΩ

Where 1/M may refer to the reduced incident photon going through the same volume of a voxel. If 1/M is the same for each voxel layer.. In addition, the volume of each attenuation unit X2 can be used to describe each voxel or part of voxel may be a whole unit of a voxel or a portion of voxel, which can be represented as proportional number of subunits within the x-ray beam path corresponding to the respective detecting region or pixel. For example, Volume size of X1 may be (0.64) Voxel (x1, y1, z1) and Volume size of Xp may be (1×Voxel (xp, yp, zp)).

Other Examples of Reconstruction Consideration

In 3D imaging, the top surface area of region of interest may be different from area of detector which measures the x-ray passing through region of interest in the subj ect.

For example, top surface dimension in one side may be 16 cm, while the minimum active area of detector may be 20 cm, which captures x-ray illuminating the entire the top surface areas in the region of interest.

If there are 1000 voxel points along the depth of the region of interest, (for example, when the depth is 20 cm,) the x-ray attenuated and passing through an area or voxel smaller than a pixel pitch at the top surface , for example.64 of a pixel pitch of the detector, will fill a complete pixel when reaching the detector. If V1 is 0.64, V1000 is 1, when the area of interest is 16 cm on the top surface and 20 cm when reaching the detector.

When x-ray source moves to a new position, exactly one pixel pitch away from the first position, the x-ray passing through the region of interest having the size adjusted for the magnification factor may land squarely and fully on the corresponding pixel on the detector

If there are 1000 data points, each adjacent voxel in the axis perpendicular to the detector may be multiplied by a magnification factor so that x-ray passing through the voxel may be a slightly larger to receive the projected x-ray beam and therefore the information encoded in the beam regarding region of interest is slighted magnified.

The xy 2D plane, in which the x-ray emitting position moves, may or may not be parallel to the detector.

In instances where low resolution 3D images are taken before high resolution 3D to be constructed, the spatial position on the 2D plane where x-ray emitted from to form the low resolution 3D can be one of spatial position used to form the high resolution 3D. Imaging at the specific x-ray emitting position may not need to be repeated, as the image taken before may be used as one of many required to construct a complete 3D in high resolution.

Various implementation may achieve 3D image reconstruction and derivation of unknown voxel values in the region of interest. Below are non-limiting examples of various combination of steps may be involved:

Step 1:

Determine the center axis line below the x-ray source or center axis of the x-ray cone beam and the pixel or pixel regions on the detector receiving the center axis signal generated due to the x-ray passing through the region of interest or the subject.

Record the relative position of the region of interest to the x-ray source and the detector, and the spatial relative position of the voxels within region of the interest to the detector and/or to the center axis and/or to the x-ray source.

Step 2: Determine the orientation by the shadow below the beam particle stopper or the beam particle stopper

For example, before placing the subject to be imaged in between the beam particle stopper plate 100 and the x-ray source, x-ray is taken with just the beam particle stopper plate in place. In some instances, x-ray is taken with such beam particle stopper plate being placed in more than one spatial locations, such as when the plate is moved in the x y plane and each beam particle stopper are placed in position where the mid point of two beam particle stoppers are on the plate previously. In some instances, x-ray source is moved as the x-ray measurements are taken, so that the location of each beam particle stopper on the plate may be derived in the 3D spatial location relative to the x-ray source or the detector or pixels on the detector, especially when the size of the beam particle stopper are known. The spatial location of the beam particle stopper plate may also be known by the position of the positioner where the beam stopper plate is placed in between the subject and the detector. As the beam particle plate dimension and exact composition and distribution of the beam particle stopper are known, each location of the beam particle stopper can be derived. Based on the measurement of the x-ray reduced significantly at the shadow location of the beam particle stopper, when the subject is placed in between the beam particle stopper plate and the x-ray source, the location of the shadow may be determined. And such a location or a set of locations may be compared to the locations of the pixels where the x-ray measurement is minimal due to lack of primary x-ray, when the subject is not in place, and the location of the x-ray source can therefore be located. The location of the x-ray source center axis and the corresponding pixel may therefore be obtained.

And the location of the scatter only pixels are derived also for deriving low resolution scatter image.

To determine the center axis of the x-ray source, for example, one x-ray measurement may be done without the subject of the beam particle stopper plate 100. The location and/or the size or dimension or thickness of each beam particle stopper may be determined. And the location of the pixel or pixel regions directly along the path of the beam particle stopper if the x-ray source is centered above the particle beam particle stopper may be determined. As the x-ray source or x-ray emitting position moves, the shadow of each beam particle stopper may change its location. The radial center axis may be determined as the center for a radial map formed by concentric circle derived from or synthesized from shadow locations.

The precise spatial location and/orientation of the x-ray emitting location may be determinedby the beam selector orientation to align with the focal point of the x-ray to receive primary x-ray in the rear detector, or an external device for sensing geometry and/orientation relative to the x-ray source and determining the thickness of the ROI or VOI, relative spatial position and geometry of VOI, location of the center axis line of the x-ray source passing through VOI, and distance of x-ray source to the detector and relative spatial position of VOI and each voxel in VOI, relative to each pixel of detector.

The apparatus can determine the thickness or exposure needed for imaging VOI , or geometry of VOI and may be an optical sensor such as a time of flight sensor for sensing distance. The apparatus can determine the distance from source to the surface of region of interest closest to the source or the distance from the source to the detector or the 3D geometry of the region of interest and relative position or distance to the detector and/or the x-ray source and therefore the x-ray emitting position.

The apparatus may be attached onto the x-ray tube or the collimator with adjustable size of illumination aperture, or the mover moving the x-ray tube, or the detector, or the apparatus may be installed in a spatial location detached from the detector 22 or the x-ray source 12.

The apparatus for measuring in determining thickness or exposure needed for imaging VOI , or geometry of VOI may be a Lidar or lidar-like device, which may be used for sensing relative distances and spatial positions and volume of interest or imaged subject spatial position and geometry. The apparatus for measuring in determining thickness or exposure needed for imaging VOI , or geometry of VOI may be an ultrasound device.

Method used in 3D Reconstruction of VOI can Include the Following:

Label each voxel or describe each voxel with x, y coordinate, which is parallel to the detector.

Label each voxel closest to the detector, immediately above the detector with x, y, z, z being, for example, layer 1. And sequentially label each layer further away from detector in the region of interest, until reach the top layer Layer p of VOI which is closest to the x-ray emitting position. Each layer has a resolution Xc along the z axis equal or approximately equal to resolution desired by the application or by the current iteration of 3D reconstruction.

Each voxel closest to the detector may be further divided into even more subunits, such as 100 or 1000 or 10,000 subunits. Such subunits and its relative position in the region of interest or relative to the x-ray source or the detector may be determined and number or noted by its spatial coordinates and as well as the fact that it is part of region of interest compared to regions outside of the region of interest. In the event new voxels unknowns are introduced or subunits unknowns are introduced, the unknowns can either be counted as now part of region of interest and/or be categorized as not within the region of interest prior to the varied x-ray emitting position.

Determine the shortest distance DB from the bottom layer of the region of interest to the detector. This may be a predetermined number or given by user selection or computer selection based on criteria set by the user or AI, or determined by measurements using apparatus.

Determine the distance from the top layer of the region of the interest to the detector. If the top layer is not parallel to the detector. Determine the distance between the plane covering the top layer and the detector D T

Determine the thickness TR of the region of interest by the user or by subtracting D T−DB.

Divide the thickness into units of voxels, each voxel is described by x, y and z. The dimension of each unit of z is the desired resolution in the z direction.

Determine the locations of the voxels VTO at the outmost rim of the region of interest in the x, y and z space at the top surface of region of interest. And determine the x-ray path from the outer most layers of region of interest in the x and y coordinate and its corresponding pixel on the detector and/or the corresponding voxels VBO in the bottom layer of the region of interest. Denote each subunit and/or denote each voxel, so that voxels and subunits of the voxels may be determined spatially relative to each other. The bottom layer voxel subunits may have larger number of subunits than each voxel of the top layer of the region of interest. Each subunit dimensions in 2D or 3D may be equivalent to each other.

In examples where there are more than top and bottom layer in the region of interest, and/or there are more than two layers or voxel layers in the region of interest, each voxel in each layer may not be the same size compared to the corresponding voxel in a different layer. each voxel contributes to the signal level on the corresponding pixel on the detector. In one example, number of subunits in each voxel are determined corresponding to each pixel on the detector in each layer between VBO and VTO.

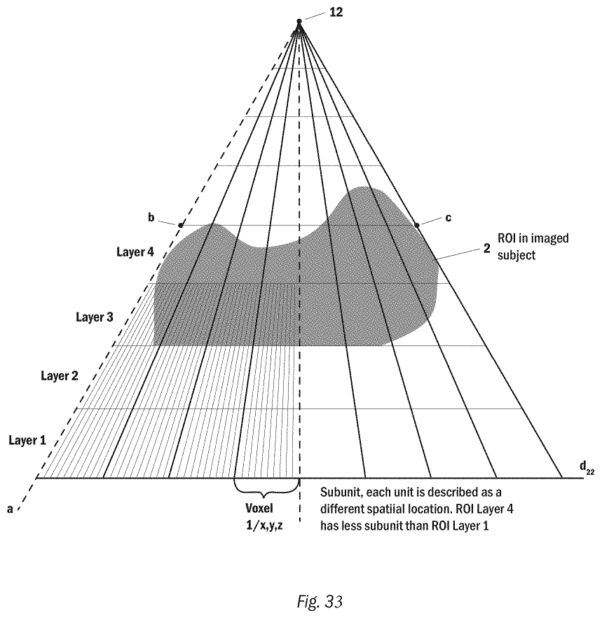

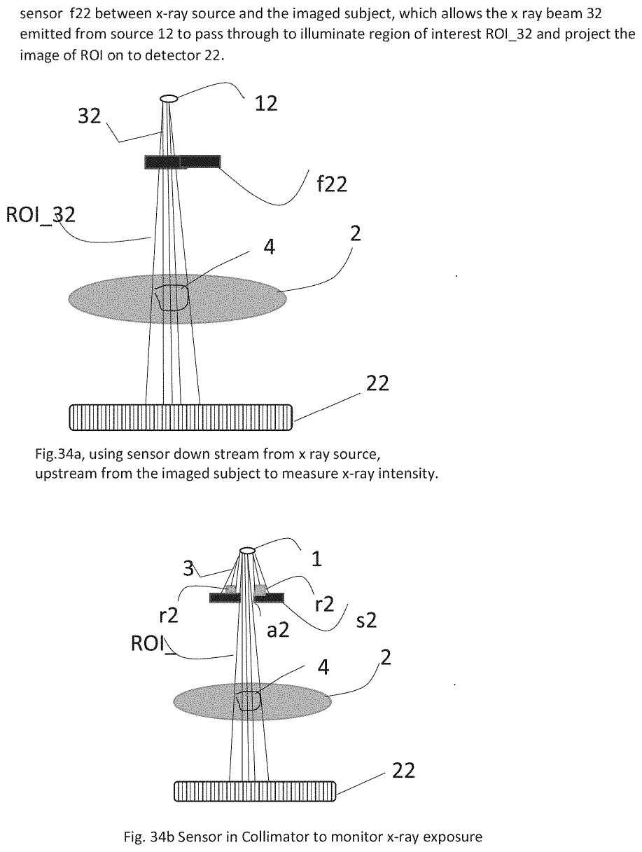

In one example, the size of voxel and/or the subunits, and/or the number of subunits in voxels are the same. However, in each measurement on the detector, a portion of each voxel- or sub-voxel, may contribute to a measured value on a corresponding pixel of the detector, as show in illustration FIG. 33.

For each x-ray measurement on each pixel of the detector which captures the signal from the region of interest, a linear equation may be created based on attenuation values of each voxel or its sub voxel, and its corresponding subunits of region of interest which contribute to the measurement collected on the corresponding pixel.

Based on the x-ray emitting position and orientation and spatial position relative to the detector, each subunit of each layer in the region of interest has a precise relative spatial location to the x-ray source illuminating the region of interest and corresponding pixel location on the detector. Attenuation of aggregated sum of the subunits in region of interest along each the x-ray path result in the corresponding pixel measurement on the detector. A spatial map of the region of interest, voxels within ROI or VOL, its sub voxel volume regions and subunits within it along each x-ray path generating signals on the corresponding pixel on the detector may be derived based on the x-ray emitting position, relative to the region of interest and relative to the detector as x-ray emitting position varies.

To determine the subunits of voxels describing a portion or a whole voxel in volume of interest illuminated by each beam path reaching corresponding pixel on the x-ray detector, calculations may be performed as the following.

If for example, SID is 1 meter, and the pixel size is 100 um, for the x-ray beam path passing through the region of interest landing immediately adjacent to the center axis, there is a volume measurements of ⅓×1000×0.1×0.1=10/3 mm 3.

V=⅓a2h where h is SID, a is the side of a pixel. And V is the entire volume of x-ray travels through before reaching the detector.

However, for each layer, along the height, the volume number is different. For the region of interest, the volume measurement is then

V=⅓(a2+ab+b2)h where a is the side of a pixel, b is the side dimension of the voxel layer , h is the depth of the region of interest.

b of each layer in the region of interest may be derived from the slanted angle of inclination of the x-ray beam and the distance of such a layer to the bottom layer or the top layer or to the x-ray emitting position. ϕ=tan-1(2h/a) where h may be the shortest distance from the source emitting position to the corresponding voxel layer along the x-ray path, a is the side of voxel at such a voxel layer in the region of interest.

For example, since the angle of the inclination stays constant for a x-ray beam path corresponding to one pixel on the detector, then as h varies for each layer of the region of interest, the dimension of the size a or dimension of a voxel described by the size a may vary as well.

For example, if h is 0.8 of SID, then a is 0.8 of the dimension of the bottom layer voxel immediately adjacent to the detector. Such dimensions may be represented by subunits, each may be identical to one another in dimension, but may be of smaller dimensions so that the difference in dimensions of voxel between even the most adjacent layers may be expressed in whole number multiples of subunits. And each subunit may be referenced in its x y z coordinate, and/or each x-ray emitting position and/or voxel which contains the subunit and relative voxel spatial position to the corresponding pixel position on the detector, which is denoted by its x y position. Each voxel may also be referenced by its x y z position and corresponding pixel position and/or relative position to x-ray emitting position or relative position to the center axis position of the x-ray cone beam.

both values of SID or pixel size may be different, the SID may be 1.2 or 0.8 or a different number. And the pixel size may be different. Or alternatively, in some cases, the resolution desired is much larger than one pixel pitch, then measurements on more than one pixels may be used or binned or integrated so that smaller set of data analysis therefore less computing power and less computing time may be needed for reconstruction.

In addition, with 3D measurements and derivation of voxel values, the thickness of certain component may be derived. For example, one component may be defined as a substance with approximately certain density or a range of densities. The determination of the subunit values or voxel values and combined volume and spatial position and the relative value of each may be used to determine the value of the thickness of the component.

In addition, when one component is less sensitive to one energy of x-ray, it may be more sensitive or attenuate more of certain different x-ray energy level, therefore the density or thickness information of certain component may be differentiated further based on measurements at various energy levels. Combined measurements at multiple x-ray energies of one component may further distinguish the component or identify the component. Spectral measurement of one substance may be characteristic of such substance, and distinguish it further from other similar materials, for example, differentiating lung tissue from heart tissue or other types of lean tissues or fat tissues.

The described example of 3D reconstruction methods above may be combined with the hardware and/or software disclosed and referenced in the aforementioned PCT Applications.

Center axis voxel path calculation will now be described.

For a VOI With a Volume calculation=100 um×100 um×1000000 um=0.333×10 10 um3 in the region of interest top layer, If H is the height of a voxel in each layer, for example 100 um, then a is the side dimension of the bottom layer of one voxel layer in the region of interest and b is the side dimension of the top layer of the voxel layer.

V=⅓(a2+ab+b2) d

The angle is ϕ=tan-1(2d SIa/a)

For center axis x-ray cone beam, if sides of the cross section of the top surface, closest to the x-ray source, are described as a, the region of interest is a×a and the depth of the region of interest is d, and the pixel region of detector which captures x-ray illuminating the region of interest are square and has a size in dimensions of b.

If the resolution of the region of interest to be acquired is Rr along the depth, the number of unknown voxels points may be approximately d/Rr.

The magnification factor Mf is x-ray cone beam magnification factor, which results in the area of dimension b×b from the a volume described for the region of region as a×a as b2=a2×Mfn

Where n is approximately d/Rr. If Mf is 1.00004, the number of x-ray subunits is set as 100,000 units for the bottom voxel immediately on top of the each pixel on the detector, there would be 330 rows and columns of subunits, each are in cubes with equal size and are equal in size with each other, contained in the voxel immediately above each pixel including the center pixel. If for example, there are dimension of b×b on the detector and b×b contains N b voxels, and each voxel may be approximately equal to or greater than a pixel pitch of the detector or the size similar to resolution desired for the depth, then the number of subunits describing the voxel layers in the bottom is 100,000×b×b. if the resolution along the z is Rr. There are Rr=g×pixel pitch of the detector. In each voxel there are g layers of subunits. In each voxel, there is a top layer side and bottom layer side. If bs×bs is the number of subunits on one side of the bottom layer and ts×ts is the number of subunits on the top layers of subunits within one voxel, then

Ts×ts×mfg=bs×bs.

Each layer of g layer has subunits within the voxel that are magnified to next layer closer to the bottom layer subunits by a factor of mf, until the bottom layer is reached or the gth layer is reached.

The subunits and voxels may be determined by methods such as described below. The center axis of x-ray source falls in the middle of exactly four pixels.

There are four identical voxels around center point on the detector corresponding to the center point of x-ray emitting position.

For the 1st x-ray position, the region on detector, R may be an integer multiple of pixel pitch, z is 1Rr

X1R Y1R, X1RY−1R, X−1RY1R, X−1R, Y−1R

for x1R y1R, there are subunits x330s, y330s, each subunit denoted as the relative spatial position in units of subunit dimensions away from the center axis point.

X1RY−1R, subunits x330s. Y-330s

Within the volume of X1R x Y1R, there are gth layer, mf=V(g&(bs*bs)/(ts*ts)) where g is Rr/pixel pitch of the detector. Therefore the second subunit layer from the bottom subunit layer in the same voxel will have total of 330×330×mf subunits, with each side of the subunit layer being in the dimension of 330×√(mf). until the gth layer. The subunit in the second voxel away from the center axis point, adjacent to the first voxels, may count its spatial location from center axis in number of subunits. To calculate the number of subunits way from the center axis, multiply the number of voxel positions, each with the subunits layer selected, with number of subunits specific for the subunit layer for each specific voxel position.

When x-ray source is moved, or the x-ray emitting position is moved, the center axis position is determined again. And the region of interest x-ray path travel through is redefined due to varied x-ray illumination path. The surface of the top layer may be changed and the number of bottom layer pixel measuring the x-ray beam passing through region of interest may be varied. Voxels from each voxel layer in the region of interest corresponding to the x-ray beam passing through and generating a signal on a specific pixel or a region of two or more pixels may be different from before and each voxel in the same layer also may be different in terms of which subunits are in its volume. Alternatively, each subunit and its relative positions to the center axis and as well its relative spatial position to the source may stay the same. Similar method may be used to name each voxel and its relative position to the center axis and determine which subunits and how many subunits are in each voxel.

If approximately d/Er number of images are taken at varied x-ray emitting positions, where x-ray emitting positions travels in an area which is approximately the distance of the depth and/or the adjacent x-ray emitting positions are approximately Xr apart, in space, then a number of linear equations are formulated corresponding to each measurement on each of the pixels measuring x-ray passing through the region of interest, and the voxels and subunits in the voxels in each x-ray beam path. The total number of measurements in each pixel or each pixel region including two or more pixels on the detectors may give rise to the total number of linear equations involving voxels and subunits in them. Some examples of such methods are detailed in the aforementioned PCT Applications.

When a pixel region is involved, the total measurements averaging to each pixel are used.

In addition, subsets of subunits may be combined to form a unit of synthesized pseudo voxel, especially if the linear equation involves each of the subsets of subunit in most calculations. In addition a fraction of voxel in each measurements may be represented by a subset of the subunits. Its attenuation value may serve as an unknown unit as well. However there may be cases where a fraction of such subset is counted, for example, if with high probability, the voxel has the same measurement as the voxel around it or slow varying or uniform in its composition, and there is no other indication in its adjacent regions to indicate why it would be different from the voxel around it or the other part of the same voxel. Iterative methods may be used to check if indeed it is the case that the subset of the voxel carries the same density value and proportional in volume to the voxel and/or one or more voxels around it. In addition, the derivation may come from the location of the voxel relative to the region of interest and other component and location of the voxel within the same component.

Smaller movement steps between x-ray emitting positions to illuminate certain region or certain part of volume in the region of interest can be if there are unknown voxels or subunits of voxels or subset of voxels are not resolved.

Measurements and same process may be carried out with smaller voxel size and with higher resolution detectors, or better resolution in the depth may be needed to further resolve values in regions not clearly resolved or if there is clear indication that such derivation or interpolation and approximation may not be done due to facts or conclusions drawn and indications given from preexisting measurements and other information and data sources.

Qualitative and Quantitative 3D, CT computation, X-ray measurements, sometimes, require large amount of memory and computation. In the nMatrix or n2Matrix method and apparatus described here, a detector module or assembly or a submodule attached onto existing detector or via wireless communication or tethered mechanisms, may contain memory storage, and/or database capability and/or database and/or microprocessor and/or for localized storage and computation at the detector side, processing and/or storage. The display may be done locally or directly from the microprocessor or remote via wireless or ethernet or other tethered communication method to a second microprocessor for display and in some cases further computation and storage

Software and/or electronics and/or image sensors with ADC resolution of 32 bit or above may be used for image processing, including fast and accurate tomographic reconstruction with some of the existing CT, or multiple dimensional x-ray imaging, 2D images acquisition, processing, and spectral imaging, dynamic image processing, tracking, multiple dimension applications due to high dynamic ranges, especially combined with a selection of VOI for tomography imaging. A high resolution image, such as in the single digit micron or sub micron range, along the z axis, for example at the dimension range of small blood capillary, may be achievable with a reasonable radiation level and speed for tomographic imaging of a large object such as a human. Previously such resolution is possible on small samples using microCT but not for medical CT.

Contrast Agents

Due to quantitative imaging and increased sensitivity in detection, existing contrast agents for x-ray or other modalities may still be used, but at lower levels than before to reduce toxicity and adverse affects on a subject. For example Gadolinium , iodinated, bismuth, barium and calcium and nanoparticles, and their derivatives such as chelated complexes, may be used in smaller amounts, down to micro, or nano, or pico or femto or lower molar levels, and its detection in applications such as density measurements may be amplified or modified in its presentation and/or presented with different color or signal levels for user or digital program or computer to improve accuracy of diagnosis, characterization of substances, monitor events in an ROI and guide procedures based on AI algorithms that are developed using deep machine learning, neural network and other methods used in other industries. Combined with quantitative measurements in density, thickness, atomic z identification, and other commonly used AI criteria, contrast agents can assist in procedures in medical, industrial and security applications.

M3- personalized CT or 3D imaging based on nMatrix method, is a customized imaging method that minimizes required radiation, toxicity of contrast agent, and provides better control of intervention devices and treatment level and quantitative assessment for each material or substance of interest in the ROI and each related procedure .

The nMatric method can include features such as distributed measurement regions, or distributed measurement time intervals, 2D spectral instead of tomography, low resolution 2D, low resolution 3D by using structure illumination or binned detector measurements at low exposure level, and larger Xc steps determined for each procedure, and highly restricted focused ROI using smaller detectors and fast acquisition detectors with higher sensitivity by using high spatial and spectral resolution detectors with reduced SPR of less than 1% or less than 5%. And such a system may be used with fast millisecond switchable or micro second switchable field emitter x-ray tube, combined with conventional tube to emit x-ray at the same emitting position at different times or different position at the same or different times.

The apparatus and methods of spectral imaging apparatus and methods, and/or multiple dimension imager or 3D imager or 6D or 7D imager and/or spectral CT or 3D systems capable of scatter removal to <1% of SPR, or less than 5% SPR, may use markers and/or contrast agents, including but not limited to molecular imaging contrast agents and markers, previously used for applications with CT or microCT or PET, or other modalities to be used with the x-ray detectors or 2D detectors or photodiodes with dimensions equal to 1600 cm2 or above, or 400 cm2 or above, or 40 cm2 or above, or 20 cm2 or above, or 10 cm2 or above, or 5 cm2 or above, or 1 cm2 or above, or 0.5 cm2 or above, or 0.1 cm2 or above, or 0.01 cm2.

Contrast agents may be conjugated in a molecular complex. For example, this may include a targeting protein, or small molecule or which may include one or more molecules, made of protein and/or lipids and/or other chemicals, sometimes present in a larger complex or complexes, such as liposome, albumin, polyclonal antibody, monoclonal antibody, synthetic antibody and antibody-derivatives or variants such as scfv, fab, minibody or nanobody, or vh domain, or DARTs, or AFP or Albumin, or a conjugating unit or conjugation chemistry such as a thiobridge, or cyPEG or maleimide or NETS-ester, or click chemistry or oxime or enzyme mediated conjugations, to connect the target protein with the contrast agent or any other elements in the complex, and/or a polymer which can stabilize or form multiple repeating units to hold contrast agents or to stabilize the molecular complex in vivo , and/or a release link such as disulfide, enzyme or pH cleavable, carbonate, or noncleavable, and/or a payload, for example, various organic or synthetic agents which may be measured and quantified differentially than other objects or tissues or other components or combinations of tissues or substances inside a body of a human or animal or ex vivo.

Such a molecular analysis system may be used in a flow cytometer, where the label is x-ray contrast agent. Instead of laser based detection, an x-ray measurement system sensitive to x-ray contrast agents is used or x-ray measurement system may be hybridized or combined with a fluorescent dye based traditional flow cytometer where both fluorescent signal and x-ray signal can be recorded.

Contrast agents used in PET or MRI, or PET /CT, Single Photo Emission Tomography, for example, which behaves like calcium biologically may be used as an x-ray imaging contrast agent. One example is the radioisotope of Strontium, which may not be needed because strontium is readily detectable by x-ray and it has low toxicity and a well-understood safety profile. Instead a non-radioactive isotope of element may be used as x-ray contrast agent.

Contrast agents designed for PET may be coupled with molecules that recognize target epitopes with high specificity on important proteins, such as proteins associated with certain illnesses. The radioactive isotope of the element may or may not be replaced with nonradioactive isotopes of the same element or substance and conjugated to protein epitope-binding proteins such as antibodies and its derivatives.

For example, these agents may be non-radioactive isotope or stable isotopes of Ca, Cr, Co, Ga, Se, Kr, Tc, In, I, Xe, Sm, Tl, or in another example, F-fluorodeoxyglucose.

Genetically engineered genes which produce proteins, subunits of proteins or antibodies or nanobodies or nucleic acid-based oligos or protein complexes or complex vesicles such as liposomes to capture Ca++or other cations and/or contrast agents which have been used before for PET, Mill, CT, including nanoparticles, barium, strontium and rubidium, which is differentiable from that of cells in normal state, may be used here.

Permeability of fluidics or blood vessels in the region of interest or in part of ROIs and flow characteristics may for instance be used for diagnosis, inspection, monitoring and tracking.

Diseased regions such as tissues with tumors, arthritic areas, fractured regions, energy treated regions such as by ultrasound, or RF or laser, are physiologically and biologically distinct from normal or untreated tissues. The x-ray imaging system and methods described may be used to detect, diagnose, monitor and survey such disease.

Prior to an intervention procedure, such as RF ablation of a cardiac tissue or renal tissue, a portion of tissue, a portion of the ROI, may have a specific permeability characteristic that may be modified and different when a treatment and procedure has been performed in the ROI. Contrast agents or labeled substances may be injected, aspirate or absorbed in a portion or complete region of ROI with permeability characteristics which may differentiate from surrounding tissue. This device combined with the contrast agents may be used for image guidance of the intervention procedure to monitor therapeutic treatment during the intervention procedure. Diagnosis, or monitoring of ablated regions with minimized toxicity may be preferred in some intervention procedures to better monitor intervention process and outcome. For example, in cardiac ablation, liquid with contrast agents may be aspirated into ablated region during procedure, and the ablated regions would have a different permeability such as pattern of permeability or speed of permeability of ablated region that is different from healthy or unablated region. This may guide the effectiveness of the treatment, adjust treatment level, reduce time required for the procedure and/or limit damage to the surround tissues during the procedure.

X-ray imaging system described here may be used as in vitro imaging system therefore providing a universal imaging and measurement system for both in vivo and in vitro measurements for drug development and life science research. When used in in vitro setting, the sample may include fluidic, or microfluidics device; the contrast label may be fixed to a surface in 1D-3D spatial positions relative to the surface. There may be one or more contrast labels conjugated with one or more molecules which may interact or being present together in a mixture of molecules, complexes of molecules or cellular mixture with molecules. The labeling may be done with contrast labeled antibody against tumor markers, such as CA19-9, in vitro and in vivo, to study tumor or region of a tumor and to help therapeutic guidance. Contrast agents may be PET contrast agents without radioactivity, or MRI contrast agents, or X-ray/CT contrast agents, or endogenous natural elements, such as calcium, zinc , iodine and their derivatives.

Tumor marker may be fixed on a surface, and the contrast labeled antibodies or small molecules may be incubated with the functionalized surface, which may be static or in motion such as on a moving particle. The particle may be an antigen, such as from internal or external pathogen or hybrid of both. One or more ingredients of the mixture may be sorted or manipulated by optical or energy tweezers or manipulators, or by weight such as on a spiral microfluidic chip, spinning motion separate particles by weight. One or more molecular assays may be performed.

Binding of the target and conjugates may be via base pair hybridization if the active regions are parts of oligonucleotides, parts of DNA or RNA or RNA like molecule or hybrid molecule or combination of antibody or oligonucleotide complexes, be it natural or synthetic.

In Vivo and Human Body Imaging Examples will now be explained.

Endogenous compound and molecules or substances or their modified versions which may be used for therapeutic purposes, may be used with x-ray system-based imaging described in this disclosure as a contrast agent for quantitative characterization of its associated molecules or tissues or component or an object, or for identification and measurements of materials in ROI, which would not immediately or be easily differentiable in an x-ray measurement or CT systems based on conventional methods. With general conventional x-ray systems, it is difficult for x-ray system to distinguish contrast agent from bone images due to overlapping attenuation features. Since these devices mostly separate components or images by anatomic markers, if the endogenous compound is embedded in a projected path, general conventional x-ray systems are not capable of quantitative measurement therefore enough sensitivity for the contrast agents. Quantitative measurements are defined as measurements of bone density or thickness and/or consistent attenuation characteristics at one or more energy levels, and therefore these systems and/or contrast agents based on endogenous molecules and substances may not be suitable in conventional imaging methods to determine the quantity or identify the presence or the quantity of the contrast agents embedded within an overlapping tissue in an object.

The x-ray imaging system of the present disclosure may be quantitative, for example when scatter is removed so that SPR is less than 1% or 5%, and/or with measurements at multiple x-ray energies, and/or with reconstruction of 3D and/or CT tomographic images.

When there may be a difference before and after in the measurements due to the presence of the contrast agent, the contrast agent can be identified and also quantified. This is especially likely because bone structure and shape in general do not change when it is static or in motion due to its rigidity. However, the contrast agent may have a set of predetermined characteristics, depending on how it administered. For example, if injected in liquid form, the contrast agent may travel or stay in fluidic form, which is significantly different from that of bone. Or if the contrast agent is made into an implant or probe, there is a set of predetermined x-ray imaging characteristics due to its design and composition, making it likely to be differentiable from bone.

In presentation and display against the rest of imaged object or ROI, a material including a contrast agent may be represented with a selected color tone or texture or a visual characteristic differentiable from that of the bone and other tissues or object in the ROI.

In flow direction characterization, one or two contrast agents may be used. For example, a calcium-based contrast agent can be first injected, followed by an iodinated contrast agent. The flow characteristics of both, and/or whether they will merge or maintain a relative position with the other over time, will determine the flow direction. Microbubble based contrast agent may also be used as one of the contrast agents used in flow direction studies.

In another example contrast agents can be measured before and after images of tomography. It is conventionally not recommended to use CT before and after procedures as too much radiation will be received by the patient, especially pediatric patients. With the disclosed imaging systems, radiation levels are extremely low compared to conventional CT. In addition, imaging is also targeted, sparing healthy tissue from unnecessary radiation exposure. The imaging systems disclosed herein can be combined with contrast agents such as calcium as diagnostic procedures in contrast to calcium with CT.

Calcium conjugated molecules such as calcium Calcium Gluconate is commonly used to treat Hypocalcemia, and sometimes, mixed with NaCl, for example, with the following dosage: 500 mg; 100 mg/mL; 650 mg; 1000 mg; 1 g/50 mL-NaCl 0.9%; 1 g/100 mL-NaCl 0.9%; 2 g/100 mL-NaCl 0.9%; 1 g/50 mL-D5%; 20 mg/mL-NaCl 0.67%; 2 g/50 mL-NaCl 0.9%; 1 g/25 mL-NaCl 0.9%; 1 g/100 mL-D5%; 2 g/100 mL-D5%; 20 g/1000 mL-NaCl 0.9%, for treatment. The same compound may be used as an angiographic contrast agent with adjusted dosage. Before or after the administration of the injected calcium gluconate solutions or before the calcium gluconated blood or body fluid reaches the ROI, bone images or calcium or microcalcium may be measured and registered or determined, and may be removed from the rest of body image or be used as a reference object. Administered calcium can be measured and accurately determined, sometimes in real time. If needed, Calcium may be used in place of iodine or iodinated , iopromide as contrast agent with much less dosage in x-ray imaging of the present disclosure.

In addition, calcium gluconate or calcium chloride can be used when treating hyperkalemic cardiac toxicity during an malignant hyperthermia crisis. For example, Calcium chloride (4-10 mg/kg) may be used. Different dosage may be needed or adjusted for safety reasons. Typically, Calcium chloride at 10 mg/kg (maximum dose 2,000 mg) or calcium gluconate at 30 mg/kg (maximum dose 3,000 mg) is used for life-threatening hyperkalemia so the safety profiles for both calcium formulations is well-established.

Same reagents such as calcium chloride, calcium salt and calcium gluconate may be used as a contrast agent for visualizing blood, in angiography or for minimum invasive surgical guidance to determine the relative location or spatial position of the blood vessel to the surgical probes or tools or biopsy probe, implant, catheter tip, or guide wire, target of energy based therapeutic treatment or chemical treatment, or diseased region, or ROI identified by various criteria or ROI, or a reference object in an x-ray imaging system or hybrid measurement and imaging system including of x-ray and other modalities.

A typical formulation may be 100 mg/mL (13.6 mEq Calcium/10 mL), for intravenous use. Osmolarity 2.04 mOsmol/mLEach mL contains: Calcium Chloride Dihydrate 100 mg in Water for Injection q.s. pH (range 5.5-7.5) adjusted with Hydrochloric Acid and/or Sodium Hydroxide. Each 10 mL contains 13.6 mEq Calcium and 13.6 mEq Chloride. The molecular weight is 147.02 and the molecular formula is CaCl2.2H2O. The formulation may be adjusted for the use as a contrast agent or may be the same depending on the requirements or sensitivity or the configuration of x-ray system.

For example, these reagents may be injected at 10 mg/ml in a 1 minute injection period.

For example, 10% Calcium Chloride Injection, USP can be administered by slow intravenous injection (for example at 1 mL/min).

Zinc injection may also be used as a contrast agents for visualizing presence of blood and blood vessels. A typical concentrated Zinc Sulfate Injection, USP is a sterile, non-pyrogenic solution intended for use as an additive to solutions for Total Parenteral Nutrition (TPN). Typically each mL contains Zinc Sulfate (Anhydrous) 12.32 mg, Water for Injection q.s. pH adjusted with Sulfuric Acid. The formula may not contain any preservatives. It delivers elemental zinc at 5 mg per mL. The amount or concentration needed for x-ray imaging as contrast agents may be experimentally determined within the safety profile and potentially adjusted for its intended purpose.

Mg or Mn conjugated solutions may be used in similar fashion. In some cases, contrast agents with varied x-ray attenuation properties may be administered sequentially or at varied times and/or through different administered routes, for example to characterize flow direction of body fluids, such as blood.

The measurement and quantification of endogenous element-based contrast agents, such as calcium may be done in a number of ways, including:
Before administration of a contrast agent or after a contrast agent has been approximately cleared out of the system, spectral imaging at one or more energy levels or spectral multiple dimensional imaging, and in some examples, segmentation image processing or digital analysis of various tissues or ROI may be done. Also, identification and quantification of bone tissue or other tissues, and/or of cation rich regions or and/or of microcalcifications, and/or calcium scoring may be performed.
After the contrast agent such as calcium chloride , is administered, within the time frame specified for the contrast agents to circulate and reach its destination or destinations in the body, measurements may be done in one, or two or three or more energy levels, to characterize all calcium rich regions. The additional measurements due to the contrast agents can be separated and positioned and compared with images or measurements without the contrast agents. For example, if measurements along one projection path vary from the measurement before administration of calcium formulation on the same projection line, the difference in measurements may indicate the presence of calcium chloride, and may be quantified using any method described in this disclosure. For example, if the thickness of calcium or bone measurements as derived is larger than the value before the contrast agent was administered, it is reasonable to assume that the difference in measurements is due to the contrast agent in the part of the blood vessel within the projected path. The thickness of the portion of the blood vessel may be derived from measurement of the same blood vessel in the adjacent regions where there are no bones in the projected path. The density of the contrast agents in the blood vessel which resulted in the measurements may then be determined.
In some cases, the relative or spatial location of the calcium rich regions, for example, from a segmented 3D reconstructed data set, may provide information as to which portion of the calcium rich region is of bone material or pre-existing in the ROI, which portion of the measurements are of ROI without bone tissue, or which portion is due to contrast agents in the tissue such as a blood vessel or heart.
In some cases, pre and post administration of contrast agents measurements need not to be done in order to identify and characterize and quantify contrast agents and its related target to be imaged. For example, the analysis of point or 1D or 2D — 7D multiple dimension images, in various spatial locations of ROI may be sufficient to identify and characterize the location of various calcium rich regions or deduce the source of measurements for example to be tissue, bone tissue, or microcalcification or blood vessel injected with contrast agents.
Methods using digital analysis which analyze measurements based on spatial location, density and slow varying nature of tissues of interest such as bone or soft tissue or blood vessel or slow varying nature of tissue as a reference object, for example a portion of the chest bone or a portion of skull or part of a tooth, may be used to locate and position one or more individual substances or component or materials in at least one portion of targeted tissue, or component or ROI. For example, the method may determine if the contrast agent rich region is within one region of soft tissue, separate from the bone, but overlapping with the bone at one region. However, since bone tissue or soft tissue spatial volume and/or density are slow varying, data deduced from one region of measurements where there is no overlap may be used to deduce the bone density at the region which overlaps with the contrast agent conjugates and its related targets. In addition, the bone geometry and spatial location and dimension may also be deduced from the measurements around the overlapping region. Therefore the data deduced from the bone tissue may be used to derive quantified value such as density and thickness of the calcium contrast agents by subtracting the contribution from the bone tissue in the measurement pertaining to calcium and bone in the projected line. For example, if it is a blood vessel, the dimensions and density of contrast agents are desired to be deduced. The spatial volume of the blood vessel overlapping with the bone tissue may be derived from regions adjacent to the overlapping area. The density of the contrast agents contained in the blood vessel may therefore be obtained this manner.
Representation of the contrast agents and its targets in imaging, when similar or approximately the same to endogenous tissues or molecule such as calcium or zinc, may be done with a distinct visual representation, such as color and/or increased contrast or dynamic range or intensity from the background, which distinguishes the volume containing or interacting with the contrast agents from that of the endogenous tissue. For example, blue or red color may be used to represent venous or arteries mixed with or containing calcium contrast agents.

To lower the toxicity of the contrast agents, for example, methods to dramatically reduce the dosage of iodinated contrast agents may be used. For example, iodinated reagents are injected to visualize blood vessels. If the dosage is dramatically reduced due to the measurements and its derived results from point, 1D, 2D to 3D or 7D measurements, especially with the assistance of AI to identify targets based on density, spatial location and thickness, and/or the relationship with other component or tissues or reference object and other measurements of different modalities, the contrast agents may be used at low dosages, and quantitative point, 1D or 2D or 3D images may be used to detect the presence of contrast agents, and in some cases, to quantify amount of contrast agents. When the contrast agents are detected and measured, quantified and are undergoing material decomposition into its pure or relative pure substance level, visually a material decomposed image may be extracted and presented. However, when the amount is very small compared to the approved dosage, for example a very small amount of contrast agents are administered into the blood vessel, the amount of contrast agents can be instantly measured and/or may be visually represented by a color which is different from or similar to or the same as the natural color of the measured ROI or the subject or contrast agent, and dosage may be adjusted to distinctly represent the contrast agent containing target or VOI against the background. The intensity and displayed dynamic range of the color chosen for display of the contrast agents and its labeled object or tissue such as blood vessels and nerve tissues may be quantitatively and proportionally related to density and thickness of the contrast agents used. In some cases, no proportional relationship between the brightness of the color displayed and density of contrast agents is required.

Additional Example of Calcium based solutions as contrast agents will now be explained.

In pediatric advanced life support, Calcium chloride 10%: is indicated in hyperkalemia, hypocalcemia, overdose of calcium channel blocker, and hypermagnesemia. During cardiac arrest, the dose should be given as a slow push at 20 mg/kg (0.2 mL/kg) IV/IO and then repeated as needed.

In addition to injection, intravascular administration, oral or rectal administration of contrast agents are all examples of route of administration for the contrast agents suitable for the disclosed x-ray imaging device described here—spectral CT in time domain, or 2D or 1D or point measurement and imaging.

Other routes of administration may include, for example, intrathecal, intra-articular, intramuscular, intradermal, intraperitoneal, intravesical and intrauterine.

Similarly, other contrast agents which have been used in other modalities as well as x-ray imaging may be used with the present x-ray system and some may be used at lower concentration than what has been used in prior art. For example, in vivo contrast agents used in MRI, CT, ultrasound and PET Gadolinium-Based Contrast Agents (GBCA) may also be used.

Tracking can be performed when using an external component or a component of known structure and density, and different from the unknown component, and the region of interest, but internal to the subject, with one of more of the following characteristics:

Known complexity—x-ray measurable or can be measured by one or more other modalities such as Optical, Sound, MM, PET, SPECT.

Spatial structure in 1 or more dimensions; Spatial microstructures in 1 or more dimensions.

One or more x-ray beams at various location is applied first and separately, measurements are taken for the measurement of volume of the component or subject or target of interest, or the measurement of multiple dimension, or prior to knowing where the component is internal of the subject of interest, or other components in the subject.

Such an external component ECR or ICR may be placed on the side of the region of interest or subject of interest, away from the illumination path which projects the region of interest on the detector, or such ECR or ICT may be placed in the illumination, but between the subject and x-ray source or subject and the detector.

In some cases, such external component ECR or ICR are not needed when other measurements such as a multiple dimensional imaging system or 3D measurements of the region of interest or measurements using modalities and systems other than described here or predetermined spatial positions are known.

In some cases, ECR or ICR are combined with other known values or measurements done with other systems and modalities and methods.

In some instances, a spatial sensor or optical or thermo camera , or a video camera is used to locate the subject spatially relative to a reference object which may be internal to the subject or external to the subject. Or such a sensor may be used to locate ROI to another ROI in the subject, or to image statically or dynamically region of interest, or the subject, and determine complete or partial geometry of the region of interest or subject, during or before and after the x-ray measurements to provide information needed to track and/or identify and/or characterize a component, or a region of interest of a subject or relative spatial or temporal relationships between or among subjects or components or region of interest, optionally a reference object.

In some instances, artificial intelligence or deep machine learning or machine learning algorithms are trained based on recorded tracking of each component, target, or region of interest or the subject based on one or more set of measurements spatially or temporally. Addition events may be triggered by the results learned from the tracking activities.

For example, during surgery, one or more surgical procedures of placing an implant or making ablation using energy such as RF or laser or ultrasound, can be tracked and analyzed using AI. Surgeons or a robotic surgery system may be guided using recommendations from AI results or automatically move and perform surgery based on training results under various scenarios.

Derivation of one or more facts such as diagnosis or drawing a conclusion may be based on one or more instances of tracking of movement or interaction molecules, such as a contrast labeled molecules or x-ray measurable molecules, or molecular complexes or one or more molecule or element defined regions or cells, such as a labeled cells or components or region of interest in a subject or the subject itself

Derivation of one or more facts such as diagnosis or drawing a conclusion be used on pattern or shapes or transformation in shapes, composition, movements or interactions of components or targets, for example, molecules, such as a contrast labeled molecules or x-ray measurable molecules, or molecular complexes or one or more molecule or element defined regions or cells, such as a labeled cells or components or region of interest in a subject or the subject itself.

Flow Tracking

When using 2D image, or sliced image 1D or point image or multiple dimensional image, or limiting the region of interest to a volume or a slice of flow volume, or to a selected volume in the subject, for example, in tracking blood flow, the region of interest to monitor can be selected as lines along the center of blood vessel. In some cases, a selected 2 D plane or selected 3D volume along the center of blood vessels is imaged or at sliced line along blood vessel or a cross-section off axis such as perpendicular to the flow direction at distributed locations. This may limit the radiation and/or may include large regions of sampled locations. Such measurements may allow analysis of flow from different regions of the subject at the same time and characterize dynamic relationships between different regions. Diagnosis, tracking and characterization of blood flow, illness, physical conditions and fluidic behavior may be includes in such x-ray measurements as one of the indicators. Multiple x-ray energy levels may be utilized for tracking different component and different element of the flow. For example, white blood cell and red blood cell may have different x-ray measurement properties, or a contrast agents targeting specific marker may be differentiated.

Automated algorithms may be used to extract cell speed from such data sets. Images may be used to identify capillaries that are deformed, have speed out of range, or stalled or having no moving markers such as red blood cells, or have flow dynamics which are disorganized such as in capillaries generated by tumor, or in vascular degeneration. Addition markers and contrast agents for x-ray may provide structural and functional measurement capabilities.

Phase contrast images may be utilized to characterize and diagnose various state of blood flow or fluidic dynamics of a fluidic systems. Statistical analysis of multiple systems and fluidics and fluids may provide data for modeling using digital program and AI to diagnose and troubleshoot or identification of one or more region of interest or one or more components or one or more target in a subject.

Colocation of two or more modalities

The x-ray system may be collocated with PET, or MM, or Optical or ultrasound modalities. Colocation may be done by measurements of one or more anatomic markers or common contrast agents and conjugated contrast agents, including at least one contrast agent for each modality.

In addition, such methods for separating and reconstructing images of objects including at least two materials or entities with varied atomic z number or tissues can be extended to multiple dimensions both in space and in time by taking additional images in space, such as using different detectors or x-ray source in a different location or moving the object in 3 D space or taking a second or more images at a different time. When x-ray images of the material in the object are derived from x-ray images taken of the object in different positions in the 3D space, the resulting images of the same material combined may give more information about position and characterization of the object and its individual composite materials in 3D space. When the x-ray images are taken at varying times, for example, when images of the objects are taken, each time, an image of the individual composite material of the object can be derived, by tracking the dynamic position of the selected point on the composite material. Information about position or function or movement characteristics of individual material in the composition of the object can be obtained. For example, spine movement dynamics such as flexion-extension motion of the lumbar spine or dynamics of head and neck movement or joint, or dynamic of heart rate may be obtained. Therefore this method allows for functional analysis including the movement and characteristics in space and time of individual composite material as well as the whole object.

Intervention

In image guidance, surgical and process guidance or auto piloted therapeutic processes can involve surgical robotics or therapeutic treatment and diagnostics such as electrophysiology, learning the process and analysis processes deriving from measurements including time sensitive measurements, at one time and/or over a period of time of real time processing, using software to guide therapeutic or treatment processes based on, evidence based guidance, computer commands, user (both human and/or machine) input, time sensitive measurements and facts derivation from the subject and/or learnt capabilities from analysis of measurements from previous procedures or simulated procedures and datasets.

Radio-opaque markers can be placed for better visualization of one or more components or targets or region of interest in a subject. For example, radio opaque markers can be placed or integrated into implants or catheter or surgical probes and biopsy probes.

Design of Implant

Catheter or an implant such as a stent or heart valve or surgical tool tip interacting with the tissue, or exterior of non contact probe or contact probe for biopsy or energy treatment may be designed to have different regions, each with the same or varied material properties such as density, or thickness or both or atomic z, or combination of materials, with specific patterns or shapes and geometric configuration measurable by x-ray. For example, different material of varied atomic z may be placed in positions to be measured by the x-ray and differentiated, so that the orientation and spatial location of the different regions and the implant or the object may be determined based on relative spatial locations and distances from each other.

An intervention device, such as a biopsyprobe, robot surgical probe or tool tip, a catheter, an implant, temperature probe, ultrasound probe, pressure sensor, energy transducer, may have portions of its device attenuatingX-ray at different levels or or have an internal component(s) such as a lumen, a guide wire, or valve driven liquid handling tube, or its sheath having different X-ray attenuation properties than the rest of the device. This integrated design of intervention design combined with x ray imaging or tomography system may allow selected portion of intervention device to be moved, controlled and monitored, in some cases with feedback from a x-ray measurement .

FIG. 41 illustrate an example of a region of catheter or an implant, such as a stent or heart valve,or energy intervention probe, designed to be better visualized by x-ray measurement and/or imaging.

There may be one or more regions on the implant or the probe 2000. For example, Region A of the implant may be made of material or synthetic material with certain measurement profile at one or more energies of x-ray. Region B may be made of a different or the same material. The lumen of the catheter 3000 and the sheath of the catheter 3001 may have differential x ray measurement properties, move independent of A or B of the implant 2000.

Distance and relative spatial orientation of A and B may be measured to determine the orientation of the implant 2000 spatially or used to monitor the movement of A relative to movement or state of region B for a user to better control the implant or for the user to monitor the dynamic spatial changes of A or B independent of each other or A relative B and vice versa, and/or A relative to other anatomic markers or reference component or reference location in the subject where A and B and the implant is placed in.

In some cases, the material in region A may be segmented by different density, which may be different at different spatial locations.

And such hardware and software systems may be integrated with optical, Mill, ultrasound, photoacoustic, magnetic particle, nuclear medicine based systems, such as PET or SPECT.

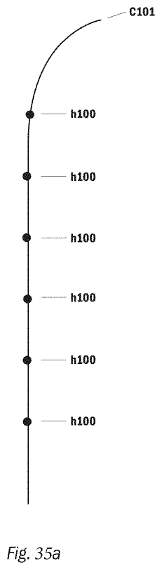

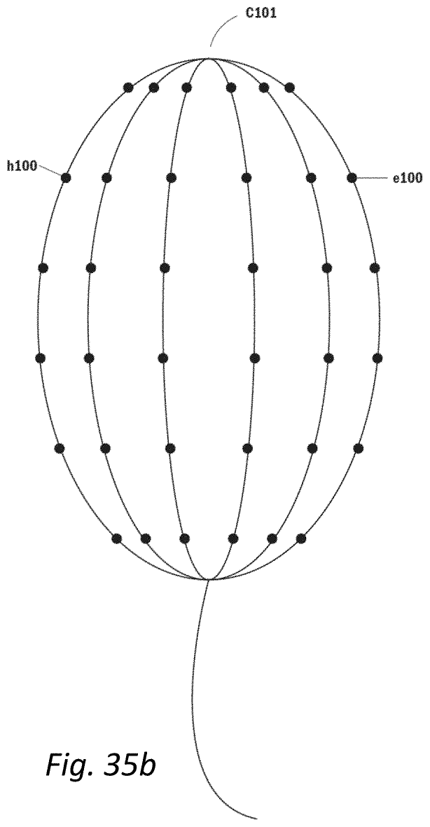

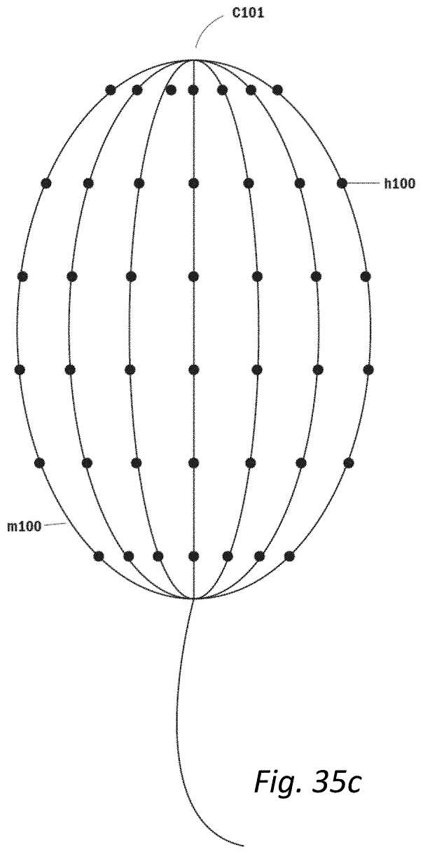

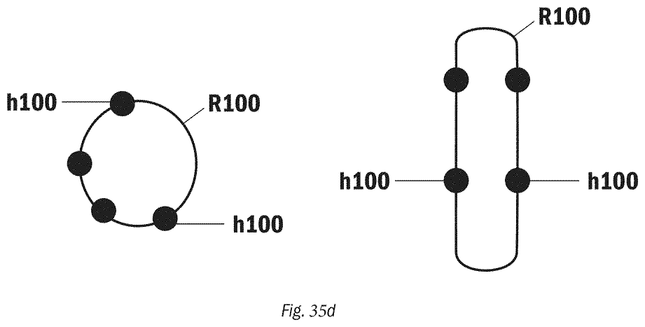

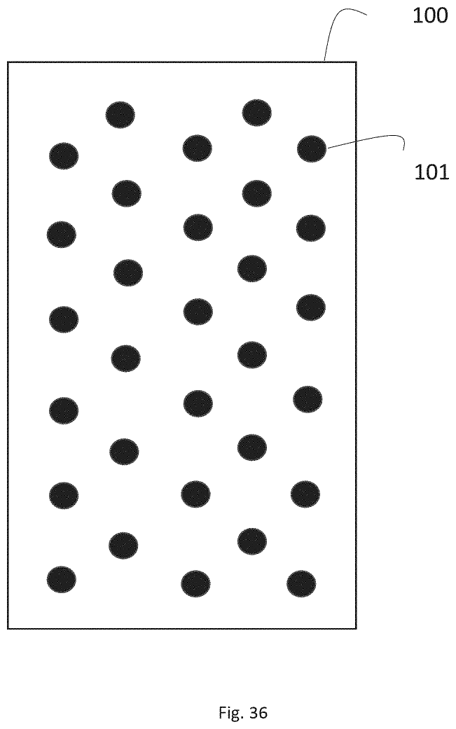

Catheter or a wired probe, or a miniaturized motorized robot for intervention procedures and computer controlled or user controlled valve for delivering therapeutic agents or diagnostic agents such as contrast solutions, can be better visualized. FIGS. 35 a, b, c and d illustrate various examples of a energy blation probe or a catheter c101 or a electrode and the opening for liquid aspiration h100or a miniaturized robot which can carry out a number of functions and controlled by computer or a user at a distal location may be inside the sheath, parallel to the tube for carrying the reagents. Different parts of the mini robot may have x ray attenuating properties to differentiate it from the rest of the intervention device. The catheter in FIGS. 35b may include components for treatment, therapeutic and diagnostic, imaging such as electrophysiology mapping and electrodes, e100 for RF ablation or ultrasound ablation, light probe. Within the catheter or the probe, there may be a sheath and/or a hardware mechanism to hold substances such as a tube including a structural means to hold liquid for example, comprised of polymer material or fiber or fiberoptic material for holding liquid or solution. In some cases, x ray attenuating material may be interlaced within the structural sufficient enough for it to be differentiable in an x ray measurement, such as spectral imaging method to be differentiable from the rest. The sheath or the holder or opening of the tube may have or may be connected to or aligned with one or more openings h100 from which the liquid, solution or the substances may be injectable or sprayable to tissue or area external to the tube, controlled from a remote location or distal location, there may be a valve, which can be closed and opened based on a digital command given by a computer or a user through a mechanical or pressure control. There may be one or multiple opening of the same or varied size to aspirate liquid. The opening h100 could be of varied orientation, to reach maximum surface area or varied density to ensure coverage.

The liquid or solution or substance may be a drug, or drug conjugates, contrast agents or conjugated contrast agents or antigen binding antibody or its conjugates, target molecules, or intermediate molecules which is used to bind, form complexes with, a marker or multiple markers which are in the tissue of ROI and/or VOI.

The opening or the hole h100 for releasing the liquid or substances may be at the tip of the catheter or the probe or any parts of the miniaturized robot where it interfaces the region outside of the robot or the catheter or the probe.

The main body or outer surface of the holder for the liquid or substance in the catheter or the probe or the tube or the robot, may be of flexible shape, for example, caged or balloon shaped as in FIG. 40b and FIG. 40c and FIG. 40d, and controllable by flexible and controllable mechanisms to ensure maximum coverage of area where the substance or the liquid can be delivered to, for example via pressure force of an injecting device or other energy, magnetic or electrical mechanisms via manual operation by a user's force or automated mechanisms where user control a digital method to control the motion involved in aspirate or inject the solution or substance.

The region of opening or the size of the opening can be adjusted by mechanical mechanisms in some cases. Alternatively the probe or catheter or the miniaturized robot may be remote controlled without a wire.

In one instance, the opening most distal to the controller end or the source of solution may have larger size than the more proximal area to ensure optimal delivery.

Ball shaped, or capsule shaped or other 3D shaped delivery vessel can include one or more holes or openings h100, in one or multiple orientation or regions of the probe, robot or catheter or the delivery tube for liquid or solutions to spray an area for permeability assessment or targeted imaging or for targeted solution delivery.

In addition, in situation of identifying region of interest for implant or surgical tool, or therapeutic regions, aforementioned probe or tube or liquid injectable device integrated with or attached with a catheter or the implant may be used identify area by injecting or spraying contrast agents functionalized with target ligands that can specifically binds to the region of interest or a portion of region of interest. The displacement of contrast agents or localization of the contrast agent binding regions may be used to guide placement of the implant of surgical tools or probes, or biopsy tools.

In some instances, the probe, the robot or catheter, implant or surgical probe or surgical tool or biopsy tool is radiopaque.

In some instances, one or more portions of the probe, the robot or catheter, implant or surgical probe or surgical tool or biopsy tool is radiopaque, can include one or more regions with similar or varied density or thickness of calcium or zinc, metals or radiopaque materials which are endogenous to a human or live subject.

FIG. 41 illustrates an example of an implant with one or more regions designed for x-ray measurements.

In some instances, the probe, the robot or catheter, implant or surgical probe or surgical tool or biopsy tool is radiopaque or attenuated at different levels in different portions or different component or different part of the probe, or catheter or guide wire. An surgical tool tip or a catheter, or biopsy probe or temperature probe or ultrasound probe and/or an implant may include a number of components, each or a portion of each may have a different attenuating property to differentiate from the background and other parts of device, so each portion may be controlled and monitored given the x-ray measurement feedback, such as in an arterial access sheath, or a portion of the sheath may have a different attenuation property than an interventional device, an internal lumen of the sheath may have a different attenuation property than the other parts of the sheath or the interventional device. For example, for delivery of a stent 2000 in FIG. 41, the catheter may have a sheath 2001 with a lumen. Within the lumen, there is a stent which is connected to a guide wire, 2002. Each of stent, or the sheath or the guide wire may have the same or different contrast agents of differentiable x-ray attenuation properties, or the same contrast agents with different density. And a portion of each, may be radiopaque, therefore enable tracking. Simulated measurements based on given specifications of the intervention device, or a portion of, may be used in cases, where lower radiation is desired for the application. In a Transcervical procedure, a number of devices or component of devices may be used for example, a catheter including a distal end and a proximal end, a blood entry port, and a first shaft portion extending proximally from said first occlusive member. The first shaft portion may have a cross-section sized to be insertable into the common carotid artery and suitable for transcervical access to the common carotid artery.

The device may include a fluid conduit in fluid communication with the blood entry port of the catheter to permit blood to flow through the catheter into the fluid conduit.

The device may include a flow sensor connected directly to the fluid conduit wherein the flow sensor senses blood flow through the fluid conduit.

The device may include a collection reservoir including a receptacle defining an enclosed chamber that collects blood and that is connected to said catheter and placed in fluid communication with said blood entry port via the fluid conduit. Fluid communication can include communication via a fluid flow path from said blood entry port to said collection reservoir without the assistance of fluid pressure sources other than arterial blood pressure.

The device may include a metering valve coupled to the fluid conduit, wherein the metering valve adjusts the flow rate through the fluid conduit.

The device may include an arterial access device adapted to be introduced into a common carotid artery, the arterial access device having a lumen that extends from a distal opening at a distal region of the arterial access device to a proximal opening at a proximal region of the arterial access device, the lumen configured for receiving retrograde blood flow through the distal opening from the common carotid artery.

The device may include a shunt having an internal shunt lumen fluidly connected to the lumen of the arterial access device at a location between the distal and proximal openings of the arterial access device, wherein the internal shunt lumen provides a pathway for blood to flow out of the internal lumen of the arterial access device at a location between the distal and proximal openings of the arterial access device.

The device may include a flow control mechanism coupled to the shunt and adapted to control blood flow through the shunt.

Each of these device components may be made of material which can be differentiated by x ray measurements with spectral imaging.

Research, NDT, High Throughput Measurements

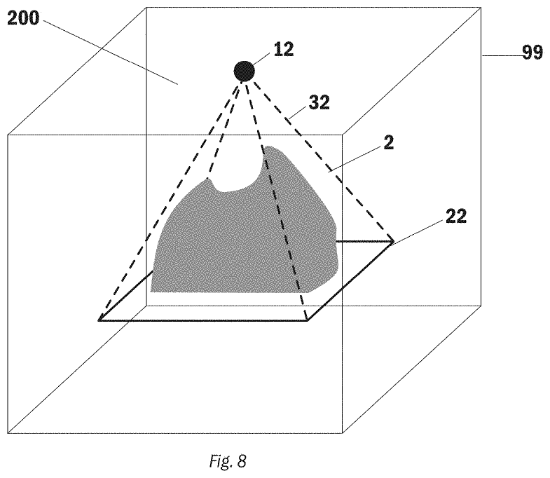

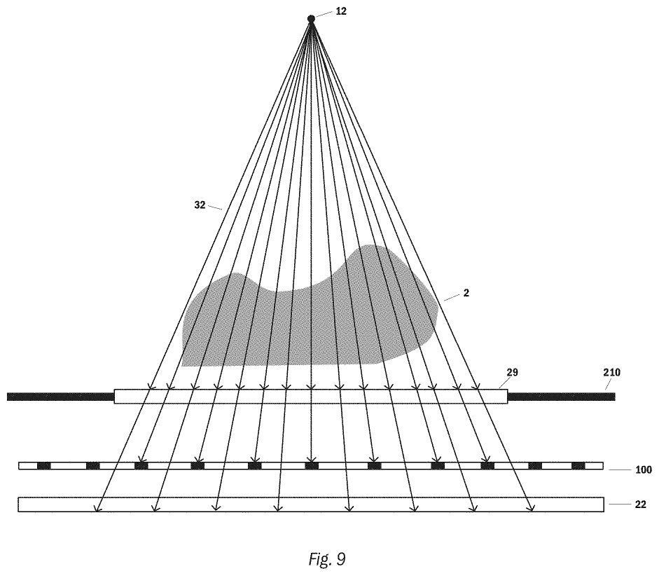

For non human applications, Enclosure may be needed for radiation safety. FIG. 8 illustrates an X-ray tomography imaging system 200 enclosed in an enclosure 99. The optical sensing or motion sensing system 200 can include a light source. A sensor can be placed inside the enclosure 99 to monitor activities of relevance inside the enclosure 99. Such an enclosure may have lead shield and or acoustic shielding. And a number of liquid handling and sample handling and storage and preservation devices may be integrated inside the enclosure as it is done in a typical micro CT, in vivo optical imaging and PET/CT device.

In drug discovery, diagnostics and life science research, quality inspection and failure analysis in ICT production and security applications, fast data acquisition on many different samples are needed to collect data for analysis and fact finding.

X-ray systems where one x-ray system or multiple systems are running at the same time on different samples of the same kind or different samples can be used, for example in 3D tissue study on a microfluidic chip, or for drug testing or lead screening on small animals or exo vivo animal tissues, or in digital pathology, for screening multiple tissues or samples from different patients at the same time.

In some instances, the detector may be capable of measuring x-ray, optical signals , such as UV, or NIR signals all at the same time. The same pixels may be used for all measurements. Different pixels of the same detector or different detectors may be used for measurements of different modalities

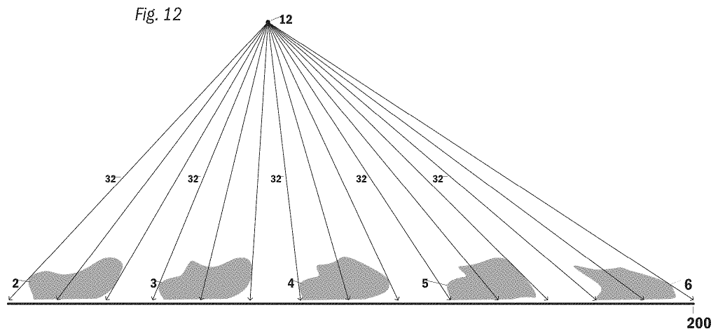

FIG. 12 illustrates a high throughput X-ray system including systems capable of 2D and/or 3D spectral measurements.

For example, selected regions of one detector measure subjects #2, 3, 4, 5, 6, respectively. Such measurements may be synchronized or not be synchronized such as, for example, in case of transmitted x-rays exiting each subject. In general, subjects are different or the same in each sample location. However, different sets of probes or molecular probes or contrasts or molecular contrast agents can be used. In some instances, subjects #2, 3, 4, 5, 6 may have different ROI s such as 2S1, 3S1, 4S1, 5S1, 6S1, correspondingly, in one subject in order to correlate and monitor components such as molecules, markers, specific material and characterize relative relationships in terms of time, location, sequence, existence and/or change and/or visibility and in some cases, interactions.

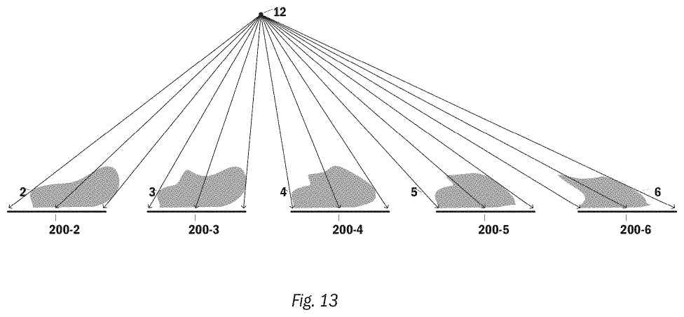

FIG. 13 illustrates another high throughput X-ray system including systems capable of 2D and/or 3D spectral measurements.. Instead of the same detector as in FIG. 12, different detectors, such as 200-2, 200-3, 200-4, 200-5 and 200-6 may be used to monitor each corresponding subject or region of interest using one radiation emitting position.

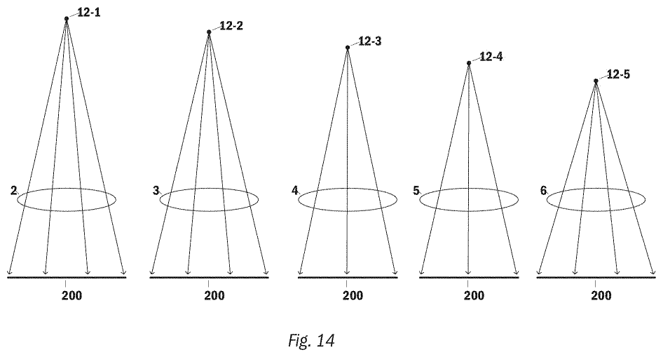

FIG. 14 illustrates a high throughput X-ray system including systems capable of 2D and/or 3D spectral measurements. Instead of one x-ray source, two or more different x-ray emitting location on one source may be used, each x-ray emitting source may be controlled independently. Such sources can be multiple pixelated source, field emitter sources, beam splitter, grating, or collimator generated x-ray emitting sources. Each source of 12-1 to 12-5 may be paired with one or more detectors.

In some cases, x-ray magnification apparatus can be placed in the x-ray illumination path, in between x-ray source and the detector, such as x-ray collimators to collimate x-ray beams, increase flux in an area and temporally, x-ray condenser, beam aperture, objective len, are used to magnify and measure small region of interest.

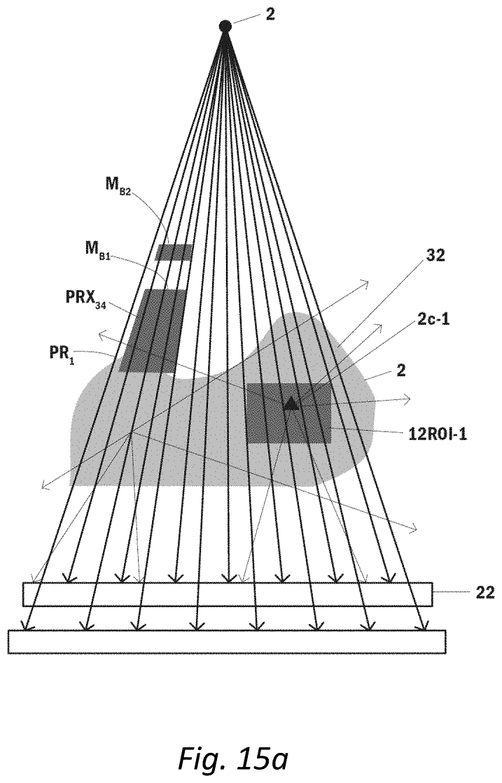

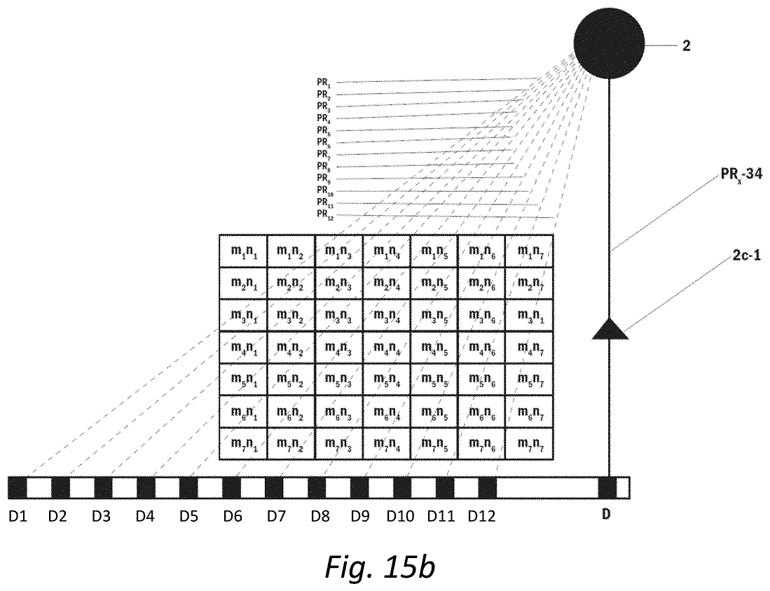

FIGS. 15a-b illustrates an example of x ray tomography device which may be used in a non human as well as human imaging setting. External Reference Component ERC, for example MBR-1 and/or MBR 2, used to spatially position a Component 2C-1 Internal to a Subject 2ROI-1 being illuminated and imaged by the detector assembly 22. Detector assembly may be one detector, or a dual detector assembly with beam selector as shown, or may include beam particle stopper and an detector, or may be a stack of detector layers, in some cases, each layer detector for an energy level (for example, top detector layer has a low energy scintilla layer or bottom with higher and higher energy levels). Primary ray PR1-34 passing through ERC, MBR-1 or 2 is spatially positioned to the primary x-ray PRx-34 which passes through 2c-1. If 2c-1 is separated from the rest of the subject through material decomposition or measurements based on x-ray measurable properties, the location of the 2c-1 can be determined. If a second measurement is done on both MBR-1 and 2c-1 at a different x-ray emitting position from the source, the relative spatial position including the coordinate axis perpendicular to the x-ray may be obtained for the component of interest, 2-1. MBR-1 and/or MBR-2 may be placed anywhere below x-ray illumination pathway. MBR-1 or MBR-2 may be internal to the subject. For example, they may be components with known density, shape and composition and spatial position relative to the source. In some cases, MBR-1 or MBR-2 is not needed.

FIG. 16 illustrates an example of x-ray tomography device, capable of integrating measurement of elasticity of ROIThe ultrasound transducer UST-1 is position next to the soft tissue to be imaged in between the source 12 and detector 29. For example, in brain imaging, the transducer can be placed right next to an opening to soft tissues inside the skull, air or eye or other openings. The detector 29 may be removed in some instances. And the scatter removal with plate 100 may be implemented with alternative apparatus and methods. And in some cases, scatter removal may not be needed. And the x-ray cone beam may be alternatively implemented by a fan beam or distributed x-ray thin beams by using collimators downstream from the source 12.

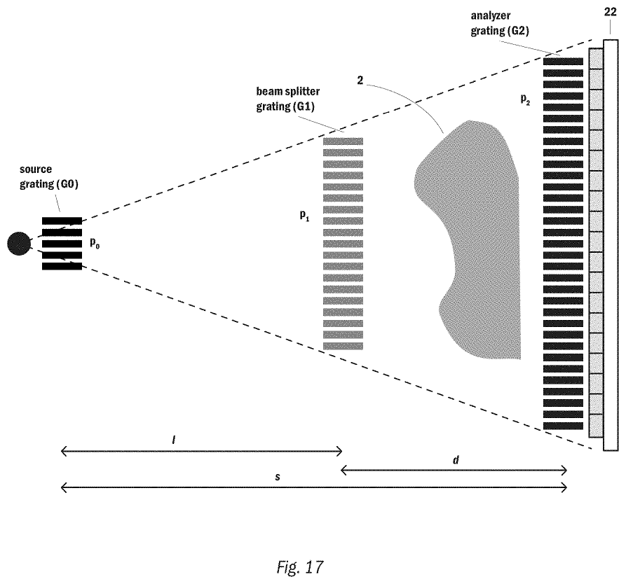

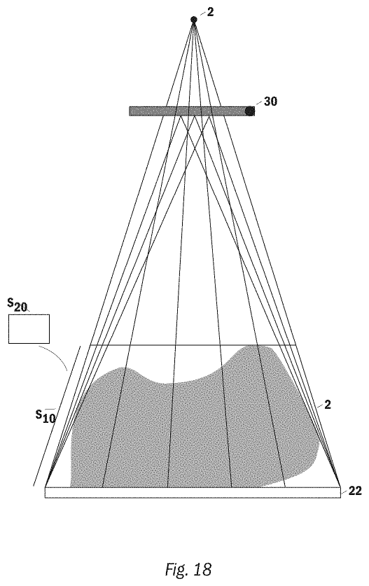

FIG. 17 illustrate an example of x-ray system with a near real time phase contrast and/or capable of Fourier transform device, in some instances, is as described in the aforementioned PCT Applications. X-ray optics such as beam splitter, gratings are used to generate interference patterns to be measured on the detectors. The x-ray system can be a device with near real time phase contrast and/or capable of Fourier transform combined with scatter removal processing technique, for example using beam particle stopper device and interpoloation to reduce scatter to primary ratio to less than 1% or less than 5% to improve measurement in the space as well as frequency domain .

FIG. 18 illustrates another example of x-ray tomography measurement device. The ultrasound transducer is position next to the soft tissue to be imaged in between the source and detector. For example, in brain imaging, the transducer can be placed right next to an opening to soft tissues inside the skull, air or eye or other openings.

The ultrasound transducer UST-1 or a 2D shear force generator such as sound generator is positioned next to, with or without contact, the soft tissue to be imaged in between the source and detector. For example, in brain imaging, the transducer can be placed right next to an opening to the region of interest in the soft tissue inside the skull, air or eye or other openings. S20 is a control box with computer processor to control the device. In some instances, such a control box is integrated with a motherboard, which controls other hardware in the imaging system. The x-ray system may be a dual detector with beam selector module, or beam particle stopper plate, 100 integrated with a detector, or one or more PMT, or photon counting detector, spectrometers, spectral absorptiometry module. And in some cases, scatter removal may not be needed. And in some cases, scatter removal is done to reduce the scatter to <1% of the primary or <5% of the primary. In some instances, x-ray beam may be one or more cone beams, or one or more fan beam or a number of distributed x-ray thing beams, which may be implemented by using a collimator downstream from the source 2 or using anode target, which has regions that do not generate x-rays or tunable anode target where only selected regions generate x-rays.

DNA or RNA Sequencing

In molecule identification and detection, for example, nanobody or small molecule tagged with contrast agents can be measurable by x-ray imaging and measurement system and methods described herein. The molecules may include amino acids based molecules and complexes, protein fragments, and/or nucleic acid based molecules, lipid, glycoprotein, rare elements and complexes or combination of above and other metal elements such as nanoparticles.

The present disclosure includes DNA and RNA sequencing imaging and measurement using X-ray measurements,specially in case of single cell DNA and/or RNA sequencing, for example, using magnet and antibody conjugates to detect single cells.

The present disclosure allows for x-ray measurements based on contrast agents to be used in place of optical measurement dyes such as fluorescent dyes and/or be combined with a molecular probe or dye for optical measurements, or mass spec or maldi for hybrid imaging systems or multimodality imaging and measurement systems.

The present disclosure allows for measurements of nanoparticles or contrast agents, which conjugates to small amount of molecules, down to picograms such as in single cell RNA or DNA sequencing. In particular, naturally a single cell produces enough RNA copies of a certain sequence, a molecule, such as x-ray measurable element or molecules that is or are linked to nanobody or antibody variant or small molecule, may bind to one or multiple molecules in the molecular pathway for transcription and translation, for example, RNA or its complementary DNA pairs, or peptides produced by the RNA, or one or multiple molecules in the molecular pathway for transcription and translation, and be detected and identified.

Such sequencing and small molecule detection may be done in vivo or exo vivo or in vitro format.

In current Single molecule detection and Single Cell RNA and DNA sequencing technology, cells or tissues are isolated by various methods, for example laser microdissection, then isolated by for example, microfluidics mechanisms, droplet microfluidics, and cDNA complementary strands are attached or amplification occurs to produce cDNA library to obtain a sample, Such samples are aligned, deduplicated, and unique reads are performed and quantified. The labels and contrast agents used in the process, may be of one or more x-ray contrast agents and unique reads may be detected by x-ray measurements instead of fluorescent labels. The x-ray measurements may be of transmitted x-ray, fluorescent x-ray, scattered x-ray, or non-linear x-ray.

In contrast, the systems described herein may be collocated with Single Photon Emission Tomography (SPECT) imaging systems and their accessory devices, Ultra-High Energy collimators (UHEC), Attenuation Correction Devices (ACD), Positron Emission Tomography (PET) imaging systems and their accessories, Coincidence Imaging Devices (CID), and Nuclear Tomography Systems (NTS) and its accessories, optical measurement system, sound measurement system, thermal based measurement systems, energy based measurement systems. It may be accomplished by using anatomic markers or position markers or location markers internal or externally. For colocation systems, contrast agents for one modality may be used for the present system or such contrast agent for one modality may be conjugated with one or more marker and/or one or more substances identified and positioned by the present system.

The present disclosure provides for Live Cell and Live Animal Imaging.

Subject is not fixed on a sample stage, rather than live and/or free roaming, for example, live animal in a cage, or a cell or molecule moving in a microfluidic channel or sample slide.

The x-ray measurements of the x-ray imaging system and apparatus disclosed herein, for example, spectral measurements or derivation values of voxels or specified regions and its or their spatial locations or measurements in time domain or frequency domain, may be used in the measurement of region of interest.

In conventional imaging systems, 3D and/or spectral methods and details embedded in a subject or an region of interest are not available.

Other in vitro measurements traditionally using contrast agents or labels or dyes used for optical measurements or spectroscopy measurement may be replaced by x-ray measurements combined with x-ray contrast agents.

For example, DNA sequencing or Protein Western Blot or any optical methods and electrochemical methods using contrast agents or indicators may be replaced by an x-ray sensitive contrast agent or contrast agents.

Measurement and detection of bound or unbound element of metals or x-ray sensitive substances, using material decomposition method, may be combined with multiple dimensional imaging and measurements.

An x-ray measurement device, or computer tomography device, may provide 3D x-ray imaging and reconstruction from 2D measurements and display of derived data from the imaging , in 2D and 3D format. The use of such a device may involve medical or imaging and measurement procedures and/or methods similar to that using conventional CT, or quantitative x-ray imaging, or densitometer, medical procedures similar to those of procedures of CT, densitometer and quantitative x-ray or hybrid system with other modalities, such as PET or optical imaging methods, photoacoustic methods. Such a device may be used in imaging methods optimized in terms of resolution, energy level, exposure, field of view and or region of interest, biomarkers, hybrid imaging with PET and other imaging and measurement modalities, contrast agents to provide CT-like or densitometer-like or quantitative x-ray like or PET/CT hybrid-like, SPECT-like or similar to X-ray hybrid or without X-ray, of measurements and/or imaging of modalities different than x-ray. For example, such an imaging or measurements may be used directly or used to train or later on to be used by digital programs to simulate the p measurements and/or imaging and/or presentation by CT, PET, SPECT, MM, Optical Imaging and analysis, photoacoustic, ultrasound, thermal imaging and others.

Such images and/or measurements and related annotation, label, text and voice marker and labels, can be for analysis and/or training and post processing by Artificial Intelligence algorithms developed for imaging and/or measurement methods and analysis of conventional CT-like images and/or measurements and/or quantitative X-ray imaging and measurements, such as densitometers or PET/CT hybrid, SPECT microscopy, optical measurements, MM and other aforementioned modalities.

Such analysis of conventional CT and other modalities include segmentation, spatial positioning of markers, quantification, tracking, fluidic dynamics, perfusion, calcium scoring, biomarker assessment, material decomposition, spectral CT, deep machine learning, volumetric measurements and annotation, label, referencing for diagnosis, prognosis and tracking and defect analysis, monitoring and surveillance.

The image and measurements may provide synthesized and/or extracted or segmented, or ROI data similar to that of conventional CT or densitometer or other quantitative X-ray imaging method and other aforementioned modalities, such as optical imaging, MM, data and image presentation, for example, one or more sliced of images at a certain thickness, approximately similar what a rotating scanner CT may present and provide approximately similar quantitative values, from 3D or multiple dimensional data derived by measurements of one or more area detectors, or photon counting detectors, or photon sensitive detectors or PMTs, or spectrometer assembly, for example, using x-ray imaging apparatus and methods described and continuation of such systems described herein. The presentation of x-ray measurements may be used to correlate the x-ray measurements with the measurements in space and time and frequency to that of a different modality or x-ray hybrid modality, simulate optical fluorescent and/or nanoparticle or quantum dot marker measurements as well as that of optical coherence tomography and/or multi-photon microscopy, raman scattering as well as that of optical spectroscopy and Mill.

Due to the experience and trained knowledge of users, such as physicians and/or radiologist and/or researchers, analysist in AI development, even if 3D x-ray image or 2D x-ray measurement and presentation is reconstructed with better resolution and spectral data, it may hard to link resultant data with prior knowledge acquired by radiologists and other professionals and there may be a learning curve. To make the x-ray system more useful during the time where it requires training and correlating results and data of prior measurement cases from imaging history using CT scanner and/or other modalities, it may be useful to present 3D data in the format which is familiar with trained professionals. However just one CT sliced image presented in the context of the new imaging system interface, which may be different from the existing interface, may not be understood unless the entire image data is presented in the image format of CT scanner.

Such an x-ray imaging system may be used to provide data to train AI programs of different modalities or to users and digital programs used in digital pathology or drug discover or research to correlate x-ray measurements or simulated measurements of different modality to cases done by actual measurements before to derive facts and establish database or relationships between modalities and measurements from a different time and place.

Database of x-ray quantitative measurements with x-ray related modalities as well that of other modalities or hybrid modalities may be established to correlate quantitatively measurements using different hardware such as x-ray source, or different detectors, and/or at different settings, such as exposure time, and current, energy level and field of view and x-ray input level for each substance, or composite substance or a component or two or more component, such that x-ray measurements and measurements of the present disclosure, conventional CT, Mill, other modalities and hybrids in different time and conditions using different settings may be correlated with quantitatively.

For example, MM measurements of heart in space and in time with and without contrast agent may be correlated with simulated x-ray measurements of the present disclosure.

Conventional CT measurements with certain settings and system design and methods may be correlated with x-ray measurements of the present disclosure.

Human vision and other sensory related measurement or digital imaging and/or measurements may be correlated with x-ray measurements of the present disclosure.

X-ray measurements of the present disclosure may synthesize images familiar to human vision and/or other digital machine vision programs and provide measurement data and/or its presentation familiar to a user or digital program for diagnosis, human decision, human machine interface, tracking, object failure analysis and defect analysis, time tracking, prognosis, monitoring and surveillance, and motion detection, characterization of fluidics, dynamic movements and other related measurements of physiological, mechanical, spatial, temporal, artificial, synthetic, relational, kinetic, energy perturbed, chemical properties, mechanical, elastic properties and other parameters.

AI programs and algorithms trained in MM and conventional CT and densitometers and quantitative x-ray measurements may be used with the measurement data provided by x-ray systems of the present disclosure, and vice versa.

The x-ray system and apparatus may be used as a standard to quantitatively relate data from various imaging and/or measurement modalities, especially of those which are of quantitative measurements.

An x-ray system may include one or more elements of the following:

one or more functions and features or apparatus and/or methods of “personal CT” or “customized CT”, which may have at a mode, or produce a set of images in the format of CT scanner Images or display images and measurements or have display setting which can be switched on or be selected by a user or digital program, for example, to display sliced images, 2D images or tomography images or segmented images and values for one or more parameters typically displayed in a CT image viewing or display or analysis software, approximately similar to convention CT scanner or spectral CT scanner, for example, with a contrast agent or without a contrast. Such images may be stored and exported as a set of files in CT scanner image format, to be read by conventional CT viewers and CT data reading and analysis programs including AI.

The x-ray system may also run on a serial 3D images mode, varied in time, such as a fluoroscope mode, which is familiar with existing radiologist or physicians using conventional apparatus and methods or displaying a series of x-ray images similar to that fluoroscope.

The x-ray system can include parts or the complete personalized CT systems or using measurements described herein.

The x-ray system may have one mode or setting or provide a format of images, which may be called, personalized CT images, or precision images or personalized precision CT images or may display the measurements, synthesized or reconstructed and constructed images directly from on the images or measurements of region of interest and/or the imaged subject. And the background of such images may be a 2D or 3D image or a multiple dimensional image.

There may be two or more detectors, one beam particle blocker plate for each detector, and one mover, for example one set of actuators for each beam particle blocker plate. In some cases, there is another set of actuators or mover that can move the detector, beam particle blocker and beam particle blocker mover, relative to the first set of detector, beam particle blocker.

Microvasculature density and morphology and fluidic dynamics, density of tumor and surrounding tissues, tumor marker targeted contrast labels, and molecular profile (molecules in the body as a whole, molecules in and outside of the cell, concentration of one or more molecules related to the tumor growth stage and metastasized stage) may be measured and used as the input for machine learning algorithms, for diagnosis, prognosis, prediction of treatment outcomes and therapeutic outcomes, treatment planning, monitoring and surveillance using AI.

Disease other than tumor may be monitored the same way.

In addition, two or more detectors may be stacked below the beam particle stopper plate, for example, one with high energy, the other low energy, or the detector may have different spectral sensitivity and frame rate being stacked. And one or more x-ray source with x-ray beam steerer or electron beam steerer or mover, may be placed at one or more locations, so that images of two or more energies may be acquired simultaneously. Each x-ray source may restrict field of view or steer field of view.

Calcium scoring may be achieved without multiple dimensional measurements or with multiple dimensional measurements or with 3D measurements. In multiple x-ray energy decomposition methods, calcium may be separated from the soft tissue. In case of using 2D dual energy or spectral imaging methods described herein, calcium content embedded in heart or other soft tissues may be separated by first removing bone, if the calcium and bone are overlapping. Due to slow varying nature of bone, bone can be separated from that of calcium in heart due to interpolation value may be derived from measurements in the adjacent pixels. In case of three 3D imaging, bone regions may be segmented out of the measured volume and calcium measurements can therefore be derived. Multiple angle measurements may be positioned at different x-ray emitting positions, to remove the projected image effect of overlapping chest bone and calcium region in heart.

Definitions of Certain Terms

“Personalized or customizable CT” system refers to a x-ray imaging including of apparatus and methods which may be capable of 99% or better, or 95% or better scatter removal or that it may reduce scatter to less than 5% of the primary or to less than 1% of the primary as aforementioned, using beam particle stopper plate, or beam selector or time of flight light source; may be capable of dual energy or spectral imaging or may be capable of multiple dimension x-ray imaging; or may be capable of time sensitive x-ray measurement and tracking or monitoring x-ray measurement in point, 1D or 2D, 3D up to 7D (6d with time), or phase contrast imaging, or imaging with other modalities and other x-ray optics added in the imaging path. The type of measurements or imaging performed each time is on a case by case basis, for example, when such a measurement is or measurements are useful and required. For example in diagnostic imaging, an x-ray beam is measured in dual or multiple energies when densitometry is needed to measure bone density, or movement of a component or area of interest embedded in a subject needs to be monitored.

In diagnostic of lung illness, such as idiopathic pulmonary fibrosis or virus or bacteria induced pathology, such as covid 19, or TB, the region of interest is only a fraction of lung needs to be imaged or quantified, instead of the entire chest. Only a fraction of lung is selected to be imaged in 3D, with selected resolution in depth based on the clinical value which may be delivered based on such measurement. Such region of interest may require 2D imaging of the entire chest at one or more energy levels and material decomposition, and/or may require a 3D low resolution imaging, or may require 1D, 2D or 3D imaging from multiple angles with one or more x-ray sources, which may be stationary or moved.

In cancer tissue imaging, there may be contrast agents, for example, molecular contrast agents specific to the tumor indicators or markers, administered prior to the imaging.

The region of interest may be defined based on one or more parameters, such as the tissue type, or vasculature and microvascular, flow dynamics of blood, or extracellular markers or intracellular markers or combination of one of more selection criteria based on quantification of density, morphology and other chemistry test and patient profile and imaging history.

In surgical guidance, the region of interest is continuously defined and modified.

In fracture of the bone, the region of interest is defined differentlyt based on the density and image of the injured bone, relative distance to adjacent bone or adjacent bones and/or that of soft tissues.

In each instance, the process of defining of region of interest may be different, for example, in procedural process and/or in image methods.

And 3D imaging resolution may be different for each application and for each imaging case.

And scatter removal process may or may not be needed for each measurement.

The selection of point, or 1d or 2D or reconstruct 3D images is different during the imaging process or for each application and/or is selected on an individual case basis.

Different component and materials may be represented in one or more visible narrow and/or broad light wavelength for better presentation and visualization in 2D and multiple dimensions.

Methods for Replacement and visualization of Over lapping Tissues

2D spectral imaging data may be presented and displayed for diagnosis and monitoring and tracking previously done by CT. However there are challenges in displaying the data due to artifacts that may be produced in display as the software tool used for image adjustment have a different set of process and criterial than density and thickness based measurements.

The following is an example of improved visual presentation of 2D quantitative data.

The present disclosure may provide methods for eliminating the bone interference on its overlapped soft tissue x-ray projection image or alternatively, remove soft tissue in the bone x-ray image. The method is based on use of a two dimensional dual-energy x-ray imaging hardware system. The method can include three major procedures: (A) Acquiring a pair of dual-energy images at a high-energy level H, and a low energy level L for the subject that contains the interested soft tissue overlapped by a bone structure through the dual-energy x-ray imaging hardware system; (B) using a dual-energy decomposition method to transform the acquired dual-energy x-ray image pair data into two material composition images: the human body soft tissue image and the bone image. This is to remove the direct interference of bone image imposed on the soft tissue image, where the desired image information resides. (C) Continue to improve the soft tissue image by compensating the indirect interference effects in connection with the bone due to empty space originally occupied by the bones inside the soft tissue. The compensation method can include two steps: (1) Replace the originally decomposed bone image with a bone-equivalent soft tissue image in a way such that the same geometric conditions are maintained, while the x-ray absorption image produced by the bone material is replaced by an x-ray absorption image predictably produced by a bone-equivalent soft tissue material on pixel-by pixel basis; (2) Reinstall the bone equivalent soft tissue image into the originally decomposed soft tissue image at the exact geometry position where the originally decomposed bone image is located.

The reverse as described above may be done for removal of soft tissue image from that of bone image, such as in cases where there are overlapping bone tissues in a projected image.

In situations where there are more components overlapping with each other, for example, when one blood vessel, one bone tissue, one nerve tissue and over lapping soft tissues are visualized, an image of the blood vessel and an image of bone tissue, and an image of nerve tissue may be derived, and using processing similar to the dual energy system, (1) each of the three tissues is being replaced by its equivalent soft tissue in such a way that the same geometric conditions are maintained, while the x-ray absorption image produced by each of the tissue material is replaced by an x-ray absorption image predictably produced by each of the specific corresponding tissue-equivalent soft tissue material on pixel-by pixel basis;

(2) Reinstall each respective tissue equivalent soft tissue image into the originally decomposed soft tissue image at the exact geometry position where each of the originally decomposed individual tissues, such as bone, blood vessel or nerve tissue image is located.

Often, when there are overlapping tissues of multiple different types, for example, based on each original image taken, anatomic markers and geometric location, position and location of each component compared to the others may be approximated prior to material decomposition based on the original measurements and images. In addition, based on reference database built upon similar anatomy or body parts or subject, other assumptions may be made.

For example, a three dimensional image of a selected region may be derived, therefore the information embedded in the third axis may be obtained for the selected region. As the human body is slow varying for most tissues, or organs, the thickness and geometric location and position of a layer of tissue or layers of different tissues may be obtained for a region or regions surrounding the 3D resolved region. For instances, one or more selected regions distributed from each other may be used to provide information for geometric location or relative position and distribution of various tissues or components throughout a subject.

In some cases, each of such 3D measurements are limited to a region as small as possible in order to reduce radiation to the subject and save time.

In addition, dual or triple or multiple energy measurements in 2D or 3D format of region of interest may supplement such derivation.

When the foreign substances are well defined, a simulated measurements either based on predicable value or based on amplification of predicated value for visual presentation and ease of use, as long as it does not distort or disturb the environment it is or mislead user, can be represented with different format of representation, including choice of color, and amplification of signal proportionally or disproportionally, or removal of signal completely as it does not interfere with the rest of ROI behavior.

For example, other than a reference object, the chest bone may be interference of the signals measured to be presented, especially for heart tracking and lung studies and for using calcium as contrast agents, or evaluating calcium scoring or microcalcification of tumor and plague. The chest bone data may be taken out and discarded or portion of it may be set aside as a reference object with anatomic markers to better position material of interest to be visualized and guided and quantified.

The x-ray imaging methods disclosed herein when there are overlapping tissues can include the following procedures:

(A) procedures for performing a dual-energy or multiple image data acquisition for two components or more components material decomposition; (B) procedures for performing a dual-energy or multiple energy or spectral image data procedures for performing a dual-energy and spectral image data decomposition; (C) procedures for compensating the interference due to the bone-removed empty space in the soft tissue or the multiple individual tissue removed empty space in the overlapping tissue.

The following is an example of dual energy method, which may be extended to multiple energy system for multiple component imagine and image separation and display for better visualization and quantification.

(A) procedures for performing dual-energy image data acquisition; For performing a dual-energy image data acquisition, the x-ray source emits a high-energy x-ray pulse, and in the meantime, the detector conducts image data acquisition at high energy; Then, the x-ray source emits a low-energy x-ray pulse, in the meantime, the detector conducts image data acquisition at low energy. If the conventional detector is used, the acquired image data are to be directly used for the procedure(B). If the detector with scatter-removing capability is used, both the front detector and the back detector conduct data acquisition. With certain additional data processing, as described herein, a pair of image data from the front detector are to be sent for the procedure(B).

(B) Procedure for performing dual-energy image data decomposition. Any dual-energy data evaluation method can be used, such as provided in the present disclosure. For illustration, two examples are briefly described. However, the procedure(A) is not limited to these methods.

(a) Dual-energy data decomposition method based on thoroughly removing scatter interference, and directly solving comprehensive dual-energy equations. The removal of scatter, and performing dual energy decomposition are inseparably combined into a single, coherent process operated through using a specially designed 3-layer x-ray detector assembly. A brief summary of the operation of the system is presented below.

The x-ray detector can include a high-resolution x-ray detector as its front (the first layer) detector, an x-ray beam selector (the second layer), and a low-resolution x-ray detector as its back(the third layer) detector. The beam selector can only allow one type of x-rays passing through it and received by the back detector: either only the scattered x-rays or only the directly transmitted x-rays without any scatter. The other type of the x-rays is substantially blocked to the back detector. As a result of a dual-energy image data acquisition, the front detector acquires a pair of high-resolution of x-ray images that contains both the directly transmitted x-rays (emitted from the x-ray source and directly transmitted through the subject strictly along a straight line) and the randomly scattered x-rays, while the back detector acquires a pair of low resolution of images given by only one type of x-rays (either transmitted-only, or scattered-only x-rays).

Through processing the image pair acquired by the back detector, the information regarding scattered x-rays on the front detector is accurately predicted, hence a pair of image data sets for the directly transmitted x-rays, free of scatter interference, on the front detector (the high-resolution detector) for the x-rays at the higher energy EH, and at the x-ray energy at the lower energy EL is accurately obtained.

Going further, based on the high-resolution image pair on the front detector obtained at the higher energy EH and at the lower energy EL, by using dual-energy data decomposition method, a pair of material composition images (the bone image and image soft tissue image) are obtained. One dual-energy data decomposition method disclosed herein can be based on direct solution of the nonlinear dual-energy equation system without relying on any simplifying assumption or any simplifying approximation. Since no important simplifying approximation is made, no important scatter interference is involved, and the solution should have the capability of providing the most accurate results.

On the other hand, there are numerous ways for solving the dual energy equation with certain simplifying assumptions, and certain simplifying approximations. Scatter interference can also be mixed in the true image data at different levels. Consequently, the accuracy of the results are degraded to different extents. A common feature of these simplifying approximations can include: (i) without removing scatter interference;(ii) using well-documented linear approximation or linear approximation with nonlinear corrections to solve the nonlinear dual-energy equations. The specific forms of the simplifying approximation methods could be numerous, the present disclosure does not limit use of any justified simplification or approximation, but contemplating all these similar methods. As an illustration, a scatter-tolerant, linearization for solving the dual energy equations is described below.

(b) Scatter-tolerant, linearization method. The first approximation assumption is to neglect the scatter interference contribution to the image signal. The second approximation assumption is all the nonlinear absorption effects of the subject can be neglected, and the x-ray source emits x-rays with only a single energy value without any spectrum distribution. Under these approximations, the nonlinear dual-energy equation system can be linearized to give a pair of simple quantities b(x,y), s(x,y), which can be found by any standard method for solving 2 linear simultaneous algebraic equations on pixel by pixel basis for each point (x,y). Linearization method can be easier to implement than nonlinear methods. When higher accuracy quantitative results are necessary, further corrections for nonlinear effects based on the disclosure herein can be made.

(C) Procedure for compensating the newly produced interference effects due to the bone-removed empty space in the soft tissue. As a result of procedure(B), a strong bone absorption image directly imposed on the soft tissue image is removed. Thus further visually discerning of the desired image information contained in the soft tissue and masked by the bone is significantly facilitated. However, another form of interference in connection with the presence and removal of bone can exist: the empty space originally occupied by the bone now gives a cavity image of the soft tissue. The cavity image plays a role of an unfavorable background pattern, and preventing further visually discerning of the information contained in the soft tissue. The objective of the procedure(B) is to compensate the bone-removed empty spaces in the chest wall soft tissue image. The compensation technique can include two steps: (i) Transforming the originally decomposed bone image into a bone-equivalent soft tissue image which is geometrically identical with the bone, but physically possessing all the x-ray absorption attributes tissue image, into the originally decomposed soft tissue image at the exact geometry position where the originally decomposed bone image was located.

With the procedure(C), the interference caused by overlapping bone to the soft tissue can be removed.

Other standard imaging processing techniques, such as contrast/brightness variation and image subtraction methods can be further used for further visually discerning the hidden information contained in the soft tissue.

Optional to the above described major compensating procedures, there are certain methods that can further improve the uniformity of the resultant soft tissue. These methods belong to higher order approximations. For example, according to physiological feature, the bone-equivalent soft tissue image for the chest wall should have a highly smooth and slowly changing spatial distribution. Solely using a multiplication factor in the step (ii), certain internal bone structure, such as local defects, or hollow core structure may be brought into the bone-equivalent soft tissue image, and may cause unnecessary confusion in analyzing the internal organ soft tissue image. To further eliminate these high order effects, the following methods can be used: (a) with reference to each specific bone structure, slightly modifying the originally obtained bone image to a desired smooth geometry. Thus, a slightly modified bone image instead of the real bone image is used in the step (B)(i), and (B)(ii). (b) Standard image smoothing, subject boundary detection methods, etc. in image processing techniques can be used to provide a bone image better for construction of the bone-equivalent soft tissue image. These and all other the high-order improvement methods are contemplated by the present disclosure.

The present disclosure further extends to subjects including two or more components or components. When at least one material or substance, namely, the first component or first substance is separated from the rest, the space occupied by the composite or the components other than the first component, may be replaced by the composite equivalent of first material for better visualization, such as when bone is replaced by soft tissue for soft tissue visualization.

Controller

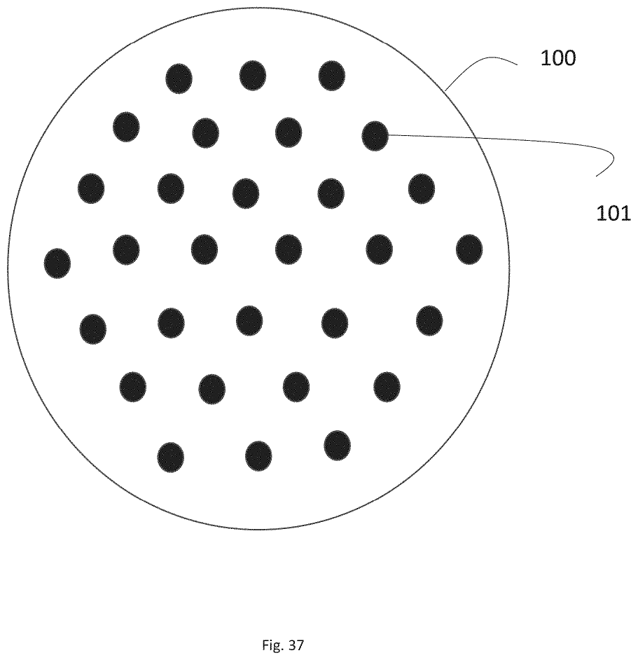

FIG. 37 illustrates a control unit within the x-ray imaging system.

A control unit 200, which is connected to some or all of the electronics driven hard ware, such as motors, x-ray collimators, generators, detectors, has a cpu or microprocessor, or embedded circuit and firm ware to control the operation and/or on and off of each hardware.

Control unit 200 may be connected in a wired connection or wirelessly such as wireless network, ethernet, or Bluetooth to a controller 1200, which may have a display panel, such as a touch screen, or a handheld display, may be connected to a joystick or computer input device, a different microprocessor power controller.

In some cases, control unit 200 is integrated with controller 1200. Controller 1200 may be used to move motors used to move detectors or x-ray source and/or operations of collimators and/or selecting volume of interest to image and/or interacting with sensor to make measurement, either to take 3D images as in a camera or to measure distances and geometry of the objects such as x-ray tube, patient, detector to make automated measurement decisions based on criteria given by the user or the computer.

Physically control unit 200 or integrated control unit of 200 and 1200 may be integrated with or attached to structural support 104, or a stand for x-ray source or part of support structure for the x-ray system or it may be separate from the detector assembly 109 or it may be separate from the x-ray source assembly, or it may be a stand alone unit, or may be integrated with a portion of the x-ray system.

Either control unit 200 or controller 1200 may have a LED display or a display panel. The display panel may display the instruction for movement, or it may have images of the imaged subject 2 and/or ROI region or computer selected ROI region on the imaged subject. A user may tap on the part of the imaged subject in the display. The x-ray source assembly 12 as well as the detector 22 assembly or 23 can be positioned to be in spatial position approximately optimized to image the selected ROI.

The selection or imaging of ROI and alignment of x-ray source and detector may be achieved automatically based on criterial determined or predetermined and stored in the work station or controller 1200 or control unit 200.

Controller unit 1200 may communicate via wireless mechanisms or wired connections to a workstation 1010 or a laptop or microprocessor with or without a display 1015 capabilities.

Controller unit 200 and/or workstation 1010 may be placed in a location different than a portion of the imaging device such as x-ray tube and detector and the imaged subj ect.

Controller unit 200 may be used by a user to move a portion of the x-ray device such as detector or the x-ray. The user may make the decision of the movement based on the direct observation of the LED light mark on the imaged object by the collimator or the x-ray source or by the information given by the sensing element.

The x-ray system disclosed herein may include Accessories and Peripheral Devices.

A dosimeter may be attached to the collimator or anywhere on the structure to monitor dose and possible exposure to the imaged subject and other subject in real time during x-ray exposure or overall time. And the electronics controlling the dosimeter, data management and/or analysis of the measurements may be done locally to the dosimeter by a microprocessor and/or by microprocessor which is used to control other components of the x-ray system and local to the x-ray system, or a microprocessor remotely. The communication method between the dosimeter to any one of the microprocessors may be wired or USB, or cable or via WIFI or blue tooth or optical or RF or other wireless methods used in communication and data transfer.

The x-ray system disclosed herein may include Automatic exposure control.

In some cases, a plurality of event detectors can be deployed relative to one another on the chip of the image sensor. In particular, an active pixel sensor (“APS”) device can be used as the image sensor. The event detectors are therefore made of additional photosite pixels that are located on the same chip of the APS sensor, preferably in the peripheral locations relative to the active pixel array therein.

A detector may be placed downstream of ROI.

Optical measurements and imaging apparatus methods, endoscope, MRI apparatus, Magnetic particle imaging, ultrasound, thermal imaging and electrophysiology may be integrated into the present disclosure by referencing or colocation of contrast agents, anatomic markers, or identifiers such as a catheter or probe in electrophysiology device, an identifiable subject, such as micromanipulators, semiconductor component, a part in an optical systems, or in a physical or chemical property measurement system, or one or more parts of therapeutic devices and energy device capable of perturbation of the sample or the subject or reference to the location of one or more internal component or marker, or one or more spatially external marker or reference object, such a marker or reference. A user controlling a software or a digital automated program may be used to make the measurement necessary to establish the colocation, or preexisting data or measurements that may be input into the microprocessor, where measurements are stored in a memory device to be used for calculating such relative spatial locations.

At least one sensor, such as a time of flight sensor, sensor, or a camera or video camera, in some instances with night vision capabilities, such as with NIR light source, may locate a selected region or a marker within a selected region of interest or a reference object in the region of interest, or external to the region of interest. Relative spatial position of the marker with region of interest or selected region measured by various modalities may be used to collocate spatially the regions of interest, the subject and the components and selected objects within the field of view of measurements.

One or more sensors may be used for thickness, or motion tracking, spatial position and/orientation or geometry or dimension measurement. In addition, AI may be used for motion tracking, human face or body part and pose recognition, such as using Face Detection to track, Face Mesh, Iris, Hands, Pose, Head Segmentation, use Object Detection, Box Tracking, and instant Motion Tracking, Objectron (3D Object Detection), KNIFT (Template-based Feature Matching),

AutoFlip (Saliency-aware Video Cropping)

AI may be used to identify region of interest and volume of interest, some times in real time, by selecting digitally or help an user to visualize, for example, by combining the shape and geometry, spatial relative position of one or more regions of interest, surface color of each region of interest, and/or of the object, and allow manual or digital selection based on one or more criteria of a volume of interest, or a region of interest, for x-ray measurements or imaging and/or for real time x-ray imaging and measurements. After the region of interest is selected, the controller sends commands to the motion system, for example a translation stage to move the x-ray tube and/or the corresponding detector to image the region of interest. The translation stage may be movable in at least one axis to 6 axes in time.

The x-ray source and detector pair may be mounted on x-ray imaging system similar to that of conventional x-ray system, C-arm, U arm, Upright, etc. x-ray source assembly capable of tomography can be integrated into an existing system. For example emitting position may be moved by an electron magnetic steerer, or a mover , same as in a U arm, ceiling mount translation stage, or a separate mover may move the x-ray source for 2D, spectral, or tomography, 3D, up to 6D and 7D in time and in multiple dimensional imaging, or combination of all of the above may be sued for a selected region of interest by the camera or video camera with AI capabilities. In some cases, markers are used to assist in 6D or 7D measurements, or at least two camera or image sensors may be used.

User may intervene or confirm selection of volume of interest prior to x-ray imaging.

The automated selection of VOI or motion tracking may be done in a series, for example, by studying the movement of the knee or spine while in motion, which may trigger digitally automated x-ray measurements in series.

In a combination of AI motion tracking with an camera, combined with x-ray imaging, images from each of two modalities may be synchronized in time, and may reduce the radiation dosage while using x-ray for characterization of body or tissue motion functions and locating ROI or VOI with much more precision than previously possible.

Combination of use of AI in optical camera collocated with x-ray measurements may increase precision and accuracy of measurements, completion of characterization of body parts and associated motion and at the same time reduce X-ray exposure, such as when the optical camera is much faster than x-ray camera.

Merging of camera measurement and x-ray measurement may also make the images more visually familiar for the viewer. AI may be used to merge such measurement to form continuity and collocation of the measurements and images taken by each modality.

Presentation of collocated object or subject or region of interest or volume of interest images may be merged to have real life presentation both using camera captured image for the exterior and for the interior volumetric measurement using x-ray. Different color schemes may be applied to different data set to represent different component or object or segment of region of interest or material decomposed substances, or extracted image data or subset of data synthesized based on the complete measurement in space and in time.

The same method and apparatus may be used in nondestructive testing of motorized machine objects object such as robots or cars or car parts.

Display and Input Device

A joystick may be used as an input device for localized control of the motion system for imaging, for example, to relatively position the detector and/or x-ray tube to be directed at the subject to be imaged.

The electronic controller or microprocessor may be wired with and/or inside a display panel box. The display box or display panel or may have mechanisms to relatively position the imaging system or submodules of the imaging system to the imaged subject. Such a display panel or display box may be touch screen.

There may be an emergency button localized to the imaging system for shutting off the system separately from the workstation or the microprocessor and the display set where the acquisition and/or viewing application resides.

Ipad or another electronic tablet device may be used as a display system.

The controller may be connected to a microphone or a speaker. Sometimes an amplifier may be used.

Sound alarm may be part of the system for warning patient or the user system failure, submodule failure, too much radiation or image not taken properly, or system is not functioning problem, warning of patient or user safety or that the system or submodules have been opened up without access authentication.

Each software activity or hardware function performed may be logged. Such a log may be presented in a report for diagnosis or maintenance or monitoring or remote monitoring in a different location or time.

Escalating warning systems may be included, such as yellow light for warning of safety related issue or hardware failure or mal-function or software errors and red indicator for catastrophic failure or malfunction which may damage the x-ray system or patient or user's safety could be endangered.

A sensing element can be used for sensing distance, geometry, or relative spatial position of various hardware elements, submodules in the x-ray imaging, and controller and display and controlling unit.

A tapped collimator may be used to reduce x-ray source focal spot size. An x-ray tomography system with a large field of view can use a tapered collimator for reduction of x-ray source focal spot size. Motor and attachment may be used to attach the tapered collimator to the x-ray tube housing or another collimator attached below the x-ray tube.

Attachment hardware may be motorized or adjusted by a mover or a robotic arm to move the tapered collimator in and out of the line of path in between the source the VOI.

Tapered Collimator may be used to reduce focal spot size

Tapered collimator may be used to restrict region of interest.

Tapered collimator may be used as a conventional collimator, which has a design similar to a shutter, to allow open and close of an aperture for adjusting exposure time.

In some cases, the tapered collimator may have stacked layers of beam attenuators, each layer having slightly larger aperture than the layer above it, forming a tapered hole for transmission.

A sensing element may be attached to the mover for x-ray tube or may be attached to the x-ray tube, or may be placed at a spatial location, separate from the x-ray tube 12 and/or the detector 22 and/or holding structure 104 or mover or a submodule involving either the x-ray source 12 or detector 22 or the detection module containing detector 22, beam particle stopper or beam particle stopper plate 100 or beam selector or one or more other detectors for imaging the imaged subject 2.

The sensing element may be an optical sensor or time of flight sensor or a camera or a lidar device, and may measure the dimension or geometry or spatial location relative to x-ray source and detector and thickness of the VOI or ROI or the imaged subject. The measurement of the thickness may be used for determining the dosage or exposure time, thereby controlling the collimator shutter, or the alignment of x-ray source and/or detector relative to the VOI or ROI, or determining number of projected images or measurements needed for complete reconstruction of the 3D image of VOI.

In some instances, an ultrasound device may be used to determine region of interest within the Imaged subject where ROI resides. For example, instead of determining the number of images needed for the entire thickness, which may include clothing, the thickness of the human body as the imaged subject may be extracted from the ultrasound measurement, to further reduce the number of images to be taken. For example the medical gown the patient wears has already predetermined spatial volume and x-ray measurement characteristics, therefore no additional x-ray images need to be taken for the layers involving the medical gown.

Voice Command and Interaction

The X-ray imaging systems disclosed herein may be integrated with a voice recognition system and/or computer generated voice communication systems for communication and interaction and control and feedback system with a user, and/or a patient and/or a microprocessor.

Virtual Reality and Hardware

The X-ray imaging systems disclosed herein may use a virtual reality viewer or virtual reality eyewear for viewing x-ray images extracted from the x-ray measurements, in some instances, in real time, and with much reduced or minimal image processing compared to conventional imaging systems.

Virtual reality images can be constructed for virtual reality viewer and/or virtual reality eyewear, a virtual reality head mount with a camera viewer, especially tomography images from CT, which may require stitching and/or interpolating to complete imaging experience. Features like Zoom in and out requires image processing prior to viewing. Generally 3D viewing is not real time using current portable CT or CT systems, unless navigation software is used. And with software assistance, the information viewed may not be precise enough or may be with artifacts.

With the present disclosure, the virtual reality viewer, for example, with a head mount and eye piece, may be integrated with minimized image processing and be presented in real time.

In scenarios where real time viewing is not required, the image data acquired by the x-ray measurement and imaging system can enable direct 3D viewing with reconstruction as described in the present disclosure, but with much reduced software image manipulation and data gap filling, and greater artifacts and noise correction as needed with conventional CT or tomosynthesis images.

In medical CT systems used in human clinics, generally the resolution is currently at 100 um at most along the axis of the thickness, for example, thickness along the center axis of the x-ray beam, perpendicular to the detector. A greater resolution may not be practically due to a large number of images that need to be taken, for example more than thousands, and the amount of radiation exposure to the subject required in order to reach any higher resolution.

However, the 3D techniques described in this disclosure can enable selected VOI to be as small as in sub millimeter range in the xy direction. In addition, the feasibility of high spectral resolution, or multiple energy which may be used to interrogate the same VOI can increase the sensitivity of the measurements. If the number of images taken is large in a small cross section, the total radiation level may be tolerated, allowing interrogation of down to the micrometer range or even smaller than micrometer range. This may allow imaging of small blood capillaries or substances very small in dimensions, which in term can improve diagnosis, monitoring and tracking capability, viewing features like zoom into small features in the micrometer range or sub micro range. Measurements of small features with or without the virtual reality viewer may be implemented. In some cases, such implementation may not need additional software processing after tomography reconstruction.

Memory or Computing Apparatus and Methods

3D, CT computation, X-ray measurements, sometimes, require large amount of memory and computation. In the nMatrix method and apparatus described here, a detector module or assembly or a submodule attached onto existing detector or detector via wireless communication or tethered mechanisms, may containing memory storage, and/or database capability and/or database and microprocessor for localized computation, processing and/or storage. The display may be done locally or directly from the microprocessor or remote via wireless or ethernet or other tethered communication method to a second microprocessor for display and in some cases for further computation and storage.

ADC conversion of 32 bit or above for detectors and its dynamic range may be used for image processing, useful for 2D images acquisition, processing, and multiple dimension applications where a high dynamic range is useful.

The microprocessor or memory or database storage and management may be local with ADC resolution of 32 bit or more, and may be used for image processing, acquisition control and AI with X-ray imaging apparatus and methods, such as illustrated in FIG. 29.

Tomography and/or spectral imaging with dual or more energy levels and image guidance and tracking applications can be suitable for the above configuration.

The localized memory and microprocessor capabilities may be used with scatter removal apparatus and/or methods, or scattered removed or x-ray image with 1% of less scatter over primary interference.

The localized memory and microprocessor capabilities may be used with x-ray imaging of more than two organs such as cardiovascular or pulmonary imaging, dynamic perfusion imaging or approximately whole body applications.

The localized memory and microprocessor capabilities, with ADC resolution of 12-16 bit or 32 bit or higher may be used in industrial applications.

Optical Imaging Assembly

For dual or multiple energy 2D and 3D imaging, or 3D imaging using nMatrix method, or scatter removal methods, or one of more combination of the above methods, instead of using an x-ray detector downstream from the region of interest of the subject, a sensing assembly including intensifier, or scintillation, optics, and optical camera may be used.

Small optical sensors may be used to image a large region of interest since X-ray sensor of large format may have limitation in speed, or resolution or performance.

For example, when a scintillator or Intensifier is placed downstream from the Volume of Interest relative to the x-ray source, the scintillator or intensifier can convert the x-ray to optical signal. A sensing assembly can include optics such as optical lens or objective lens to focus the image from intensifier to an optical sensor.

The optical lens could be doublet or includ multiple lenses. The optical lens may be automatically adjusted to zoom in or zoom out to visualize optical light coming from one or more regions of intensifier.

A slanted (for example, at 45 degree) mirror may be used to decrease the distance between the image formed at intensifier to the sensing plane of the sensor. The sensor may be a 2D optical sensor.

A negative lens may be used to further focus the optical image converted from the x-ray image collected by the intensifier to the input aperture of the sensing assembly.

Two or more such sensing assemblies may be used or moved into a region of interest on intensifier where VOI has a projected image. Such sensing assemblies may be different from each other in size or frame rate or energy sensitivity.

A mover is used to place each of the assemblies so that the input aperture and sensor image plane may capture the image formed by intensifier due to the X-ray illuminating VOI, projected on the intensifier. Alternatively, an objective turret may be used to placeto position two or more optical assemblies to collect x-ray signals from the region of interest or volume of interest.

In some instances, the intensifier of appropriate size may be coupled to the input aperture of the optical sensing assembly.

And such an assembly may be moved with an intensifier or move relative to the intensifier to image region of interest.

X-ray beam Chopper

Beam chopper may be used to illuminate one or more distributed ROI or it may be used to track a ROI at one or multiple spatial locations. The purpose may be one of the following:

reduce radiation , for example, chopper may be made of collimator with distributed holes, this may reduce radiation or produce structure illumination of the ROI or filters to reduce energy levels, for instance to filter out soft tissue sensitive low energy x-rays to image bone only.

reduce focal spot size of the source, for example, using one or more motorized tapered collimator in and out the beam path.

reduce radiation exposure time, for example, reduce radiation exposure time at one or more x-ray emitting location.

Shorter x-ray pulses may be produced using an x-ray beam chopper. A fast, economical, and compact x-ray beam chopper with a small mass and a small moment of inertia whose rotation can be synchronized and phase locked to an electronic signal from an x-ray source and be monitored by a light beam is disclosed. X-ray bursts shorter than 2.5 microseconds can be produced with a jitter time of less than 3 ns.

A device for chopping x-ray beams emanating from a source can include a rotating disk, said disk defining a channel extending along a diameter of said disk wherein the source is pulsating, wherein the angular position of said channel on said rotating disk can be phase locked to said pulsating source, and wherein the variation in the time required for each revolution in the rotational speed of the channel may be less than 5ppm.

A device for chopping x-ray beams can include a rotating disk having a first side and a second side, said disk defining a passage extending along the entire diameter of the disk.

A device for chopping x-ray beams can include:

a disk having a channel extending along a diameter of said disk and positioned at a specific height relative to a base face of said disk;

a shaft connecting said disk to a motor;

a first electrical circuit for powering said motor and a second electrical circuit for controlling said motor's speed;

an optical sensor for determining a speed associated with said disk and for communicating said speed to said second electrical circuit;

an x-ray beam incident on said disk where said x-ray beam strikes said disk at a specific incident frequency and at a position on said disk coincident with said position of said channel relative to said base of said disk.

The x-ray beam chopper fixture may be attached to the collimator or directly below, alternatively x-ray beam chopper may be in between the collimator and x-ray emitting position.

Multi-disc chopper may be used. Each disk may have different spot size to create different focal points depends on, for example, some of which may be smaller than the original focal spot size, especially for high resolution Xc in the z axis direction.

To create an x-ray beam, a first set of magnetic elements can transport an electron beam into a magnetic field created by a second set of magnetic or electromagnetic elements, wherein the magnetic field created by said second set of magnetic or electromagnetic elements can cause said electron beam to generate an x-ray beam in positions different from the positions of x-ray emitting position created by the first set of electromagnetic or magnetic elements. The movement of emitting position is for 3D imaging reconstruction. Alternatively, the movement is for illuminating a selected region of interest. If the target includes various materialsthe electron beam can be moved to a different target region which may include a different target material and the x-ray source may generate x-rays of a different energy level, allowing for rapid switching of different energy level x-rays in multiple spectral measurements.

In some cases, the rotating beam chopper may not need to be moved.

Alternatively. A spinning disk with transmissive holes may be used as a beam chopper. The center axis of the spinning disk is preferable parallel and side by side with the center of the cone beam.

Such a beam chopper can be fairly lightweight, therefore portable.

Cloud Computing

Acquisition, analysis, processing and viewing of X-ray measurements disclosed in the the x-ray imaging system and apparatus and methods can be used to generate point, 1D, or 2D, or multiple dimension or 3d image construction, image processing and/or segmentation and/or annotation, and/or presentation and AI and related analysis and data mining processes and functions. The processing may be done on one or more local computing devices or microprocessors local work stations at the site of measurements or on one or more cloud locations or at a remote server.

Updating and fixing of software errors and bugs may be done locally where x-ray image is acquired and/or stored at the detector location, and/or the microprocessor connected with the display device and/or at a remote server or remote processor.

Gated Measurements.

Delayed enhanced or multiple phase imaging with contrast, and diffusion or gated measurements used in other modalities may be used with the present x-ray measurement using similar external modulation with chemistry or electric, electromagnetic and other energy sources.

Acoustic Noise

To cancel and reduce acoustic noise from background and/or from an x-ray machine and related motors, one or more acoustic cancelling materials may be used to shield the system or be placed at various locations to cancel or reduce acoustic noise. For example, the materials can include foams or nanoparticles or nanochannels distributed within a foam, or self-assembled graphene foam. Examples also include using a ring-like structure. The ring can include helical channels along its thickness.

Active noise control may be used to reduce or cancel noise.

Acoustic noise reduction system can be in an open loop or closed loop system where the noise is recorded prior or in real time. Noise control can be used to generate or inject a synthesized anti-phase signal, which is generated by inverting the phase of the major frequencies components of the recorded acoustic noise signal. This synthesized signal can be synchronized with the scanner sound with a trigger generated by the scanner computer to align with the pulse sequence.

In one example, a pair of piezoelectric speakers can be placed near the patient or the user. An adaptive controller with a multi-channel filtered-x least mean squares (FXLMS) algorithm can be used to generate the noise canceling acoustic signal.

In one example, a feedback controller system with a cascaded neural-network architecture can achieve the reduction of x-ray system acoustic noise. This system was tested using a loudspeaker that presented pre-recorded noises.

In one example, a feedforward ANC system in a communication system can utilize an optoacoustical (that is, a piezoelectric speaker driven by optical signals) for noise canceling.

In one example, a hybrid controller, in which both feedforward and feedback loops are embedded, may be used.

Portability

Current X-ray imaging system may be portable, by pushing charts or wheels, which has limited portability, due to Size: for example in a CT, dimensions and size is too big, for 3 D imaging or Availability of motorized systems.

Conventional mobile x-ray, or portable x-ray systems, or x-ray systems with wheels have limited capabilities, such as having no scatter removal methods using beam selector or beam particle stopper, or multiple dimension imaging capabilities or spectral or dual energy imaging capabilities as described herein.

Portable or mobile and/or Autonomous or remote controlled portable X-ray Machine will now be explained.

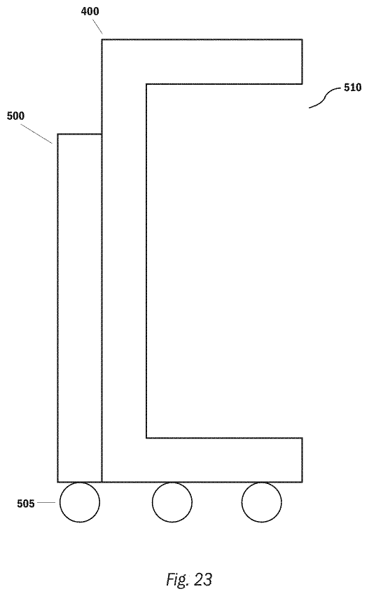

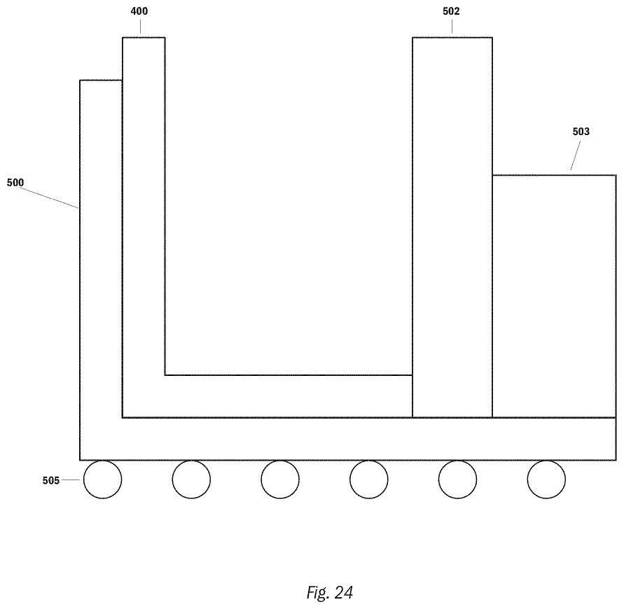

Examples are as illustrated in FIG. 23 and FIG. 24. In FIGS. 23-24, a mobile x-ray machine, 510, may be capable of autonomous self-moving or self-driving or remote controlled, or airborne via a plane mechanism or helicopter mechanism or a space vehicle or may be controlled by a human driver or a pilot or an astronaut.

FIG. 23 illustrates a Portable x-ray system or its submodule attached to or integrated with a motorized gear, which may be an electric powered gear steerable by one user or more users or may be a self-driving device or may be a remote controlled gear for movement in hospital or outside of the clinic. Such a portable system may simply be manually pushed by a user. Such a system may include a CT system, or general x-ray system or a system which is CT tomography, general x-ray as well as personalized precision CT imaging.

FIG. 24 illustrates a portable x-ray system that includes additional areas or gantry to allow for portability of additional hardware, for example, accessories and submodules of x-ray or one or more trailer compartment.

The x-ray imaging apparatus and methods disclosed herein may include using remote controlled mobile systems 500, or autonomous driving robots, or motorized robots or mobilized system to attach on to the x-ray system 400 mechanically or magnetically or integrated with the x-ray system by mechanical parts.

The x-ray system includes a portable system which allows portability for example, having two or more wheels 505 or moving mechanisms attached to the x-ray system.

The portable x-ray imaging apparatus and methods may include x-ray systems 400 with scatter removal capabilities such as time or frequency or spatial methods as described, and spectral or dual energy imaging capabilities.

The portable system may have addition room for additional compartment 502 or mechanical attachment for additional compartment for accessories or submodules associated with the x-ray system, such as a generator 503 or a display 502 or a computer 503 or hardware associated with or part of the x-ray system.

Such a mobile system, or portable system or self-driving or remote controlled system with x-ray system may be compact and may fit through a standard door or an elevator, or patient door, or operating room door. For example, the portable x-ray system can be approximately 35 inch wide and 6 feet 8 inches height.

The portable x-ray system may be remote controlled by user outside the room where the system is stored or may be controlled by user A inside the room, sometimes such a system may be shielded by a x-ray shield, including an apron or a lead panel. Such lead panel may be removable or may be attached to the x-ray system.

The portable x-ray system may have one or more position sensor or one or more cameras to image the subject and position the subject.

FIGS. 25 illustrate an Apparatus 602 for imaging of various body parts or tissues or organs with the large field of view x-ray system, which may be capable of imaging an entire human body. The apparatus 602 may be attached to the x-ray system with the source or the detector or may be a standalone device. And the apparatus may be placed on or attached to an additional support device such as a patient chair. X-ray source 12 is attached to an upright stand 400. Detector and/or detector module including multiple detectors 22 may be attached to another stand 401

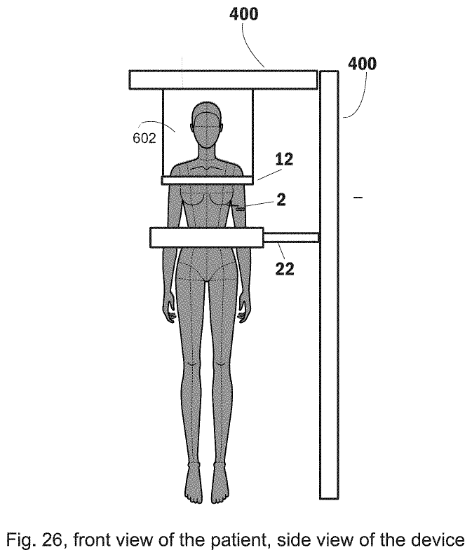

FIG. 26a illustrates a Front view of a mammography device 602 attached to or detached from the x-ray system with large field view or multiple purpose of x-ray mammography or tomography or CT tomography or personalized or customizable CT.

FIG. 26b illustrates a side view of a spectral tomographic mammography device.

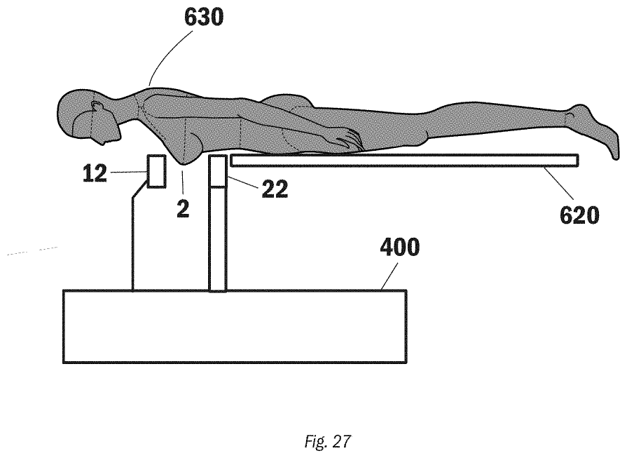

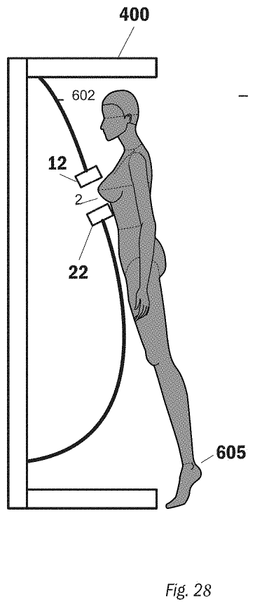

FIG. 27 side view of a mammography support device 620 attached to or detached from the x-ray system capable of 3D imaging, CT or personalized and customizable CT system capable imaging of regions other than that of mammography.

A speaker and/or microphone may be attached to the controller unit 200 as illustrated in FIG. 37. The speaker or the microphone may be attached to a stationary x-ray system or the portable x-ray system or may be separated from part of x-ray system. Such a device is used as a communication system between the user and the machine, the operator and the machine and/or user and the patient, especially when the patient and x-ray system are in a separate location than the user or the digital computer workstation. The user or the computer may instruct the patient to a desired position, or to engage in x-ray imaging related movement, activities and communications. The device may have a microprocessor. The microprocessor may have voice recognition capabilities and sound conversion system, which can digitize sounds or record sounds or digital command from the patient or a user in the same location as the patient, instructing the patient, such as by converting sound wave signals to digital signals and transmitting via a ethernet, fiberoptic network, wirelessly to a computer or a phone with computer capability either in the same location as the patient or at a remote location.

User at a remote location and/or a microprocessor may be able to understand such information based on the patient or the user A's original intent, and communicate back through using digital command by using a software and/or hardware or using voice, or sound which is converted to digital information and transmitted back to the device or the x-ray system in sound or digital form, either using remote communication mechanisms, such as cellular communication, or using satellite or internet or a landline phone. X-ray system may have voice recognition or taking capability and/or may process digital information translated or converted from the voice or sound or direct digital information from user A and/or user B and/or microprocessor input at a remote location or locally.

The user A at the location remote from the x-ray system and/or the patient or the imaged subject may be able to see the optical or thermal image or measurement of the imaged subject via optical or other methods or the x-ray image, via internet or via other remote communication methods and may be able to direct or communicate to the user B located approximately at or near the x-ray system location and/or the communication device at the x-ray system location based on the image or images shown in a display device, such a phone or a TV or computer monitor or a digital display or projected image.

The said x-ray images are taken with a digital area detector or photon counting detector and/or with scatter removed using a beam particle stopper plate 100 or beam selector or a primary frequency modulator or using a time of flight x-ray source. The images derived may be a raw image to be processed at the location of x-ray imaging system or processed at a location different than the x-ray imaging system. The reconstructed image or real time measurements may be transmitted via wife, bluetooth and wired connections to be further analyzed digitally for diagnosis or image guidance or processed or displayed at a remote location with one or more microprocessors and/or a user.

Alternatively, all the image processing or reconstruction or analysis for diagnosis or image guidance may be done at the location of the x-ray system by a microprocessor or two or more microprocessors and/or a user.

The detector or two or more layer stacked of detectors may have a mover attached for moving a beam particle stopper plate or a beam selector in between two detectors. And a mechanical component such as a holder may have stoppers and brackets or frames to hold each of detectors, or beam particle stopper plate or the beam selectors in place. And there may be beam stoppers all around the assembly and its frame to reduce ambient interference.

On top of the each detectors, there may be protective layers, such as carbon or polymers to keep the detector from being damaged. The layers are optional.

A patient table or surgical table or sample table may be placed between the subject or the patient and the detector gantry, where the beam particle stopper plate 100 and related assembly, the detector 22 and the rest of mover hardware resides.

Each part of the x-ray system may be powered by a battery or plug into the wall or a central power device, plugged into the a power outlet in the room. The power device has one or more power plugs so that one or more part of x-ray system which requires power may plug into it via electric wire and plug or plugs. One or more part of the x-ray system, such as the generator, or the heat exchanger or the x-ray tube or mover or movers to move each element of the hardware, such as a mover for one detector, or the detector assembly, or a collimator, the PCI card, the controller, or communication device and/or a microprocessor for image processing and/or image storage, may be battery powered with one or more battery in series or in parallel, or may be plug into the wall socket. The battery or batteries may be rechargeable battery or rechargeable batteries. there may be a rechargeable station connected with the x-ray system or detached from the x-ray system as a separate unit.

The autonomous driving unit ADU-1 or automated guiding device, or automated moving device may be integrated with x-ray system or detached from the x-ray system, but has mechanical attachment or magnetic attachment, such as an joining device between train compartment or a clamp or mechanical buckle to be connected with the x-ray system or part of the x-ray system and moved with the attached system from location to location.

The driving unit ADU-1 may be part of a mobile vehicle such as a van.

The driving unit ADU-1 may be have one or more the following:

Camera.

position and motion sensors and driving gear.

Lidar or laser or camera other sensors for sensing distances to nearby obj ects.

A microprocessor which stores the dimension and 3D shape of the x-ray system and its associated device. A microprocessor that may have a voice processing microprocessor to process commands from a user or a computer. A microprocessor that may have a speaker to release sound and comment and sounds or sound alarm or speak when necessary. A microprocessor that may be connected to sensors, which may be able to sense and calculate relative spatial position between the x-ray system or the container in which x-ray system is contained relative to its surroundings to adjust speed and make turns and steer and change directions, reverse, go forward, brake and avoid collisions along the movement.

A software which enables machine learning algorithms.

Combine sensors and software to control, navigate, and drive the vehicle. Such software may include one of the main tasks of any machine learning algorithm in the self-driving car, such as a continuous rendering of the surrounding environment and the prediction of possible changes to those surroundings. These tasks are mainly divided into four sub-tasks:

Object Detection

Object Identification or Recognition Object Classification

Object Localization and Prediction of Movement

Machine learning algorithms may be divided into regression algorithms, pattern recognition, cluster algorithms and decision matrix algorithms.

Integrated with the x-ray system is a portable robot that follows along marked long lines or wires on the floor, or uses radio waves, vision cameras, magnets, or lasers for navigation. Such a robot may have steer control systems and/or path decision features. Such a robot may be integrated with x-ray system and/or attached with a trailer, in which one or more parts of the x-ray system may be placed in the trailer, and/or accessories of external objects may be placed in the trailer.

Such a portable x-ray system may be remotely controlled at a location nearby, for example within a line of the sight of the operator or of a remote microprocessor and/or by an operator with an microprocessor and display which displays the images or location or location measurement of the x-ray system spatially, and/or a microprocessor, at a remote location or a remote room and/or self-driving and/or having autonomous driving microprocessor and sensors for traveling to a site or location not accessible, or moving in a field of hazard areas or in a room not suitable for human operators, or at Point of Care, in a patient's room, on a sports' field or stadium.

The updating and upgrading and fixing of software errors of such x-ray systems, and location or position moving related software and operation of the x-ray system may be done via a cloud server or a remote server wirelessly, for example, Bluetooth, WiFi, cellular or satellite mechanisms or through a wired connection, for example Ethernet cable or fiber optic cable, or USB cable or a local microprocessor, automatically via a software or by a user using software.

Generally any portion of the present disclosure may be added to one or more existing system to accomplish the intended purpose without having to offer the described apparatus and method in its entirety.

x ray system structure may have spatially adjustable support or legs to allow the legs to be placed in adjustable positions, to ensure portability and compactness. For example, some legs or support structure may be folded in or include movable features to ensure compactness preferred during transportation or movements.

Adjustable height features of the structure , for example, using telescoping design, may adjust the overall height of the structure either during imaging or for imaging at different Source to Detector distances and/or for stability and compactness preferred or required during transportation. In some cases, such adjustment or design may not be needed.

One or more detectors may be moved by the same motor that moves the detector 22 or detector assembly. Mechanical structure made designed so that it supports the weights of the system and contributes to the stability of the x-ray system so that while x-ray sourced is being moved and/or detector is being moved, there are no or limited destabilization of the x-ray system. Vibration and/or movement of the entire system is limited, or does not interfere with the imaging process.

In one example, one mover such as a motorized actuator AI, or more movers may move the beam particle stopper plate 100, which is placed in front of the detector, facing the region of interest or the subject. If two movers are used, one may be placed on a first side and the other motor may be mounted on a second, opposite side of beam particle plate 100. The two movers may be approximately on the same plane of the beam particle topper plate and/or the plane immediately below the beam particle stopper.

In one example, a mechanical device such as a plate 300, which may be made of aluminum, is used to mount the detector into the mover 400 (see FIG. 30) or translation stages or a gantry mounted on the translation stage. Such a mover may be enclosed by an enclosure 405.

And there may be one or more wheels 108 or a moving gear mounted on to the x-ray system, to one or more parts of the x-ray system, situated below the x-ray system. the wheels may be made to move by manually by one or more users, or motorized by a powered motor or powered gear, either case, the user is to steer the movement. Alternatively, part of or the approximately whole system of x-ray is motorized by autonomous moving and steering devices or remote controlled autonomous moving devices, which is attached to part of or the whole x-ray system.

The detector may be mounted on to an aluminum plate. On either side of the detector, there may be a motor A1 and/or A2 mounted, for example to the aluminum plate 300. A moving mechanism of the motor, such as a carriage of each motor can be mounted to the beam particle plate which is on the side of detector, opposite to aluminum plate. The motors can move the beam particle stopper plate 100 in the x and y plane parallel to the detector. Each motor may be battery operated or driven by being plugged into a power supply connected to a wall plug or a distributed power plug or a power source or plugged into a device that can supply power as part of the x-ray system or detached from the x-ray system.

The motors may be working in parallel. Each may drive the beam particle stopper plate by a distance.

Electrical, magnetic or optical position sensors may be used together with the motor or motors to home the location of the motor.

To derive the complete image without the information gap, at least one movement in at least once in linear fashion can be made so that the beam particle stopper can be displaced from its earlier position.

Alternatively, any type of driving mechanism or movement or in any direction may be sufficient so long as the position of each beam particle stopper blocking the x-ray illuminating the region of interest can be shifted from the earlier position.

Point of Care Tomography or Spectral Tomography or POC Scatter Removed X-ray Imager

X-ray imaging system disclosed herein can be portable and available at Point of Care. Parts of or a portion or a complete system may be mobile and may be available at point of care due to portability and compactness. Such a system may be a multiple dimension imaging device or a dual energy or multiple energy or spectral imaging device and/or 3D imaging or 4D imaging or up to 7D imaging device, and/or may be of an imager of more than one body section, for example, approximately whole body imaging. A 7D device is an imaging system capable of tracking in 6 dimension information in time.

The current electronics can allow fluoroscopic images and all rapid storage of images and transfer to a computer distant from the detector. At point of care or in the field, the x-ray detector or x-ray detector assembly, for example, a beam selector sandwiched between two detectors or a beam particle stopper plate sandwiched between two detectors, or simply one detector with a beam particle stopper plate or a simply a detector or a detector assembly involving at least one detector, may have a memory storage capability which can store 20 images or more. Such detector and storage device may be wireless connected through internet WIFI or Bluetooth or other wireless communication methods, or may be directly connected via an integrated circuit or fiber optic or Ethernet cable or RS232 or other connected mechanisms.

For identification of region of interest (ROI), one or more 2D images of the subject taken at various angles may be sufficient to identify region of interest. The identification of ROI can optimize the imaging process so that the VOI or region of interest are imaged only when necessary and if possible, minimized in dimensions to reduce dosage to the subject.

In some cases, the imaged region of interest may be presented in a background with dimensions of the subject larger than ROI, therefore provide the reference needed to locate or orient the ROI. The background image may have higher or lower resolution and/or varied spectral sensitivity.

The detector may be placed between the patient and where the patient sleeps on or sit on or rest on, such as a hospital bed, x-ray table, or surgical table 40 in FIGS. 52a-e or any surface which may be or may be connected with a support such as a bed.

Previously handheld x-ray source is used without scatter removal, therefore has limited application. In the present disclosure, spectral imaging x-ray imaging with a handheld x-ray source allows for tissue densitometry, tissue differentiation and better diagnosis of illnesses with higher resolution, use of quantitative imaging methods and Artificial Analysis in for example, diagnosis of illnesses, such as in dental applications, in sports medicine.

Handheld x-ray imaging system can remove scatter using interpolated methods as disclosed herein.

Handheld x-ray imaging system can have spectral imaging capabilities, using multiple energy x-ray source with flat panel detectors or energy sensitive detector sets, for example, repeating units of x-ray detecting regions including two or more energy selective detectors. Such an imaging system may have scatter removal apparatus, and is portable and can be used in one or more settings or different rooms in a hospital or dentist office.

Handheld x-ray source can be capable of 3D imaging and/or spectral imaging by using an electromagenetic steerer or pixelated x-ray source.

Flexibility and Versatility of the x-ray Systems Disclosed Herein

At least two x-ray sources may be used, as illustrated in FIGS. 1-4. Each source may move in and out of an emitting position, which is able to illuminate region of interest. The projected signal can be captured by a first detector. Such sources may be of multiple energy sources or single energy source, or quasi monochromatic source. Such sources may be of different energy levels. For example, one may be at 40-150 KeV, the second may be at 20-40 Key.

The mover to move the x-ray sources in and out of the emitting position or emitting positions may include a rotating turret or a linear stage, or two dimensional stage or three or more dimensional stage, a rotating moving stage. The x-ray sources may be modulated to move in and out an emitting position by steering the electron beam, for example, via electron beam deflection, by, for instance, a set of electrooptic lens or electromagnetic or by magnetic methods such as magnetic plates or solenoid coil.

In FIGS. 5-8, in the examples illustrated, the x-ray imaging system and apparatus disclosed here have one or more detectors, which may be referred to as second detector or detectors, downstream or upstream of a first detector relative to the source or the volume of interest or the imaged subject.

The second or third or fourth detector may be moved in and out of the emitting position where it may illuminate the region of interest, and measurements may be taken based on the application. Such detectors may be mounted on a stage, manual or motorized, and/or may be rotated to reach each quadrant downstream or upstream the first detector 22. In each quadrant, the detector or detectors may be moved with a linear, or 2D or multiple dimensional translation stage within the quadrant. Such detectors may be without scatter removal devices, or may be used with a beam particle stopper plate 100 downstream from VOI or upstream of the subject. Such detectors be moved into the position of the first detector after the first detector is moved out of illumination path of the VOL In some cases, the second or third detectors may be used, and each may include a beam selector sandwiched between two detectors as described herein.

One example of the present system includes various mechanisms to move one or multiple detectors relative to the source and/or relative to the subject.

Both the source and the corresponding detecting module may move in sync.

There are various mechanisms such as mechanical, electrical, energy driven to move one or multiple x-ray source relative to the detector and/or relative to the subject and/or relative to the subject. In some cases, the movement of x-ray source and detector may be synchronized with each other.

Hardware and software can be used to synchronize the movement of treatment probe or device with that of the source and/or detector or that of field of view selected and/or the subject relatively or independently. Corresponding algorithms may be derived to incorporate the relative movement or absolute movement data corresponding to x-ray measurements in registration, capturing, recording, and saving time and image reconstruction of x-ray measurements and images. Deductions of the measurements and images may give rise to facts for further analysis regarding property of subject and components in the subject.

The present system may include sensing x-ray measurements above certain threshold of measurements at various locations and positions in 6D space and in time, and in turn triggering one or more events or activities about x-ray measurements and analysis of one or more selected regions, components , targets or subjects.

The measurements of the present system and measurement processing methods can determine and derive one or more facts, organize and structure data and to assess probabilities of one or more facts to determine and predict one or more facts by analysis, create scenarios or develop theories or construct possible conclusions and characterize, identify, state, subject and/or monitor components and subjects and for image guidance.

In some cases, such measurements, analysis and measurement process methods are combined with data from a user input or digital input and data from measurements by the same or other modalities.

The x-ray tomography system may be combined with other form of tomography devices, for example: when x ray tomography is reconstructed based on rotational move to generate varied illumination path through a voxel in ROI. Minimized rotation steps may be used similarly to translation movement, in that as long as the rotational movement of the source in a circulator projectory around the VOI generates new projection path different by at least substantially one voxel, then if there source rotates around VOI in two axis or in least one axis combined with a translation linear movement, or in a helical projectory, approximately similar number of total projections may be needed to generate essentially a complete tomography image of VOL This is a little more complex than moving the source in xy plane. However, in situations where radiotherapy is administered, combinational projectory imaging may be needed. In reconstruction of a combinational projectory, additional vectors may be be formed to allow representation of the combinatory projection geometry.

X ray tomography imaging device may be combined with the following imaging method, for example, when mounted on a C arm or 0 ring, or tomosythesis, for image reconstruction, system matrix may have adjusted number of coordinates or vectors to adjust number of degree of freedom to represent such radiation projection geometry for each component generating projection image data, for example in a system where

A method of imaging an object using radiation can include the following: obtaining projection data from at least one detector array, the at least one real detector array obtaining projection data at two or more positions of relative x-ray emitting position to the object, optionally at least one detector array and/or x-ray source emitting location has two or more positions, having a geometry that is neither equilinear nor equiangular; reprojecting the projection data onto a virtual detector array that has a geometry that is either equilinear or equiangular; and reconstructing the reprojected data from the virtual detector array.

Optionally, at least one real detector array can include two or more detectors configured to obtain projection data at two or more positions.

Optionally, the at least one real detector array can include at least one detector that is movable to obtain projection data at two or more positions.

Optionally, the method can further include projecting radiation from a source onto the at least one real detector array.

Optionally the radiation can include x-ray radiation.

In the method, the virtual detector array is equilinear or the virtual detector array is equiangular.

The projecting the projection data onto a virtual array may include: allocating a virtual array that can include virtual pixels that are equally- spaced in distance or angle; for each virtual pixel, determining a corresponding real detector pixel in a real detector array that is intersected by a line connecting the virtual pixel to the source of projected radiation; and using a radiation amplitude value detected at the corresponding real detector pixel to determine a radiation amplitude value for the virtual pixel.

To determine a radiation amplitude value for the virtual pixel, a value from the radiation amplitude values of the corresponding real detector pixel and neighboring real detector pixels may be interpolated.

The method may filter the data from the virtual detector array, and backproject data from the virtual detector array.

The method may have at least one real detector array including at least one one-dimensional line detector, or at least point detector or at least one two-dimensional flat panel detector.

Such a method may operate independently or combine with an x-ray imaging system described in the present disclosure where at least the x-ray emitting position moves relative to the object.

Multifaceted Identification

Multiple factors can be used for diagnosis, for example in certain brain related illness, one or more of the following aspects, such as

alterations in grey and white matter, ventricular volume, structural and functional connectivity and neurotransmitter levels, may contribute to or indicate certain illnesses.

And in addition, additional information, such as gut pathology and alteration may contribute to illnesses elsewhere, for example, due to immune system modulation via the gut.

Each of these factors may be measured with or without contrast agents attached to antibody, such as nanobody or small molecule, to form a molecular complex which can easily pass brain blood barrier.

FIG. 42 illustrates examples of multiple emitting positions in one x-ray source. The x-ray source module 12 can include a plurality of x-ray emitting positions 12-1, 2, 3, 4 or two or more x-ray sources, 12-1, 2, 3, 4, distributed spatially, for example, in a xy plane. The entire module 12 may move from position P1 to position P2, in effect creating multiple different x-ray emitting positions with reduced number of movement steps and thereby reduce time required for image acquisition.

An x-ray source 12 which may include one or two or more x-ray sources or emitting positions, each is at a spatial position different than that of the other. In some cases, such sources are located so that it is as if the x-ray is emitted from a location on the same xy plane. The source 12 may be moved in the xy place to allow for multiple x-ray emitting positions. x-ray can be emitted sequentially from one of the sources, the x-ray source module 12, or the two or more emitting positions. As the x-ray source 12 moves from position P1 to position P2, the distance between P1 and P2 may be Xc, the resolution desired in the z axis or axis perpendicular to detector. If the source 12 has, for example, four emitting positions, as the source moves to position p2, additional different four emitting positions are generated, each varing by about Xc from the P1 of the same source, P1 which is approximately the previous position of the source. The four sources 12-1, 12-2, 12-3, and 12-4 can be distributed from each other, in some cases with a distance approximately equal or greater than >2 Xc, with Xc being the resolution desired in the axis perpendicular to the detector.

In some cases, the distances between the emitting positions of the source or sources within the source model 12 may be less than a pitch of the detector, or Xc.

In some instance, if the mover of the x-ray emitting position move more than Xc, the number of images needed to reconstruct a complete 3D image may be increased, although the increase would be acceptable in computational complexity.

The mover or electron beam steerer may move in units or steps of d12 with a distance of 4×pixel pitch of the detector plus 10 um, when the desired Xc is either greater or smaller than d12. To minimize the number of images taken to achieve minimized radiation, the number of x-ray images taken may be more than the number of images taken to reconstruct a complete 3D image when d12=Xc. However, it may be acceptable, especially when radiation is not an issue and/or the mover can only moves in limited increments or the electron beam steerer is limited in the steering capability required by the optimized imaging routine and specifications for a 3D reconstruction.

In some cases, the distances between the emitting positions may be adjustable, for example, by a moving stage.

In some cases, combination of imaging methods in tomography as disclosed herein, 2D, multiple energy or tomosynthesis, or multiple dimension imaging may be used.

In some cases, more than 1-5 degree may be used for imaging. 1-10 degrees may be used in imaging when precision, accuracy, or minimization of radiation or imaging time or complexity is not a performance priority.

In some cases, the total emitting positions needed may be multiples of the number of emitting sources. For example if 100 images need to be taken to completely reconstruct a 3D image, only four sources are in the source model 12. The total area around each of the four sources may be more than or approximately 2×100/4×Xc^Xc, which is the area size away, or approximate 10 Xc or more apart from its adjacent x-ray emitting source, for example, between 12-1 to 12-2 or 12-3 to 12-4. The source module, 12 with four sources may move in steps of Xc approximately 25 or 24 steps, or move a area of 5Xc×5 Xc or 4×6.

In some cases, emitting location 12-1 may be next to other emitting position such as 12-2, or 12-3 or 12-4, the entire source including of the four sources may be move to an area location not overlapping with its previous position.

One benefit of the multiple emitting position design is to speed up imaging speed for multiple dimensional imaging.

In some instances, each emitting position in one source 12 may emit x-ray of various energy levels or varied parameter values. For example, emitting position 12-1 may emit x-ray beam with a peak between 40 Kev-60 Kev; 12-2 may emit, respectively, with a peak between 20-40 Kev , 12-3 may emit respectively with a peak between 80-100 Key, 12-4 may emit respectively with a peak between 110-145 KevEach movement, therefore each location of the source, may allow one or multiple x-ray energy levels to illuminate approximately the same or different volumes of interest at the same or different times.

X-ray source 12 may be one or more nanotubes, field emitter based, cold cathode source, or pixeled source with one or more pixels.

As illustrated in FIG. 1, X-ray source module 12 may have more than one source, for example dual filament 12-1 and 12-2 or more filament, emitting from a single position. Each source can have a different focal spot size than the other and/or generate various energy levels than the other.

One or more detectors may be used upstream or downstream of a first detector or first detector assembly. Each detecting mechanism may have a dual detector assembly as illustrated FIG. 8 or may have a beam particle stopper plate 100 place upstream of the detector.

Additional x-ray optics used to amplify or miniaturize or adjust or modulate the size of x-ray image generated, or modulate x-ray signal or generate x-ray interferogram or produce modulated x-ray signals at different wavelength. The additional optics may be placed in between the x-ray source and the detector or between x-ray source and the subject, or between subject and detector or downstream of the detector.

In the present disclosure, the aforementioned detectors can be a photodiode, or photon multiplier tube, or photon counter, or x-ray detectors, silicon drift detectors, or any or optical detectors and sensors with deposited scintillation layer or scintillation materials such as fiber optic plate or scintillation crystals. In some instances, such detectors have a beam particle stopper plate 100 or may be in the format of dual detector assembly with beam selector sandwiched in between as described herein.

In some instances, an C arm or U arm is integrated with the x-ray source 12 and in some cases attached to a collimator for selecting field of view or steering for x-ray illumination or for selecting ROI, and for steering one or more corresponding detector modules 22 and 23 such as illustrated in FIG. 30. An x-ray table 400 including x-ray translucent material, or a sample support surface 400, such as illustrated in FIG. 30, may be used to position and support the imaged subject or the sample.

In some instances, the x-ray source and detector are in a portable format such as in hospitals portable x-ray source systems, and may be moved around in a case supported by four wheels. The detector or detector assemblies can be stored in a slot and when needed and the detector module can be placed manually in the position to allow image capture of a subject.

In some instances, the x-ray systems disclosed herein can be made into a table top format, where one or more small animals or in vitro samples, or ex vivo samples, or samples in non destructive testing or security scanning can be placed inside a cased container or an enclosure. The case or the enclosure may be lined with lead. The samples can be placed in a conveyer belt or a sample holder. The sample holder can be motorized. Samples may be placed in the sample holder either from an opening which made the sample holder accessible to the user or from a sample handling robot. Such sample holder may be motorized to be in positions protruding from the case or inside of the case, and samples may be placed on the sample holder while the sample holder is located external to the case. Such sample holder may be motorized to move inside the case, and in some instances, be in the x-ray projection pathway for the sample to be imaged. The sample may be placed in a microfluidic chip or on a lab on a chip device or a tissue chip or a petri dish or a microscope slides or tissue or animal or cell holding or support device, or a device conducive to tissue growth or cell growth.

The case, or the enclosure or one or more sides or parts and/or all of the case or the enclosure may be translucent to light, such as visible light, and at the same time, attenuates x-ray. For example one side of the enclosure may contain a window, including for example, lead glass or Borosilicate glass. Such a window can be in any shape, in preferable implementation it is in a rectangular or round or square shape.

In another implementation, inside such enclosures, there may be one or more light sources, one or more sensors or camera for detecting or sensing movement of the sample or a component of the sample or monitoring one or more regions inside the enclosure.

In some cases, such case or enclosure may have up to six sides. The enclosure may be a room or a part of a building. In some cases, such enclosures can be in any spatial configuration and dimensions, for example, a sphere shape or buck minister fuller sphere shape or cylindrical or rectangular volume or cubic volume. In one implement, such an x-ray imaging system in an enclosure is place on four wheels to allow portability and in other cases, such an x-ray imaging and measurement system with enclosures, may be lined with x-ray attenuation materials and may be placed on a tabletop for use.

The enclosure may include a number of vital sign, temperature, humidity, pressure and physiological monitoring devices and/or other modalities to measure and image objects placed inside the enclosure.

Hardware or modules downstream of the x-ray source may be integrated to restrict or select x-ray illumination on the subject, such as a motorized collimators with one or more filters. It is an aspect of the prevent disclosure to include light or laser markers for positioning the sample relative to x-ray source, or detector or other optics in the system.

The x-ray system disclosed herein can be foldable to a pelican brief case, with power supply, x-ray tube, detector or detector assembly, and in some instances, additional sensors, or detectors, or optical devices, or x-ray optics or collimators or laser marking system for positioning subjects, detector and other modules or subassemblies.

The x-ray imaging apparatus and methods may include two or more x-ray sources or dual or more filament sources, or sources with varied focal spots or focal locations, sizes and/or other varied values in its parameters such as preparation time, exposure, speed, power, energy level, number of energy levels, spectral wave form characteristics, pulse duration, pulse characteristics and/or form factor in approximately one or more x-ray emitting locations. An X-ray imaging system may include one or more detectors movable upstream or downstream of an x-ray detector. Detectors may be the same or different from each other in resolution, speed, spectral value, wavelength, sensitivity, dynamic range, photon sensitivity, quantum efficiency, spectral sensitivity or other characteristics which may differentiate the detector from the others.

In one example, the x-ray system may include at least one computer or microprocessor, at least one display monitor, a hand switch and/or a foot pedal or foot switch for the user or programs to physically triggers the measurement activities, and/or one or more integrated chip with assembled connector and connections to a number of hardware components and electronics, for example, one or more controllers which may control one or more of the following items. The motion or modulating mechanisms may move the x-ray source or x-ray emitting position, the collimator for adjusting the region of interest or field of view for the x-ray imaging system, the motion system for the beam selector or beam particle stopper plate. The motion or modulating mechanisms may move motion system and/or modulating program for various x-ray optics. The motion or modulating mechanisms may move each of the detectors, the control unit for each of the detectors or sensors for data acquisition, the software unit for the synchronizing two or more aspect of x-ray measurements, and/or a control unit for one or more sample manipulation and perturbation or physical or chemical measurement systems. Various components in the system may be linked to the central computer unit or the integrated circuit chip by one or more wires, and/or one or more cables, fiber optic cables or wireless mechanisms including wireless communication via wifi, optical mechanisms or antennas , or ethernet, or Bluetooth. Such a system may be linked to a PAC system or medical record system or a database by wireless or via internet or wired protocols, hardware and methods.

An x-ray measurement device, or computer tomography device, in addition to present images and measurements and features based on capabilities of new functions, features and resolutions, may provide a display mode and present values and images on user interface.

A x-ray imaging system may include two or more x-ray sources or dual or more sources with varied focal sizes and/or other varied values in its parameters such as preparation time, exposure, speed, power, energy level, number of energy levels, spectral wave form characteristics, pulse duration, pulse characteristics and/or form factor in approximately one or more x-ray emitting locations. An X-ray imaging system may include one or more detectors movable upstream or down stream of an x-ray detector. Detectors may be the same or different from each other in spatial resolution, speed, sensitivity to spectral values, sensitivity to one or dual or multiple wavelengths or different energy levels, programmability, dynamic range, photon sensitivity, quantum efficiency, spectral sensitivity or hardware design and other characteristics which may differentiate the detector from the others.

For example, the first detection system can be placed between the beam stopper particle plate 100 and the subject. The second detector is placed downstream from the beam stopper particle plate. The first detecting system may be a point detector, or a linear detector, 2D detector or spectrometer.

In another example, between x-ray source and first detection system, there is no a beam stopper particle plate. This is due to the fact that the second detector or detector system may have a beam stopper particle plate upstream, and the scatter signals on the second detector are interpolated from the low resolution scatter measurements on the second detector. As a result, the high resolution primary x-ray on the second detector can be derived. As the primary signals on a corresponding position of the first detector is correlated to the primary signals on the second detector at various thickness using different material of interests, each material is calibrated by a thin beam at each of the energy levels selected for material decomposition for the subject, or the region of interest or the components. Low resolution and high resolution scatter signals on the first detector can be calculated and derived. In the final step, the primary signal of the first detector can be derived by subtracting high resolution scatter from the composite measured signal. The beam stopper particle plate upstream of the first detector therefore may not be needed. The primary signals at one or dual or a set of energy levels at a particular position can correspond to a derived value of a particular tissue with certain density and thickness, or simulated value or measured data from a reference database. The reference database can be established by multiple imperial measurements of two or more materials at each of two or more energy levels on both first and second detectors. The density and thickness data can be derived due to interpolation of six or more corresponding measured data at each energy at each datapoints. When the number of measurements are increased for each material, the predicated value may not deviate from the actual measurements by 0.05% at a set of energy level. As a result, the rest of the value at various set of energy levels can be interpolated and stored in the reference database. The first detector and second detector may each have different detector response functions for a specific material at various energy levels. The present disclosure extends such methods to three or more energy levels corresponding to three or more materials.

Methods for better visualization and quantification of individual component in a target in a region of interest in a subject, for example, of an individual tissue or object in a body including bone and soft tissue or at least two components, each with different atomic z number or different values of at least one x-ray measurable properties in a subj ect.

The interference of images from the second component or tissue in the x-ray image can be removed from the image of a different component or components. For example, this methods disclosed herein can eliminate the bone interference on its overlapped soft tissue x-ray projection image. The method can based on use of a two-dimensional dual-energy or triple energy or multiple energy. In one preferred example, where a dual energy system x-ray imaging system is used, the present disclosure provides a method including the following three procedures: (A) Acquiring a pair of dual-energy images at a high-energy level H, and a low energy level L for the subject that contains the interested soft tissue overlapped by a bone structure through the dual-energy x-ray imaging hardware system; (B) using a dual-energy decomposition method to transform the acquired dual-energy x-ray image pair data into two material composition images: the human body soft tissue image and the bone image. This is to remove the direct interference of bone image imposed on the soft tissue image, where the desired image information resides. (C) compensating the indirect interference effects in connection with the bone due to empty space originally occupied by the bones inside the soft tissue. The compensation method can include two steps: (1) Replace the originally decomposed bone image with a bone-equivalent soft tissue image (2) Reinstall the bone-equivalent soft tissue image into the originally decomposed soft tissue image at the exact geometry position where the originally decomposed bone image is located.

Apparatus and methods to improve x-ray imaging of soft material, or soft media or soft tissue imaging X-ray systems, for example, may be a general x-ray system, or CT scanner or a 3D or multiple dimensional x-ray system different than a CT scanner, or an x-ray system with scatter removal apparatus and methods including of beam selector or beam particle stopper plate, and/or interpolation methods, which may be capable of spectral measurements, or multiple dimensional imaging, or spectral imaging or point, 1D, 2D and 3D tracking. The system can be integrated with or attached with mobile or portable systems with moving mechanisms, such as wheels, sometimes with self driving or remote controlled moving capabilities. Such systems may be compact enough to fit through a door, with approximately for example, 35 inches wide and 6 feet and 8 feet height, or medical door,

To extend the capabilities of x-ray systems due to limitation of hardware, such as limited wavelength, or frame rate or resolution, or sensitivity or energy sensitivity or quality, a system configuration can accommodate the use of new hardware, such as nanotube or field emitter based tube together with that of a conventional hot filament based tube. One example can move the selected x-ray tube and/or corresponding detector optimize the position for the measurement. A second measurement may be done with the same detector but a different tube can be moved to the same x-ray emitting position. Both the x-ray tube and detector pair may be moved together as in a c arm or something similar or independently. Different detectors may be used in the place of the first imaging position or a different spatial position on the same subject and/or the ROI.

As shown in FIG. 1, X-ray can be generated from x-ray source module 12, and can illuminates the subject, 2, passing though a beam particle stopper plate, 100, and being collected by the detector 22. In some instances, mechanisms to support the subject, such as x-ray translucent plate, may be placed between the subject and the beam particle stopper plate and the detector.

In instances where the x-ray detector may include dual detector and a beam particle stopper plate, the beam particle stopper plate 100 may be moved by an actuator so that the missing data of the illuminated subject 2 due to beam particle stops can be obtained in a second image. In some cases, the second image may be taken at a different energy level.

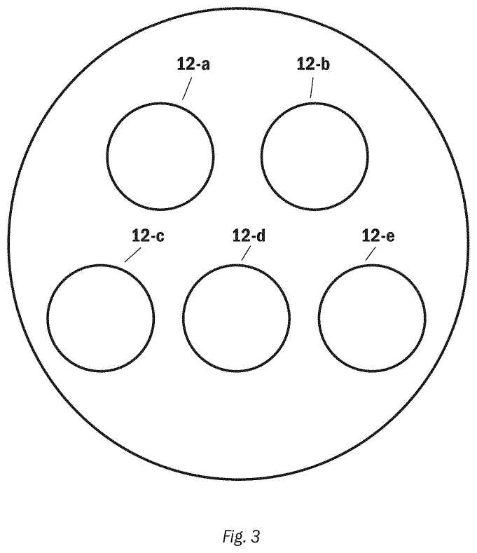

In FIG. 2 and FIG. 3 multiple x-ray sources 12-a . . . 12-e can be used for imaging. In a preferred implementation, x-ray source can be placed in a turret so that each source can be moved in and out of the illumination path for the subject by rotation.

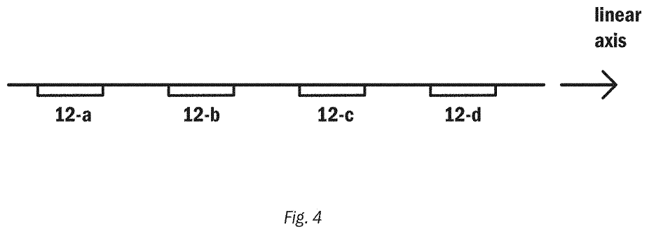

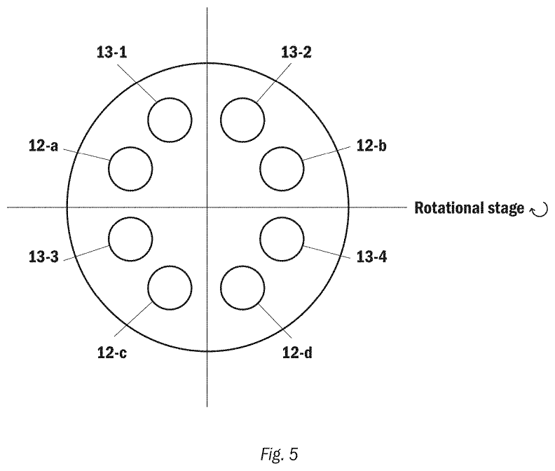

FIG. 4 illustrates a mechanism to include two or more x-ray sources and moving such sources in a linear axis.

FIG. 5. illustrates an implementation where a rotating motion device such as rotational stage moves x-ray sources along a rotating axis

FIG. 6 illustrates that the sources are on a xy plane, can be moved by xy translation stage as well as a rotational stage. FIG. 6 shows the examples of multiple x-ray and detector pairs, for example, one or more x-ray source and detector pairs. The source 13 and its corresponding detector 27 may be placed in the same plane as that of source 12 and detector 22 pair. The x-ray source 13 may be placed up to 90 degrees from the center axis of the original x-ray source 12 and detector 22 pair. X-ray source 13 may pass through a beam particle stopper plate 100-2 to reach detector 27.

Such a setup can increase the speed of image acquisition or to give another geometric information of the subject, or increase the accessibility of certain region of interest in the subject. As such additional x-ray source and detector may have different or same parameter values such as in resolution, image acquisition speed, focal spot size, mobility, form factor, or spectral wavelength or energy level or additional hardware pieces or additional x-ray optics or two or more combinations of all of the above.

There may be two or more detectors placed on various angle relative to each other. They may share one x-ray source.

Data acquired by one detector can guide the data acquisition process and method of the second or other detectors and vice versa. Summation of x-ray measurements and image sets from all detectors may be required for data analysis.

This is different from conventional imaging methods where different detectors are used for multiple dimensional image reconstruction by providing an image at a varied angle and combined images from multiple locations to reconstruct one single tomography image. In the present disclosure, each x-ray source is capable of x-ray tomography measurements, and multiple sources at different spatial locations can combine measurements, especial spectral measurements to ensure fast acquisition, or for measurements of multiple dimensions. Multiple x-ray sources may be moved at the same time again to increase the speed of tomography, for example, by combining the number of measurements. One difference from tradition tomography or tomosynthesis methods is that the x-ray source and detector pair center axis of one x-ray source or emitting position are preferably close to each other, for example, by less than 10 or 5 or 4 or 3 or 2 or 1 degree, relative to the ROI and center axis of x-ray source and detector.

When using hte beam particle stopper plate 100, image data gaps exist due to the region of the subject illuminated by the x-ray beams which are attenuated by the x-ray beam particle stopper. Having another detector or another set of x-ray source and detector at a different angle can capture the missing data information by illuminating the subject or VOI at a different angle. At least some if not all of the missing data can be retrieved. In scanning for presence at a location of a specific component of diseased tissue or contrast labeled tumor or stem cell, if a some small amount of area is missing, a second set of data may be acquired of the region contributing to the missing information at the same time, thereby reducing the system motion requirement and increasing data type flexibility and the likelihood of additional type of information being obtained.

FIG. 7 illustrates two or more detectors 44, in one preferred example, (each detector having a varied value of its performance parameter, for example, frame rate, or resolution) positioned downstream from the a first detector 22, where the detectors are placed in stages with movement in at least one axis spatially and/or rotational stages. In another implementation, other type of robotics or movement mechanisms may be used to move detector. FIG. 7 illustrates a configuration where a rotational stage such as R1, are placed downstream of the front detector, 22. One or more detectors 29 can be moved by the rotational stage. Each can be moved to a location to collect x-ray measurements of a region of interest in the subject 2.

Depending on the resolution required and frame rate required, an x-ray source and in some instances, a detector can be selected to image the selected region of interest in the subject.

The present disclosure may allow, for example, zoom in or zoom out or explore one or more region of interest for measurements and imaging or to explore in detail characteristic and properties of the sample, and/or a target and/or a component in a sample, using the same or varied set of hardware and/or software algorithms options . For example, for tracking, sometimes low resolution measurements may be sufficient, but faster frame rates for measuring one or more selected region of interest may be needed. The present system allows for one or a set of first x-ray images or measurements to be taken first, and one image or more second images be taken using different hardware and/or software features depending on the user input and the application requirements. In a present disclosure, an x-ray system can accommodate requirements of varied imaging demands for selected region of interest and/or allow for flexibility of options in various measurements in speed, resolution, spectral information or timing or various combination of options. In addition, if a certain growth or cellular event or intercellular events or physiological events or chemical occurs, it results in measurements or values of measurements in x-ray or other measurement modality such as optical methods, or electrical or energy measurements to be detected and defined as a triggering activity. Such activity then triggers the software program or a user to authorize or command the computer to drive the x-ray system to do additional measurements or imaging. For example, in an monitoring measurement, when certain cellular interactions or tumor growth or stem cell engraft growth or tissue growth or disease is measured to be present, additional measurements or imaging may be done to further analyze the region where the activities occur to further characterize such activity or activity affected areas or further monitor the cascade of events or activities in a region of interest, optionally in a time sensitive manner.

Typically the front detector 22 captures images called a first image or first set of images and/or measurements. From the first image, one or more regions of interest can be selected in the subject. Either a 3D image is taken and reconstructed, or a second detector or detecting module either down stream or upstream of the detector 22 is positioned to capture x-ray measurement of the region of interest in format of point, or 1D, or 2D or 3D. The detecting module may be an absorptiometry device or a spectrometer or a beam selector sandwiched by a pair of detectors or a beam particle stopper plate and a detector, in some cases, the beam particle stopper plate may be supported by hardware such as a set of posts and clamps to position the beam particle stopper plate slighted elevated than the detector, leaving some spacing such as in the range of um to mm to cm or inches from the detector surface. And in some instances, actuators or linear translation stage or two axis or more translation stage may be used to move the beam particle stopper plate 100 relative to the subject 2 and/or the detector 22 and/or the x-ray source 12

The x-ray imaging apparatus and methods may integrate with apparatus, software, methods and optical imaging and optical measurements methods in cell sorting, cell isolation and/or cell manipulation devices, tissue engineering and growth devices, exo vivo tissues and in vivo procedures.

In one example, at least one x-ray source and one detector are placed in a geometry without the view obstruction from other hardware in the sample and the x-ray illuminates the region of interest in the subject, such as cell samples.

Components, targets and region of interest can be measured by using single, or more energy levels, or monochromatic or polychromatic 2 D projection image, or point, or 1D projection measurements, to reconstruct a multiple dimension or approximately complete 3 D image. Therefore, the shape, thickness, and spatial position and 6D measurements and in some instances temporal characteristics and flow dynamics of each component, or target or region of interest, may be determined. In some instances, contrast agents are used to label such component or anatomic marker or anatomic markers of such a component, or such a component may include radio-opaque regions.

Depending on the complexity of the subject or region of interest, measurement of spatial position, thickness and shape using X-ray phase radiography with grating interferometry and the reverse projection technique at single or multiple energy levels may be used. Measurement of thickness may be achieved by moving x-ray sources or having multiple x-ray sources. Measurement of thickness may be achieved by moving x-ray sources or having one or more x-ray sources, each have one energy level or more energy levels. Multiple dimension image without complete 3D reconstruction may be sufficient to measure thickness and/or shape of a component. Low-resolution 3D image may be used to measure shape, spatial position and thickness of a component. Anatomic markers may be used to calculate thickness and shape of a component. Different regions of bone and its spatial proximity with anatomic markers of bone tissue or other type of tissues may be used.

With one or more energy measurement at one x-ray emitting position, a database with one or more density with corresponding thickness and therefore 3D shape can be established in the region of interest. Measurements at one or more different x-ray source emitting position can be performed. Measurements may be at single energy or more energy levels, and may be used to match the database.

Each voxel in the 3D or multiple dimension data set may be measured and therefore characterized by spectral response, such as weighted spectral response or weighted attenuation value or attenuation value at one or more wavelength or elastograph measurements, or before after ablation or energy modulation or phase contrast measurements. The thickness data and spatial location of certain segment of slow varying density may be derived by analyzing and draw conclusion based on density and attentuation values in each or more voxels and category or segment based on a set of criteria given by a computer, or by looking up in a reference database or given by a user.

Single, two or more grating based phase contrast and dark field imaging may be used with single energy or more level measurements to achievement thickness and shape measurements. A database with corresponding thickness and shape or density measurements can be established separately from the measurements. Single energy, or more energy, or phase contrast measurements, in one or more x-ray emitting locations, in some instances with external ERC or IRC can be used to determine the thickness and shape of the component.

In addition, single, or two or more grating systems based interferometer using x-ray optics mirrors can generate a delay line separate from the x-ray beam passing through the subject and combined at the detector to form interference pattern, which may be indicative of the structure, shape and thickness and position in space.

Reference database with predicated data and/or imperial measurements may be used for determination of the structure, shape, thickness and density and position in space and time.

The reference database may be based on real time simulated data combining one or more tissue types based on preexisting data and measured data on the present subject. For example, in bone density measurement, density of the soft tissues immediately adjacent to the bone tissue may be measured. If the tissue variation is slow varying, more accurate prediction of the thickness of the soft tissue may be derived by taken one measurements and matched with a database or taken two images to determine the thickness. As the thickness and density of the soft tissue are determined, and geometric shape for exterior of the subject is determined, the thickness and/or density of the bone may be determined.

Most of the images or measurements may have x-ray scatter removed as described herein or have an subject with low x-ray scatter properties. And/or the measurements can be done with x-ray thin beams which do not generate enough scatter in the detector pixel region of interest. Such x-ray thin beams are distributed far apart from adjacent x-ray beams so as to generate scatter signal or generate negligible scatter signal for each projected x-ray measurements on the detector.

Generation, for example, using x-ray filters, and/or determination and/or identification of region of interest for x-ray measurement may be real time based on computer analysis based on one or more sets of criteria or may be predetermined.

Region of interest or component or target may be determined and identified continuously based on user input or digital input or simulated according to one or more criteria from the computer throughout the imaging process.

For example, as 3D images of the component are reconstructed, facts may be derived due to such information. Adjacent component or region of interest or target may now become region of interest to further characterize or tracking one or more component in the region.

Alternatively, low resolution 3D images may be taken to determine new region of interest or more regions of interest, so that higher resolution images can be taken or higher frames rate images can be taken of a smaller volume for further detailed measurements.

Such process may be iterative.

The opposite may be done, when high resolution image of a small region of interest are taken, a user or computer may determine whether to take a larger region of interest to characterize a macro environment, or a region of interest in a different spatial location to further characterization or analysis. For example, when a cancer cell or tissue region is characterized, blood vessels and capillaries surround the cancer regions may need to be characterized and monitored to further study angiogenesis. And cellular matrix and cells adjacent to the cancer cell regions may be further studied to understand the effect of the cancer cells and determine regions where a surgical incision may be need. In neuron circuit analysis, a larger field of view may be needed to assess interaction between cells once individual cells are spatially positioned and identified. In radiation therapy and robotic surgery or image guided surgery and energy-based ablation, regions of interest can be continuously identified based on the after image during the surgery and/or surgical plan or results of the surgery.

2D, multiple dimensional measurements and/or 3D measurements as described herein may use x-ray optics and/or intensifier or scintillator plus optics for magnification and/or manipulation or steering or miniaturization of x-ray images and measurements.

Using x-ray optics such as an focusing element or collimating element, or polycapillary optics or Ellipsoidal or single paraboloidal, achromatic or KB mirrors, Twin paraboloidal, ellipsoidal, or Wolter-type , achromatic or monochromatic collimating or focusing, or magnification or demagnification, or others, can direct x-ray to the back aperture of an x-ray focusing lens and a beam filter and pin hole element to generator a beam aperture which is the size of the focal point upstream of a subject or region of interest.

Mirrors, attenuators and/or filters and/or grating and/or beam splitter and/or optics or crystals or prisms or refract or defract optics and optical modules or electrooptics modules in the optical wavelength range and x-ray optics may be used in the pathway to steer, or select, filter, manipulate and form interference for different analysis such as interference, synchronized or desynchronized measurements, selectively manipulate light and optics modules, or electro beam manipulators, or mechanical or energy-based modulators or motion systems for manipulating each of optics and submodules in the x electron beam generator, electron beam, x-ray generator, x-ray optics, x-ray elements, energy generator, detectors in the optical path way or purturbation of samples or region of interest.

With variations on the detection and beam steering, the x-ray output from the subject may be collected by a intensifier combined with an optical camera, in some instances, with optics in between for demagnifying or focusing or collimating or steering and/or large format x-ray detector or a large format scintillator plus an image sensor or x-ray optics or optics coupled sensor, for instance, using a fiber optic taper to taper down to an image sensor with or without scintillator. Alternatively an objective lens (x-ray optics or optical optics for light in UV to NIR wavelength) to taper the x-ray down to a size where a digital x-ray detecting element may be used or a combination of scintillator and optical sensor may be used.

Scintillator or intensifier may be placed upstream of the optics and sensor or may be placed downstream of the x-ray optics and upstream of an image sensor.

In the present disclosure, using the imaging with spectroscopy or optical biopsy using non linear microscopy, in some cases, combined with super resolution imaging such as using structural illumination or hyperspectral spectroscopy and imaging, one or more details and facts can be identified, characterized and lead to diagnosis, or for bases for AI algorithms for machine training, machine learning, image guidance or facts finding. In addition, region of interest may be further identified and analyzed.

In pre-alignment tumor lesions, there may be multiple markers and abnormal vascular growth. However, the prognosis for malignancy may be not determined unless further tests are done and taken into account other factors. To further analyze and image single molecule, single cell, or cell clusters, investigate kinetics of molecular events and interactions, high resolution functional imagine can be performed using optical methods, such as silicon photonics endoscope, monolithic photonic circuits based imaging apparatus, and microscopy. The images can be zoomed in the details or provide another data point for the analysis of samples in vivo.

A dual-energy, or triple energy or spectral x-ray imaging system, as shown in FIG. 1, can include a multiple-energy x-ray source of equal or greater than two energy levels 12, a two-dimensional x-ray detector 22. The subject 2 under examination can be a body that contains an interested region, which may includesoft tissue overlapped by accompanying bones. In human or animal chest projection imaging, the subject contains the chest cage wall bones, which includes rib, sternum, and spine bones and the accompany soft tissue in the chest cage wall, the internal organs, such as the lung, the heart, the liver, the spleen, the chest diaphragm etc. All the internal organs are made of soft tissue. The exact composition of soft tissue for different internal organs, and for the chest wall muscle have slightly different x-ray absorption coefficient, but can be all approximately treated as the same, such as having an averaged common x-ray absorption coefficient μs(E) as a function of x-ray energy E. The image subject 2 can be located between the x-ray source 12 and the x-ray detector 22. The x-ray source can have the capability of emitting two or more pulsed x-rays with controllable energies. The x-ray source can emit two consecutive pulses, a high-energy pulse at an average energy level H followed by a low-energy pulse at an average energy level L. Each pulse can have a single, reproducible energy spectrum. The x-ray detector 22 can be any two-dimensional digital x-ray detector which converts a two-dimensional x-ray image information into a set of digital data suitable for being transmitted to a computer. Currently there are two types of 2D x-ray detectors: a conventional 2D x-ray detector, and a 2D x-ray detector assembly with the capability of scatter removal, as described in U.S. Pat. Nos. 5,648,997 and 5,771,269, the entirety of each of which is incorporated herein by reference and should be considered part of the specification. The conventional two dimensional(area) x-ray detector inevitably receives certain amount of random-scattered x-rays mixed in its output signals as an inseparable scatter interference. U.S. Pat. Nos. 5,648,997 and 5,771,269 provide effective ways for eliminating scatter interference by using an improved three-layer detector assembly structure. For achieving high-quality results, the instructions of these patents can be followed for construction of the x-ray detectors and for the data processing procedures. Other methods of scatter removal involving x-ray source, primary x-ray modulator or single collimator, or one beam particle stopper plate are disclosed herein. On the other hand, the present disclosure does not exclude the use of any conventional two-dimensional x-ray detectors. Scatter interference adversely affects the quality of the results. However, if the x-ray detector receives only a sufficiently small amount of scatter interference, under certain circumstances, a qualitatively correct, yet possibly quantitatively inaccurate imaging results can still be obtained. The extent to which the scatter interference can be acceptable, is case-dependent, and can be decided by case-specific analysis.

A system for imaging an object using radiation can include a source of radiation; at least one real detector array that obtains projection data at two or more positions and has a geometry that is neither equilinear nor equiangular; and a data processer for reprojecting the projection data onto a virtual detector array that has a geometry that is either equilinear or equiangular, and for reconstructing the reprojected data from the virtual detector array.

A imaging system can include a structure to support and/or a mover for x-ray source, and/or collimator, and/or the same structure or a separate structure support or moves, the x-ray detector, or x-ray detectors. The line of illumination of x-ray source and x-ray exiting out of the object can be collected by the detector. The system may further include a moving mechanism, such as electromagnetic steerer and/or a motor to move the x-ray emitting position, a power supply and/or controlling circuit, a mechanical mechanism for moving the flat panel detector closer to and further from the ROI and/or the examination or inspection region. A timing and control circuit can control a motor that indexes the inner race around the examination region, an x-ray power supply that pulses the x-ray tube in sequential steps at selected positions around the examination region, and a read-out circuit that reads out a frame of data after each pulse of the x-ray tube. The read-out frames of data can be stored in a frame memory and reconstructed by a reconstruction processor into a volumetric image representation for storage in a volume image memory. A processor can extract data or 2D presentation frame memory or the image memory or selected portions of volume image representation from the volume image memory into appropriate format for display on a monitor. A field of view or volume of interest can coordinate adjustment of the collimator and/or movement of the flat plate detector and/or the movement of x-ray tube toward and away from the examination region and ROI.

FIG. 30 illustgrates a multiple detector imaging system with the use of a separate beam particle stopper plate for each detector. Imaging of one detector can help to select for ROI and/or for ROI to be measured by a second detector.

The system in FIG. 30 includes a beam particle stopper plate on top of a detector. The first detector 22 may be a large detector, and the second detector 23 may be a detector of a different size. A beam particle stopper plate 100 may be placed above each detector. The beam particle stoppers for the detectors may be different from each other, or it may be the same piece.

The mover for the beam particle stopper may be be a spinner, where a beam particle stopper plate is a disk which spins over the top of the detector.

In one instance, the mover may be an actuator 900 to move the second detector 23 and may be on one or two sides of the detector 23 instead of attaching to the bottom of the detector 23

Beam particle stopper 100, may not be needed when the detector is small in dimensions.

For example, a tapered collimator, with the opening close to the x-ray source may be small or further away from the larger source, geometrically producing a smaller focal spot 12fs.

The cone beam produced or beam volume producedmay illuminate a smaller volume in imaged subjectthan the volume which can be illuminated by the original x-ray source. The x-ray beam or primary x-rayilluminates the subject and passes through the sample holder 40 and reaches the detector assembly 22 contained in the detector gantry, which may be moved with translation stage to different region of interest on the subject 2. X-ray source 12 is mounted onto the structure and translation stage for moving the source 12 to target the subject to illuminate.

The collimator may be mounted onto the x-ray tube housing or the hardware which holds the x-ray tube and housing in place, or there may be a standard collimators with for example, filters and/or shutters mounted in between the collimator and source 12. Collimator or the additional collimator may be used to select the region of interest or volume of interest. And collimator may be combined with a conventional collimator which has filters and hardware and/or shutters, and may be moved into place when it is needed. This movement and design may be similar to what is used for a shutter or filter.

Structure hardware 125 and Support hardware 130 can hold the source 12 and detector and its assemblies in place and give the mechanical support and stability. Wheels 108 can be used for portability and may be retractable.

The x-ray beam focusing mechanisms, instead of being collimator, may be other mechanical or energy or optics based mechanisms, for example, MEM-based or diffraction grating based optics, or zonal plate or other x-ray focusing optics. In some cases, a beam aperture may be used. In addition, Laue lens, which uses Bragg diffraction in order to focus beam (usually using a tilted crystal) can be used. Multilayer Laue lenses (MEL) may be used as a focusing element.

In some cases, an appropriate X-ray phase plate into the optical path can achieve diffraction-limited focusing. The phase plate operates in transmission and is based on refraction. Hence, the phase plate is largely insensitive to small shape and surface inaccuracies in the range of um and can correct residual aberrations originating from surface errors of reflective optics, zone deformations in diffractive optics and accumulated surface errors in larger refractive lens stacks.

Detector 22 may be a part of detector assembly with a beam selector or beam particle stopper plate 100 or beam particle stopper 22b. And there may be two or more detector or detectors below the sample holder 40 or the patient table 40.

A second or third source assembly including an x-ray tube, housing assembly and/or collimator assembly which may include collimator may be mounted on the support 120. The second resource may be moved to illuminate the selected region of interest and/or similar imaged subject 2. The second source assembly may be mounted in a separate structure supported by a different structure than the support 120 and 125.

Electro focusing lens can focuse electron beam to have smaller x-ray focal point in a conventional x-ray tube.

The x-ray system may include one or more LED displays for warning or alerting, which can be triggered by an event related to the x-ray system, such as radiation level getting high or malfunction of hardware system or software system. The x-ray system may include one or more emergency buttons. User may push or use other mechanical mechanisms to shut off the system. The button may be a bright color or any color and/or be visually easy to spot.

The x-ray system controller may include master power control to bring the generator out of the standby mode.

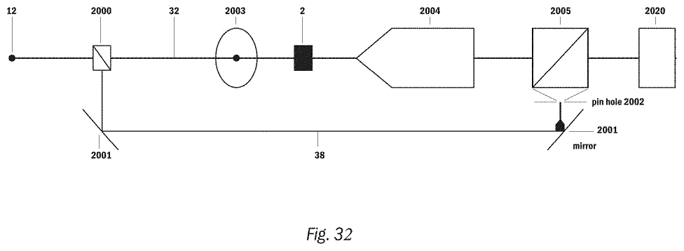

FIG. 32 illustrates an example of holographic x-ray system.Source 12 may emit x-ray, in some instances, coherent x-ray. X-ray reaches an element 2000, such as an x-ray beam splitter, generates two beams. One beam 32 can pass through the sample or imaged subject 2 and a second beam 38 can serve as the reference arm. There may be a focusing element, such as zonal plate, or there may not be. There may be before the sample a pin hole 2002, or beam aperture. The imaged subject 2 is placed downstream of the beam splitter 2000, focusing lens 2003, and upstream of an objective 2004. In some cases, such an objective is not needed. the reference beam 38 meanwhile is being steered by x-ray optics, such as x-ray mirrors, 2001 and is to combined with another beam coupler or combiner element 2005. An interference pattern is formed on detector 2020. Depending on the beam width of 32, there may be a beam particle stopper plate 100 and there may not be to remove scatter.

In some cases, dual detector and a beam selector assembly may be used in place of the detector 2020. There may be a scintillator or image intensifier in between the objective 2004 and the imaged subject 2, or there may be a scintillator in front of detector 2020, which may be a optical camera. There may be a pin hole placed in between mirror 2001 and the beam combining element 2005. The interference pattern can be Fourier transformed and/or analyzed for derivation of phase information of x-ray wavefront going through the imaged subject. Reconstruction and analysis methods and algorithm of holographic imaging or microscopy in the optical wavelength may be applied similarly. Such an imaging system or phase imaging system may be used to measure a selected region of interest selected using the aforementioned x-ray imaging system, such as spectral, or 3D or scatter removed systems and methods.

Phase Imaging using x-ray as described will provide another layer of information for the analysis of imaged object 2 in addition to transmission x-ray imaging modality.

Holographic x-ray imaging of phase and/or temporal transmission images may be derived.

A sensing element for sensing distance, geometry or relative spatial position of various hardware elements, submodules in the x-ray imaging, and controller and display and controlling unit., may be mounted near x ray tube

Motor and attachment may be used to attach tapered collimator to the x-ray tube housing or another collimator attached below the x-ray tube.

Attachment hardware may be motorized or adjusted by a mover or a robotic arm to move tapered collimator in and out of the line of path in between the source the VOI.

Taper Collimator may be used to reduce focal spot size. Tapered collimator may be used to restrict region of interest. Tapered collimator may be used a conventional collimator, which has a design similar to a shutter, to allow open and close of an aperture for adjusting exposure time. In some cases, the tapered collimator may have stacked layer of beam attenuators, each layer has slightly larger aperture than the layer above it, forming a tapered hole for transmission.

A sensing element may be attached to the mover for x-ray tube or may be attached to the x-ray tube, or may be placed at a spatial location, separate from the x-ray tube 12 and/or the detector 22 and/or holding structure or mover or a submodule involving either the x-ray source 12 or detector 22 or the detection module containing detector 22, beam particle stopper or beam particle stopper plate 100 or beam selector or one or more other detectors for imaging the imaged subject 2.

The sensing element may be an optical sensor or time of flight sensor or a camera or a lidar device, may measure the dimension or geometry or spatial location relative to x-ray source and detector and thickness of the VOI or ROI or the imaged subject. The measurement of the thickness may be used for determining the dosage or exposure time thereby the control of the collimator shutter, or the alignment of x-ray source and/or detector relative to the VOI or ROI, or determining number of projected images or measurements needed for complete reconstruction of the 3D image of VOI.

As x-ray beam are in a cone shape, the region of interest closest to the x-ray source may be smaller than that of region of interest closest to the detector. Or x-ray illuminating the voxel or voxels from region of interest closet to the x-ray source, may project to a larger area on the detector when measured. For example, a region of interest ROltotal (x, y) may include a volume expands from the surface of the subject closet to the source to the Sensors and/or cameras.

One or more sensors such as Time of Flight Senor (TOF) may be used to measure thickness of the object to determine the number of images to be taken for tomography acquisition.

Sensors may be used to estimate exposure needed for each image or each measurement of ROI.

Sensor may be used to determine and guide the movement and/or alignment of x-ray emitting position and/or of detector, for instance, moving both x-ray source and detector so that ROI is in the imaging path, and properly aligned.

Radiopaque Markers

Radiosensitive or radioopaque markers and/or optical markers or reflectors, may be placed in different areas within the x-ray illumination path and/or off the path, to serve as reference point to properly align imaging hardware and as well as for image reconstruction with improved and more accurate spatial positioning.

A x-ray imaging system can include one, two or more x-ray sources or dual or more sources with varied focal sizes and/or other varied values in its parameters such as preparation time, exposure, speed, power, energy level, number of energy levels, spectral wave form characteristics, pulse duration, pulse characteristics and/or form factor in approximately one or more x-ray emitting locations. An X-ray imaging system with one or more detectors movable upstream or down stream of an x-ray detector. Detectors may be the same or different from each other in spatial resolution, speed, sensitivity to spectral values, sensitivity to one or dual or multiple wavelengths or different energy levels, programmability, dynamic range, photon sensitivity, quantum efficiency, spectral sensitivity or hardware design and other characteristics which may differentiate the detector from the others.

For example, the first detection system can be placed between the beam stopper particle plate 100 and the subject. And the second detector can be placed downstream from the beam stopper particle plate. The first detecting system may be a point detector, or a linear detector, 2D detector or spectrometer.

In another example, between x-ray source and first detection system, there is no a beam stopper particle plate. This is due to the fact that the second detector or detector system may have a beam stopper particle plate upstream, the scatter signals on the second detector can be interpolated from the low resolution scatter measurements on the second detector. As a result, the high resolution primary x-ray on the second detector is derived. As the primary signals on a corresponding position of the first detector is correlated to the primary signals on the second detector at various thickness using different material of interests, each material can be calibrated by a thin beam at each of the energy levels selected for material decomposition for the subject, or the region of interest or the components. Low resolution and high resolution scatter signals on the first detector can be calculated and derived. In the final step, the primary signal of the first detector can be derived by subtracting high resolution scatter from the composite measured signal. The beam stopper particle plate upstream of the first detector is therefore not needed.

The primary signals at one or dual or a set of energy levels at a particular position can correspond to a derived value of a particular tissue with certain density and thickness, or simulated value or measured data from a reference database. The reference database can be established by multiple imperial measurements of two or more materials at each of two or more energy levels on both first and second detectors. The density and thickness data can be derived due to interpolation of six or more corresponding measured data at each energy at each datapoints. When the number of measurements are increased for each material, the predicated value may not deviate from the actual measurements by 0.5% at a set of energy level. As a result, the rest of the value at various set of energy levels can be interpolated and stored in the reference database. The first detector and second detector may each have different detector response function for a specific material at various energy levels. The present disclosure extend such methods to three or more energy levels which corresponding to three or more materials.

Methods are provided for better visualization and quantification of individual component in a target in a region of interest in a subject, for example, of an individual tissue or object in a body including bone and soft tissue or at least two components, each with different atomic z number or different values of at least one x-ray measurable properties in a subject.

The interference of images from the second component or tissue in the x-ray image can be removed from the image of a different component or components. For example, this disclosure can eliminate the bone interference on its overlapped soft tissue x-ray projection image. The method can be based on use of a two-dimensional dual-energy or triple energy or multiple energy, also named spectral x-ray imaging hardware system. In one preferred example, where a dual energy system x-ray imaging system is used, the present disclosure of method can include three procedures: (A) Acquiring a pair of dual-energy images at a high-energy level H, and a low energy level L for the subject that contains the interested soft tissue overlapped by a bone structure through the dual-energy x-ray imaging hardware system; (B) using a dual-energy decomposition method to transform the acquired dual-energy x-ray image pair data into two material composition images: the human body soft tissue image and the bone image. This is to remove the direct interference of bone image imposed on the soft tissue image, where the desired image information resides. (C) compensating the indirect interference effects in connection with the bone due to empty space originally occupied by the bones inside the soft tissue. The compensation method can include two steps: (1) Replace the originally decomposed bone image with a bone-equivalent soft tissue image (2) Reinstall the bone-equivalent soft tissue image into the originally decomposed soft tissue image at the exact geometry position where the originally decomposed bone image is located.

The Apparatus and methods disclosed herein can improve x-ray imaging of soft material, or soft media or soft tissue imaging.

X-ray systems, for example, may be a general x-ray system, or CT scanner or a 3D or multiple dimensional x-ray system different than a CT scanner, or an x-ray system with scatter removal apparatus and methods including of beam selector or beam particle stopper plate, and/or interpolation methods, which may be capable of spectral measurements, or multiple dimensional imaging, or spectral imaging or point, 1D, 2D and 3D tracking can be are integrated with or attached with mobile or portable systems with moving mechanisms, such as wheels, sometimes with self driving or remote controlled moving capabilities. Such systems may be compact enough to fit through a door, with approximately for example, 35 inches wide and 6 feet and 8 feet height, or medical door, or emergency room door, patient door or medical room door or operating room door.

The systems and methods disclosed herein can improve resolution of x-ray measurement by reducing focal spot of x-ray emitting area via for example, tapered collimator structure, or focusing optics or electromagnetic focusing lens.

X-ray measurements may be done to measure the permeability of contrast agents or solutions containing contrast agents during a procedure in different portions of the tissue. The procedure may be of energy treatment or biopsy, or other therapeutic or drug delivery method. Catheter with contrast aspiration capabilities, such as a capillary tube to aspire liquid of contrast agent solutions to the ROI can be used. The measurements over time may be used to measure and differentiate permeability for diagnosis at one time or over time, and may be used to measure permeability before, during and post intervention.

X-ray exposure measurements are done with sensors prior to x-ray reaching the imaged subject using sensors of x-ray or optical wavelengths coupled with x-ray optics, scintillators or without scintillators.

X-ray systems and related tomography system or related scatter removed system may be resistant to deposit of pathogens, or reduce pathogen life time, or resistant to retaining of pathogen, such as being copper coated or silver coated.

X-ray system may have two or more configuration on one machine. For example, one may be used for transportation, one for operation, one for storage, or one for one application, such as spine imaging, another for another application, such as for cardiovascular or pelvic imaging, with variations and adjustability in height or travel distance of the x-ray tube or detector, distance between the x-ray tube and the detector, choice of component and structural. as the system may have a folded supporting structure for a compact configuration to reduce dimensions in one or more axis. The folded structure may be temporary, for purpose other than imaging or in between imaging operations or during imaging operations or during a procedure.

The system may also have settings for the optimized image acquisition and processing and presentation methods, using nMatrix and related data presentations, in numerical format, such as density measurements, dimensions, shapes, patterns, or 1D to 7D dimensions, 7D being 6D with a temporal marker, or varied resolution customized for application needs.

X-ray source may be conventional x-ray source or, cold cathode, field emitter source, time of flight, linear accelerator based or liquid metal jet or light based.

To extend the capabilities of x-ray systems such as , due to limitation of hardware, such as limited wavelength, or frame rate or resolution, or sensitivity or energy sensitivity or quality, a system configuration can accommodate the use of new hardware such as nanotube or field emitter based tube together with that of a conventional hot filament based tube. One example is to use to move the selected x-ray tube and/or corresponding detector optimize the position for the measurement. A second measurement may be done with the same detector but a different tube to be moved to the same x-ray emitting position. Both the x-ray tube and detector pair may be moved together as in a C arm or the like or moved independently. Different detectors may be used in the place of the first imaging position or a different spatial position on the same subject and/or the ROI.

Other measurement modalities and therapeutic and diagnostic procedures, such as energy based intervention and measurement tools such as optical spectroscopy or temperature sensor measurements may be used to further interrogate a selected ROI or deploy a intervention procedure.

The design of various intervention procedures may be optimized by having differentiable x-ray measurement properties such as atomic z, attenuation coefficient, thickness, or better visualized and responded to x-ray guided procedures or measurement methods.

2D or 3D fast or real time tracking, for example less than is or less than 100 ms or less than 10 ms or less than lms for dynamic tracking in 2D, 3D or up to 7D such as 6D tracking in time, or 3D or up to 6D fluoro, with approximately 2× less radiation dosage for the complete procedure, with 5× less radiation during the procedure, or with 10× less radiation, or with 25× less radiation dosage, and/or with 100× less radiation, or with 1000× less radiation , or with 10,000× less radiation, or with 100,000× less radiation with approximately 1000.000× less radiation dosage on the subject containing ROI and/or ROI, may achieve approximately similar imaging goals with similar amount of total dosage but less exposure for a specific region or much less total dosage. This may be accomplished by measuring with a point, or multiple point, one region or multiple region, 1D, 2D, 3D up to 6D in time. Each complete cycle of measurement is less than 1s or less than 30 s, and/or a selectively choose resolution required for the application at different times of the measurements or procedure.

Diagnosis based on imaging of a selected ROI can produce equal or better resolution than convention CT systems in medical clinics, for example, along the z direction, in equal or less than time required for conventional CT scanner, and/or with approximately 2×, or 3× or 4×, 5× or 10× or 100× Or 1000× or 10,000× or 100,000 or 1000,000× less radiation dosage compared to that of conventional CT scanner system.

Detector measurements or detector measurement over a white image for each pixel at two or more energy levels can be captured for samples of materials with known physical properties. This can allow interpolation and establishment of an energy response function system where detector measurements on each pixel are uniquely correlated with attenuation or transmission of two or more materials of certain density and thickness. At least one material or substance can be separated from the rest using dual or multiple energy measurements by solving a dual energy primary x-ray imaging equation system by using a linearization approximation method with correction for beam hardening effects. Material decomposition can also be based on the dual energy decomposition method which can be iterative to solve the energy response function equation system with correction for beam hardening effects. The measurements at multiple energies used to solving the energy response function equation system may be normalized and/or have signals subtracted and/or spurious filter signals applied and/or defective pixels identified and replaced with adjacent pixel measurement or measurement at a different time at the same pixel location.

Tomography disclosed herein can include the construction of complete volumetric data n2 tomography or 3D measurements.

Images can be taken at a number of X-ray emitting positions, moved by for example, a motorized stage or electromagnetic mechanisms or solenoid coil. The emitting positions are spaced by approximately resolution Xc desired in the z axis, which is approximately perpendicular or axial to the detector or the source pair center axis. The number of images can be approximately determined by the thickness P of the sample over the z axis resolution Xc. The x-ray positions can be in a xy plane perpendicular to the z-axis or in a xyz volume or linear. Additional measurements may be taken by emitting positions within the same traveled area or volume to derive more accurate measurement of voxels in the volume.

The total movement of the x-ray emitting position relative to the center axis of detector passing through VOI at a center location, may be less than 10 degrees, or less than 5 degrees, or less than 4 degrees, or less than 3 degrees, or less than 2 degrees or less than one degree

An X-ray imaging system disclosed herein can include one or more of the following:

a generator,

a digital switch which may be or may not be embedded in a controller,

a syn program stored in a microprocessor, which may or may not be embedded in a controller,

at least one controller with at least one microprocessor, which syncs x-ray detector image acquisition or shutter open time with the generator which is digitally switched on. The controller may or may not be locally in the system, or may be at the computer connected with at least one display system.

At least one motion system to move the x-ray tube, or one or more detectors.

the controller may control the motion system and/orthe generator.

at least one memory storage hardware and software with or without a database,

a microprocessor with a memory storage,

at least one microprocessor to control the controller,

at least one microprocessor connected with at least one display that wirelessly or wired,

scatter removal hardware such as a beam particle stopper plate and related motion system,

motion system used for scatter removal hardware,

software used for image processing including scatter removal,

software used for image processing including material decomposition, density, thickness derivation, and/or interpolation algorithms and programs, and/or image reconstruction,

software used for control electro magnetic steerer,

software used for control motion system for 3D acquisition,

software used for moving one or more sources to image ROI,

software used for control one or more sources illuminate ROI,

Software or electronic circuit for sync during the image acquisition process, which may include one or more of the following:

High voltage generator, for example, 0 to 500 KeV
high voltage supply, a low voltage driver, electron emitter
High voltage condenser with an electon trigger circuit, such as a switch, low voltage power supply and a high voltage transformer
High voltage x-ray generator from us per frame to 1 s per frame.

Nano second to microsecond to milli second to one second pulsed electron emitter, which may be cold cathode, field emitter, or hot filament, conventional x-ray tube.

A switching unit that may be a digital switch, for one or more pulses generation by switching on and off by controlling the low voltage driver, or a triggering circuit to ramp up the generator to one or more desired voltages and for tuning down the generator to one or more desired voltages and back to zero.

At least one Controller C1 in communication with the switch Timer and/or a synchronizer for synchronize signals for controlling the C1, and High Voltage generator and/or the detector shutter for measurement and/or a shutter controller to control the shutter of the detector, for example to control the following:
phase lock the master clock frequency of the x-ray emitter with the opening of the shutter of the detector.
shutter signal including a series of signals generated at a regular time intervals and fixed shutter and pulse duration.
an electronic signal from an x-ray source and monitored by a light. X-ray bursts in microseconds or microseconds may be captured by a triggered shutter, operable to a pulse trigger by a shutter pulse. Shutter operation of the x-ray detector can be phase locked with the master lock of the x-ray source or electron emitter trigger circuit or the high voltage generator

In addition, syncing may involve software synchronization and processes for controlling the following:

motion systems for moving x-ray tube and/or the detector to image ROI controlling at least one collimator, for example, for selecting region of interest, and/or at least one filter, and/or shutter controlling x-ray emitting position controlling electron beam steerer such as an electromagnetic steerer

The system may also include one or more auxiliary hardware, such as steering activity including position, frequency and power of one or more electro magnetic steerers or at least one detector, for example, by controlling detector shutter open and close, digital switch, low voltage pulse generator, low voltage circuit, high voltage storage device, high voltage amplifier, high voltage applicator, field emitter.

The system can perform image processing and/or image storage and/or Image transfer and/or image buffering, communication software.

Auxiliary hardware may include further one or more of the following:

At least one time of flight sensor, which may be used for measurement of dimensions or distance from ROI to the source or the time of flight sensor, and subtracted from the distance from the source to the detector, the thickness of the ROI is derived. The sensor data may be used to decide how many images therefore how many steps are needed for the x-ray emitting position movement for x-ray multiple dimensional image reconstruction. The measured data from the sensor such as thickness may also be used to determine the thickness and density of each individual component or substance when using interpolated plot and inverse energy response function system.

At least one camera for measurement of patient or visual presentation of the imaged object for at least one physical parameter measurement such as thickness, dimension or geometry of region of interest and/or remote control or operation of the x-ray system and/or interacting with patient and/or controlling of the imaged object

At least one sensor for measurement of emitted x-ray intensity or real time exposure or dosage

At least one sensor for geometry measurement, and/or At least one camera for measurement of the position of the x-ray system or at least one or more portion of the x-ray system and/or its (their) relative position(s) to one or more reference object.

Axxiliary hardward may further include indicators including auditory indicators, such as sound or voice with certain phrases or visual indicators, such as text or graphics on a LED display, or on and off switch on a membrane display or optical indicatorssuch as LED light on and off, switches, displays on or off, digital signals on a computer display, switch, power on and off or other commonly used hardware which may not commonly be used in image acquisition process but can serve as support function, or remote communication, wired communication, digital and electrical or optical communication, and auto save, any other hardware and software in an imaging system.

The software can interact with the user interface for image acquisition and procedures.

The visual presentations may include measurements, digital display in digits, text displays, warning signals triggered by temperature or hardware maintenance software, and failure trigger, software failure, hardware trigger, etc.

Controlling Additional systems to can be integrated or hybridized with the x-ray imaging system including:

Energy treatment device,

A different imaging modality or measurement device such as a optical imaging or measurement system, photoacoustic system, ultrasound system,

Manipulator,

Probes of surgical tools,

Radiation therapy device,

Biopsy device and the movement of its probes,

Robotics probes and systems,

Cathether and/or guidewire, and/or other minimum invasive surgical instruments.

A multispectral x-ray source disclosed herein can include the switchable x-ray emission system, wherein the high voltage generator is configured and operable to vary as a function over time and the switch is able to trigger an electronic circuit to drive the generation of the short pulses in ms range at times selected such that electrons are emitted with a required accelerating voltage thereby emitting x-rays with a required voltage, ideally between 0- 500 KeV, suitable for body imaging.

There may be a master clock which synchronizes the acquisition or repeated acquisition by the detector. The operation of the master clock may include the following possibilities:

1. Detector continuous operation mode with internal timing. Once receiving a control message or signal, the detector operates with its internal timing. There may be a master clock that synchronizes the detector acquisition of images with that of x-ray emission of x-ray, or the generator, and/or x-ray emitting position mover such as a motion system, or the electron steering device and/or the generator operation. Internal trigger may trigger the controller to control other hardware such as the motion system for the source or the mover for the source and detector.

2. The detector may be in continuous operation mode with external synchronization, signal from generator, or x-ray emission location mover are synchronized and operate to have exposure while the detector continue to acquire images at the interval specified. For example, if each exposure is 100 ms, the generator may generate x-ray precisely synchronized with, for example the mover, when x-ray tube is stationary for a period of time, or the x-ray tube emits the x-ray during the dwell time at each location. The dwell time can allow the duration of x-ray emission to have enough exposure, or is within the timed shutter open or continuous operation mode of the detector. Exposure time may be controlled by the generator switch. There may be a master clock to time and synchronize action of each component in this acquisition process.

3. Noncontinuous operation mode with external synchronization: a master clock may also be used to synchronize all parts or a part of the controller to switch the generator on and off to control the timing of image acquisition and in turn, trigger the movement of the mover and detector shutter open and close.

In all of the modes, the user can enable/disable the triggering out an/ord select if the trig out polarity is low level or high level. The output trigger active time may be the readout time.

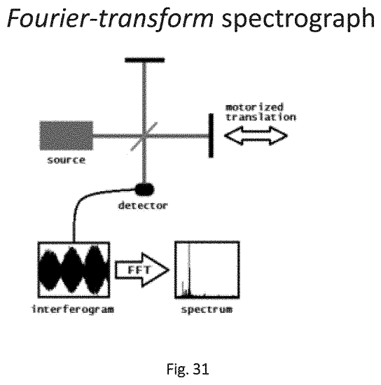

FIG. 31 illustrates a fourier transform device integrated with the x ray spectral imaging and or tomographic device 2D projection measurement for a region of interest can be measured using one or more x-ray sources, and x-ray detectors. An x-ray optics such as a beam splitter can split the beam into two, one illuminating the subject, the other traveling to an x-ray optics to reflect the x-ray beams onto a path which can form an interference pattern on the x-ray detector. X-ray optics may be a mirror, or spectral grating. There may be a delay line which allow the x-ray reflecting optics to move so that the measurements in the temporal domain may be translated into spatial domain, for example, by Fourier transform.

One task in spectroscopy is to characterize the spectrum of a light source: how much light is emitted at each different wavelength. One way to measure a spectrum is to pass the light through a monochromator, an instrument that blocks all of the light except the light at a certain wavelength (the un-blocked wavelength is set by a knob on the monochromator). Then the intensity of this remaining (single-wavelength) light is measured. The measured intensity directly indicates how much light is emitted at that wavelength. By varying the monochromator's wavelength setting, the full spectrum can be measured. This scheme describes how some spectrometers work in simplified manner.

Fourier-transform system can receive as input a beam containing a plurality of different wavelengths of x-ray beam at once, and measure the total beam intensity. Next, the beam is modified to contain a different combination of wavelengths, giving a second data point. This process is repeated a number of times. Afterwards, a computer takes all this data and works backwards to infer how much light there is at each wavelength.

In some instances, between the x-ray source and the detector, there is a certain configuration of x-ray mirrors that allows some wavelengths to pass through but blocks others (due to wave interference). The beam is modified for each new data point by moving one of the mirrors, changing the set of wavelengths that can pass through.

Computer processing may be needed to turn the raw data (light intensity for each mirror position) into the desired result (light intensity for each wavelength). An algorithm such as Fourier transform is used. The raw data may be “interferogram”. Such interferogram may be acquired at varid x ray emitting positions, a tomographic interferogram may be formed for additional analysis of ROI. For low energy measurements, inteferogram may revealing information not available. With less than 1% of SPR or less than 5% SPR, such measurements may detail features hidden in soft tissue previously not visible. Combined with spectral imaging, additional information can be further revealed. The “raw data” can be Fourier-transformed into an actual spectrum. The peak at the center is the ZPD position (“zero path difference”): Here, all the x-ray passes through the interferometer because its two arms have equal length.

The method of Fourier-transform spectroscopy can also be used for ab sorption spectroscopy.

For instance, x-ray absorption spectroscopy can measure how well a sample absorbs or transmits light at each different wavelength. Although absorption spectroscopy and emission spectroscopy are different in principle, they are closely related in practice; any technique for emission spectroscopy can also be used for absorption spectroscopy. First, the emission spectrum of a broadband x-ray source is measured (this is called the “background spectrum”). Second, the emission spectrum of the same source shining through the subject is measured (this is called the “sample spectrum”). The sample will absorb some of the light, causing the spectra to be different. The ratio of the “sample spectrum” to the “background spectrum” is directly related to the sample's absorption spectrum.

Accordingly, the technique of “Fourier-transform spectroscopy” can be used both for measuring emission spectra (for example, the emission spectrum of a subject), and absorption spectra (for example, the absorption spectrum of the subject).

The Fourier-transform spectrometer is an example of a Michelson interferometer, Isuch as a continuous wave-Michelson interferometer) except that one of the two fully reflecting mirrors is movable, allowing a variable delay (in the travel time of the light) to be included in one of the beams.

An x-ray beam from the source is split into two beams by a half-silvered mirror, one is reflected off a fixed mirror and another one is reflected off a movable mirror, introducing a time delay. The beams interfere with each other, allowing the temporal coherence of the light to be measured at each different time delay setting, effectively converting the time domain into a spatial coordinate. By making measurements of the signal at many discrete positions of the movable mirror, the spectrum can be reconstructed using a Fourier transform of the temporal coherence of the light.

X-ray projection images may be done without the subject first, at various x-ray emitting positions or x-ray source positions, where the same set of positions or x-ray emitting locations will be used to illuminate the region of interest when the subject is placed the illumination path.

As the x-ray source or emitting location on the source moves in at least two dimensional plane, multiple dimension images can be reconstructed based the measurements. The movement of the x-ray source or emitting position may be done as described herein.

For example, electromagnetic mechanisms, such as current coils which generates tunable magnetic forces, may be used to steer the electron beam as it hits the anode target. Alternatively, a mechanical mechanism, such as a motorized mover may move the x-ray source or anode target, so that x-ray emitting position may be varied in at least a 2D plane or more dimensions,

Each step of the movement may be approximately the resolution desired to be achieved in the third axis. In some instances, the measurements can be done at higher or smaller step size.

For each voxel, a Fourier Transform measurement may be resolved using spectral multiple dimensional measurements described herein.

The 3D or multiple dimensional imaging is reconstructed by takeing x-ray measurements, 2D or 1D or point at selected wavelengths. Each voxel in the region of interest, or the target or the component may be resolved to have a known value at each wavelength. Fourier transform of the value at each wavelength for each voxel can be obtained to further characterize material and substance of the component and the structure or microstructure of the component, target or the region of interest.

Combination of dataset other than imaging or property measurements will now be described.

Molecular measurements, or property measurements and macroenvironment assessment relevant to the subject at hand may be used in presenting facts for a user to draw conclusion, derive facts or make deterministic decision or conclusion based on probability or form data models for analysis or speculation or theory forming or data presentation and for selection of certain data set, unstructured and/or structure and criteria for presentation and consensus, or diagnosis and preplanning.

For example, in diagnosis of illness or selection of drug target, or for statistical analysis or modeling, data relating to immune profile, such as monitoring or temporal monitoring of antibody level, before or after an illness or external disturbance, profile of specific or nonspecific immune response, can be included for evaluating a subject or monitoring or assessment or disease surveillance or post therapeutic monitoring. In addition bio marker or image markers or change in cell signal or molecular pathway, concentration of certain molecules or characteristic of mixed molecular environment, physiological condition, environment related elements, personal genomics or gene data of the subject and history data or that of his or her relatives or measurements in MRI, Ultrasound, Sound, Elastograph, Optical Modality or mechanical properties and dynamic properties and component and mixtures or compositions, can be used together as part of dataset. X-ray imaging disclosed herein may be used together with each or combination of one or more type of data. And x-ray imaging may be used to acquire one or more type of data disclosed here as part of the data set.

Collimator based beam filtering technique can be used to remove scatter, for example, when taking a primary x-ray measurements of the subject in distributed spatial locations, to generate low resolution primary x-ray signals at selected regions.

An x-ray generating mechanism with selected regions for generating x-ray beams, such as a anode target with configurations on the surface when electron beams interacts with the targetmay only generate x-rays at selected regions . The rest of regions can either absorb electron beam, generating no x-ray beam, or redirect or steer an electron beam or x-ray beam in spatial domain or delay the electron beam or x-ray beam in time domain or both.

A beam selector or collimator with transmissive regions for selected wavelength and/or energy level or broadband beam, and opaque regions for selected x-ray beam can be placed between the x-ray source and the subject or between subject and detector to let primary x-ray pass in distributed spatial locations where such rays are transmissive.

To derive low resolution scatter signals at selected regions, low resolution primary x-ray at selected regions can be subtracted from x-ray measurements of selected regions resulted from taking a second x-ray measurements of the subject without blocking of primary x-rays or primary plus scatter signals, for example, by removing the beam selector or collimator, or displacing and modify or modulate anode target region.

The low resolution scatter signal can be interpolated to the entire imaging area on the detector to derive high resolution scatter.

High resolution scatter can be subtracted from the second x-ray measurements to generate high resolution primary image.

Alternatively, there may be a beam particle stopper plate 100 between the subject and the source or the subject and a detectorA beam particle stopper plate may include round shaped x-ray beam attenuation materials, such as tungsten or alloys, or lead or copper, or zinc or mixed alloys. Such materials can be distributed on a plate with largely x-ray transmissive regions. The location of such materials may be so that at selected locations, primary x-ray s are blocked, and only scatter signals are collected on the detector. When such scattered signals are interpolated to the entire image, a high resolution scattered signals can be derived. And a high resolution primary x-ray may be derived by subtracting the high resolution scatter image from the measurements. Such a system is illustrated in FIG. 20.

The detectors used may be a single detector, or a stacked detector, each of the stacked detectors may be the same or different from each other in energy or spectral sensitivity or photon sensitivity or speed or may be x-ray or optical detectors.

The scintillators may allow for a top, low energy detector and a bottom high or medium energy detecting, a triple or more layered detection system where each layer detector is for one or more energy level or x-ray wavelength or optical wavelength.

X-ray energy level or speed may change from pulse to pulse. For example, if the top layers can detect 20-70 KeV, and bottom can detect from 70 key to 150 KeV, then, in the first pulse, an x-ray pulse with pulse peaks at 30Kev and 80 KeV may be generated. The stacked detector can detect at the same time. Scatter may be removed using methods described herein. And the second x-ray generation, x-ray may contain energy peaks at 50 Key and/or 90 Key. Four energy levels are measured instead of two. Alternatively, photon counting detectors may be used in top or bottom layer.

Alternatively, after the scintillation layer, a transparent optical detector may be used to allow variation in wavelength sensitivity and speed to be detected second optical detector. And beam selector may be used to allow only primary x-ray generated optical light to pass through to the second detector. Similar methods in removal of scatter can be used using optical detectors

If a large field of view is required, multiple detector and source set may be used, or one source with a beam splitter to generate x-ray sources targeted different regions of subject may be used. For example, in whole body imaging, one or multiple x-ray sources or source sets can be used in distributed spatial location or at the same location. A steering element either in the x-ray range or optical range may be used. Other x-ray optics or optical optics or modules may be used to manipulate the electromagnetic wavelength to generate one or more images of the subject with larger field of view. Such images may be complete image or segmented image in 1D, 2D or 3D, stitched together from images of small field of view. Such acquired image may be scatter removed to below 1% or below 5% or between 10% of primary, or a combination of scatter removed image and images without scatter removal imaging processing. An example of such whole body imaging device is illustrated in FIG. 37

One example of scatter removal is provide below:

Scatter removal may include the steps of illuminating the subject with x-rays from the x-ray source, producing a low-resolution primary x-ray image at the rear detector, calculating a low-resolution primary image at the front detector, reading a high-resolution image from the front detector, producing a low-resolution image at the front detector from the front high-resolution image, subtracting the front low-resolution primary image from the front low-resolution image to determine the front low-resolution scatter image, smoothing the low-resolution scatter image by removing the high-spatial-frequency components, calculating a high-resolution scatter image by interpolation of the smoothed low-resolution scatter image, and subtracting the high-resolution scatter image from the high-resolution image to yield the high-resolution primary x-ray image at the front detector.

Standard image processing procedures can be used to smooth the low-resolution scatter image by removing the high-spatial-frequency fluctuations in the scatter image and to ensure only the low-spatial-frequency signals that is consistent with the nature of scatter can exist in the image. According to Compton scatter theory and experimental data, the image produced by the scatter on a two-dimensional x-ray detector is quite smooth, or has only a relatively low spatial frequency.

By using the single energy method, the relationship between the primary image of the front detector D.sub.fPl (x(i),y(j)) and the primary image of the rear detector D.sub.rPl (i,j) can only be established approximately. There is always a slight deviation between the calculated primary front detector x-ray image and the true primary image of the front detector. The difference between the true primary x-ray image and the calculated primary x-ray image is an image with a spatial frequency much higher than that due to scatter. Thus, even though the exact deviation in the primary image is not known, by removing the high-frequency component from the calculated scatter image, the accuracy of the approximation can be significantly improved.

Next, the smoothed low resolution scatter image can be interpolated for those front detector cells not on the selected projection lines, yielding the high-resolution scatter image. Finally, the image of high resolution scatter can be subtracted from the composite front detector image to yield an image including primarily primary x-ray, which is a full two-dimensional image of the subject at the front detector after scatter x-rays have been substantially eliminated.

A X-ray system for x-ray imaging of multiple subjects or multiple regions of interest in one subject can include one or more x-ray sources, or x-ray source modules, for example, 12-1 or 12-2, or 12-3, or 12-4, or 12-5 as illustrated in FIG. 14, each having two or more x-ray sources or x-ray emitting device with relatively fixed positions, for example in a pixelated x-ray source, or a single, dual or multiple filament x-ray source, or x-ray source with adjustable focal spot size or one or more focal spot sizes, or x-ray source of linear accelerator or synchrotron source. Each of X-ray source module may be moved relative to each other, or relative to the subject, for example by a mechanical or electrical, electromechanical, magnetic or energy based moving mechanisms.

The detection mechanisms of the x-ray system may include one or more detectors, or detector assemblies, each detector or detector set including more than one detector, receiving x-ray from its corresponding x-ray source or sources. In some cases, multiple sources at various locations may generate x-ray that illuminates the subject or the region of interest, and projects on to at least one detector. There can be one or more regions on detectors. Each region can correspond to one or more x-ray source. Measurements acquired by such x-ray source and detector sets or regions on detector may be synchronized, with measurements acquired by different x-ray sources, or may be asynchronized, based on the measurements obtained from region of interest. For example, paths of imaging or processes of imaging by the same x-ray and detector pair, and where to image in a region of interest may have various versions, and which path or process to take may be determined on the earlier measurements.

X-ray source module may include x-ray source ultrafast in nature. Ultrafast x-ray pulses in ns, ps, or femto second or higher pulse speed, and frequency of hz, khz, mhz and higher. Such ultrafast pulses may be generated by light, LED light, laser light, going through photo multiplier tube, and generate electrons and thereby x-ray pulses at ultrafast pulse rate or by particle accelerator linear accelerator, electron modulated by ultrafast laser and thereby generating ultrafast x-rays or using ultrafast laser, plasmon laser, hitting nanowire or nanomaterials, generating x-ray. Ultrafast laser modulated electrons and interacting with target generating x-ray may be used as a source. Liquid metal-based x-ray source may be used.

X-ray source may generate one or more discrete energy levels, for example, using a broadband source and one or more monochromators such as one or more x-ray filter, which may be coded aperture kedge filters. Thex-ray sources may be a broadband source having one or more energy peaks that may be separated by time or simultaneously.

X-ray source may generate ultrafast pulses and/or discrete energy levels or spectral x-ray levels, with one or more energy peaks, which may or may more vary in time.

X-ray sources may include field emitter or cold cathode sources, synchrotron or linear accelerator sources, linear accelerator based sources, conventional x-ray tube sources, ink jet based sources, and/or x-ray sources with one or more focal points, some with varied diameter in focal points.

It is one aspect of the present disclosure to have a motorized and manual mechanisms to move x-ray optics, including grating, beam splitter, and x-ray optics used in interferogram, or focusing beams or beam splitters, Mems, x-ray optics and detectors and x-ray sources in x-ray microscopy systems in and out of the illumination pathway of each of the sources based on the application requirements. The movement may be x y z or rotating.

Alternatively, the sample may be moved relative to the source or detector using similar mechanisms.

X-ray optics configurations and/or one or more x-ray or optical optics elements used for magnifying x-ray images or miniaturizing x-ray images or manipulating x-ray beam or illumination or steerinh x-ray beams or electron beam to generate x-ray may be used in the beam path between x-ray source and detector based on application needs in tomography, 3D, 2D or 1D or point measurements.

In addition, as illustrated in FIG. 1, any elements in the beam path other than the ROI, in between the x-ray source and the detector, for example, table top 400 and beam particle stopper plate 100 may attenuate x-ray and perturb x-ray beams. Such an effect may need to be taken into account when using x-ray measurements from the detector for quantitative analysis, such as spectral imaging, tracking and tomography. The effect may be similar to a filter and can be measured and characterized at a time different than the measurement of ROI for each quantitative measurement and analysis of ROI, for calibration of data and measurements necessary for quantitative analysis in single energy, spectral measurements, scatter removal and/or tomography or multiple dimension reconstruction and analysis and/or AI analysis needed for diagnosis, treatment/therapeutic procedures and intervention procedures and monitoring and tracking.

Surgical tools guidance or external objects to the subject to be measured, such as a catheter probe or a biopsy probe, probe for energy modulation such as laser or ultrasound or electric probe, may be used and designed with various x-ray attenuation properties in all or regions of the object, and may be separately imaged and tracked in 7 D dimensions such as 6D spatially in x, y z, pitch, yaw roll plus the 7th dimension, which is time.

The probe, the robot or catheter, implant or surgical probe or surgical tool or biopsy tool may attenuate x-ray beams of one or more energy levels at different regions of its body differently.

One or more portions of the probe, the robot or catheter, implant or surgical probe or surgical tool or biopsy tool may attenuate x-ray beam, and/or may attenuate x-ray beams with different levels of attenuation, and/or may attenuate x-ray beams of different wavelength at different levels.

Such an x-ray from the first emitting position may be steered to a second director by x-ray optics, such as x-ray mirrors, diffraction grating or mem device.

The collimator downstream from the second source or downstream from a moved x-ray source may be omitted.

First detector can be defined as the first detector to measure x-ray illuminating and passing through VOI.

To image the region of interest, or volume of interest, or selected VOI within a Volume of Interest of the last x-ray measurement or prior x-ray measurements or selected by the user with different x-ray sources or different detectors or different x-ray source and detector pairs , x-ray detector of various features and functions may be place in the first detector location to collect x-ray pass through VOI, or may be placed upstream or downstream of the first detector, after the first detector 22, and the detectors 29 are moved rotationally or linearly in the xy plane parallel to the first detector 22. Two or more detectors 29 with varying frame speed and pixel formats may be placed downstream from the first detector and in the projected line of sight from the first sources. Detector 29 may be a flat panel detector, multiple pixel energy sensitive detector, or small area detector or point detector, or 1D detector, or photodiode, or photomultiplier tubes or point detection systems or spectrometer including of x-ray optics such as a diffractive grating for dispersing x-ray beam onto an energy sensitive grating, with thex-ray beam then in turn to be collected by a spatially sensitive detector or detector array.

Detector 29 may be placed upstream of the first detector 22, and/or upstream of beam particle stopper plate 100.

There may be an optional patient tabletop or sample holder in between patient and the detector assembly which may include detector 22 and/or 29 and/or beam particle stopper plate 100

The resolution of the x-ray images may be extended beyond the measurement. One method is increase resolution beyond pixel pitch so as to capture of features beyond the physical pixel size using scanning protocols to produce an increased effective resolution, enhancing the image quality.

higher resolution can be obtained in spatially and in frequency. For example, the spatial domain super resolution such as Patch-based super resolution (PBSR) is a method where spatial features from a high resolution modality are used as references to guide the reconstruction of low resolution images from the second modality using image redundancy. The method finds a similar patch in an image and then attempts to reconstruct pixels by using information from similar neighboring pixels. In a parallel clinical setting, PBSR can be applied in the medical imaging field, where low resolution magnetic resonance spectroscopy images can be reconstructed in the spatial domain using high resolution magnetic resonance images as reference.

Other measurement modalities and therapeutic and diagnostic procedures, such as energy based intervention and measurement tools such as optical spectroscopy or temperature sensor measurements, may be used to further interrogate a selected ROI or deploy a intervention procedure.

The design of various intervention procedures may be optimized by having differentiable x-ray measurement properties such as atomic z, attenuation coefficient, thickness, or better visualized and responded to x-ray guided procedures or measurement methods.

Energy modulated measurement may be added for example, shear wave generator may be used to image certain region of interest.

Micromanipulator or other mechanical tool or energy probe, may be used for further manipulation of the subject and subsequent imaging and measurements.

Subject may be modified by energy mechanisms, such as RF, radiotherapy, electromagnetic energy, sound or chemical mechanisms pre, during or post x-ray imaging. x-ray imaging may be used to plan and guide procedures or monitor or surveying the subject over time.

Energy stimulating mechanisms such as acoustic ultrasound transducer or sound generator, transducer or thermo based ultrasound transducer may be implemented to characterize the subject or region of interest by measurement of mechanical properties, such as stiffness, dynamic mechanical analysis. Storage modulus (E′) is a measure of elastic response of a material. It measures the stored energy. Loss modulus (E″) is a measure of viscous response of a material.

Passive driver or pillow like passive drive used in MR measurements can induce mechanical waves, or generate shear wave propagating in tissue or a subject. The stimulation may be a two dimensional finite element simulation, and x-ray may be used to evaluate mechanical wave propagation patterns or displacement pattern in the subject. The stiffness map is called elastogram and may be used to differentiate subject or tissues at different states, such as diseased and normal, or characterize stages or developmental progress of a physiological change of a tissue or a component or the subject.

Phase Contrast x-ray may be implemented to improve sensitivity and resolution such as for soft media imaging.

In some cases, such methods can be combined with energy stimulating system and/or non rotational 3D tomography or scatter removal method to further improve the x-ray imaging of the prior art by increasing speed, lower radiation and increase sensitivity and resolution.

Energy Modulated X-ray Imaging with Temporal and/or spatial perturbation or shaping can be performing by the x-ray apparatus and methods disclosed herein. Conventional imaging systems, such as the CT, can be relative limited in identification and characterization of soft material such as soft biological tissue.

In brain imaging, to see the effect of brain trauma on blood vessels, capillaries, other tissues and matter in the brain , including neurons and the cellular matrix, elasticity measurements can be useful in characterization, diagnosis, monitoring and/or guidance of biopsy, imaging tools, surgery and therapeutic mechanisms including energy based treatment and drug delivery.

Diseased tissues, such as tumor, cyst or a normal tissue or its surrounding microenvironment and vasculature or angiogenesis may be characterized and identified by elastography as described to improve sensitivity.

Material, such as soft media, or soft material, for example, a biological tissue, may be be characterized further by its mechanical properties such as elasticity.

Acoustic mechanisms may be thermal based or magnetic or photoacoustic based. Deformation sources, such as vibrator, static compressor, air-puff and acoustic radiation force can create material displacements and x-ray imaging system and/or hybrid modality involving optical systems and methods may track these displacement. Propagatied shear or guided waves as aforementioned can be tracked to map the elasticity of a subject, sometimes with increased sensitivity and spatial resolution. Energy apparatus and methods for modulation of the subject, for example, acoustic or shear wave perturbation of the component, or the target or the subject at one or more frequencies, in for example, Hz, KHz or MHz, or GHz, and/or in time, and/or in space, can lead to dynamic elastography or mechanical or strain property measurements by one of more variations in x-ray measurement in point, 1D or 2D or 3D or more dimensions, and/or dimensions in time and/or frequency and/or in space, for example in displacement, or in density of component of interest or substance of interest. Non-contact energy or mechanical stimulation of soft media with precise spatial and temporal shaping can be used to measure and track dynamic elastography. Such stimulation may be acoustic mechanisms, for example, using ultrasound coupled with air and focused to induce mechanical displacement of a region of a material, such as a soft material using reflection based radiation force. The stimulation can be combinedwith high speed, multiple dimensional x-ray measurements, for example, scatter removal method and/or multiple dimensional x-ray measurements and/or spectral x-ray measurements or imaging, and/or phase contrast x-ray imaging and Fourier transform x-ray imaging, or ultra low radiation tracking as described herein.

Elastography approach may be implemented with direct contact with the material of interest or non-contact for excitation and/or detection of mechanical waves such as illustrated in FIG. 16.

Air-coupled US beam reflected from an air/soft medium interface can generate shear displacement through reflection based acoustic radiation force (ARF). ARF technical may be implemented using acoustic loss and scattering mechanism.

Non-contact mechanical excitation may be performed with a piezoelectric transducer launching through air an Ultrasound beam focused onto the air-medium interface. Beam reflection at this interface produces significant ARF toward the medium, inducing a transient displacement at the surface, including a shear one and generating a propagating mechanical wave in the lateral, transverse to the surface normal direction. The action is similar a hammer tapping wood or stick beaming drum where a localized, transient force on the target creates significant displacement in a transverse direction to that force. Due to the large difference in acoustic impedances of air and soft media such as tissue, the efficiency of acoustic energy conversion approaches one hundred percent. The transient displacement may be in single digit micron and the acoustic pressure may be a few kPa, a level far below any potential damage thresholds for tissue and is considered non-invasive.

For instance, a 1 MHz US pulse through air to the tissue surface may be accomplished by an air coupled piezo electric transducer that can efficiently transfer through air to the tissue surface with sufficient acoustic energy to launch a few kHz bandwidth mechanical waves with um-scale displacements easily captured by a high frame rate x-ray imaging system.

For instance, the x-ray system described herein may be used to capture mechanical wave propagation over a tissue volume in a fraction of a second to reconstruct 3D elasticity map from a single micro tap excitation by acoustic mechanisms per plane within the volume.

One or more x-ray thin beams may track and capture mechanical wave propagation over a tissue volume, or a 1D, 2D or 3D measurements. Such a system may be based on scatter removal methods and apparatus using a beam stopper particle plate 100 as described herein.

As illustrated in FIG. 18, a phase contrast x-ray imaging system including a grating, such as a transmissive grating 30, may be used to track and capture mechanical wave propagation over a tissue or tissue volume. Such a system may be near real time or ultrafast based on the source and detector used, and may be combined with scatter removal methods.

Contrast agents may be used to distinguish tracked substance or soft material in this energy stimulation based elasticity measurements. In some cases, contrast agents are omitted.

Strain elastography, point shear wave elastography, using acoustic radiation force impulse and 2D shear wave elastography, for example, UST1, may be implemented to generate displacement or shear patterns and measured by x-ray imaging system and methods in this disclosure.

Propagation-based imaging (PBI) is a phase-contrast 3D imaging method, wherein the phase shifts resulting from refraction within the object are converted to intensity variations via propagation between the object and the detector, with no optical components required along the path. As the wavefront propagates, small differences in phase accumulate between contrasting materials and/or changing thicknesses so that Fresnel fringes become clearly visible at the detector. The setup for PBI differs from conventional X-ray absorption imaging in the distance between the imaged object and the detector and in the requirement of a source with sufficiently high spatial coherence. Using one, two or more material phase retrieval algorithm as commonly known in conventional CT phase retrieval algorithm, adjusted for the 3D reconstruction method and apparatus described herein, a complete phase contrast multiple dimension system capable of near real time or ultrafast measurements and tracking can be implemented.

Alternatively, grating, beam splitters, in come cases, additional grating elements are used to construct a phase contrast system as illustrated in FIG. 17. Such a system may be combined with scatter removal methods as described herein.

Examples of x-ray optics based phase contrast systems as illustrated in FIG. 17 include two types of interferometers, the Talbot-type interferometer which uses two gratings (G1 and G2), and the Talbot—Lau-type interferometer, which uses an additional grating (G0). The beam splitter grating, or G1, is a phase grating which generates a periodic interference pattern of period p2 with maximum intensity oscillations at the distances d=dm, For q=1, G1 periodically shifts the phase by π/2 and zero, and thus it is referred to as a π/2-shifting phase grating. For q=2, G1 introduces a periodic phase shift of π and zero

The source grating, or G0, can be used if the spatial coherence of the beam is too small for the interference formation (Talbot—Lau configuration). G2 is the analyser grating with periodic absorbing structures of period p2, translating slight changes of the interference fringes into intensity changes at the detector. The geometric relation between G1 and G2 can be given by using a grating interferometer, and the measurement of these signals can be achieved by analysing a periodic interference pattern for any changes caused by a displacement (refraction, phase shift) or an amplitude reduction (scattering).

Although phase-shift interactions have a higher cross-section than interactions by absorption for hard X-rays, the eventual gain in the contrast-to-noise ratio (CNR) of two materials strongly depends on the imaging technique itself. For grating interferometry, this gain is mainly dependent on the beam coherence, but also on the spatial sampling (for example, the pixel size) of the signals.

The polychromatic characteristics of a region of interest by a grating interferometer can be measured by deriving the energy-dependent and the energy-integrated (polychromatic) interference fringe visibility at higher bandwidths. In general, a π-shift phase grating is more favourable for X-ray beams with a broad bandwidth, except for the first fractional Talbot order (m=1), where a it/2-shift grating slightly outperforms the π-shift grating.

In the phase-stepping approach of grating interferometry, the phase-stepping curve Ip(x) is acquired by moving one of the gratings in the transverse beam direction over a grating period and by recording the pixel intensity at multiple, equidistant steps. The projected source profile S′(x) can be either the shape of the focal spot in an X-ray tube (Talbot-type interferometer) with G0 or an array source, generated by a source grating (GO of a Talbot—Lau-type interferometer).

The polychromatic performance of the grating interferometer was studied quantitatively by analytically deriving the relationship between interference fringe visibility and photon energy. The spectral acceptance, which is the width of the spectrum between the two zero crossings of the fringe visibility around the design energy, is an indicator of the polychromatic performance. However, side lobes of the fringe visibility showed that the photons with energies outside the spectral acceptance also contribute to the signal and have to be taken into account for a polychromatic beam. For a π/2-shift grating, a fringe contrast inversion occurs at wavelengths outside of the spectral acceptance and for m>1, which leads to a rapid decay.

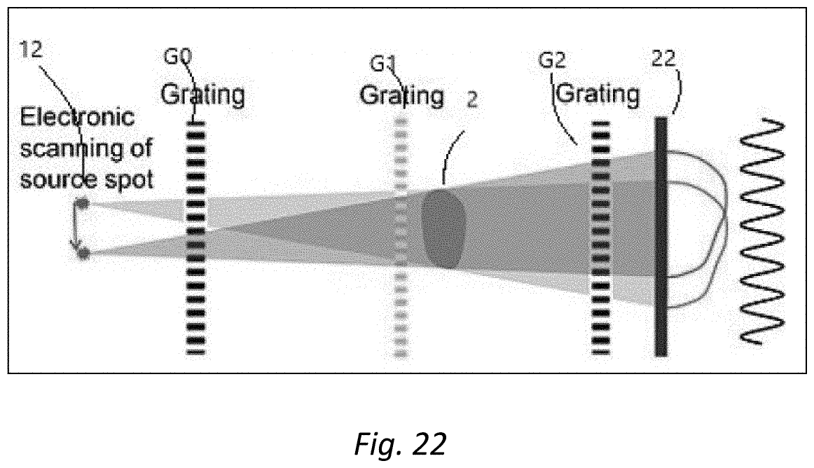

Electronic Phase Stepping or electron beam steering in an x-ray source is illustrated in FIG. 22, which showsan example that eliminates mechanical scanning of the grating and still retains the maximum spatial resolution is electronic phase stepping. The example electronic phase stepping scans the source spot of the x-ray tube with an electro-magnetic field, such as using a solenoid coil. This causes the projection of the object to move in the opposite direction, and also causes a relative movement between the projection and the Moire fringes. The images are digitally shifted to realign the projections. The end result is that the projection of the object is stationary, while the Moire fringes move over it. This technique effectively synthesizes the phase stepping process,

Electronic Phase Stepping, for example, as illustrated in FIG. 22, uses electron magnetic steerer such as current generating solenoid coil to steer the electron beams and therefore the location of the focal spot on the anode in an x-ray tube. The x-ray generated may be at different phase relative to x-ray generated from other focal spots. G1 and/or G2 may be omitted in some instances.

The same apparatus and methods may be used for steering the focal spot for multiple dimension and 3D imaging as described herein. Both mechanisms and implementations may be combined to achieve both 3D and/or phase contrast imaging and/or phase stepping, with one or more electromagnetic steering devices.

This apparatus and methods may also be used to generate thin x-ray beams for very small region of interest, especially in the x y direction, parallel to the detector. Where there is spectral x-ray beam generated or sequential monochromatic x-ray beam generated, high resolution spectral measurements or high resolution in spatial measurements, or high resolution in speed, may be achieved, as small detectors with smallest pixel sizes or photon counting detectors or PMT or photo diodes or silicon shift detector, or point detectors, or linear detectors, may be used.

X-ray Measurements using electromagnetic steerer may be combined with shear force generating device, sound as a sound device or ultrasound generating device to measure elasticity of a sample.

Using an electromagnetic steerer such as a solenoid coil moves electron beams and its focal point of x-ray source in two or multiple dimensions to generate variable illumination paths passing through the region of interest. Solving for the unknown voxels along the axis perpendicular to the detector and reconstructing the 3-dimensional distributions of the real and imaginary part of the refractive index. Tomographic reconstruction of the dark-field signal may be implemented for the phase-stepping technique and for the Moire pattern approach as well.

It has also been demonstrated that dark-field imaging with the grating interferometer can be used to extract orientational information of structural details in the sub-micrometer regime beyond the spatial resolution of the detection system. While the scattering of X-rays in a direction perpendicular to the grating lines provides the dark-field contrast, the scattering in a direction parallel to the grating lines leads to blurring in the image, which is not visible at the low resolution of the detector. This intrinsic physical property of the setup is utilized to extract orientational information about the angular variation of the local scattering power of the sample by rotating the sample around the optical axis of the set-up and collecting a set of several dark-field images, each measuring the component of the scattering perpendicular to the grating lines for that particular orientation. This can be used to determine the local angle and degree of orientation of bone and could yield valuable information for improving research and diagnostics of bone diseases like osteoporosis or osteoarthritis.

The configuration requires spatial coherence of the source and consequently is limited to high brilliant synchrotron radiation sources. This problem can be handled by adding a third grating close to the X-ray source, known as a Talbot-Lau interferometer. This source grating, which is usually an absorption grating with transmission slits, creates an “array of individually coherent but mutually incoherent sources”. As the source grating can contain a large number of individual apertures, each creating a sufficiently coherent virtual line source, standard X-ray generators with source sizes of a few square millimeters can be used efficiently and the field of view can be significantly increased.

Since the position of the interference fringes formed behind the beam-splitter grating is independent of wavelength over a wide energy range of the incident radiation, the interferometer in phase-stepping configuration can still be used efficiently with polychromatic radiation. For the Moire pattern configuration the constraint on the radiation energy can be stricter, because a finite bandwidth of energy instead of monochromatic radiation causes a decrease in the visibility of the Moire fringes and thus the image quality, but a moderate polychromaticity is still allowed. A great advantage of the usage of polychromatic radiation is the shortening of the exposure times and this has recently been exploited by using white synchrotron radiation to realize the first dynamic (time-resolved) Phase contrast tomography.

An x-ray far-field interferometer using only phase gratings is based on the phase moire effect. The mid grating forms Fourier images of the first grating. These images beat with the third grating to produce broad moire fringes on the detector at the appropriate distance. Phase shifts and de-coherence of the wave front by the object causes fringe shifts and attenuation of the fringe contrast.

The grating fabrication challenge was eased by the discovery of a phase moire effect which provides an all-phase-grating interferometer that works with compact sources, called the polychromatic far-field interferometer (see figure on the right). Phase gratings are easier to make when compared with the source and analyzer gratings mentioned above, since the grating depth required to cause phase shift is much less than what is needed to absorb x-rays. Phase gratings of 200-400 nanometer periods can be used to improve phase sensitivity in table-top PFI imagers. In PFI a phase grating is used to convert the fine interference fringes into a broad intensity pattern at a distal plane, based on the phase moire effect. Besides higher sensitivity, another incentive for smaller grating periods is that the lateral coherence of the source needs to be at least one grating period. As scatter is removed and the tomographic image produced combined with phase stepping can give reveal additional information about VOI previously not available.

An example of X-ray Tomography System

The x-ray tomography systems or an x-ray spectral imaging systems and/or an x-ray scatter moved systems can include a microprocessor, with or without one or more computer display systems and one or more microprocessors from different perspectives. The system can include a mover for x-ray imaging with large field of view, for example, approximately whole body imaging and/or for tomography support structure, column support, detector gantry assembly. A patient table can be rolled to fit to the side of detector gantry. Alternatively, a patient may be directly lie or sit on the detector gantry assembly which may have a surface to support the patient.

An x-ray CT system or spectral imaging system or scatter removed system, can include:

an x-ray source;

a moving mechanism for moving the source with one or x-ray emitting positions, or the x-ray emitting positions,

Optionally a rotor,

Optionally, a collimator,

Optionally, a second moving mechanism for moving the detector independently. Both detector and the x-ray source may be attached to a structure column or mounted separately. For example, the source can be mounted to the ceiling, or a upright stand, and the detector correspondingly may be placed in a support structure such as a table, or the detector and motor may be mounted on a translation stage attached to a structural or support device, such as an x-ray table, surgical table, or simply an anchor mechanisms to the floor, such as a gantry supported by wheels or an upright stand, facing the x-ray emitting position of the x-ray source placed on an upright stand.

Optionally, a central imaging bore defined by which the rotor rotates around the bore;

Optionally an x-ray source mounted to the rotor;

Optionally an x-ray source with a moving mechanism mounted on the rotor;

A detector system mounted to the rotorcan include: a detector chassis including a rigid frame; a plurality of detector elements within the detector chassis and arranged on the rigid frame; and a processing unit located on or in the detector chassis and including a plurality of processing cores for performing parallel processing of image data received from the plurality of detector elements to generate a scatter removed measurement, substantially primary x-ray measurements with scatter removed to less than 1% or less than 5% of Scatter to primary ratio, and/or material decomposed measurements or images, and/or three-dimensional tomographic reconstruction of an object located in the bore or in between the source and the detector or on a table or a holder in between the source and detector.

In the x-ray CT or scatter removed x-ray imaging system with scatter to primary ratio of less than 1% or less than 5%, and/or spectral imaging system, the processing unit may include a graphics processing unit (GPU). The GPU may can include an internal memory of at least 2 gigabytes and at least 2048 processing cores.

The tomographic reconstruction, and/or material decomposition, and/or scatter removal image processing and/or data analysis results for diagnosis, tracking, pre treatment planning, monitoring, surveillance and, image guidance are at least partially generated when the x-ray emitting position is moved relative to the object.

The x-ray imaging system further can include a wireless and/or wired transmitter coupled to the processing unit that transmits the tomographic reconstruction from the image acquisition location and/or the rotor to a location or device away from the rotor.

The x-ray imaging system, or x-ray tomography system, or spectral x-ray tomography system, or spectral x-ray system or scatter removal x-ray system, or structural illumination x-ray imaging system can further include a collimator to adjust field of view FOV for the radiation emitted from the x-ray tubesource, or to restrict the region of interest ROI or to place selected filters down stream from the source.

The detector module or detector modules can include a plurality of detector modules arranged on the rigid frame, containing motors to move each of the detectors in and out of the imaging path, each detector module including an array of detector elements of the plurality of detector elements and associated electronic components configured to read out image data from the array of detector elements.

The aforementioned x-ray system can include the plurality of detector modules that may be independently movable or stacked, and/or further include a beam selector or a beam particle stopper plate/array in between stacked detector modules, or having a beam particle stopper plate upstream of the detector module.

Each detector module can include a processor configured with processor-executable instructions to read out image data from the array of detector elements on the module and transmit the image data from the detector module in response to receiving at least one of a clock signal from a master clock or a motion system indicating the source and/or detector are in the selected location and/or from collimator, indicating the selected field of viewFOV and/or region of interestROl are selected and available for imaging, and/or a selected filter or filters are in place, and/or the generator sync signal or trigger signal, or in response to processor instruction after an imaging acquisition step prior to the present imaging acquisition step.

The processor of each detector module can be configured with processor-executable instructions to combine image data from the detector module with image data received from one or more detector modules of thelocated in the same gantry before transmitting the combined image data from the detector module. The aforementioned x-ray system can transmit the combined image from all of the imaging detector modules to the processing unit of the detector system to generate the tomographic reconstruction, and/or spectral image or the measurement with material decomposed to at least one substance or component, and/or a measurement of essentially primary x-rays and/or an image-processed measurement with scatter to primary ratio of less than 1% or less than 5%.

The aforementioned x-ray systemcan further include a reference detector including a detector element that is positioned to measure a flux of photons emitted by the x-ray source before the photons impinge on the object located in the imaging path. The reference detector can be located at least partially outside of the detector system and can include an electronics module that generates an electronic representation of the measurement of the flux of photons emitted by the x-ray source, wherein the electronic representation of the measurement of the flux of photons is sent to the processing unit in the detector system for use in generating the tomographic reconstruction.

The reference detector can include a housing containing the detector element located proximate to an x-ray beam outlet port of the x-ray source and a fiber optic cable coupled to the detector element to transmit an optical signal from the detector element to the electronics module.

Optionally, the detector element is a direct converted x-ray detector.

Optionally, the electronics module and/or the reference detector is located in or by the collimator.

Optionally, an energy filter can allow at least one x-ray of one or more energy levels to pass through allowing it to reach the reference detector,

Optionally this energy filter may be physically attached to the reference detector.

The electronics module can include a photodiode that converts the optical signal from the detector element to an electronic signal that is transmitted to the processing unit in the detector system.

The reference detector can include a temperature sensor that generates an electronic signal indicative of a temperature within the x-ray source, wherein the electronic signal indicative of a temperature within the x-ray source is sent to the processing unit in the detector system for use in generating the tomographic reconstruction.

X-ray imaging of the present disclosure, for example, spectral x-ray imaging, scatter removed x-ray imaging, 3D x-ray imaging can be used to locate the region of interest and guide the endoscope or biopsy needle or treatment probe, or optical imaging focal point to the site of interest or of region of interest, such as a tumor site inside of the body. X-ray imaging can the be used to activate fluorescent dye for endoscope or an optical senor to image, with quantitative measurements and specificity, either surgical guidance or treatment guidance or diagnosis and monitoring, of fluorescent labeled regions or fluorescent imaging assisted procedures or for dual modality imaging of x-ray imaging and optical imaging.

Structural illumination as described herein may be used for x-ray activating of contrast agents at selected region of interest.

Free radical fluorescent sensors may be used to monitor x-ray activated activities and enable diagnosis such as tumor quantification and diagnosis.

Alternatively, optical imaging or ultrasound or MRI may collocate or guide or provide information to direct x-ray imaging to the region of interest for diagnosis or monitoring.

For example, PET or MM may locate tumor location in the diagnostic stage, and x-ray imaging of the present disclosure with spectral or multiple dimensional imaging or scatter removal methods and apparatus, and may track and locate and monitor tumor size in real time, or over time, with specificity by using target specific contrast agents or tumor markers without contrast agents.

In conventional methods, a single energy is used to select region of interest, then CT scan of ROI. The limitation of this method can include overlapping tissues, and scatter, many details bieng missed, therefore the selection of region of interest being not optimized. By using spectral imaging or for example, low resolution 3D imaging, or contrast labeled x-ray imaging with scatter removed and material decomposed, the region of interest may be selected. High radiation and long imaging time is required for CT imaging as the entire region surround the ROI needs to be imaged even though the analysis of CT images will be focused on the selected ROI.

The present disclosure includes the methods for determining ROI such as spectral imaging, scatter removed imaging and 3D imaging or CT imaging may take place in real time and multiple ROIs or smaller and smaller ROI can be determined in real time throughout the imaging process. In other words, determination of ROI can be iterative or repeated as new information are learnt with each image or measurement taken.

The following disclosure presents several examples of Customized Imaging and Spectral Tomography.

Illustrated in FIG. 1-8, various x-ray sources are positioned to move in and out of the x-ray emitting position aligned with the detector for the imaging of VOL Or , each may stay at a fixed position but apart from others, to illuminate the same subject or VOI or a portion of VOI with using various, different parameters such as, for example, focal spot, field of view FOV, pulse speed, energy levels, various pulse characteristics, modulation characteristics or they may be manipulated differently by various optics or x-ray optics.

As illustrated in FIG. 1, two or more sources that emit x-rays from approximately the same x-ray emitting position at the same time or at various time frames but may have the same or varied characteristics. Ffor example, the source may vary in source focal size, beam spatial and temporal characteristics such as structural illumination, field of viewFOV, frame rate, or energy levels, varied pulse characteristics and/or pulses modulated in phase and amplitude, coherence or partially coherent sources. Alternatively, such sources may be contained in the same x-ray tube, or each x-ray tube may be separated or moved together by mechanical devices or robotics or motion systems or be steered by modulated energy sources and methods such as ultrasound, electromagnetic forces such as, for example, solenoid coil around the x-ray vacuum tube or electro-optical forces or electronically-controlled devices such as MEM mirrors.

Each of the sources may be configured in a source assembly to allow for spectral imaging and/or tomography, and/or complete 3D tomosynthesis, or multiple dimensional imaging. The assembly may include apparatus and methods to manipulate beam profiles in space and time, split electron and/or x-ray beams, generate additional signals to be modulated, and/or be moved or steered by energy mechanisms such as electromagnetic steerer or, electro-optic lens.

Reconstruction may also be accomplished using Distance-driven projection and backprojection in three dimensions.

A Mobile and/or stationary method of performing medical imaging with scatter removal processing to reduce scatter to primary ratio to less than 1%, or less than 5%, or, spectral 2D or 3D imaging system and/or tomography and/or spectral tomographyillustrated in FIG. 35, 37, 48 and FIG. 52, 54, 55, 28, 30, can include:

transporting an imaging system mounted to the mobile base by operating a motor geared into one or more wheels of the mobile base to propel the mobile base across a floor;

transporting a patient support to the imaging system so that volume of interest in patient is in the imaging path using a mobile cart and/or a portable patient support or portable surgical table;

obtaining image data of a patient on the patient support using an image collection apparatus on the imaging system;

move the patient support from the imaging system; and

transporting the patient support away from the imaging system using the mobile cart, and

optionally, moving the surgical table with one or more wheels away from the imaging system.

A method for generating an x-ray CT reconstruction with an imaging system including an x-ray source and a detector system mounted to at least one translation movers, can be extended with the the recontruction method of an 0-ring system. The following includes rotary movements to access regions with multiple complex materials from an angle of optimized imaging perspective for n²Matrix method.

generating an electronic representation of image data received at a plurality of detector elements of the detector system while the rotor rotates;

determine a region of interest;

a translation mover can move the x ray emitting position in at least one linear axis, relative to the VOL The rotor moves the translation stage or an electromagnetic steering device for x ray emitting position movement as well as an x ray source for personalized tomography method

sending the electronic representation of image data to a processing unit located on or in the detector chassis while images are acquired;

performing parallel processing of the image data using a plurality of processing cores of the processing unit to generate a three-dimensional tomographic reconstruction; and

transmitting the tomographic reconstruction from the imaging location to an entity off the rotor.

The aforementioned x-ray system, wherein the processing unit can include a graphics processing unit (GPU).

The aforementioned x-ray system, wherein the GPU can include an internal memory of at least 2 gigabytes and at least 2048 processing cores.

The aforementioned x-ray system further including generating the tomographic reconstruction while the image is acquired and/or x-ray emitting position moves.

The aforementioned reconstruction method may include algorithms used in conventional CT systems, such as using calculation of geometric matrix combined algorithms to correlate projection measurements at different geometric locations of x-ray source relative the object and/or the detector, solving for unknown voxels in the volume of the interest in the object through a series or a set of projection measurements of the VOL A second geometric matrix may be created, or a new coordinate or new vector formed to combine both tomography methods.

The algorithms can be used for reconstruction based on derivations of attenuation values from the measurements and/or calculations of photon flux for each voxel, for each layer of ROI and for a projection path and measured data on a corresponding pixel or pixel region on a detector. Iterative algorithms and/or model based iterative algorithms may be used to improve convergence of simulated measurement data from direct solving of knowns based on for a reconstruction model, such as ART or Monte Carlo and that of the pixel or pixel regions in the projected path of the voxels being determined, as part of VOI.

The reconstruction algorithms of the aforementioned x-ray system may be the same, or similar to, or derivatives of the tomosynthesis and/or CT and/or spectral CT algorithms with exceptions of the description of geometric matrix transformation, which may be used to describe the relative movement of x-ray emitting position to the objects, for example, the steps of small distance movement on a xy plane, or in a xyz volume or in 6D dimensions, and in some cases, the corresponding detector movement in some system configurations.

Due to the substantially absent and/or removed scatter x-ray interference, and/or substantial completeness of data set and/or dual or multiple energy material decomposition methods, limited motion movement requirements or non moving nature of x-ray source emitting positions in some examples, the reconstruction time using the same or similar or derivatives of CT and tomosynthesis algorithms may be much shorter in time.

Reconstruction algorithms or steps to correct geometric errors in tomosynthesis and CT tomography algorithms in reconstruction due to robotics movement in image acquisition process, and/or noise or artifacts exist due to differences in spatial geometric and movement configurations may not be used. Number of iterations may be reduced or removed. Method to recognize shape or markers and/or interpolation methods for reconstruction may not be used if the analysis is based on measurements and facts derived of such measurements on a pixel-by-pixel basis.

Reconstruction algorithms may emphasize data-driven knowledge-enhancing abilities as the strength of deep learning-based reconstruction.

From the missing data in the projection domain, a two-step deep learning architecture can be implemented. In the first step, compensation weights can be learnt that account for the missing data in the projection domain and correct for intensity changes. In the second step, the image restoration problem can be formulated using a variational network to eliminate coherent streaking artifacts.

Such missing data compensation method may be used in the following examples:

When low radiation is desired, for example, low resolution in Z axis perpendicular to the detector is acceptable, such missing data compensation may be used.

For the newly introduced unknowns in the regions outside of region of interest, such as when the detected region is limited geometrically to recover new unknown voxels in the new rows and columns introduced in the regions outside of region of interest while acquiring projection measurements for multiple dimensional image reconstruction.

When the beam stopper particle plate/array is used. When dual or multiple energy level x-ray imaging is performed, the x-ray attenuating regions on the beam stopper particle plate may be moved at unique locations relative to each of the x-ray project path collected by a corresponding pixel or pixel region for each energy. Rather than repeating measurements at different energies for the same beam stopper particle plate position relative to its relative pixel or pixel regions, the imaging data missed due to the primary x-ray blocked by the x-ray attenuation regions can be compensated or derived by either direct derivation at measurements of other energy level or other energy levels when the beam stopper particles are at different locations or by deep learning AI algorithms to reconstruct compensation data from measurements at other energy level or other energy levels for the same projection line of VOI.

In one example, the measurement is on a gated x-ray imaging system for tomography and/or spectral imaging, and/or spectral CT and/or Spectral tomosynthesis, and/or tomosynthesis or scatter removal processed image, such as primary x-ray imaging.

Such a gated system may be a respiratory, or motion or cardiac motion cycle gated imaging system and/or an ECG or other measurement or intervention procedure, monitoring and diagnostic device based on a signal processing gated imaging system.

Parallel iterative CT reconstruction based on local reconstruction algorithm may be used where subregions of ROI are reconstructed at the same time.

Customized Reconstruction refers to when the region of interest are reconstructed, during any time within the reconstruction process, selection criteria are inputted by a user or predetermined, and controlled by digital program, such as recognition of a diseased region and/or anatomic markers or spatial location relative to a reference. Regions of focus may further identified and selected, and reconstruction algorithms and iterative algorithms or other CT or tomosynthesis related algorithms may be used for reconstruction, thereby having a priority to the other regions and may in turn speed up reconstruction for the prioritized region or may be prioritized against other regions in terms of reconstruction and image processing. This process may increase the speed of reconstruction to acquire data for the most critical or focused or customized and/or personalized imaging. If image reconstruction is done at the same time as image acquisition, the prioritization or customization of region of focus may be done during the image acquisition process.

Deep learning algorithms may be used to optimize data, measurement, image acquisition performance by one or more microprocessors and reduction of artifacts, noise and speeding up of relevant data registration, collection and processing and/or iteration of such a process by identifying and selecting regions as well as reconstruction processes during and after image acquisition. FIG. 11 illustrates examples of data and image processing before, during and post acquisiton and or construction.

Customized reconstruction may be accomplished by two steps. In the first step, measurement, image acquisition is performed, including for example, controlling the use of single, dual or multiple energy x-ray source, moving one or more x-ray source and/or x-ray emitting location and/or detector to the imaging path of the object and/or region of interest or volume of interest, using collimator to select one or more area for field of view and/or region of interest, using motorized filter to filter energy levels, using beam chopper to select region of interest or moving x-ray source, and/or other x-ray beam, electron beam and/or optical beam magnification, concentration, diffraction, refracting, splitting and collimating and/or steering and/or modulating or manipulating size, quantity, dimension, intensity, phase in time, and frequency and space domain. In the second step, reconstruction is performed using one or more spectral tomography and tomosynthesis and/or 2D images at one or multiple energy levels and/or data set reconstruction algorithms or processes or steps without but with user guided optimized process. The procedure and process for image acquisition and/or reconstruction of a object to be customized and/or optimized based on the optimized process can be developed with or without user interference. Deep learning algorithms can be used to train and learn the process of acquisition and reconstruction on raw images as well as processed images.

Deep learning algorithms can be used to control the image acquisition by determining factors to minimized the region of reconstruction and/or of measurements and/or resolution of the measurements and/or sparsity of the measurements and/or compressed configuration of the measurements, and/or reconstruction process, such as prioritization of selected region of ROI, during image acquisition and/or reconstruction and/or data derivation.

Multigrid reconstruction may be used for tomographic reconstruction for the present x-ray system.

A number of projection images can be acquired, therefore resolutions along the Z may be adjusted. When less than what is needed for complete 3D reconstruction, various methods or known methods in CT tomography reconstruction may be used to fill in the gap of the missing data if there are truncated missing data or the acquired data are too sparse.

Image reconstruction methods may use either direct methods and/or variational (regularized, iterative) methods. Direct methods are derived in the continuous domain; they are fast to apply and give good results when the number of measurements is high. Reconstruction in the time, or space or frequqnecy domain may be used.

Variational methods involve minimizing an objective function that usually includes a data term and one or more regularization terms.

The x-ray systems disclosed herein can further be modified by retrofit or replacement or add on or a kit or one or more set of elements of the x-ray measurement system.

Each of the hardware piece or modules or relevant software component or algorithms for image processing or controlling or integrating hardware components described herein may be added on as a retrofit item or replacement item or modular item.

At least one element of such an imaging or more may be used as a kit to supplement other parts of system to provide a complete system for imaging.

Payment Method

X-ray Imaging record keeping and payment process apparatus and methods will now be described.

An imaging taking apparatus including the following methods and apparatus can include One or more microprocessors, Wired or wireless communication device and protocol and software process, and, Cloud, server or hardware storage locally or remotely.

The microprocessor can contain Method for recording number of images, or number of procedures, which may be acquired, processed or not processed, extracted, selected, and/or each may be traced to a reimbursement code existed, or may be created in the future.

The microprocessor can contain one or more database, or database structure to store and categorize each image based on one or more criteria, such as studies, or type of studies, or images, type of images, procedure utilizing images, or measurements related to a procedure, or extracted image from measurements and reconstructed images, extracted data from measurements and reconstructed images.

The microprocessor may be associated with a software, or algorithm using to label or time stamp an image.

The microprocessor can create a time stamp for each image taken, either 2D or 3D, or more multiple dimensional, such as attach a DICOM label or adding a time label, store in the database which is stored in the microprocessor.

The microprocessor can identify the image or image set. For example, each image can be labeled with at least the name and/or a description of the subject or the region of interest, or at least a unique identification number or binary identification number, or all of the aforementioned ID information.

The microprocessor can record the number of images taken per subject based on DICOM labels or unique identifier for each imaging process or each imaging session or each study or treatment or diagnostic or monitoring or therapeutic planning, or research project or tracking period.

The microprocessor can record and tally number of images taken and/or processed for each x-ray system including the computer, the x-ray hardware and the software; and a memory storage unit, electronically store one or more documents. Each memory storage unit can have reports or up to date records of number of images taken during a time frame such as a day or a month or a year or since the system has been in use; the report or the document can be accessed by either physically accessing the computer and its associated x-ray imaging system or the electronic memory storage unit remotely via internet or intranet or direct physical access for example a memory stick or security key capable of storing and process digital information. The x-ray system can include a computer that is programmed to generate a report based on the document, stores the report electronically, and periodically automatically sends the report to the predetermined recipient via email or hardcopy or other electronic mechanisms for example, stored on a server, password protected, accessible for the predetermined recipient who can access by login to access the record by using a password either at the x-ray system location, and/or at a remote location.

The apparatuses disclosed herein can include a storage and/or a database as illustrated in FIG. 8, which stores images produced by the apparatuses disclosed herein and/or using the imaging methods disclosed herein. Each image or a dataset including images and/or data can be associated with a time label at time t=tO, tl, t2, the units of the time may be in seconds, or minutes, or hours, months or years, or any range from sub-seconds to years. Such time label can be associated with the time at which the image or data is acquired. Each image or dataset may or may not be acquired at the same facility. The time sensitive database may store images of the subject from one or more locations or facilities or different imaging sites, such as location 1 or 2 or 3 in FIG. 8, which may be linked with unstructured and structured data other than x-ray images relating to or of the subject with the same identifier or related identifier. Such data may be labeled with a time label at time t=t0, t1, t2 . . . Such database may contain unstructured and structured data relating to a fact extracted from the data and/or the images and/or associated with a specific time. Such a system allows for tracking and monitoring of the images of the same region of interest of the subject overtime.

The apparatuses disclosed herein can generate time sensitive scatter removed x-ray images and their post-processed images, for example, after material decomposition. Such images can be labeled with a time specifier, generally the time when the images are taken. Such images and related image set taken of a subject spatial and/or temporally may be labeled with a time stamp and/or a unique identifier to associate with a specific time for each image or image set, and an identifier associated with the subject. One or more facts may be extracted from such database, including time sensitive data.

The label and database system described above may incorporate any features of DICOM labels, including but not limited to a custom DICOM (Digital Imaging and Communications in Medicine) label. In some instances, such a label with specific time and an unique identifier may be made with a second ID, for example, a social security number of the subject (that is, a human patient), which is relatively permanent, or an identifier chosen by the subject. Such identifiers can be integrated with a random number to generate an encryption. The identifier may be one fact relating to the subject or one set of two or more facts relating to the subject. The identifier may be a second fact or a second key about the subject or a set of two or more facts or numbers assigned to or chosen by the subject, so that the first identifier or first set of identifiers may not be made public, or may be hidden when accessing the image or the image set of subject. The second key or second identifiers can include additional security measures of using a second identifier, which may enable retrieving of images and/or linking continuity of images of a particular subject without having to access private information. The second identifier may be a number or a method of access such as a physical key or an apparatus such as cell phone.

The database may not contain private information of the subject, but rather a key assigned to the subject or chosen by the subject or associated with the subject, such as, unique identifier for the subject, which may be social security number in the US. The subject and/or designated entities can have access to confirm or further validate the permission to access. Different combinations of second identifiers may be used together to increase the security of access. The database may include some or the complete private information relevant to the subject. In the case where there is no private information or partial private information, an encryption or access or tracking methods can be used to ensure continuity of the image data and other data relating to the subject over time. One or more of the following methods may be used, such as random number mixing with a secondary key; a second access apparatus, remote and/or on-site; and/or a second access component from the same apparatus. The secondary key may be of long term and non-changing nature, such as a social security number. The second access apparatus may be a physical key or wireless or wired apparatus may be used on site. Alternatively or additionally, an apparatus can be used remotely if there is Internet or Intranet communication to the database.

The database system can therefore enable linking, retrieving, and/or storing image data continuously and/or intermittently over time for a subject. For example, to diagnose, treat, and/or post-therapeutic monitor a disease or health state of a patient, such a system allows for accessing and evaluating images of the patient over time.

The database may containing a record of the number of images taken from one or multiple locations during a specific time frame. The calculation of how many images or measurement or facts derived from the measurements or images at one time or over time may be taken in real time as new measurements or images are taken or over time, from a fact and data storing database which may containing a tally or record or actual data. The calculated tally or data may be stored in a database in a local microprocessor as part of the x-ray imaging acquisition system or a part of image display system with graphic cards and display or a central database to include record of other types including patient electronic record, history, diagnosis and personal information, or the database may be used to tally the number of categorize images or measurements or facts derived from the measurements.

For example, the database may be constructed to collect digitally from one or more, or several image acquisition systems with at least one microprocessor. The database may be stored locally in an image acquisition system, and the database may be stored in microprocessor in a separate location or in a server, or in a cloud storage device. The number of images taken per one specific imaging acquisition system or for at least one image acquisition system from one facility may be recorded in real tiem or over a period of time.

A payment apparatus and method associated images taken or studies and patient including the images taken or the number of images taken may exist based on per image taken, based on subscription or based on an upfront payment made, in addition to pay per image at a less amount.

Generally, CT system or a general x-ray system or spectral imaging system are sold as capital equipment. Given that the costs of good sold of the apparatus and methods described here may be in the general radiology level, due to less complexity and less robotics required, the hospitals or clinics which previously could not afford a CT system, can now afford to purchase a unit. In order to make it more accessible for patients and physicians who are interested in using it, a new payment process may be used.

A membership based fee, or a subscription purchase may be charged for using the whole system or parts of or some of the apparatus and methods, hardware and software used described for x-ray imaging, measurement or analysis.

An automated payment process using checking or saving account or a credit card or scheduled wire transfer or direct deposit from a bank account may be done for the regular payments.

For example, a customer may pay a yearly or monthly flat fee for a number of images or a number procedures involving imaging guidance using x-ray imaging and measurement. There are various levels of subscription and levels of payment corresponding to likely volumes or use of system. When the system determines that the usage is higher than what was expected, the customer, such as a clinic or urgent care or surgical center, may be notified through the software via an email or an electronic msg. The buyer then may use a payment of choice, for example, use online payment process to pay for the additional amount for the month or upgrade the subscription level to a higher level. Services on hardware and software and free updates may be included in the subscription. There may be no upfront payment or a small flat fee in the beginning, paid through electronically or by a check. The electronic payment is done electronically online or through a check or wired transfer or direct deposit each month.

As an example of how the database, or calculated number of images or measurements or the facts extracted from measurements, and/or categorized data derived from or based on images and measurements can be provided for a pay per procedure method or pay per image or a set of images or purchase an analysis or image processing service, the clinic or the hospital can pay or compensate the seller of the device or the seller of the imaging service a cash or cash equivalent compensation.

The service can be listed on the internet or web store or mobile app based store, or market place.

Below is an example of how the image acquisition, viewing and measurement presentation and related or derived data may be provided in a bundled service along with other products and services.

A user may purchase of the imaging service and products on internet or by one or a few clicks on internet, similar to purchasing a book but only here to purchase an imaging or diagnostic or analysis service or product. The purchase can also be done via mobile phone or mobile phone app or web based app. The result of the purchase triggers a software to send a message to the warehouse of the seller or seller associated partners and installation service providers of the seller and/or its associated partner. An electronic msg generated by a software can be sent over the internet and/or a telephone call can be made to the buyer to confirm the sale and/or arrange for installation.

One or more imaging system and/or related viewer, and storage and communication hardware and software may be installed at the preferred or buyer designated site. Such a system may be stand alone or connected via cloud and intranet or local network.

The present methods and apparatus may be purchased and sold on the internet or via mobile platform using digital methods, for example, using only one click to purchase, or two or more clicks to purchase. The purchase methods may be using currency, or blockchain or cryptocurrency, or credit card, or bank account, or other acceptable methods by both the purchaser and the seller.

Payment may be made in crypto currency and seller and buyer agreed upon currency or the exchange of goods and services which are equivalent, online via mobile or internet or controlled network mechanisms.

For upgrade or renewal of services, or adding a new imaging and diagnostics and procedural service, a user may login to the seller's website, and make a payment. Or such a payment portal may be directly linked with the user's purchasing network. Different tiers of services may be listed, optimized service models may be suggested based the user's usage history, or preference.

The suggested model may be based on a list of questionnaires provided by the software, either online or via a workstation application. The user can select from a multiple choices question or provide answers in numbers or words or phrases. Automated software and/or assisted from a partner or representative online or in person may assist in answering the questions. The user or purchasing may choose to skip one or more questions presented by the software.

Entire purchase process and/or transaction may be encrypted or performed in a secured portal.

Direct deposit or fund transfer may be used. And for prequalified buyer, the payment transaction may be delayed for a term specified by the seller, this process may be managed by the software.

The seller may be the manufacturer or a customer or partner or both of a manufacturer which provides the imaging service.

The seller may provide B to B as well as B to C product and services via digital bank and/or digital wallet service.

B to B stands for business to business, for example, from the seller, for example, to a hospital or a clinic

B to C stands for business to consumer, for example, from the provider of imaging service or diagnostic service to a patient or an individual person and/or from the provider of instrument, via the clinics or hospitals, and/or imaging service provider, for example, by installing an imaging unit onsite of a customer or partner such as clinics and/or hospital, and/or directly offering imaging services to an individual person who is also a patient of the clinic or hospital.

Typically, a payment hub may be used to process payment transactions. The payment hub may generate interchange revenue share from electronic payment due to the amount of fees needed to be paid in additional to the merchant bank. The fees and related charges are typically paid in large amount, by the customer—hospitals/clinics/healthcare organizations/imaging centers—to the transaction handling merchant bank to cover handling costs, fraud and bad debt costs and the risk involved in approving the payment. In addition, for large dollar amount transactions, the time required for each transaction may be longer due to a lengthy process.

To reduce the cost and time required and increase efficiency of financial transaction in purchasing an x-ray imaging subscription service, or x-ray imaging system or x-ray imaging service, the seller of the x-ray imaging system and/or related product and services may partner with a bank or digital wallet service. The seller of the imaging service or equipment may become a digital bank, by providing a digital bank and/or e-wallet or digital wallet software platform and/or related financial services to customers. The digital bank status may be achieved through a banking license or an E-money license or a license of a third party which a license as a service model.

The digital bank can allow a customer to sign up with a user name and password, and/or telephone number or email and/or tax id, and/or social security number or other identifier information to have a bank account.

The seller of x-ray imaging device and related product and services may provide a digital wallet, or e-wallet, which allows the customer store money on an electronic device, or remotely on a server, creating Digital wallet, e-Wallet, or digital account via a software on mobile phone, a mobile wallet or desk top , or a wireless device or an online interface. The digital bank account and/or the digital wallet may be connected with one or more bank accounts and credit cards, support a number of features such as switching between bank accounts and credit cards, and/or allow deposit or storage of currency, transfers, payment transactions.

The software platform methods for digital bank may include:

the front end, including a thin presentation layer of information, for example, a mobile app, the app or the web portal which allows username and password input and sign in and registration and related information, Developer portal.

the back end including a product layer where the core banking system, client data and other back-offices related processes sits.

The middle-ware including an intermediary layer orchestrating information between the front end and the back end and API layer. The middle ware may contain a sub-layer, called the API layer enabling all connections to external/3rd party applications, which may enrich the service offering, other financial and product service providers, or accounting software. The middle ware may also contain customer accounts, loans, payments, market place, digital onboarding, payment networks, cards and card management.

The platform may allow for decoupling distribution channels, products and customer/client data, all connected by APIs enabling resilience to future changes.

A compliance software may be used to monitor serious potential risks. An intelligent customer support tools such as ERP or CRM software may be used to optimize channel management, for example, through email campaigns, video chat, social media features.

Digital bank accounts and/or digital wallets allow the user to make payments using the following methods:

using mobile phone by using near field communication (NFC) if NFC is equipped on such mobile phones, finger print and/or iris scanner or biometrics may be used to ensure total security.

Using cloud based technology, such as Optical/QR code generated by customer's hardware device or mobile gadget or sellers or partners of seller's sales outlet. The customer's gadget or hardware device may operate online or offline. Offline examples include a barcode reader or card reader that can read such a gadget to process payment.

Digital online delivery technology that is encrypted software applications where payment are made through internet.

SMS based payment, where the account is managed using SMS commands (to confirm payment); the payment sometimes can be madewith out internet access, and the customer may inform a representative of service provider, which is the seller, or a partner of the seller, about the phone number and payment confirmation code.

Such transaction and transaction record may be used with a delivery technology, operating in one of the following type of networks:

designed exclusively for the network of the seller or the manufacturer, or the network of the seller's partner in banking and payment transactions, such a network supporting a market place, providing a number of products or services including subscription of x-ray imaging device, related product and services, subscription of cloud computing services for image processing, viewing, and storage services, PAC services, medical record storage services, diagnostic services and teleradiology services. Alternatively, such a network may be dedicated exclusively for the imaging services or pay per scan or per procedure services and/or subscription services of imaging equipment and/or purchasing of imaging equipment.

Half closed type, where the customer can use the digital wallet apps when visiting a hospital or clinic or imaging center if there is an agreement between the hospital / clinic/imaging center and the digital banking or digital wallet service provider who is the partner of the seller and/or manufacturer of the x-ray imaging system and related product and services.

Integrated type where the purchasing network or healthcare vendor payment program of the hospital/clinic/imaging center or imaging service providers are integrated with the digital bank network of the seller and/or manufacturers of the equipment and related product services, and/or subscription services and/or pay per procedure or scan services. The portal interfaces with customer and/or seller's existing accounting systems, aligning supply chain processes and automating payment processes. Payment hub may be used to creates automated payment strategy through optimization of supplier payment types (card, ACH, check, specialty), while providing an opportunity for monthly revenue share. The payment hub may start with a single consolidated payment file, may facilitates payment and corresponding details including email remittance, and a reconciliation report is generated. However, now, since the supplier or the seller of the imaging product or imaging service provider may be the bank or have integrated with the financial network of a bank which handles the payment transaction and related financial processing requests, the payment transaction cost involved may be reduced dramatically. The amount of transaction and payment handling fee therefore may be reduced significantly. The seller may charge little or approximately zero for the transaction from the purchasing party, therefore there is not a need for record keeping of merchant bank's transaction fee from customer side.

Digital bank discloed herein allows the customer to have an MAN to receive payments or make direct payment to the seller or the digital bank, which could be the bank as well.

Digital wallet discloed may be an encrypted app runs on online, or mobile device and/or kiosk. Digital wallet may allow a customer to store and deposit prepaid “cash” in a variety of currencies such as US Dollars, Euros, crypto currency, such as Bitcoin,

Ethereum and other crypto currencies in the digital wallet, and one may even be able to pay with them in certain places.

Bitcoin is being stored in the blockchain network. The digital wallet may contain private and public keys and makes it possible to work with them. Example of digital wallets serves as cryptocurrency wallets such as desktop wallets, hardware wallets which use a hardware data storage device, online digital wallets, mobile digital wallets.

The digital wallet or digital currency or the digital bank account user may have two or more following components:

software component ensures security and strong data encryption, information component, containing a database including business customer data (name, bank account details, payment options, address), profile of the customer including contact information, type of imaging equipment and/or device and product and services subscription services, information component, containing a database including a personal data user data (name, card details, payment options and so on),

Information component may be connected to a network connected to containing a database with a medical record database for the patient including prescriptions and/or other relevant information regarding imaging services and/or medical record.

Various level of imaging products and/or associated medical related product and services, may be available to purchase directly from the digital wallet through or digital bank account.

The customer can select a digital payment system, via software interface on a desk top or smart phone or an online kiosk. The digital payment system may be pre-paid and password-protected account for storing the currency for any future online transaction. The user or customer can connect payment cards to the customer's account.

One benefit of the seller provide digital bank services is to lower the transaction cost, improve business efficiency and transaction speed for the customers to purchase product and services, such as x-ray imaging device, and related images and/or subscriptions for imaging services.

Along with the subscription or pay for procedure method via transaction handling through digital bank and/or digital wallet, a online questionnaire can be included prior to imaging procedure or after the imaging procedure given to the patient, either in the image acquisition software or image viewer that which allows the patient to assign access rules for their medical data, for example, the opt in to store their x-ray imaging data, or a portion of the imaging data and in some cases, medical record or part of the medical record in a server or blockchain managed and/or maintained and sometimes owned by the provider of the imaging subscription service or pay per procedure or per image service. The block chain technology and as well as the seller or service provider allows patients to assign access rules for their medical data, for example, permitting specific researchers to access parts their data for a fixed period of time.

In some instances, tag information, instead of the medical data itself may be stored in data blocks. And the actual medical data including x-ray measurements and images may be stored in an off-chain storage, in a relational database, managed by either the hospital or the seller depends on the access rule set by the patient.

The on-chain data can store metadata about this off-chain data, together with pointers to where the actual data resides, and hash codes that may be used to verify the integrity of the off-chain data. The technology can also be used for identify and access control, in other words as a mechanism to control access privileges to this data stored off-chain.

AI may be used for customer service. Transaction details can be recorded as digital evidence onto blockchain.

Big data can be used to do precision underwriting.

Open banking may be used in certain part of the digital banking and digit wallet transaction process.

A customer may download the digital wallet application, or access on line web portal, purchase digital currency through or transfer from a financial network, stored in the wallet the digital currency and then make payment and perform other digital wallet featured financial transactions as a stand-alone app or through the web portal.

A method for reducing transaction cost and improving transaction speed for payment of subscription x-ray imaging services, x-ray imaging system, related products and servicescan include the payment occurring within a financial network, said network including a payment processing process based on a financial processing request automated to be send out at a predefined time period during a time interval, for example , first day of a month.

The payment processing software looks up a database created to look up the customer information, track the level of subscription services the customer has signed up, look up the total number of images or procedures during the subscription period a periodic payment is to be made for, compare the subscription level to the total number images or imaging procedures taken by one or more x-ray imaging systems installed at one or more imaging sites of the customer, match the subscription level and the payment level, process the payment and send an email report of the payment, total number of images or procedures taken, generate revenue sharing information if there is any based on reimbursement codes. If there is any difference between the subscription level and/or payment level and actual total number of images taken in the payment period.

The total number of images taken may be tallied by the image acquisition system, stored in a database in a local microprocessor, send via network to the payment processing software by the imaging product and service provider via an automated email, or by an administrator or by a x-ray technician on a periodic basis. The local microprocessor may be at the x-ray detector controlling unit location or at the work station for image processing, which may also be connected to the display hardware and other control units such as membrane controller for controlling the x-ray system via push buttons, or a touch screen controlling display or a computer with a monitor with desk top software app. A user may be able to access the computer or the microprocessor by a desktop app, which contains password authentication method to access the computer. The computer may contain the image acquisition or viewing software which also contains the database storing information on total number of images taken. The user may also be able to access the database by using a hardware authentication method, a physical key.

The payment processing app function and related software may compare the tally of number of images or procedures in the database, and subscription level, and generate automated email or phone text messages to inform the customer of the result of comparison and amount paid. The software can generate a bill for additional charges for the additional images not covered by the subscription.

The customer can pay for the difference via the same payment network or login to the system to pick a higher level subscription service for the next payment period.

If the customer does neither, the payment processing app may apply the additional images not paid though subscription to the tally for the next period in the database. The same process will go on for a predefined number of times.

A customer service representative may be sent a report of warning that if such process goes on without be remedied. The customer may determine an appropriate response to said warning in accordance with predefined policies and procedures.

Described above is a fast and cost effective digital payment method for purchasing x-ray imaging system, subscription of x-ray imaging service and pay per procedure and per image by integrating x-ray imaging market place with a secure and compliant digital bank method and system

Terms Used

Beam particle stopper“Beam Particle Blocker Plate”, “Beam Absorber Plate”, “Beam Particle Stopper Plate” refers to the hardware piece where a number of distributed x-ray attenuating pieces, including of, for example, spheres or other shapes, placed on top of or embedded in an x-ray transmissive plate including for example, beryllium or polymer.

Component

“Component” or “material” or “substance” is referred to an element which can be measured by x ray and differentiated from the background. Examples of component is a component of an intervention device, such as fluid conduit, contrast agents, metal, or bone or tissue, or a part of heart or blood vessel.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this disclosure may include, additional to its essential features described herein, one or more features as described herein from each other embodiment of the disclosure disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication. For example, actions such as “illuminating a subject” include “instructing illumination of a subject.”

All of the methods and tasks described herein may be performed and fully automated by a computer system. The computer system may, in some cases, include multiple distinct computers or computing apparatuses (e.g., physical servers, workstations, storage arrays, cloud computing resources, etc.) that communicate and interoperate over a network to perform the described functions. Each such computing apparatus typically includes a processor (or multiple processors) that executes program instructions or modules stored in a memory or other non-transitory computer-readable storage medium or apparatus (e.g., solid state storage apparatuses, disk drives, etc.). The various functions disclosed herein may be embodied in such program instructions, and/or may be implemented in application-specific circuitry (e.g., ASICs or FPGAs) of the computer system. Where the computer system includes multiple computing apparatuses, these apparatuses may, but need not, be co-located. The results of the disclosed methods and tasks may be persistently stored by transforming physical storage apparatuses, such as solid state memory chips and/or magnetic disks, into a different state. In some embodiments, the computer system may be a cloud-based computing system whose processing resources are shared by multiple distinct business entities or other users.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. An improved computed tomography imaging system, comprising:
at least one x-ray source configured to produce a plurality of divergent beams;
a plurality of detectors configured to receive x-ray beams emitted from a plurality of emitting positions and attenuated by at least a portion of a subject to be imaged, wherein the plurality of emitting positions comprise a first positions relative to a volume of interest (“VOI”) in the subject, the beams emitted from the first emitting position being projected onto at least one x-y plane two axis out of a 6D space or any possible projection geometry combined.

2. The system of claim 1, wherein the plurality of emitting positions comprises a second position, beams emitted from the second position being projected onto the at least one plane or another 2D or 3D dimension, at least one voxel in the VOI being in on a projection path traveled by the beams emitted from the first position, wherein a distance between the first and second emitting positions is approximately equal to a resolution desired in a z-axis.

3. The system of claim 1, wherein approximately the voxels in the VOI are located in the projection path.

4. The system of claim 1, wherein the plurality of emitting positions comprise a third position.

5. The system of claim 4, wherein beams emitted from the third position_are configured to follow a trajectory outside of a 6D space required for tomography and to increase a size of r of a field of view of the subject so that a different VOI can be selected.

6. The system of claim 4, wherein beams emitted from the third position_are configured to follow a trajectory outside of a 6D space needed to reconstruct a complete image, and are configured to provide a different angle in a sparse projection situation or for projection from the x-ray source that has at least one different energy level, and/or different focal spot sizes or different field of view, different frame rate or modulated differently by energy means or electronics means or optical means.

7. The system of claim 1, wherein a path traveled by beams emitted from one or more of the plurality of emitting positions is traveled by beams emitted from a different x-ray source, wherein the different x-ray source has a plurality of different energy levels and focal spot sizes, or a plurality of different frame rates, or comprises a different type of source.

8. The system of claim 1, further comprising a controller that includes:
an acquisition system configured to acquire from a plurality of detector x-ray attenuation data; and
an image reconstructor configured to receive a first data set derived from the x-ray attenuation data and perform algorithms to reconstruct a first reconstructed image.

9. The system of claim 8, wherein the first data set includes primary x-ray data with Scatter to Primary of less than 1% or less than 5%.

10. The system of claim 8, wherein the first data set includes primary x-ray data derived from scatter removed data using a scatter removal method that includes time of flight x-ray measurements where primary x-ray is separated from scatter in the time domain.

11. The system of claim 8, wherein the first data set includes primary x-ray data with less 1% or less than 5% SPR derived from using movable a beam particle stopper array and/or a adjustable or movable beam selector and using interpolation of low resolution scatter to give rise to high resolution scatter images.

12. The system of claim 8, wherein the first data set includes primary x-ray data with less 1% or less than 5% SPR derived from a front detector, a beam particle stopper array and a rear detector using interpolation of low resolution scatter to give rise to high resolution scatter images at the front detector or the rear detector.

13. The system of claim 12, wherein the front detector is a movable front detector.

14. The system of claim 8, wherein the first data set includes data derived from projection imaging data_by the plurality of detectors corresponding to the plurality of emitting positions and VOI.

15. The system of claim 8, wherein the first data set includes data derived from projection imaging data from a dual energy material decomposed substance dataset, which is derived from inverse energy function system look-up measured by the selected detector regions at one or both of the first or second positions.

16. The system of claim 8, wherein the first data set includes a Hounsfield value derived from a dual energy material decomposed substance dataset, which is derived from inverse energy function system look-up measured by the selected detector regions at two or more of the plurality of emitting positions.

17. The system of claim 8, wherein the controller is further configured to execute a material decomposition to provide attcntiation attenuation data for at least one substance.

18. The system of claim 8, wherein the controller is further configured to generate a material decomposition based on 2D dual energy or multiple energy measurements of the VOI from x-ray emitted at one or both of the first or second emitting positions.

19. The system of claim 18, wherein the material decomposition method includes using measurements from a time of flight sensor or a camera or a previous x ray exposure for measurement of a VOI thickness.

20. The system of claim 19, wherein the time of flight sensor and or controller is configured to determine an exposure level of x-ray measurements generating at least some of the first set data and/or a second data set.

21. The system of claim 8, wherein the reconstruction method comprise algorithms or derivatives of the algorithms for tomographic reconstruction for CT, tomosynthesis, MRI, electron tomography, optical tomography, thermo imaging, PET, or SPECT.

22. The system of claim 8, wherein the first reconstructed image is reconstructed using a reconstruction method_an original or derivatives of fouricr Fourier transform, ray tracing method, model or contour based iterative reconstruction, material decomposed method based, spectral CT, ART, Monte Carlo Simulation based, non space based reconstruction method, iterative algorithms and their derivatives, filtered methods, method at least one modified_dual variable, or a splitting-based subproblem method.

23. The system of claim 8, wherein the controller is configured to generate the first reconstructed image by:
backprojecting the x-ray attenuation data for each beam to form an array of data points therealong,
weighting each backprojected data point by a weighting factor w(r), where r is the distance between the backprojected data point and a source location of the divergent beams to form weighted backprojected data points,
Fourier transforming and processing an array of data which includes the weighted backprojected data points to form an acquired k-space data set;
aligning the acquired k-space data set with a reference k-space, and
reconstructing an image from the referenced k-space data by performing an inverse Fourier transformation thereon.

24. The system of claim 1, wherein said system is integrated with an autonomous driving device.

25. The system of claim 1, wherein said system is configured to fit through a standard door, the plurality of detectors configure to be placed between a patient and a patient bed, surgical table, or imaging table.

26. The system of claim 1, wherein said system is a spectral tomographic mammography system.

27. The system of claim 1, wherein said system further comprises a hand switch, a display, handheld display, foot pedal, display membrane, joy stick, voice recognition rccognization, speaker, acoustic noise hardware and electronics and software, the controller configured to control some of the hardware and sync software for integrating hardware and software processes.

28. The system of claim 1, wherein said system or its components is a portion of a kit.

29. The system of claim 1, wherein said system comprises methods, software, and hardware to decompose metal materials.

30. The system of claim 1, wherein said system include methods and hardware to material compose intervention devices or one or more portion of such device, implant or contrast agents, microcalcification, contrast labeled blood vessels, plaster cast mixed with contrast agents.

31. The system of claim 30, wherein the contrast agents comprise barium or bismuth.

32. The system of claim 30, wherein the contrast agents are administered at concentration levels and/or molarity levels at 2× to 1000,000× less than that of contrast agents used in conventional CT and general x-ray and MM and PET and/or magnetic particle based imaging.

33. The system of claim 30, wherein the contrast agents comprise calcium chloride, calcium gluconate, iodinated reagents, barium, bismuth, strontium, gadolinium, the contrast agents used in PET and/or MRI.

34. The system of claim 30, wherein the intervention device comprises an artificial heart valve, an RF ablation catheter, a cage, a stent, an implant, or surgical tool.

35. The system of claim 1, wherein said system comprise a C arm, U arm, CT system, or has a foot print similar to that of a general x-ray or tomosynthesis system.

36. The system of claim 1, comprising a first system matrix configured to integrate one or more of the x-ray sources and one or more of the plurality of detectors.

37. The system of claim 1 wherein the first position is in area of less than 2 cm ², or less than 5 cm² or less 1 degree , or less than 2 degrees, or less than 3 degrees, or less than 4 degrees, or less than 5 degrees, or less than 6 degrees, or less than 7 degrees, less than 8 degrees or less than 10 degrees, from a center axis connecting original positions of the plurality of detectors and the at least one x-ray source.

38. The system of claim 1, wherein the distance is less than 1 um, or less than 5 um, or less than 10 um, or less than 50 um or less than 100 um, or less than 160 um, or less than 250 um, or less than 500 um, or less than 1 mm, or less than 2 mm. or less than 5 mm, or less than 1 cm or less than 2 cm, or less than 5 cm.

39. The system of claim 8, wherein the controller is configured to generate the first reconstructed image in_less than 10s, or less than 5s or less than 2.5s, or less than ls.

40. The system of claim 1, wherein the system is configured to reduce radiation exposure by 2×, or by 5× or 10×, or 100×, or 1000× or 10,000× or 100, 000, or 1000,000× compared to conventional CT.

41. The system of claim 8, comprising a second system matrix configured to integrate additional imaging modalities including optical, thermo, PET, SPECT, ultrasound_and/or

42. Said The system of claim 41, wherein the reference detector is placed in the x-ray beam path.

43. The system of claim 42, wherein the first data set and the second dataset are used to train AI algorithms for reconstruction and determining said VOI for data acquisition.

44. The system of claim 41, wherein the controller is configured to use the second data set, either after or during the reconstruction of the first image.

45. The system of claim 44, wherein if the second data set is used after the reconstruction of the first image, the first reconstruction provides model or contour or data which is used in a second reconstruction incorporating the second data set.

46. The system of claim 45, wherein if the second data set is used during the reconstruction of the first image, the controller is configured to use the same or different system matrix and modified variable and split subproblem method.

47. The system of claim 43, wherein the second data set comprises data derived from a different detector of the plurality of detectors taking at the same time as time of acquisition for one or more x-ray images generating the first data set.

48. The system of claim 47, wherein the different detector includes at least one detector placed upstream or downstream or at the same spatial location of the first detector from which the first data set was acquired.

49. The system of claim 43, wherein the second data set comprises data from x-ray measurements taken at a time different from the time of acquisition for one or more x-ray images generating the first data set.

50. The system of claim 43, wherein the second data set comprises data taken at a different time by the first detector from which the first data set was acquired.

51. The system of claim 43. wherein the first and/or second data sets are configured to be denoised during, before, or after image reconstruction on a case by case basis.

52. The system of claim 51, wherein the denoising process is selectively done on a substance or the VOI.

53. The system of claim 43, wherein the first and/or second data sets are normalized.

54. The system of claim 8, wherein the acquisition system is configured to selectively acquire data during image reconstruction.

55. The system of claim 54, wherein the selective data acquisition is based on a reconstruction result of first data set, or a selected VOI, wherein the reconstruction is prioritized for the selected VOI.

56. A payment and transaction electronic system for an x-ray imaging, and related product and services, the system comprising:
a software platform for purchaser and users including:
an electronic database containing metered information for x-ray images or related procedures taken at at least one location;
data encryption mechanisms configured to encrypt data, and currency transfer, and communication;
digital currency or exchange media agreed by a buyer and a_seller, the digital currency comprising cryptocurrency;
a server configured to collect the meter information from at least one facility;
data collection mechanisms configured to the meter information onsite of the imaging location or via cloud,
wherein an amount charged in digital currency periodically is based on a subscription and/or pay per image model out of purchaser's account.

57. The system of claim 56 further comprising a software platform for seller including:
a front end presentation comprising a mobile app, the desk top app or the web portal which allows username and password input and sign in and registration and related information, and a developer portal;
a back end comprising a product layer where sits a core banking system, client data and other back-offices related processes;
a middle-ware comprising an intermediary layer orchestrating information between the front end and the back end and API layer.

58. The system of claim 57, wherein the software platform for sellers is configured to enable connections to external and/or third party applications include accounting software, customer and/or user accounts, loans, payments, market place, digital onboarding, payment networks, cards and card management.

59. The system of claim 56, wherein the seller is a digital bank or has partnered with a digital bank to enable wiring, ACH transfer, and/or digital bank transfer via email, phone based on a user and/or customer's account number.

60. The system of claim 56, wherein the x-ray images include images produced by scatter removed x-ray imaging system, spectral x-ray imaging system, CT, spectral CT, spectral CT with one or more radiology services, AI related software, pac, image storage, and/or image processing.

61. A method of reconstructing a 3D image of a VOI of an object using an x-ray system, the x-ray system comprising at least one x-ray source and at least one detector, the method comprising:
translating and/or rotating the at least one x-ray source and/or one or more of the plurality of detectors;
correlating projection measurements with various positions of the at least one x-ray source and at least one detector using a system matrix,
wherein for at least a one 2D projection image, the at least one x-ray source is configured to emit beams illuminating at least a majority of or approximately an entirety of the VOI so that for each voxel within the VOI, there is new projection path reaching one of the plurality of detectors, and
wherein there are m×n projection paths approximately, with each movement between the emitting positions, the movement being approximately a resolution desired in along an axial axis connecting an x-ray tube of the at least one x-ray source and the at least one detector passing through the VOI, so that the new projection path is different from a remainder of the m×n projection path by at least approximately one voxel, or each voxel within VOI has a projection path differ than other path by at 1-eat—least approximately one voxel.

62. The method of claim 61, wherein a total number of projections is approximated by a thickness of the VOI.

63. The method of claim 61, wherein a total number of projections is approximated by a geometry measurement of a sensor, a camera or an x-ray image exposure value, or a time of flight sensor, the approximation comprising:
determining at least a distance from a top of the subject containing the VOI to the at least one source, and
subtracting the distance from the top of the subject to the at least one x-ray source from a source-to-detector distance (“SID”); and
deriving the thickness of VOI.

64. The method of claim 63, wherein an x-ray exposure level is approximated by an automatic exposure method and apparatus, the time of flight detector, and/or a reference detector.

65. The method of claim 63, wherein the total rotational x-ray emitting position angle from the center axis by less than 5 degrees or, less than 4 degrees, or less than 3 degrees or less than 3 degrees or less than 2 degrees or less than 1 degree.

66. The method of claim 61, wherein the method is configured to be combined with another movement trajectory, tube rotating angle, or detector angle to either expand a field of view of an x-ray emitting beam volume or to combine projected images, and/or to expand flexibility of movement due to pre-existing application requirement.

67. The method of claim 66, wherein the requirement comprises angular and translational movement of the subject or movement of the VOI.

68. The method of claim 61, wherein each movement is configured to introduce a new projection path for each voxel of the VOI.

69. The method of claim 61, wherein the x-ray is emitted from the same location or a different emitting location.

70. The method of claim 61, wherein the x-ray system comprises more than one source, each source is capable of tomography.

71. The method of claim 70, wherein the more than one source are configured to be used and represented in the same system matrix, each source having a plurality of emitting positions or are configured to move to generate projecting images of the VOI, wherein the projected images are combined with other images to reconstruct the 3D image of the VOI.

72. The method of claim 71, wherein each source is configured to project projected images of at least one portion of VOI, and a 3D reconstruction is derived from two or more set of projected images, each set produced by at least each source.

73. The method of claim 72, wherein the same system matrix includes different sources, the measured data being combined to establish a more accurate provisional 3D reconstruction.

74. The method of claim 61, wherein the 3D reconstructed image comprises the VOI, which is determined through earlier 3D reconstruction of different resolution, or energy level or spectral imaging or single energy image or 3D reconstruction at at least one or more different x ray emitting positions.

75. The method of claim 74, wherein the projected image is imaged processed with a scatter removal method involving interpolation in the spatial domain and/or using a movable beam particle stopper array and/or stacked detector method with a beam particle stopper plate or movable beam selector.

76. The method of claim 74, wherein the attenuation value and or density information derived for at least one substance of interest or composite substance of interest is in reconstruction of the 3D image.

77. The method of claim 61, wherein a final 3D reconstruction is used to determine the VOI.

78. The method of claim 61, wherein the x-ray system is mounted upright.

79. The method of claim 78, wherein the x-ray system is mounted in an C arm or U arm.

80. The method of claim 72, wherein the projected images are located at a different VOI on the subject combined 3D reconstructed image resulting in a 3D image with a larger volume.

81. An x-ray imaging apparatus, comprising:
a controller configured to:
obtain projection data representing an intensity of radiation having illuminated a VOI and exited out of VOI of an object detected at a plurality of detectors, or a ratio of the intensity over a radiation intensity entering the VOI derived from radiation detected in a first detector and radiation detected at a reference detector, and
generate the first data sets and at least a second dataset based on the obtained projection data, wherein thcfirst the first data sets comprises data generated by the first detector, and the at least a second data set comprises data generated by the first detectors or a second detector, wherein the projection data is from a different radiation emitting position, energy level, exposure level, and/or different system configurations.

82. The apparatus of claim 81, wherein the controller is configured to generate more data set comprising data generated by the same first detectors, or the same second detectors or additional detectors.

83. The apparatus of claim 81, comprising a single radiation source, which have different emitting position, different focal spot sizes, and/or different fields of view due to a field of view restricting device or collimators.

84. The apparatus of claim 81, comprising first and second radiation source, the second radiation source being a different radiation source than the first radiation source but travelling in the same area of emitting positions of the first radiation source, wherein the radiation emitted by the second source is of a different focal size, and/or different energy level and/or speed of pulse generation.

85. The apparatus of claim 84, comprising first and second detectors, the first detectors having a different detector configuration than the second detectors.

86. The apparatus of claim 85, comprising a 3rd or more detectors, wherein the respective detector configurations of the first detectors and second detectors, and the third or more detectors are determined by a detector type.

87. The apparatus of claim 84, wherein a projection geometry and/or pixel elements are arranged within the respective first detectors and second detectors, and the controller is configured to reconstruct a combined image using the plurality of datasets.

88. The apparatus of claim 87, wherein each dataset of the plurality of datasets corresponds to a respective system-matrix equation representing respective projection geometries corresponding to the plurality of datasets.

89. The apparatus of claim 87, wherein each dataset of the plurality of datasets corresponds to approximately the same or similar system matrix equation or a different system matrix equation representing respective projection geometries corresponding to the plurality of datasets.

90. The apparatus of claim 87, wherein the image is reconstructed using the same system matrix for a plurality of datasets comprising data with scatter to primary ration less than 1% or less than 5%, by one or more of:
a low scatter VOI,
using time of flight primary measurement by removal of scatter in the time domain,
using scatter removal method comprising primary x-ray image derived from subtraction of high resolution scatter derived from interpolation of low resolution scatter image,
using ART or its derivative algorithms, and/or iterative methods.

91. The apparatus of claim 87, wherein the image is reconstructed using different system matrices for a plurality of datasets, at least one modified-dual variable and using a splitting based subproblem method.

92. The apparatus of claim 87, wherein the image is reconstructed using the same system matrix for a plurality of datasets, at least one modified-dual variable and using a splitting based subproblem method.

93. The apparatus of claim 81, wherein subproblem are performed on the datasets separated by time of data generation.

94. The apparatus of claim 81, further including at least one more addition dataset .

95. The apparatus of claim 88, wherein the system matrix incorporates the use of optical sensors and camera, guided by AI to use surface image and AI to select the ROI.

96. The apparatus of claim 81, comprising AI software used to reduce noise.

97. The apparatus of claim 81, wherein the images are scatter removed to less than 1% SPR or less than 5% SPR, avoiding a need to consider scatter in simulation.

98. The apparatus of claim 81, wherein a distance moved by an x-ray source from a first position to a second position is less than 5 cm, or /or less than 2 cm squared or less than 5 cm squared or less than 1 cm squared and less than 4 cm squared or less than 3 cm squared and/or less than 3 cm squared from the first positions.

99. The apparatus of claim 98, wherein x-ray emitted at the second position is configured to travel in the same volume or 6D spatial position as x-ray from the first position.

100. The apparatus of claim 98, wherein the x-ray source is field emitting to emit x-ray at the same spatial position as the x-ray filament tube or other type of x-ray source, or the various type of source or its modulated version with same or different parameters including focal spot size, energy level, frame rate, and/or geometry, or manipulated by different x-ray optics or steered by different mechanisms may be used, wherein a same spatial matrix, a modified dual or multiple variable method, or a split subproblem method is used.

101. The apparatus of claim 88, wherein an optical method is used in conjunction with the present x-ray systems, using the system matrix.

102. The apparatus of claim 88, wherein vectors are used in the system matrix.

103. The apparatus of claim 81, wherein the controller is configured to use dual energy or multiple energy x-ray to determine an approximate area and distribution in the projected image on a pixel by pixel basis.

104. The apparatus of claim 81, wherein the data sets are used to reconstruct a 3D image.

105. The apparatus of claim 81, wherein the controller is configured to segment out the material volume and space distribution, and/or perform material decomposition.

106. The apparatus of claim 81, wherein the controller is configured to determine the ROI before and/or after reconstruction for further spectral imaging.

107. The apparatus of claim 81, wherein the controller is configured to combine movement of source and/or detector with that of a tomography system.

108. The apparatus of claim 81, wherein the controller is configured to perform Contrast Agent decomposition.

109. The apparatus of claim 81, wherein the controller is configured to perform dual energy or multiple energy decomposition to distinguish an X-ray absorbing material.

110. The apparatus of claim 109, wherein the x-ray absorbing material comprises:
a metal or plaster cast mixed with barium,
a catheter and/or implant with one or more materials and/or having lumen and sheath made of different x ray absorbing properties or atomic z, or made with distributed x-ray absorbent material at certain spatial locations interlaced with x-ray transparent material, sufficient to determine its spatial distribution compared to the background and other segments in the same catheter or implant, or including well-characterized x-ray absorption properties on a pixel basis, sufficient to differentiate one segment to another segment
A plaster cast,
a blood vessel,
a contrast labeled blood vessel,
microcalcification, and/or
contrast-agent labeled molecules.

111. The apparatus of claim 81, wherein the controller is configured to denoise_using AI software trained to remove noise.

112. The apparatus of claim 81, wherein the controller is configured to use data generated in training of an AI algorithms for reconstruction.

113. The apparatus of claim 81, wherein the apparatus is part of a tomography device.

114. The apparatus of claim 113, wherein the subject is loaded on a table or bed which is x-ray transmissive, the table or bed being placed on top of a detector gantry of the tomography device.

115. The apparatus of claim 113, wherein a patient is configured to lay on a surface of a detector gantry, which is transparent to x-ray.

116. The apparatus of claim 113, wherein the device or a portion thereof is portable by connecting to an autonomous driving device to be transported inside a clinic or to remote location outside the hospital.

117. The apparatus of claim 113, wherein the device is less than dimensions of an opening of a standard door.

118. The apparatus of claim 113, wherein the device is used as a point of care device, and/or used in a patient's room.

119. The apparatus of claim 113, wherein the device comprises a detector module that is movable and can be placed in between the patient's bed and the patient.

120. The apparatus of claim 81, wherein the controller is configured to perform material decomposition using a beam particle stopper reconstruction method.

121. The apparatus of claim 120, wherein the beam particle stopper reconstruction methods comprises filling a data gap from an image taken at the same x-ray emitting position and with a different beam particle stopper array position where primary x-rays are blocked.

122. The apparatus of claim 121, wherein the beam particle stopper reconstruction methods comprises filling the data gap during the reconstruction process, each projection path which is missed from the beam particle stopper being described as having no data input, therefore requiring extra projection data to be generated from the same x-ray emitting position or using sparse data 3D reconstruction algorithms.

123. The apparatus of claim 120, wherein the material decomposition is performed for metal and/or other absorbing material in a catheter or an implant comprising one or more substances overlapping each other, if the controller knows the approximate density and/or thickness of the catheter or the implant.
